(19) United States
(12) Patent Application Publication
(10) Pub. No.: US 2024/0358689 A1
(43) Pub. Date: Oct. 31, 2024

(54) Modulators Of The Beta-3 Adrenergic Receptor Useful For The Treatment Or Prevention Of Renal Cystic Disease and Cardiorenal Syndrome

(71) Applicant: Arena Pharmaceuticals, Inc., New York, NY (US)

(72) Inventor: John William Adams, La Jolla, CA (US)

(73) Assignee: Arena Pharmaceuticals, Inc., New York, NY (US)

(21) Appl. No.: 18/685,281

(22) PCT Filed: Aug. 23, 2022
(86) PCT No.: PCT/IB2022/057885

§ 371 Date: Feb. 21, 2024

Related U.S. Application Data
(60) Provisional application No. 63/237,271, filed on Aug. 26, 2021.

(51) Int. Cl. A61K 31/438; A61K 31/4709; A61K 31/5383

(52) U.S. Cl. CPC A61K 31/438; A61K 31/4709; A61K 31/5383

(57) ABSTRACT

The present invention relates to methods of treating renal cystic disease and/or cardiorenal syndrome by administrating certain compounds that modulate the activity of the beta-3 adrenergic receptor.

Renal cysts occur in one third of people older than 50 years. While most are simple cysts, renal cystic disease has multiple etiologies. Broad categories of the cystic disease include the following:
Congenital—Congenital cystic dysplasia;
Genetic—Autosomal recessive polycystic kidney disease (ARPKD), autosomal dominant polycystic kidney disease (ADPKD), nephronophthisis-medullary cystic kidney disease complex (NMCD);
Acquired—Simple cysts, acquired cystic disease;
Cysts associated with systemic disease—Von Hippel-Lindau syndrome (VHLS), tuberous sclerosis (TS) and
Malignancy—Renal cell carcinoma (RCC).

The most common larger cysts are acquired cysts, simple cysts, and cysts with ARPKD. Smaller cysts are associated with ARPKD, NMCD, and medullary sponge kidney (MSK). In adults, renal angiomyolipomas and RCC also may demonstrate cystic lesions.

Polycystic kidney disease (PKD) describes several conditions in which fluid-filled cysts form in the kidneys. Cysts generally develop in weak segments of the tubules that carry urine from the glomeruli. The cyst's growth displaces healthy kidney tissue. The kidneys expand to accommodate the cyst, which can weigh as much as 20 pounds. There are many forms of PKD, both inherited forms and noninherited.

Autosomal dominant PKD (ADPKD) is the most common, inherited form. Symptoms of ADPKD usually develop between the ages of 30 and 40, but they can begin earlier, even in childhood. About 90 percent of all PKD cases are autosomal dominant PKD. ADPKD results from mutation in the PKDI gene that encodes polycystin-1 (85% of the cases) or PKD2 gene that encodes polycystin-1 (15% of the cases). Autosomal recessive PKD (ARPKD) is a rare, inherited form. Symptoms of autosomal recessive PKD begin in the earliest months of life, even in the womb.

When PKD causes kidneys to fail, which usually happens after many years, the patient requires dialysis or kidney transplantation. About one-half of people with the major type of PKD progress to kidney failure. PKD can cause cysts in the liver and problems in other organs, such as the heart and blood vessels in the brain. These complications distinguish PKD from the usually harmless “simple” cysts that often form in the kidneys in later years of life.

In the United States, about 600,000 people, and world wide about 12.5 million people have PKD, and it is a leading cause of kidney failure. At this time, PKD has no cure. The treatments for PKD include medicine and surgery to reduce pain, antibiotics to resolve infections, dialysis to replace functions of failed kidneys and kidney transplantation.

Cardiorenal syndrome (CRS) encompasses a spectrum of disorders involving both the heart and kidneys in which acute or chronic dysfunction in 1 organ may induce acute or chronic dysfunction in the other organ. It represents the confluence of heart-kidney interactions across several interfaces. These include the hemodynamic cross-talk between the failing heart and the response of the kidneys and vice versa, as well as alterations in neurohormonal markers and inflammatory molecular signatures characteristic of its clinical phenotypes.

Medical management of patients with CRS is often challenging as focus on treatment of one organ may have worsening outcome on the other. It is known that many of the medications used to treat heart failure may worsen kidney function. In addition, many trials on heart failure excluded patients with advanced kidney dysfunction. Therefore, understanding of CRS management is still limited to this date

Therefore, there is a need for developing more efficient treatments of renal cystic disease, including PKD, and cardiorenal syndrome.

### SUMMARY OF THE INVENTION

Provided is a method of treating renal cystic disease and/or cardiorenal syndrome in an individual, comprising administering to the individual in need thereof, a therapeutically effective amount of a compound of formula (Ia)

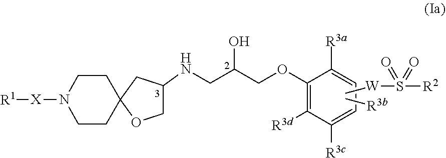

wherein:
X is —SO₂—, —C(═O)—, or —CH₂C(═O)—;
W is absent or C₁-C₃ alkylene;
R¹ is aryl or heteroaryl, wherein each is optionally substituted with one or more substituents selected from: C₁-C₆ alkoxy, C₁-C₆ alkyl, amino, cyano, C₃-C₇ cycloalkyl, C₁-C₆ haloalkyl, halogen, hydroxyl, oxo, and sulfamoyl; and wherein said C₁-C₆ alkyl and C₃-C₇ cycloalkyl are each optionally substituted with one or more substituents selected from: amino, C₁-C₆ alkoxy, C₁-C₆ alkylcarboxamide, —Y—C₃-C₇-cycloalkyl, —Y—C₁-C₆-alkylene-Z, C₁-C₆ alkylamino, C₁-C₆ haloalkylamino, and heterocyclyl;
Y is independently selected from: —O—, —NH—, and —N—(C₁-C₄ alkyl)-;
Z is independently selected from: hydroxyl, C₁-C₆ alkoxy, amino, C₁-C₆ alkylamino, and C₂-C₆ dialkylamino;
R² is selected from: C₂-C₆ alkenyl, C₁-C₆ alkyl, C₃-C₇ cycloalkyl, heterocyclyl, and C₁-C₆ haloalkyl; each optionally substituted with one or more substituents selected from: C₁-C₆ alkoxy,
C₁-C₆ alkylenehydroxyl, amino, aryl, C₃-C₇ cycloalkyl, cyano, C₃-C₇ halocycloalkyl, hydroxyl, and oxo; and
R^{3a}, R^{3b}, R^{3c}, and R^{3d} are each independently H or halogen.

Also provided is a method of treating renal cystic disease and/or cardiorenal syndrome in an individual, comprising administering to the individual in need thereof, a therapeutically effective amount of a compound selected from compounds of Formula (IIa) and pharmaceutically acceptable salts, solvates, hydrates, and N-oxides thereof:

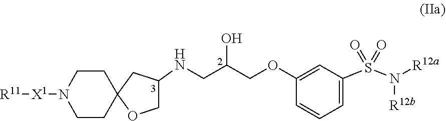

wherein:
X¹ is —SO₂— or absent;
R¹¹ is selected from: aryl, C₁-C₆-alkylene-aryl, C₁-C₆-alkylene-heteroaryl, C₃-C₇ cycloalkyl, heteroaryl, and heterocyclyl; each optionally substituted with one or more substituents selected from: C₁-C₆ alkoxy, C₁-C₆ alkoxycarbonyl, C₁-C₇ alkyl, C₁-C₆ alkylamino, C₁-C₆ alkylcarboxamide, C₁-C₆ alkylsulfonamido, C₁-C₆ alkylsulfonyl, amino, aryloxy, arylsulfonyl, carboxamide, carbamimidoyl, carboxy, cyano, C₃-C₇ cycloalkyl, C₂-C₈ dialkylamino, C₂-C₈ dialkylsulfamoyl, C₁-C₆ haloalkoxy, C₁-C₆ haloalkyl, halogen, heterocyclyl, hydroxycarbamimidoyl, hydroxyl, oxo, and sulfamoyl; and wherein said C₁-C₆ alkoxy, C₁-C₇ alkyl, C₁-C₆ alkylamino, aryloxy, C₃-C₇ cycloalkyl, and C₂-C₈ dialkylamino are each optionally substituted with one or more substituents selected from: amino, C₁-C₆ alkoxy, C₁-C₆ alkylamino, C₁-C₆ alkylcarboxamide, carboxy, —Y¹—C₁-C₆-alkylene-Z¹ optionally substituted with oxo, C₃-C₇ cycloalkyl, cyano, C₂-C₆ dialkylamino, C₁-C₆ haloalkyl, C₁-C₆ haloalkylamino, heterocyclyl, hydroxyl, oxo, and phenyl;
Y¹ is selected from: —O— and —NH—;
Z¹ is selected from: C₁-C₆ alkoxy, amino, C₁-C₆ alkylamino, cyano, C₂-C₆ dialkylamino, hydroxyl, and phenyl;
R^{12a} is H or selected from: C₁-C₆ alkoxy, C₁-C₆ alkyl, C₃-C₇ cycloalkyl, and heterocyclyl; each optionally substituted with one or more substituents selected from: C₁-C₆ alkoxy, C₁-C₆ alkylamino, C₁-C₆ alkyl, C₁-C₆ alkylenehydroxyl, amino, C₃-C₇ cycloalkyl, cyano, C₂-C₈ dialkylamino, heterocyclyl optionally substituted with one oxo group, halogen, hydroxyl, and oxo; and
R^{12b} is H or C₁-C₆ alkyl.

These and other aspects of the invention disclosed herein will be set forth in greater detail as the patent disclosure proceeds.

### DETAILED DESCRIPTION OF THE INVENTION

### Definitions

For clarity and consistency, the following definitions will be used throughout this patent document.

As used herein, “administering” refers to providing a compound of the invention or other therapy, remedy or treatment to the individual in need of treatment in a form that can be introduced into that individual's body in a therapeutically useful form and therapeutically useful amount, including, but not limited to: oral dosage forms, such as tablets, capsules, syrups, suspensions, and the like; injectable dosage forms, such as IV, IM, or IP, and the like; transdermal dosage forms, including creams, jellies, powders, or patches; buccal dosage forms; inhalation powders, sprays, suspensions, and the like; and rectal suppositories. A health care practitioner can directly provide a compound to an individual in the form of a sample, or can indirectly provide a compound to an individual by providing an oral or written prescription for the compound. Also, for example, an individual can obtain a compound by themselves without the involvement of a health care practitioner. When the compound is administered to the individual, the body is transformed by the compound in some way. When a compound of the invention is provided in combination with one or more other agents, “administration” is understood to include the compound and other agents are administered at the same time or at different times. When the agents of a combination are administered at the same time, they can be administered together in a single composition or they can be administered separately.

The term “antagonist” as used herein “refers to a moiety that can competitively bind to the β₃-adrenergic receptor as an agonist (for example, the endogenous ligand) but does not activate or substantially reduces the intracellular response compared to an agonist, and can thereby inhibit the intracellular responses by an agonist or partial agonist. An “antagonist” does not diminish the baseline intracellular response, or does so to a negligible extent, in the absence of an agonist or partial agonist.

The term “composition” refers to a compound or crystalline form thereof, including but not limited to, salts, solvates, and hydrates of a compound of the present invention, in combination with at least one additional component, such as, a composition obtained/prepared during synthesis, preformulation, in-process testing (i.e., TLC, HPLC, NMR samples), and the like.

The term “hydrate” as used herein means a compound of the invention or a salt thereof that further includes a stoichiometric or non-stoichiometric amount of water bound by non-covalent intermolecular forces.

The term “in need of treatment” and the term “in need thereof” when referring to treatment are used interchangeably to mean a judgment made by a caregiver (e.g. physician, nurse, nurse practitioner, etc. in the case of humans; veterinarian in the case of animals, including non-human mammals) that an individual or animal requires or will benefit from treatment. This judgment is made based on a variety of factors that are in the realm of a caregiver's expertise, but that includes the knowledge that the individual or animal is ill, or will become ill, as the result of a disease, condition or disorder that is treatable by the compounds of the invention. Accordingly, the compounds of the invention can be used in a protective or preventive manner; or compounds of the invention can be used to alleviate, inhibit, or ameliorate the disease, condition, or disorder.

The term “individual” refers to any animal, including mammals, such as, mice, rats, other rodents, rabbits, dogs, cats, swine, cattle, sheep, horses, primates, and humans. In some embodiment “individual” refers to humans.

The term “pharmaceutical composition” refers to a specific composition comprising at least one active ingredient; including but not limited to, salts, solvates, and hydrates of compounds of the present invention, whereby the composition is amenable to investigation for a specified, efficacious outcome in a mammal (for example, without limitation, a human). Those of ordinary skill in the art will understand and appreciate the techniques appropriate for determining whether an active ingredient has a desired efficacious outcome based upon the needs of the artisan.

The phrase “pharmaceutically acceptable salts, solvates, and hydrates” when referring to a compound/compounds as described herein embraces pharmaceutically acceptable solvates and/or hydrates of the compound/compounds, pharmaceutically acceptable salts of the compound/compounds, as well as pharmaceutically acceptable solvates and/or hydrates of pharmaceutically acceptable salts of the compound/compounds. It is also understood that when the phrase “pharmaceutically acceptable solvates and hydrates” or the phrase “pharmaceutically acceptable solvate or hydrate” is used when referring to a compound/compounds as described herein that are salts, it embraces pharmaceutically acceptable solvates and/or hydrates of such salts. It is also understood by a person of ordinary skill in the art that hydrates are a subgenus of solvates.

The term “prescribing” refers to order, authorize, or recommend the use of a drug or other therapy, remedy, or treatment. In some embodiments, a health care provider orally advises, recommends, or authorizes the use of a compound, dosage regimen, or other treatment to an individual. The health care provider may or may not provide a written prescription for the compound, dosage regimen, or treatment. Further, the health care provider may or may not provide the compound or treatment to the individual. For example, the health care provider can advise the individual where to obtain the compound without providing the compound. In some embodiments, a health care provider can provide a written prescription for the compound, dosage regimen, or treatment to the individual. A prescription can be written on paper or recorded on electronic media. In addition, a prescription can be called in (oral) or faxed in (written) to a pharmacy or a dispensary. In some embodiments, a sample of the compound or treatment is given to the individual. As used herein, giving a sample of a compound constitutes an implicit prescription for the compound. Different health care systems around the world use different methods for prescribing and administering compounds or treatments, and these methods are encompassed by the disclosure herein.

A health care provider can include, for example, a physician, nurse, nurse practitioner, or other health care professional who can prescribe or administer compounds (drugs) for the disorders disclosed herein. In addition, a health care provider can include anyone who can recommend, prescribe, administer, or prevent an individual from receiving a compound or drug, including, for example, an insurance provider.

The terms “prevent,” “preventing,” and “prevention” refer to the elimination or reduction of the occurrence or onset of one or more symptoms associated with a particular disorder. For example, the terms “prevent,” “preventing,” and “prevention” can refer to the administration of therapy on a prophylactic or preventative basis to an individual who may ultimately manifest at least one symptom of a disorder but who has not yet done so. Such individuals can be identified on the basis of risk factors that are known to correlate with the subsequent occurrence of the disease, such as the presence of a biomarker. Alternatively, prevention therapy can be administered as a prophylactic measure without prior identification of a risk factor. Delaying the onset of the at least one episode and/or symptom of a disorder can also be considered prevention or prophylaxis.

The terms “renal cystic disease” and “cystic diseases of the kidney” refer to certain diseases and conditions characterized by cysts that develop on or around the kidneys. They can be classified as either (1) hereditary or acquired or (2) systemic or renal confined diseases that have the common feature of multiple renal cysts. The diseases and conditions included are polycystic kidney disease, e.g., autosomal-dominant polycystic kidney disease and autosomal-recessive polycystic kidney disease, unilateral renal cystic disease (localized cystic disease), renal simple cysts, multicystic dysplastic kidney, pluricystic kidney of the multiple malformation syndromes, juvenile nephronophthisis and medullary cystic disease, medullary sponge kidney, primary glomerulocystic kidney disease, and glomerulocystic kidney associated with several systemic disorders mainly of genetic or chromosomal etiology, cystic kidney in tuberous sclerosis, and in von Hippel-Lindau syndrome, cystic nephroma, cystic variant of congenital mesoblastic nephroma, mixed epithelial stromal tumor of the kidney, renal lymphangioma, pyelocalyceal cyst, peripylic cyst and perinephric pseudocyst, acquired renal cystic disease of long-term dialysis, and cystic renal cell carcinoma and sarcoma.

The term “cardiorenal syndrome” or “CRS” refers to a physiologic relationship between the heart and kidney that manifests as a tight coordination between renal and cardiac functions in subjects suffering from heart failure. The term has been used to define different clinical conditions in which heart and kidney dysfunction overlap. Type 1 CRS (acute cardio-renal syndrome) is characterized by acute worsening of cardiac function leading to acute kidney injury (AKI) in the setting of active cardiac disease such as acute decompensated heart failure, while type 2 CRS occurs in a setting of chronic heart disease. Type 3 CRS is closely link to AKI, while type 4 represent cardiovascular involvement in chronic kidney disease (CKD) patients.

Type 5 CRS represent cardiac and renal involvement in several diseases such as sepsis, hepato-renal syndrome and immune-mediated diseases. A recent discussion of cardiorenal syndrome may be found in Rangaswami et al, “Cardiorenal Syndrome: Classification, Pathophysiology, Diagnosis, and Treatment Strategies: A Scientific Statement From the American Heart Association,” Circulation 139:e840-e878 (2019) and in Di Lullo et al. “Pathophysiology of the cardio-renal syndromes types 1-5: An uptodate”, Indian Heart J. 2017; 69(2):255-265, each of which is incorporated herein by reference in its entirety.

The term “solvate” as used herein means a compound of the invention or a salt thereof that further includes a stoichiometric or non-stoichiometric amount of a solvent bound by non-covalent intermolecular forces. Preferred solvents are volatile, non-toxic, and/or acceptable for administration to humans in trace amounts.

The terms “treat,” “treating,” and “treatment” refer to the administration of therapy to an individual who already manifests, or who has previously manifested, at least one symptom of a disease, disorder, condition, dependence, or behavior. For example, “treating” can include any of the following with respect to a disease, disorder, condition, dependence, or behavior: alleviating, abating, ameliorating, improving, inhibiting (e.g., arresting the development), relieving, or causing regression. “Treating” can also include treating the symptoms, preventing additional symptoms, preventing the underlying physiological causes of the symptoms, or stopping the symptoms (either prophylactically and/or therapeutically) of a disease, disorder, condition, dependence, or behavior. For example, the term “treating” in reference to a disorder means a reduction in severity of one or more symptoms associated with a particular disorder. Therefore, treating a disorder does not necessarily mean a reduction in severity of all symptoms associated with a disorder and does not necessarily mean a complete reduction in the severity of one or more symptoms associated with a disorder.

The term “therapeutically effective amount” refers to the amount of active compound or pharmaceutical agent that elicits the biological or medicinal response in a tissue, system, animal, or human that is being sought by an individual, researcher, veterinarian, medical doctor, or other clinician or caregiver, which can include one or more of the following:
(1) preventing the disorder, for example, preventing a disease, condition, or disorder in an individual who may be predisposed to the disease, condition, or disorder but does not yet experience or display the relevant pathology or symptomatology;
(2) inhibiting the disorder, for example, inhibiting a disease, condition, or disorder in an individual who is experiencing or displaying the relevant pathology or symptomatology (i.e., arresting further development of the pathology and/or symptomatology); and
(3) ameliorating the disorder, for example, ameliorating a disease, condition, or disorder in an individual who is experiencing or displaying the relevant pathology or symptomatology (i.e., reversing the pathology and/or symptomatology).

### Chemical Group, Moiety or Radical

The term “C₂-C₆ alkenyl” denotes a radical containing 2 to 6 carbons wherein at least one carbon-carbon double bond is present. Some embodiments contain 2 to 5 carbons. Some embodiments contain 2 to 4 carbons. Some embodiments contain 2 to 3 carbons. Some embodiments contain 2 carbons (i.e., —CH═CH₂). Both E and Z isomers are embraced by the term “alkenyl.” Furthermore, the term “alkenyl” includes di- and tri-alkenyls.

The terms “C₁-C₆ alkylene” and “C₁-C₄ alkylene” refers to a straight or branched, saturated aliphatic, divalent radical having the defined number of carbons, 1 to 6 carbon atoms or 1 to 4 carbon atoms respectively. Some embodiments contain 1 to 2 carbons. Some embodiments contain 1 to 5 carbons. Some embodiments contain 1 to 4 carbons. Some embodiments contain 1 to 3 carbons. Some embodiments contain 1 or 2 carbons. Some embodiments contain 1 carbon atom (i.e., —CH₂—). Examples include, but are not limited to, methylene, ethylene, n-propylene, isopropylene, n-butylene, s-butylene, isobutylene, t-butylene, pentylene, isopentylene, t-pentylene, neopentylene, 1-methylbutylene [i.e., —CH(CH₃)CH₂CH₂CH₃], 2-methylbutylene [i.e., —CH₂CH(CH₃)CH₂CH₃], n-hexylene, and the like.

The term “amino” refers to the group —NH₂.

The term “aryl” refers to a ring system containing 6 to 12 carbon atoms that may contain a single ring, two fused rings, or two rings bonded by a single bond (i.e., biphenyl) and wherein at least one ring is aromatic. Examples include phenyl, biphenyl, indanyl, tetrahydronaphthalenyl, naphthalenyl, and the like. Examples of biphenyl include: [1,1′-biphenyl]-2-yl (i.e., biphenyl-2-yl), [1,1′-biphenyl]-3-yl (i.e., biphenyl-3-yl), or [1,1′-biphenyl]-4-yl (i.e., biphenyl-4-yl) with the following structures respectively:

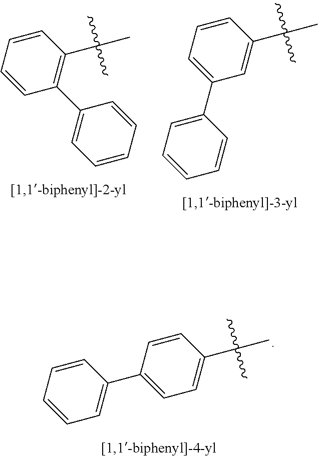

When a substituent is present on the aryl ring, the substituent can be bonded at any available ring carbon.

The term “C₁-C₆ alkoxy” refers to a radical comprising a C₁-C₆ alkyl group attached directly to an oxygen atom, wherein C₁-C₆ alkyl has the same definition as found herein. Some embodiments contain 1 to 5 carbons (i.e., C₁-C₅ alkoxy). Some embodiments contain 1 to 4 carbons (i.e., C₁-C₄ alkoxy). Some embodiments contain 1 to 3 carbons (i.e., C₁-C₃ alkoxy). Some embodiments contain 1 or 2 carbons. Examples include, but are not limited to methoxy, ethoxy, n-propoxy, isopropoxy, n-butoxy, t-butoxy, isobutoxy, s-butoxy, and the like.

The term “C₁-C₆ alkyl” refers to a straight or branched carbon radical containing 1 to 6 carbons. Some embodiments are 1 to 5 carbons (i.e., C₁-C₅ alkyl), some embodiments are 1 to 4 carbons (i.e., C₁-C₄ alkyl), some embodiments are 1 to 3 carbons (i.e., C₁-C₃ alkyl), and some embodiments are 1 or 2 carbons. Examples of an alkyl include, but are not limited to, methyl, ethyl, n-propyl, isopropyl, n-butyl, sec-butyl, isobutyl, tert-butyl, pentyl, isopentyl, tert-pentyl, neo-pentyl, 1-methylbutyl [i.e., —CH(CH₃)CH₂CH₂CH₃], 2-methylbutyl [i.e., —CH₂CH(CH₃)CH₂CH₃], n-hexyl and the like.

The term “C₁-C₆ alkylamino” refers to mean a radical comprising one C₁-C₆ alkyl group attached to an NH group, wherein C₁-C₆ alkyl has the same meaning as described herein. Some embodiments are “C₁-C₂ alkylamino.” Some examples include methylamino, ethylamino, n-propylamino, isopropylamino, n-butylamino, s-butylamino, isobutylamino, t-butylamino, and the like.

The term “C₁-C₆ alkylcarboxamide” refers to mean a single C₁-C₆ alkyl group attached to either the carbon or the nitrogen of an amide group, wherein C₁-C₆ alkyl has the same definition as found herein. The C₁-C₆ alkylcarboxamido group may be represented by the following:

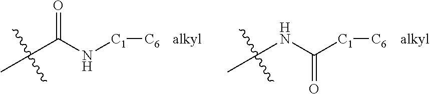

Examples include, N-methylcarboxamide, N-ethylcarboxamide, N-n-propylcarboxamide, N-isopropylcarboxamide, N-n-butylcarboxamide, N-s-butylcarboxamide, N-isobutylcarboxamide, N-t-butylcarboxamide, and the like.

The term “cyano” refers to the group —CN.

The term “C₃-C₇ cycloalkyl” refers to a saturated ring radical containing 3 to 7 carbons. Some embodiments contain 3 to 6 carbons. Some embodiments contain 3 to 5 carbons. Some embodiments contain 5 to 7 carbons. Some embodiments contain 3 to 4 carbons. Examples include cyclopropyl, cyclobutyl, cyclopentyl, cyclohexyl, cycloheptyl, and the like.

The term “C₂-C₆ dialkylamino” refers to a radical comprising an amino group substituted with two alkyl groups, the alkyl groups can be the same or different provided that two alkyl groups do not exceed a total of 6 carbon atoms between the two alkyl groups. Some embodiments are C₂-C₄ dialkylamino. Some examples include dimethylamino, methylethylamino, diethylamino, methylpropylamino, methylbutylamino, methylpentylamino, methylisopropylamino, ethylpropylamino, ethylisopropylamino, dipropylamino, propylisopropylamino, and the like.

The term “C₁-C₆ haloalkylamino” refers to a radical comprising one C₁-C₆ haloalkyl group attached to an NH group, wherein C₁-C₆ haloalkyl has the same meaning as described herein. Some embodiments are “C₁-C₂ haloalkylamino.” Some examples include 2-fluoroethylamino, 2,2,2-trifluoroethylamino, (1,1,1-trifluoropropan-2-yl)amino, 3,3,3-trifluoropropylamino, 2,2,2-trifluoropropylamino, and the like.

The term “C₁-C₆ haloalkyl” refers to a radical comprising a C₁-C₆ alkyl group substituted with one or more halogens, wherein C₁-C₆ alkyl has the same definition as found herein. The C₁-C₆ haloalkyl may be fully substituted in which case it can be represented by the formula CₙL₂ₙ₊₁, wherein L is a halogen and “n” is 1, 2, 3, 4, 5, or 6. When more than one halogen is present then they may be the same or different and selected from: fluorine, chlorine, bromine, and iodine. In some embodiments, haloalkyl contains 1 to 5 carbons (i.e., C₁-C₅ haloalkyl). In some embodiments, haloalkyl contains 1 to 4 carbons (i.e., C₁-C₄ haloalkyl). In some embodiments, haloalkyl contains 1 to 3 carbons (i.e., C₁-C₃ haloalkyl). In some embodiments, haloalkyl contains 1 or 2 carbons. Examples of haloalkyl groups include fluoromethyl, difluoromethyl, trifluoromethyl, chlorodifluoromethyl, 1-fluoroethyl, 2,2,2-trifluoroethyl, pentafluoroethyl, 4,4,4-trifluorobutyl, and the like.

The term “C₃-C₇ halocycloalkyl” refers to a radical comprising a C₃-C₇ cycloalkyl group substituted with one or more halogens, wherein C₃-C₇ cycloalkyl has the same definition as found herein. Examples of halocycloalkyl groups include 2,2-difluorocyclopropyl, 1-fluorocyclopropyl, 4,4-difluorocyclohexyl, and the like.

The term “halogen” refers to fluoro, chloro, bromo, or iodo group. In some embodiments, halogen is fluoro, chloro, or bromo. In some embodiments, halogen is fluoro or chloro. In some embodiments, halogen is fluoro.

The term “heteroaryl” refers to a ring system containing 5 to 14 ring atoms, that may contain a single ring, two fused rings, two rings bonded by a single bond, or three fused rings, and wherein at least one ring atom is a heteroatom, such as, O, S, and N, wherein N is optionally substituted with H, C₁-C₄ acyl, or C₁-C₄ alkyl and at least one ring is aromatic. When a heteroaryl group is substituted with an oxo group, the oxo group can be on any available ring atom, for example, a ring carbon to form a carbonyl group, a ring nitrogen to form an N-oxide, and a ring sulfur to form either a sulfoxide (i.e., —S(═O)—) or a sulfone (i.e., —S(═O)—). Some embodiments contain 5 to 6 ring atoms for example furanyl, thienyl, pyrrolyl, imidazolyl, oxazolyl, thiazolyl, isoxazolyl, pyrazolyl, isothiazolyl, oxadiazolyl, triazolyl, tetrazolyl, thiadiazolyl, pyridinyl, pyrazinyl, pyrimidinyl, pyridazinyl, triazinyl, and the like. Some embodiments contain 8 to 14 ring atoms for example quinolizinyl, quinolinyl, isoquinolinyl, cinnolinyl, phthalazinyl, quinazolinyl, quinoxalinyl, triazinyl, indolyl, isoindolyl, indazolyl, indolizinyl, purinyl, naphthyridinyl, pteridinyl, carbazolyl, acridinyl. phenazinyl, phenothiazinyl, phenoxazinyl, benzoxazolyl, benzothiazolyl, 1H-benzimidazolyl, imidazopyridinyl, benzothienyl, benzofuranyl, isobenzofuran, 2,3-dihydrobenzofuranyl, 4H-benzo[1,3]dioxinyl, 3,4-dihydro-1H-isoquinolinyl, 1,4,6,7-tetrahydro-imidazo[4,5-c]pyridinyl, 7,8-dihydro-5H-[1,6]naphthyridinyl, 5,6-dihydro-8H-[1,2,4]triazolo[4,3-a]pyrazinyl, benzo[1,3]dioxolyl, pyrazolo[1,5-a]pyrimidinyl, 1,2,3,4-tetrahydroquinolinyl, and the like. When the “heteroaryl” is a ring system containing two rings bonded by a single bond it is understood that the two rings can be bonded at any available ring carbon or available nitrogen atom. Some embodiments include 3-(1H-pyrazol-4-yl)phenyl, 3-(pyridin-4-yl)phenyl, 3-(pyridin-2-yl)phenyl, 5-phenylthiophen-2-yl, 3-(pyridin-3-yl)phenyl, 3-(pyrimidin-5-yl)phenyl, 5-(phenyl)pyridin-3-yl, 5-(1H-pyrazol-4-yl)pyridin-3-yl, 4-(pyridin-3-yl)phenyl, 4-(pyridin-4-yl)phenyl, 4-(pyridin-2-yl)phenyl, (corresponding to the following chemical structures) and the like.

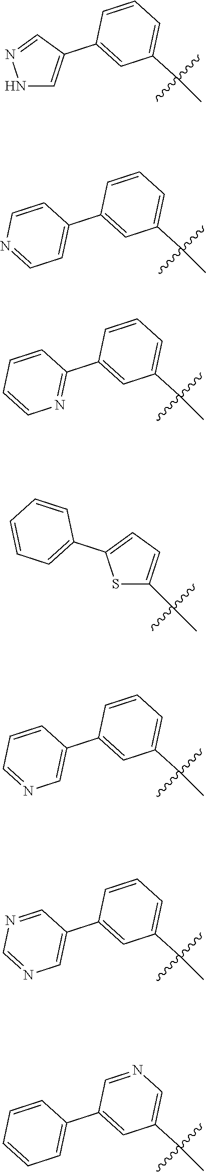

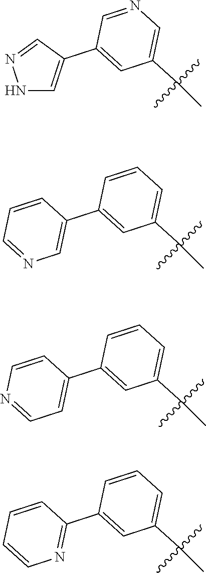 [SMILES: *c1cncc(-c2cn[nH]c2)c1 ; *c1cccc(-c2cccnc2)c1 ; *c1cccc(-c2ccncc2)c1 ; *c1cccc(-c2ccccn2)c1]

In some embodiments, “heteroaryl” is selected from the group: (1H-pyrazolyl)phenyl, (1H-pyrazolyl)pyridinyl, (pyridinyl)phenyl, (pyrimidinyl)phenyl, 1,2,3,4-tetrahydropyrido[3,2-b]pyrazinyl, 1,2-dihydroquinolinyl, 1,4-dihydroquinolinyl, 1H-benzo[d]imidazolyl, 1H-indazolyl, 1H-indolyl, 1H-pyrazolo[4,3-b]pyridinyl, 1H-pyrazolyl, 1H-pyrrolo[2,3-b]pyridinyl, 1H-pyrrolo[3,2-b]pyridinyl, 2,3-dihydro-[1,4]dioxino[2,3-b]pyridinyl, 2,3-dihydro-1H-imidazo[4,5-b]pyridinyl, 2,3-dihydro-1H-pyrido[2,3-b][1,4]oxazinyl, 2,3-dihydro-1H-pyrrolo[2,3-b]pyridinyl, 2,3-dihydrobenzofuranyl, 3,4-dihydro-2H-benzo[b][1,4]oxazinyl, 3,4-dihydro-2H-pyrano[2,3-b]pyridinyl, 3,4-dihydro-2H-pyrido[3,2-b][1,4]oxazinyl, 3H-imidazo[4,5-b]pyridinyl, (phenyl)pyridinyl, 5,6,7,8-tetrahydroquinolinyl, 6,7-dihydro-5H-pyrrolo[3,4-b]pyridin-3-yl, benzo[c][1,2,5]oxadiazolyl, benzofuranyl, chromanyl, isoquinolinyl, isoxazolyl, phenylthiophenyl, pyridinyl, pyrrolo[1,2-a]pyrimidinyl, quinolinyl, and thiazolyl. In some embodiments, “heteroaryl” is selected from the group: 1,2,3,4-tetrahydropyrido[3,2-b]pyrazin-7-yl, 1,2-dihydroquinolin-6-yl, 1,4-dihydroquinolin-3-yl, 1H-benzo[d]imidazol-5-yl, 1H-indazol-5-yl, 1H-indol-2-yl, 1H-indol-3-yl, 1H-indol-5-yl, 1H-indol-6-yl, 1H-pyrazol-4-yl, 1H-pyrazolo[4,3-b]pyridin-6-yl, 1H-pyrrolo[2,3-b]pyridin-3-yl, 1H-pyrrolo[3,2-b]pyridin-6-yl, 2,3-dihydro-[1,4]dioxino[2,3-b]pyridin-7-yl, 2,3-dihydro-1H-imidazo[4,5-b]pyridin-6-yl, 2,3-dihydro-1H-pyrido[2,3-b][1,4]oxazin-6-yl, 2,3-dihydro-1H-pyrido[2,3-b][1,4]oxazin-7-yl, 1,4-dihydroquinolin-3-yl, 2,3-dihydro-1H-pyrrolo[2,3-b]pyridin-5-yl, 2,3-dihydrobenzofuran-5-yl, 3-(1H-pyrazol-4-yl)phenyl, 3-(pyridin-2-yl)phenyl, 3-(pyridin-3-yl)phenyl, 3-(pyridin-4-yl)phenyl, 3-(pyrimidin-5-yl)phenyl, 3,4-dihydro-2H-benzo[b][1,4]oxazin-6-yl, 3,4-dihydro-2H-benzo[b][1,4]oxazin-7-yl, 3,4-dihydro-2H-pyrano[2,3-b]pyridin-6-yl, 3,4-dihydro-2H-pyrido[3,2-b][1,4]oxazin-7-yl, 3H-imidazo[4,5-b]pyridin-5-yl, 3H-imidazo[4,5-b]pyridin-6-yl, 4-(pyridin-2-yl)phenyl, 4-(pyridin-3-yl)phenyl, 4-(pyridin-4-yl)phenyl, 5-(1H-pyrazol-4-yl)pyridin-3-yl, 5-(phenyl)pyridin-3-yl, 5,6,7,8-tetrahydroquinolin-3-yl, 5-phenylthiophen-2-yl, 6,7-dihydro-5H-pyrrolo[3,4-b]pyridin-3-yl, benzo[c][1,2,5]oxadiazol-4-yl, benzofuran-2-yl, benzofuran-5-yl, chroman-6-yl, chroman-7-yl, isoquinolin-5-yl, isoxazol-4-yl, pyridin-2-yl, pyridin-3-yl, pyrrolo[1,2-a]pyrimidin-3-yl, quinolin-3-yl, quinolin-6-yl, quinolin-7-yl, and thiazol-4-yl. When referring to a heteroaryl group, it is understood that the terms thiophenyl, thiophen-2-yl, thiophen-3-yl, and the like, refer to the following heteroaryl groups respectively:

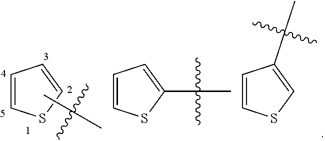

The term “heterocyclyl” refers to a non-aromatic ring radical containing 3 to 8 ring atoms, wherein one, two, or three of the ring atoms are heteroatoms selected from, for example: O, S, and N, wherein N is optionally substituted with H, C₁-C₄ acyl, or C₁-C₄ alkyl. In some embodiments, “heterocyclyl” refers to a non-aromatic ring radical containing 3 to 8 ring atoms, wherein one or two of the ring atoms are heteroatoms selected from, for example: O, S, and NH. Examples of a heterocyclyl group include aziridinyl, azetidinyl, piperidinyl, morpholinyl, oxetanyl, imidazolidinyl, piperazinyl, pyrrolidinyl, thiomorpholinyl, [1,4]oxazepanyl, azepanyl, tetrahydrofuranyl, tetrahydropyranyl, tetrahydrothiopyranyl, and the like.

The term “hydroxyl” refers to the group —OH.

The term “C₁-C₆ alkylenehydroxyl” refers to a radical consisting of a hydroxyl group bonded to a C₁-C₆ alkylene radical, wherein hydroxyl and C₁-C₆ alkylene have the same definitions as described herein. Examples include hydroxymethyl, 2-hydroxyethyl, 1-hydroxyethyl, and the like.

The term “oxo” refers to the diradical ═O.

The term “sulfamoyl” refers to the group —S(═O)₂NH₂.

### Compounds of the Invention

One aspect of the present invention encompasses, inter alia, certain 1-oxa-8-azaspiro[4.5]decan-3-yl-aminopropanyl-ether derivatives selected from compounds of Formula (Ia) and pharmaceutically acceptable salts, solvates, and hydrates thereof:

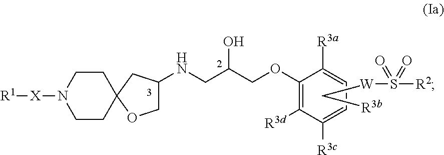

wherein: R¹ (as well as Y and Z that are both related to R¹), X, W, R², R^{3a}, R^{3b}, R^{3c}, and R^{3d} all have the same definitions as described herein, supra and infra

In some embodiments, compounds of the present can have the following defined stereochemistry as shown in Formula (Ia¹):

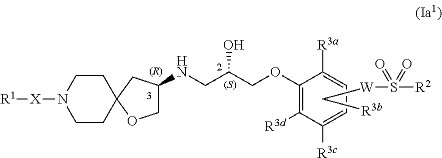

wherein: R¹, X, W, R², R^{3a}, R^{3b}, R^{3c}, and R^{3d}, have the same definitions as described herein, supra and infra, and wherein the carbon designated as C(3) of the oxa-azaspiro[4.5]decanyl group bonded to the nitrogen has the (R) stereochemistry and the carbon designated as C(2) of the propyl group bonded to the hydroxyl group has the (S) stereochemistry.

In some embodiments, compounds of the present can have the following defined stereochemistry as shown in Formula (Ia²):

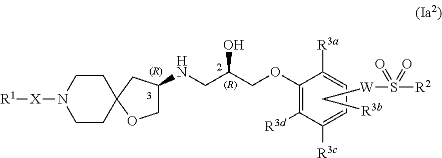

wherein: R¹, X, W, R², R^{3a}, R^{3b}, R^{3c}, and R^{3d}, have the same definitions as described herein, supra and infra, and wherein the carbon designated as C(3) of the oxa-azaspiro[4.5]decanyl group bonded to the nitrogen has the (R) stereochemistry and the carbon designated as C(2) of the propyl group bonded to the hydroxyl group has the (R) stereochemistry.

In some embodiments, compounds of the present can have the following defined stereochemistry as shown in Formula (Ia³):

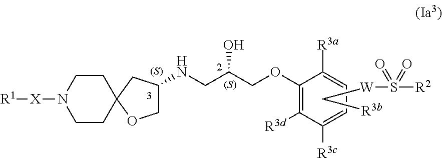

wherein: R¹, X, W, R², R^{3a}, R^{3b}, R^{3c}, and R^{3d}, have the same definitions as described herein, supra and infra, and wherein the carbon designated as C(3) of the oxa-azaspiro[4.5]decanyl group bonded to the nitrogen has the (S) stereochemistry and the carbon designated as C(2) of the propyl group bonded to the hydroxyl group has the (S) stereochemistry.

In some embodiments, compounds of the present can have the following defined stereochemistry as shown in Formula (Ia⁴):

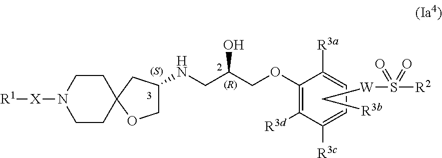

wherein: R¹, X, W, R², R^{3a}, R^{3b}, R^{3c}, and R^{3d}, have the same definitions as described herein, supra and infra, and wherein the carbon designated as C(3) of the oxa-azaspiro[4.5]decanyl group bonded to the nitrogen has the (S) stereochemistry and the carbon designated as C(2) of the propyl group bonded to the hydroxyl group has the (R) stereochemistry.

It is understood that any formulae described herein for which the stereochemistry is not specifically shown can be written to specifically show the stereochemistry as (R) and (S), (R) and (R), (S) and (S), or (S) and (R) for C(3) and C(2) respectively in a similar manner as Formulae (Ia¹), (Ia²), (Ia³), and (Ia⁴) shows the respective stereochemistry for Formula (Ia), supra. Similarly, any formulae described herein for which the stereochemistry is not specifically shown can alternatively be defined using the language as described for Formulae (Ia¹), (Ia²), (Ia³), and (Ia⁴), supra, to define the stereochemistry as (R) and (S), (R) and (R), (S) and (S), and (S) and (R) respectively.

Accordingly, in some embodiments, the stereochemistry for the C(3) carbon of the oxa-azaspiro[4.5]decanyl group bonded to the nitrogen is (R) and the stereochemistry for the C(2) carbon of the propyl group bonded to the hydroxyl group is (S). In some embodiments, the stereochemistry for the C(3) carbon of the oxa-azaspiro[4.5]decanyl group bonded to the nitrogen is (R) and the stereochemistry for the C(2) carbon of the propyl group bonded to the hydroxyl group is (R). In some embodiments, the stereochemistry for the C(3) carbon of the oxa-azaspiro[4.5]decanyl group bonded to the nitrogen is (S) and the stereochemistry for the C(2) carbon of the propyl group bonded to the hydroxyl group is (S). In some embodiments, the stereochemistry for the C(3) carbon of the oxa-azaspiro[4.5]decanyl group bonded to the nitrogen is (S) and the stereochemistry for the C(2) carbon of the propyl group bonded to the hydroxyl group is (R).

It is understood that compounds of Formula (Ia) and formulae used throughout this disclosure represent all individual enantiomers and mixtures thereof, unless specifically stated or shown otherwise.

### The X Group

In some embodiments, X is —SO₂—, —C(═O)—, or —CH₂C(═O)—.

In some embodiments, X is —SO₂—.

In some embodiments, X is —C(═O)—.

In some embodiments, X is —CH₂C(═O)—.

### Ring W

In some embodiments, W is absent or C₁-C₃ alkylene.

In some embodiments, W is absent.

In some embodiments, W is C₁-C₃ alkylene.

In some embodiments, W is —CH₂—.

### The Y and Z Groups

The Y and Z groups are related to certain substituents on R¹ where the substituent is selected from C₁-C₆ alkyl and C₃-C₇ cycloalkyl group and each can be further optionally substituted with one or more substituents selected from a group consisting of the following that contain either the Y group or both the Y and Z groups: —Y—C₃-C₇-cycloalkyl and —Y—C₁-C₆-alkylene-Z.

In some embodiments, Y is independently selected from: —O—, —NH—, and —N—(C₁-C₄ alkyl)-.

In some embodiments, Z is independently selected from: hydroxyl, C₁-C₆ alkoxy, amino, C₁-C₆ alkylamino, and C₂-C₆ dialkylamino.

It is understood that when more than one —Y—C₃-C₇-cycloalkyl and/or —Y—C₁-C₆-alkylene-Z group is present then Y and Z may be the same or different.

In some embodiments, Y is —O—.

In some embodiments, Y is —NH—.

In some embodiments, Y is —N—(C₁-C₄ alkyl)-.

In some embodiments, Z is independently selected from: C₁-C₆ alkoxy, amino, and C₁-C₆ alkylamino.

In some embodiments, Z is hydroxyl.

In some embodiments, Z is C₁-C₆ alkoxy.

In some embodiments, Z is amino.

In some embodiments, Z is C₁-C₆ alkylamino.

In some embodiments, Z is C₂-C₆ dialkylamino.

### The R¹ Group (Aryl and Heteroaryl)

In some embodiments, R¹ is aryl or heteroaryl, wherein each is optionally substituted with one or more substituents selected from: C₁-C₆ alkoxy, C₁-C₆ alkyl, amino, cyano, C₃-C₇ cycloalkyl, C₁-C₆ haloalkyl, halogen, hydroxyl, oxo, and sulfamoyl; and wherein said C₁-C₆ alkyl and C₃-C₇ cycloalkyl are each optionally substituted with one or more substituents selected from: amino, C₁-C₆ alkoxy,

C₁-C₆ alkylcarboxamide, —Y—C₃-C₇-cycloalkyl, —Y—C₁-C₆-alkylene-Z, C₁-C₆ alkylamino, C₁-C₆ haloalkylamino, and heterocyclyl.

In some embodiments, R¹ is aryl or heteroaryl, wherein each is optionally substituted with one or more substituents selected from: C₁-C₆ alkoxy, C₁-C₆ alkyl, amino, cyano, C₃-C₇ cycloalkyl, C₁-C₆ haloalkyl, halogen, hydroxyl, oxo, and sulfamoyl; and wherein said C₁-C₆ alkyl and C₃-C₇ cycloalkyl are each optionally substituted with one or more substituents selected from: amino, C₁-C₆ alkoxy,

C₁-C₆ alkylcarboxamide, —NH—C₃-C₇-cycloalkyl, —NH—C₁-C₆-alkylene-NH₂, —NH—C₁-C₆-alkylene-O—C₁-C₆-alkyl, —NH—C₁-C₆-alkylene-NH—C₁-C₆-alkyl, C₁-C₆ alkylamino, C₁-C₆ haloalkylamino, and heterocyclyl.

In some embodiments, R¹ is selected from: (1H-pyrazolyl)phenyl, (1H-pyrazolyl)pyridinyl, (pyridinyl)phenyl, (pyrimidinyl)phenyl, 1,2,3,4-tetrahydropyrido[3,2-b]pyrazinyl, 1,2-dihydroquinolinyl, 1,4-dihydroquinolinyl, 1H-benzo[d]imidazolyl, 1H-indazolyl, 1H-indolyl, 1H-pyrazolo[4,3-b]pyridinyl, 1H-pyrazolyl, 1H-pyrrolo[2,3-b]pyridinyl, 1H-pyrrolo[3,2-b]pyridinyl, 2,3-dihydro-[1,4]dioxino[2,3-b]pyridinyl, 2,3-dihydro-1H-imidazo[4,5-b]pyridinyl, 2,3-dihydro-1H-pyrido[2,3-b][1,4]oxazinyl, 2,3-dihydro-1H-pyrrolo[2,3-b]pyridinyl, 2,3-dihydrobenzofuranyl, 3,4-dihydro-2H-benzo[b][1,4]oxazinyl, 3,4-dihydro-2H-pyrano[2,3-b]pyridinyl, 3,4-dihydro-2H-pyrido[3,2-b][1,4]oxazinyl, 3H-imidazo[4,5-b]pyridinyl, (phenyl)pyridinyl, 5,6,7,8-tetrahydronaphthalenyl, 5,6,7,8-tetrahydroquinolinyl, 6,7-dihydro-5H-pyrrolo[3,4-b]pyridin-3-yl, benzo[c][1,2,5]oxadiazolyl, benzofuranyl, biphenyl, chromanyl, isoquinolinyl, isoxazolyl, naphthalenyl, phenyl, phenylthiophenyl, pyridinyl, pyrrolo[1,2-a]pyrimidinyl, quinolinyl, and thiazolyl; wherein each is optionally substituted with one or more substituents selected from: 2-methylpropan-2-yl, amino, bromo, chloro, cyclopropyl, ethoxy, ethyl, fluoro, hydroxy, isopropoxy, methoxy, methyl, oxo, propan-2-yl, propan-1-yl, sulfamoyl, and trifluoromethyl; and wherein said 2-methylpropan-2-yl, cyclopropyl, ethyl, methyl, and propan-2-yl are each optionally substituted with one or more substituents selected from: 2,2,2-trifluoroethylamino, 2-aminoethylamino, 2-methoxyethylamino, 3-aminopropylamino, acetamido, amino, azetidin-1-yl, butylamino, cyclobutylamino, ethylamino, isobutylamino, isopropylamino, methoxy, methylamino, morpholino, propylamino, tert-butylamino, and tert-pentylamino.

In some embodiments, R¹ is selected from: (1H-pyrazolyl)phenyl, (1H-pyrazolyl)pyridinyl, (pyridinyl)phenyl, (pyrimidinyl)phenyl, 1,2,3,4-tetrahydropyrido[3,2-b]pyrazinyl, 1,2-dihydroquinolinyl, 1,4-dihydroquinolinyl, 1H-benzo[d]imidazolyl, 1H-indazolyl, 1H-indolyl, 1H-pyrazolo[4,3-b]pyridinyl, 1H-pyrazolyl, 1H-pyrrolo[2,3-b]pyridinyl, 1H-pyrrolo[3,2-b]pyridinyl, 2,3-dihydro-[1,4]dioxino[2,3-b]pyridinyl, 2,3-dihydro-1H-imidazo[4,5-b]pyridinyl, 2,3-dihydro-1H-pyrido[2,3-b][1,4]oxazinyl, 2,3-dihydro-1H-pyrrolo[2,3-b]pyridinyl, 2,3-dihydrobenzofuranyl, 3,4-dihydro-2H-benzo[b][1,4]oxazinyl, 3,4-dihydro-2H-pyrano[2,3-b]pyridinyl, 3,4-dihydro-2H-pyrido[3,2-b][1,4]oxazinyl, 3H-imidazo[4,5-b]pyridinyl, (phenyl)pyridinyl, 5,6,7,8-tetrahydronaphthalenyl, 5,6,7,8-tetrahydroquinolinyl, 6,7-dihydro-5H-pyrrolo[3,4-b]pyridin-3-yl, benzo[c][1,2,5]oxadiazolyl, benzofuranyl, biphenyl, chromanyl, isoquinolinyl, isoxazolyl, naphthalenyl, phenyl, phenylthiophenyl, pyridinyl, pyrrolo[1,2-a]pyrimidinyl, quinolinyl, and thiazolyl; wherein each is optionally substituted with one or more substituents selected from: (2,2,2-trifluoroethylamino)methyl, (2-aminoethylamino)methyl, (2-methoxyethylamino)methyl, (3-aminopropylamino)methyl, (butylamino)methyl, (cyclobutylamino)methyl, (ethylamino)methyl, (isobutylamino)methyl, (isopropylamino)methyl, (methylamino)methyl, (propylamino)methyl, (tert-butylamino)methyl, (tert-pentylamino)methyl, 1-amino-2-methylpropan-2-yl, 1-aminocyclopropyl, 2-acetamidoethyl, 2-aminoethyl, 2-aminopropan-2-yl, 2-methoxyethyl, amino, aminomethyl, azetidin-1-ylmethyl, bromo, chloro, cyano, cyclopropyl, ethoxy, ethyl, fluoro, hydroxy, isopropoxy, methoxy, methyl, morpholinomethyl, oxo, propan-1-yl, sulfamoyl, and trifluoromethyl.

In some embodiments, R¹ is selected from: 1,2,3,4-tetrahydropyrido[3,2-b]pyrazin-7-yl, 1,2-dihydroquinolin-6-yl, 1,4-dihydroquinolin-3-yl, 1H-benzo[d]imidazol-5-yl, 1H-indazol-5-yl, 1H-indol-2-yl, 1H-indol-3-yl, 1H-indol-5-yl, 1H-indol-6-yl, 1H-pyrazol-4-yl, 1H-pyrazolo[4,3-b]pyridin-6-yl, 1H-pyrrolo[2,3-b]pyridin-3-yl, 1H-pyrrolo[3,2-b]pyridin-6-yl, 2,3-dihydro-[1,4]dioxino[2,3-b]pyridin-7-yl, 2,3-dihydro-1H-imidazo[4,5-b]pyridin-6-yl, 2,3-dihydro-1H-pyrido[2,3-b][1,4]oxazin-6-yl, 2,3-dihydro-1H-pyrido[2,3-b][1,4]oxazin-7-yl, 2,3-dihydro-1H-pyrrolo[2,3-b]pyridin-5-yl, 2,3-dihydrobenzofuran-5-yl, 3-(1H-pyrazol-4-yl)phenyl, 3-(pyridin-2-yl)phenyl, 3-(pyridin-3-yl)phenyl, 3-(pyridin-4-yl)phenyl, 3-(pyrimidin-5-yl)phenyl, 3,4-dihydro-2H-benzo[b][1,4]oxazin-6-yl, 3,4-dihydro-2H-benzo[b][1,4]oxazin-7-yl, 3,4-dihydro-2H-pyrano[2,3-b]pyridin-6-yl, 3,4-dihydro-2H-pyrido[3,2-b][1,4]oxazin-7-yl, 3H-imidazo[4,5-b]pyridin-5-yl, 3H-imidazo[4,5-b]pyridin-6-yl, 4-(pyridin-2-yl)phenyl, 4-(pyridin-3-yl)phenyl, 4-(pyridin-4-yl)phenyl, 5-(1H-pyrazol-4-yl)pyridin-3-yl, 5-(phenyl)pyridin-3-yl, 5,6,7,8-tetrahydronaphthalen-2-yl, 5,6,7,8-tetrahydroquinolin-3-yl, 5-phenylthiophen-2-yl, 6,7-dihydro-5H-pyrrolo[3,4-b]pyridin-3-yl, benzo[c][1,2,5]oxadiazol-4-yl, benzofuran-2-yl, benzofuran-5-yl, biphenyl-3-yl, biphenyl-4-yl, chroman-6-yl, chroman-7-yl, isoquinolin-5-yl, isoxazol-4-yl, naphthalen-2-yl, phenyl, pyridin-2-yl, pyridin-3-yl, pyrrolo[1,2-a]pyrimidin-3-yl, quinolin-3-yl, quinolin-6-yl, quinolin-7-yl, and thiazol-4-yl; wherein each is optionally substituted with one or more substituents selected from: (2,2,2-trifluoroethylamino)methyl, (2-aminoethylamino)methyl, (2-methoxyethylamino)methyl, (3-aminopropylamino)methyl, (butylamino)methyl, (cyclobutylamino)methyl, (ethylamino)methyl, (isobutylamino)methyl, (isopropylamino)methyl, (methylamino)methyl, (propylamino)methyl, (tert-butylamino)methyl, (tert-pentylamino)methyl, 1-amino-2-methylpropan-2-yl, 1-aminocyclopropyl, 2-acetamidoethyl, 2-aminoethyl, 2-aminopropan-2-yl, 2-methoxyethyl, amino, aminomethyl, azetidin-1-ylmethyl, bromo, chloro, cyano, cyclopropyl, ethoxy, ethyl, fluoro, hydroxy, isopropoxy, methoxy, methyl, morpholinomethyl, oxo, propan-1-yl, sulfamoyl, and trifluoromethyl.

In some embodiments, R¹ is selected from: (R)-1,3-dimethyl-2,3-dihydro-1H-pyrido[2,3-b][1,4]oxazin-7-yl, (S)-1,3-dimethyl-2,3-dihydro-1H-pyrido[2,3-b][1,4]oxazin-7-yl, 1-(2-methoxyethyl)-2,3-dihydro-1H-pyrido[2,3-b][1,4]oxazin-7-yl, 1,3,3-trimethyl-2,3-dihydro-1H-pyrido[2,3-b][1,4]oxazin-6-yl, 1,4-dimethyl-1,2,3,4-tetrahydropyrido[3,2-b]pyrazin-7-yl, 1,6-dimethyl-2,3-dihydro-1H-pyrido[2,3-b][1,4]oxazin-7-yl, 1,8-dimethyl-2,3-dihydro-1H-pyrido[2,3-b][1,4]oxazin-7-yl, 1-ethoxynaphthalen-2-yl, 1-ethyl-2,3-dihydro-1H-pyrido[2,3-b][1,4]oxazin-7-yl, 1-ethyl-4-oxo-1,4-dihydroquinolin-3-yl, 1-ethyl-5-methyl-1H-pyrazol-4-yl, 1-ethyl-6-fluoro-4-oxo-1,4-dihydroquinolin-3-yl, 1-ethyl-6-methyl-4-oxo-1,4-dihydroquinolin-3-yl, 1-ethyl-7-fluoro-4-oxo-1,4-dihydroquinolin-3-yl, 1-ethyl-7-methyl-4-oxo-1,4-dihydroquinolin-3-yl, 1-ethyl-8-fluoro-4-oxo-1,4-dihydroquinolin-3-yl, 1-ethyl-8-methyl-4-oxo-1,4-dihydroquinolin-3-yl, 1H-benzo[d]imidazol-5-yl, 1H-indazol-5-yl, 1H-indol-2-yl, 1H-indol-3-yl, 1H-indol-5-yl, 1H-indol-6-yl, 1H-pyrazolo[4,3-b]pyridin-6-yl, 1H-pyrrolo[2,3-b]pyridin-3-yl, 1H-pyrrolo[3,2-b]pyridin-6-yl, 1-methyl-2,3-dihydro-1H-pyrido[2,3-b][1,4]oxazin-7-yl, 1-methyl-4-oxo-1,4-dihydroquinolin-3-yl, 2,3-dihydro-[1,4]dioxino[2,3-b]pyridin-7-yl, 2,3-dihydro-1H-pyrido[2,3-b][1,4]oxazin-7-yl, 2,3-dihydrobenzofuran-5-yl, 2-aminothiazol-4-yl, 2-oxo-2,3-dihydro-1H-imidazo[4,5-b]pyridin-6-yl, 2-oxo-2,3-dihydro-1H-pyrrolo[2,3-b]pyridin-5-yl, 3-(1-cyclopropyl-1H-pyrazol-4-yl)phenyl, 3-(1-ethyl-1H-pyrazol-4-yl)phenyl, 3-(1H-pyrazol-4-yl)phenyl, 3-(1-methyl-1H-pyrazol-4-yl)phenyl, 3-(1-propyl-1H-pyrazol-4-yl)phenyl, 3-(2-methylpyridin-4-yl)phenyl, 3-(3-fluoropyridin-2-yl)phenyl, 3-(4-methylpyridin-2-yl)phenyl, 3-(5-methylpyridin-2-yl)phenyl, 3-(6-(trifluoromethyl)pyridin-2-yl)phenyl, 3-(6-aminopyridin-3-yl)phenyl, 3-(6-fluoropyridin-2-yl)phenyl, 3-(6-methylpyridin-2-yl)phenyl, 3-(pyridin-2-yl)phenyl, 3-(pyridin-3-yl)phenyl, 3-(pyridin-4-yl)phenyl, 3-(pyrimidin-5-yl)phenyl, 3-(trifluoromethyl)phenyl, 3,4-dihydro-2H-pyrano[2,3-b]pyridin-6-yl, 3,5-dimethylisoxazol-4-yl, 3-bromo-2-methylphenyl, 3-bromo-4-methoxyphenyl, 3-bromophenyl, 3-chlorophenyl, 3-cyanophenyl, 3-fluorophenyl, 3-methoxyphenyl, 3-methyl-3H-imidazo[4,5-b]pyridin-5-yl, 3-methyl-3H-imidazo[4,5-b]pyridin-6-yl, 3-oxo-3,4-dihydro-2H-benzo[b][1,4]oxazin-6-yl, 4′-((2,2,2-trifluoroethylamino)methyl)biphenyl-3-yl, 4′-((2-aminoethylamino)methyl)biphenyl-3-yl, 4′-((2-methoxyethylamino)methyl)biphenyl-3-yl, 4′-((3-aminopropylamino)methyl)biphenyl-3-yl, 4′-((butylamino)methyl)biphenyl-3-yl, 4′-((cyclobutylamino)methyl)biphenyl-3-yl, 4′-((ethylamino)methyl)biphenyl-3-yl, 4′-((isobutylamino)methyl)biphenyl-3-yl, 4′-((isopropylamino)methyl)biphenyl-3-yl, 4′-((methylamino)methyl)biphenyl-3-yl, 4′-((propylamino)methyl)biphenyl-3-yl, 4′-((tert-butylamino)methyl)biphenyl-3-yl, 4′-((tert-pentylamino)methyl)biphenyl-3-yl, 4′-(1-amino-2-methylpropan-2-yl)-4-ethoxybiphenyl-3-yl, 4′-(1-amino-2-methylpropan-2-yl)biphenyl-3-yl, 4′-(1-aminocyclopropyl)-2-methylbiphenyl-3-yl, 4′-(1-aminocyclopropyl)-4-ethoxybiphenyl-3-yl, 4′-(1-aminocyclopropyl)-6-fluorobiphenyl-3-yl, 4′-(1-aminocyclopropyl)-6-methoxybiphenyl-3-yl, 4′-(1-aminocyclopropyl)biphenyl-3-yl, 4′-(2-acetamidoethyl)-4-ethoxy-biphenyl-3-yl, 4′-(2-acetamidoethyl)-biphenyl-3-yl, 4′-(2-aminoethyl)-4-ethoxybiphenyl-3-yl, 4′-(2-aminoethyl)-6-methoxybiphenyl-3-yl, 4′-(2-aminoethyl)biphenyl-3-yl, 4′-(2-aminopropan-2-yl)-4-ethoxybiphenyl-3-yl, 4′-(aminomethyl)-2-methoxybiphenyl-3-yl, 4′-(aminomethyl)-2-methylbiphenyl-3-yl, 4′-(aminomethyl)-3′-fluorobiphenyl-3-yl, 4′-(aminomethyl)-4-ethoxy-3′-fluorobiphenyl-3-yl, 4′-(aminomethyl)-4-ethoxybiphenyl-3-yl, 4′-(aminomethyl)-4-fluorobiphenyl-3-yl, 4′-(aminomethyl)-4-isopropoxybiphenyl-3-yl, 4′-(aminomethyl)-5-methoxybiphenyl-3-yl, 4′-(aminomethyl)-6-ethoxybiphenyl-3-yl, 4′-(aminomethyl)-6-fluorobiphenyl-3-yl, 4′-(aminomethyl)-6-methoxybiphenyl-3-yl, 4′-(aminomethyl)biphenyl-3-yl, 4′-(aminomethyl)biphenyl-4-yl, 4′-(azetidin-1-ylmethyl)biphenyl-3-yl, 4′-(morpholinomethyl)biphenyl-3-yl, 4-(pyridin-2-yl)phenyl, 4-(pyridin-3-yl)phenyl, 4-(pyridin-4-yl)phenyl, 4′-(sulfamoyl)biphenyl-3-yl, 4-bromo-3-methylphenyl, 4-ethoxy-4′-((isopropylamino)methyl)biphenyl-3-yl, 4-hydroxy-6-methylquinolin-3-yl, 4-hydroxy-7-methylquinolin-3-yl, 4-hydroxy-8-methylquinolin-3-yl, 4-hydroxyquinolin-3-yl, 4-methoxyquinolin-3-yl, 4-methyl-2-oxo-1,2-dihydroquinolin-6-yl, 4-methyl-3,4-dihydro-2H-benzo[b][1,4]oxazin-6-yl, 4-methyl-3,4-dihydro-2H-benzo[b][1,4]oxazin-7-yl, 4-methyl-3,4-dihydro-2H-pyrido[3,2-b][1,4]oxazin-7-yl, 4′-methylbiphenyl-3-yl, 4-oxo-1,4-dihydroquinolin-3-yl, 5-(1-methyl-1H-pyrazol-4-yl)pyridin-3-yl, 5-(4-(aminomethyl)phenyl)pyridin-3-yl, 5,6,7,8-tetrahydronaphthalen-2-yl, 5,6,7,8-tetrahydroquinolin-3-yl, 5-bromo-6-chloropyridin-3-yl, 5-bromopyridin-3-yl, 5-chloronaphthalen-2-yl, 5-oxo-6,7-dihydro-5H-pyrrolo[3,4-b]pyridin-3-yl, 5-phenylthiophen-2-yl, 6-chloronaphthalen-2-yl, 6-fluoro-4-hydroxyquinolin-3-yl, 7-chlorobenzo[c][1,2,5]oxadiazol-4-yl, 7-fluoro-4-hydroxyquinolin-3-yl, 8-fluoro-4-hydroxyquinolin-3-yl, benzofuran-2-yl, benzofuran-5-yl, chroman-6-yl, chroman-7-yl, isoquinolin-5-yl, m-tolyl, naphthalen-2-yl, phenyl, pyridin-2-yl, pyridin-3-yl, pyrrolo[1,2-a]pyrimidin-3-yl, quinolin-3-yl, quinolin-6-yl, and quinolin-7-yl.

### The R¹ Group (Aryl)

In some embodiments, R¹ is aryl, wherein each is optionally substituted with one or more substituents selected from: C₁-C₆ alkoxy, C₁-C₆ alkyl, amino, cyano, C₃-C₇ cycloalkyl, C₁-C₆ haloalkyl, halogen, hydroxyl, oxo, and sulfamoyl; and wherein said C₁-C₆ alkyl and C₃-C₇ cycloalkyl are each optionally substituted with one or more substituents selected from: amino, C₁-C₆ alkoxy, C₁-C₆ alkylcarboxamide, —Y—C₃-C₇-cycloalkyl, —Y—C₁-C₆-alkylene-Z, C₁-C₆ alkylamino, C₁-C₆ haloalkylamino, and heterocyclyl.

In some embodiments, R¹ is aryl optionally substituted with one or more substituents selected from: C₁-C₆ alkoxy, C₁-C₆ alkyl, cyano, C₃-C₇ cycloalkyl, C₁-C₆ haloalkyl, halogen, and sulfamoyl; and wherein said C₁-C₆ alkyl and C₃-C₇ cycloalkyl are each optionally substituted with one or more substituents selected from: amino, C₁-C₆ alkylcarboxamide, —NH—C₃-C₇-cycloalkyl, —NH—C₁-C₆-alkylene-NH₂, —NH—C₁-C₆-alkylene-O—C₁-C₆-alkyl, C₁-C₆ alkylamino, C₁-C₆ haloalkylamino, and heterocyclyl.

In some embodiments, R¹ is selected from: 5,6,7,8-tetrahydronaphthalenyl, biphenyl, naphthalenyl, and phenyl; wherein each is optionally substituted with one or more substituents selected from: 2-methylpropan-2-yl, bromo, chloro, cyano, cyclopropyl, ethoxy, ethyl, fluoro, isopropoxy, methoxy, methyl, propan-2-yl, sulfamoyl, and trifluoromethyl; and wherein said 2-methylpropan-2-yl, cyclopropyl, ethyl, methyl, and propan-2-yl are each optionally substituted with one or more substituents selected from: 2,2,2-trifluoroethylamino, 2-aminoethylamino, 2-methoxyethylamino, 3-aminopropylamino, acetamido, amino, azetidin-1-yl, butylamino, cyclobutylamino, ethylamino, isobutylamino, isopropylamino, methylamino, morpholino, propylamino, tert-butylamino, and tert-pentylamino.

In some embodiments, R¹ is selected from: 5,6,7,8-tetrahydronaphthalenyl, biphenyl, naphthalenyl, and phenyl; wherein each is optionally substituted with one or more substituents selected from: (2,2,2-trifluoroethylamino)methyl, (2-aminoethylamino)methyl, (2-methoxyethylamino)methyl, (3-aminopropylamino)methyl, (butylamino)methyl, (cyclobutylamino)methyl, (ethylamino)methyl, (isobutylamino)methyl, (isopropylamino)methyl, (methylamino)methyl, (propylamino)methyl, (tert-butylamino)methyl, (tert-pentylamino)methyl, 1-amino-2-methylpropan-2-yl, 1-aminocyclopropyl, 2-acetamidoethyl, 2-aminoethyl, 2-aminopropan-2-yl, aminomethyl, azetidin-1-ylmethyl, bromo, chloro, cyano, ethoxy, fluoro, isopropoxy, methoxy, methyl, morpholinomethyl, sulfamoyl, and trifluoromethyl.

In some embodiments, R¹ is selected from: 5,6,7,8-tetrahydronaphthalen-2-yl, biphenyl-3-yl, biphenyl-4-yl, naphthalen-2-yl, and phenyl; wherein each is optionally substituted with one or more substituents selected from: (2,2,2-trifluoroethylamino)methyl, (2-aminoethylamino)methyl, (2-methoxyethylamino)methyl, (3-aminopropylamino)methyl, (butylamino)methyl, (cyclobutylamino)methyl, (ethylamino)methyl, (isobutylamino)methyl, (isopropylamino)methyl, (methylamino)methyl, (propylamino)methyl, (tert-butylamino)methyl, (tert-pentylamino)methyl, 1-amino-2-methylpropan-2-yl, 1-aminocyclopropyl, 2-acetamidoethyl, 2-aminoethyl, 2-aminopropan-2-yl, aminomethyl, azetidin-1-ylmethyl, bromo, chloro, cyano, ethoxy, fluoro, isopropoxy, methoxy, methyl, morpholinomethyl, sulfamoyl, and trifluoromethyl.

In some embodiments, R¹ is selected from: 1-ethoxynaphthalen-2-yl, 3-(trifluoromethyl)phenyl, 3-bromo-2-methylphenyl, 3-bromo-4-methoxyphenyl, 3-bromophenyl, 3-chlorophenyl, 3-cyanophenyl, 3-fluorophenyl, 3-methoxyphenyl, 4′-((2,2,2-trifluoroethylamino)methyl)biphenyl-3-yl, 4′-((2-aminoethylamino)methyl)biphenyl-3-yl, 4′-((2-methoxyethylamino)methyl)biphenyl-3-yl, 4′-((3-aminopropylamino)methyl)biphenyl-3-yl, 4′-((butylamino)methyl)biphenyl-3-yl, 4′-((cyclobutylamino)methyl)biphenyl-3-yl, 4′-((ethylamino)methyl)biphenyl-3-yl, 4′-((isobutylamino)methyl)biphenyl-3-yl, 4′-((isopropylamino)methyl)biphenyl-3-yl, 4′-((methylamino)methyl)biphenyl-3-yl, 4′-((propylamino)methyl)biphenyl-3-yl, 4′-((tert-butylamino)methyl)biphenyl-3-yl, 4′-((tert-pentylamino)methyl)biphenyl-3-yl, 4′-(1-amino-2-methylpropan-2-yl)-4-ethoxybiphenyl-3-yl, 4′-(1-amino-2-methylpropan-2-yl)biphenyl-3-yl, 4′-(1-aminocyclopropyl)-2-methylbiphenyl-3-yl, 4′-(1-aminocyclopropyl)-4-ethoxybiphenyl-3-yl, 4′-(1-aminocyclopropyl)-6-fluorobiphenyl-3-yl, 4′-(1-aminocyclopropyl)-6-methoxybiphenyl-3-yl, 4′-(1-aminocyclopropyl)biphenyl-3-yl, 4′-(2-acetamidoethyl)-4-ethoxy-biphenyl-3-yl, 4′-(2-acetamidoethyl)-biphenyl-3-yl, 4′-(2-aminoethyl)-4-ethoxybiphenyl-3-yl, 4′-(2-aminoethyl)-6-methoxybiphenyl-3-yl, 4′-(2-aminoethyl)biphenyl-3-yl, 4′-(2-aminopropan-2-yl)-4-ethoxybiphenyl-3-yl, 4′-(aminomethyl)-2-methoxybiphenyl-3-yl, 4′-(aminomethyl)-2-methylbiphenyl-3-yl, 4′-(aminomethyl)-3′-fluorobiphenyl-3-yl, 4′-(aminomethyl)-4-ethoxy-3′-fluorobiphenyl-3-yl, 4′-(aminomethyl)-4-ethoxybiphenyl-3-yl, 4′-(aminomethyl)-4-fluorobiphenyl-3-yl, 4′-(aminomethyl)-4-isopropoxybiphenyl-3-yl, 4′-(aminomethyl)-5-methoxybiphenyl-3-yl, 4′-(aminomethyl)-6-ethoxybiphenyl-3-yl, 4′-(aminomethyl)-6-fluorobiphenyl-3-yl, 4′-(aminomethyl)-6-methoxybiphenyl-3-yl, 4′-(aminomethyl)biphenyl-3-yl, 4′-(aminomethyl)biphenyl-4-yl, 4′-(azetidin-1-ylmethyl)biphenyl-3-yl, 4′-(morpholinomethyl)biphenyl-3-yl, 4′-(sulfamoyl)biphenyl-3-yl, 4-bromo-3-methylphenyl, 4-ethoxy-4′-((isopropylamino)methyl)biphenyl-3-yl, 4′-methylbiphenyl-3-yl, 5,6,7,8-tetrahydronaphthalen-2-yl, 5-chloronaphthalen-2-yl, 6-chloronaphthalen-2-yl, m-tolyl, naphthalen-2-yl, and phenyl.

### The R¹ Group (Heteroaryl)

In some embodiments, R¹ is heteroaryl, wherein each is optionally substituted with one or more substituents selected from: C₁-C₆ alkoxy, C₁-C₆ alkyl, amino, cyano, C₃-C₇ cycloalkyl, C₁-C₆ haloalkyl, halogen, hydroxyl, oxo, and sulfamoyl; and wherein said C₁-C₆ alkyl and C₃-C₇ cycloalkyl are each optionally substituted with one or more substituents selected from: amino, C₁-C₆ alkoxy, C₁-C₆ alkylcarboxamide, —Y—C₃-C₇-cycloalkyl, —Y—C₁-C₆-alkylene-Z, C₁-C₆ alkylamino, C₁-C₆ haloalkylamino, and heterocyclyl.

In some embodiments, R¹ is heteroaryl optionally substituted with one or more substituents selected from: C₁-C₆ alkoxy, C₁-C₆ alkyl, amino, C₃-C₇ cycloalkyl, C₁-C₆ haloalkyl, halogen, hydroxyl, and oxo; and wherein said C₁-C₆ alkyl is optionally substituted with one or more substituents selected from: amino and C₁-C₆ alkoxy.

In some embodiments, R¹ is selected from: (1H-pyrazolyl)phenyl, (1H-pyrazolyl)pyridinyl, (pyridinyl)phenyl, (pyrimidinyl)phenyl, 1,2,3,4-tetrahydropyrido[3,2-b]pyrazinyl, 1,2-dihydroquinolinyl, 1,4-dihydroquinolinyl, 1H-benzo[d]imidazolyl, 1H-indazolyl, 1H-indolyl, 1H-pyrazolo[4,3-b]pyridinyl, 1H-pyrazolyl, 1H-pyrrolo[2,3-b]pyridinyl, 1H-pyrrolo[3,2-b]pyridinyl, 2,3-dihydro-[1,4]dioxino[2,3-b]pyridinyl, 2,3-dihydro-1H-imidazo[4,5-b]pyridinyl, 2,3-dihydro-1H-pyrido[2,3-b][1,4]oxazinyl, 2,3-dihydro-1H-pyrrolo[2,3-b]pyridinyl, 2,3-dihydrobenzofuranyl, 3,4-dihydro-2H-benzo[b][1,4]oxazinyl, 3,4-dihydro-2H-pyrano[2,3-b]pyridinyl, 3,4-dihydro-2H-pyrido[3,2-b][1,4]oxazinyl, 3H-imidazo[4,5-b]pyridinyl, (phenyl)pyridinyl, 5,6,7,8-tetrahydroquinolinyl, 6,7-dihydro-5H-pyrrolo[3,4-b]pyridin-3-yl, benzo[c][1,2,5]oxadiazolyl, benzofuranyl, chromanyl, isoquinolinyl, isoxazolyl, phenylthiophenyl, pyridinyl, pyrrolo[1,2-a]pyrimidinyl, quinolinyl, and thiazolyl; wherein each is optionally substituted with one or more substituents selected from: amino, bromo, chloro, cyclopropyl, ethyl, fluoro, hydroxy, methoxy, methyl, oxo, propan-1-yl, and trifluoromethyl; and wherein said ethyl and methyl are each optionally substituted with one or more substituents selected from: amino and methoxy.

In some embodiments, R¹ is selected from: (1H-pyrazolyl)phenyl, (1H-pyrazolyl)pyridinyl, (pyridinyl)phenyl, (pyrimidinyl)phenyl, 1,2,3,4-tetrahydropyrido[3,2-b]pyrazinyl, 1,2-dihydroquinolinyl, 1,4-dihydroquinolinyl, 1H-benzo[d]imidazolyl, 1H-indazolyl, 1H-indolyl, 1H-pyrazolo[4,3-b]pyridinyl, 1H-pyrazolyl, 1H-pyrrolo[2,3-b]pyridinyl, 1H-pyrrolo[3,2-b]pyridinyl, 2,3-dihydro-[1,4]dioxino[2,3-b]pyridinyl, 2,3-dihydro-1H-imidazo[4,5-b]pyridinyl, 2,3-dihydro-1H-pyrido[2,3-b][1,4]oxazinyl, 2,3-dihydro-1H-pyrrolo[2,3-b]pyridinyl, 2,3-dihydrobenzofuranyl, 3,4-dihydro-2H-benzo[b][1,4]oxazinyl, 3,4-dihydro-2H-pyrano[2,3-b]pyridinyl, 3,4-dihydro-2H-pyrido[3,2-b][1,4]oxazinyl, 3H-imidazo[4,5-b]pyridinyl, (phenyl)pyridinyl, 5,6,7,8-tetrahydroquinolinyl, 6,7-dihydro-5H-pyrrolo[3,4-b]pyridin-3-yl, benzo[c][1,2,5]oxadiazolyl, benzofuranyl, chromanyl, isoquinolinyl, isoxazolyl, phenylthiophenyl, pyridinyl, pyrrolo[1,2-a]pyrimidinyl, quinolinyl, and thiazolyl; wherein each is optionally substituted with one or more substituents selected from: 2-methoxyethyl, amino, aminomethyl, bromo, chloro, cyclopropyl, ethyl, fluoro, hydroxy, methoxy, methyl, oxo, propan-1-yl, and trifluoromethyl.

In some embodiments, R¹ is selected from: 1,2,3,4-tetrahydropyrido[3,2-b]pyrazin-7-yl, 1,2-dihydroquinolin-6-yl, 1,4-dihydroquinolin-3-yl, 1H-benzo[d]imidazol-5-yl, 1H-indazol-5-yl, 1H-indol-2-yl, 1H-indol-3-yl, 1H-indol-5-yl, 1H-indol-6-yl, 1H-pyrazol-4-yl, 1H-pyrazolo[4,3-b]pyridin-6-yl, 1H-pyrrolo[2,3-b]pyridin-3-yl, 1H-pyrrolo[3,2-b]pyridin-6-yl, 2,3-dihydro-[1,4]dioxino[2,3-b]pyridin-7-yl, 2,3-dihydro-1H-imidazo[4,5-b]pyridin-6-yl, 2,3-dihydro-1H-pyrido[2,3-b][1,4]oxazin-6-yl, 2,3-dihydro-1H-pyrido[2,3-b][1,4]oxazin-7-yl, 2,3-dihydro-1H-pyrrolo[2,3-b]pyridin-5-yl, 2,3-dihydrobenzofuran-5-yl, 3-(1H-pyrazol-4-yl)phenyl, 3-(pyridin-2-yl)phenyl, 3-(pyridin-3-yl)phenyl, 3-(pyridin-4-yl)phenyl, 3-(pyrimidin-5-yl)phenyl, 3,4-dihydro-2H-benzo[b][1,4]oxazin-6-yl, 3,4-dihydro-2H-benzo[b][1,4]oxazin-7-yl, 3,4-dihydro-2H-pyrano[2,3-b]pyridin-6-yl, 3,4-dihydro-2H-pyrido[3,2-b][1,4]oxazin-7-yl, 3H-imidazo[4,5-b]pyridin-5-yl, 3H-imidazo[4,5-b]pyridin-6-yl, 4-(pyridin-2-yl)phenyl, 4-(pyridin-3-yl)phenyl, 4-(pyridin-4-yl)phenyl, 5-(1H-pyrazol-4-yl)pyridin-3-yl, 5-(phenyl)pyridin-3-yl, 5,6,7,8-tetrahydroquinolin-3-yl, 5-phenylthiophen-2-yl, 6,7-dihydro-5H-pyrrolo[3,4-b]pyridin-3-yl, benzo[c][1,2,5]oxadiazol-4-yl, benzofuran-2-yl, benzofuran-5-yl, chroman-6-yl, chroman-7-yl, isoquinolin-5-yl, isoxazol-4-yl, pyridin-2-yl, pyridin-3-yl, pyrrolo[1,2-a]pyrimidin-3-yl, quinolin-3-yl, quinolin-6-yl, quinolin-7-yl, and thiazol-4-yl; wherein each is optionally substituted with one or more substituents selected from: 2-methoxyethyl, amino, aminomethyl, bromo, chloro, cyclopropyl, ethyl, fluoro, hydroxy, methoxy, methyl, oxo, propan-1-yl, and trifluoromethyl.

In some embodiments, R¹ is selected from: (R)-1,3-dimethyl-2,3-dihydro-1H-pyrido[2,3-b][1,4]oxazin-7-yl, (S)-1,3-dimethyl-2,3-dihydro-1H-pyrido[2,3-b][1,4]oxazin-7-yl, 1-(2-methoxyethyl)-2,3-dihydro-1H-pyrido[2,3-b][1,4]oxazin-7-yl, 1,3,3-trimethyl-2,3-dihydro-1H-pyrido[2,3-b][1,4]oxazin-6-yl, 1,4-dimethyl-1,2,3,4-tetrahydropyrido[3,2-b]pyrazin-7-yl, 1,6-dimethyl-2,3-dihydro-1H-pyrido[2,3-b][1,4]oxazin-7-yl, 1,8-dimethyl-2,3-dihydro-1H-pyrido[2,3-b][1,4]oxazin-7-yl, 1-ethyl-2,3-dihydro-1H-pyrido[2,3-b][1,4]oxazin-7-yl, 1-ethyl-4-oxo-1,4-dihydroquinolin-3-yl, 1-ethyl-5-methyl-1H-pyrazol-4-yl, 1-ethyl-6-fluoro-4-oxo-1,4-dihydroquinolin-3-yl, 1-ethyl-6-methyl-4-oxo-1,4-dihydroquinolin-3-yl, 1-ethyl-7-fluoro-4-oxo-1,4-dihydroquinolin-3-yl, 1-ethyl-7-methyl-4-oxo-1,4-dihydroquinolin-3-yl, 1-ethyl-8-fluoro-4-oxo-1,4-dihydroquinolin-3-yl, 1-ethyl-8-methyl-4-oxo-1,4-dihydroquinolin-3-yl, 1H-benzo[d]imidazol-5-yl, 1H-indazol-5-yl, 1H-indol-2-yl, 1H-indol-3-yl, 1H-indol-5-yl, 1H-indol-6-yl, 1H-pyrazolo[4,3-b]pyridin-6-yl, 1H-pyrrolo[2,3-b]pyridin-3-yl, 1H-pyrrolo[3,2-b]pyridin-6-yl, 1-methyl-2,3-dihydro-1H-pyrido[2,3-b][1,4]oxazin-7-yl, 1-methyl-4-oxo-1,4-dihydroquinolin-3-yl, 2,3-dihydro-[1,4]dioxino[2,3-b]pyridin-7-yl, 2,3-dihydro-1H-pyrido[2,3-b][1,4]oxazin-7-yl, 2,3-dihydrobenzofuran-5-yl, 2-aminothiazol-4-yl, 2-oxo-2,3-dihydro-1H-imidazo[4,5-b]pyridin-6-yl, 2-oxo-2,3-dihydro-1H-pyrrolo[2,3-b]pyridin-5-yl, 3-(1-cyclopropyl-1H-pyrazol-4-yl)phenyl, 3-(1-ethyl-1H-pyrazol-4-yl)phenyl, 3-(1H-pyrazol-4-yl)phenyl, 3-(1-methyl-1H-pyrazol-4-yl)phenyl, 3-(1-propyl-1H-pyrazol-4-yl)phenyl, 3-(2-methylpyridin-4-yl)phenyl, 3-(3-fluoropyridin-2-yl)phenyl, 3-(4-methylpyridin-2-yl)phenyl, 3-(5-methylpyridin-2-yl)phenyl, 3-(6-(trifluoromethyl)pyridin-2-yl)phenyl, 3-(6-aminopyridin-3-yl)phenyl, 3-(6-fluoropyridin-2-yl)phenyl, 3-(6-methylpyridin-2-yl)phenyl, 3-(pyridin-2-yl)phenyl, 3-(pyridin-3-yl)phenyl, 3-(pyridin-4-yl)phenyl, 3-(pyrimidin-5-yl)phenyl, 3,4-dihydro-2H-pyrano[2,3-b]pyridin-6-yl, 3,5-dimethylisoxazol-4-yl, 3-methyl-3H-imidazo[4,5-b]pyridin-5-yl, 3-methyl-3H-imidazo[4,5-b]pyridin-6-yl, 3-oxo-3,4-dihydro-2H-benzo[b][1,4]oxazin-6-yl, 4-(pyridin-2-yl)phenyl, 4-(pyridin-3-yl)phenyl, 4-(pyridin-4-yl)phenyl, 4-hydroxy-6-methylquinolin-3-yl, 4-hydroxy-7-methylquinolin-3-yl, 4-hydroxy-8-methylquinolin-3-yl, 4-hydroxyquinolin-3-yl, 4-methoxyquinolin-3-yl, 4-methyl-2-oxo-1,2-dihydroquinolin-6-yl, 4-methyl-3,4-dihydro-2H-benzo[b][1,4]oxazin-6-yl, 4-methyl-3,4-dihydro-2H-benzo[b][1,4]oxazin-7-yl, 4-methyl-3,4-dihydro-2H-pyrido[3,2-b][1,4]oxazin-7-yl, 4-oxo-1,4-dihydroquinolin-3-yl, 5-(1-methyl-1H-pyrazol-4-yl)pyridin-3-yl, 5-(4-(aminomethyl)phenyl)pyridin-3-yl, 5,6,7,8-tetrahydroquinolin-3-yl, 5-bromo-6-chloropyridin-3-yl, 5-bromopyridin-3-yl, 5-oxo-6,7-dihydro-5H-pyrrolo[3,4-b]pyridin-3-yl, 5-phenylthiophen-2-yl, 6-fluoro-4-hydroxyquinolin-3-yl, 7-chlorobenzo[c][1,2,5]oxadiazol-4-yl, 7-fluoro-4-hydroxyquinolin-3-yl, 8-fluoro-4-hydroxyquinolin-3-yl, benzofuran-2-yl, benzofuran-5-yl, chroman-6-yl, chroman-7-yl, isoquinolin-5-yl, pyridin-2-yl, pyridin-3-yl, pyrrolo[1,2-a]pyrimidin-3-yl, quinolin-3-yl, quinolin-6-yl, and quinolin-7-yl.

### The R² Group

In some embodiments, R² is selected from: C₂-C₆ alkenyl, C₁-C₆ alkyl, C₃-C₇ cycloalkyl, heterocyclyl, and C₁-C₆ haloalkyl; each optionally substituted with one or more substituents selected from: C₁-C₆ alkoxy, C₁-C₆ alkylenehydroxyl, amino, aryl, C₃-C₇ cycloalkyl, cyano, C₃-C₇ halocycloalkyl, hydroxyl, and oxo.

In some embodiments, R² is selected from: 1,1-difluoroethyl, 1-fluoroethyl, 2-methylpropan-2-yl, 3,3,3-trifluoropropyl, 4,4,4-trifluorobutyl, azetidin-3-yl, cyclobutyl, cyclopentyl, cyclopropyl, ethyl, fluoromethyl, isobutyl, isopentyl, isopropyl, methyl, oxetan-3-yl, propan-1-yl, sec-butyl, and vinyl; each optionally substituted with one or more substituents selected from: 2,2-difluorocyclopropyl, amino, cyano, cyclobutyl, cyclohexyl, cyclopropyl, ethoxy, hydroxy, hydroxymethyl, methoxy, oxo, and phenyl.

In some embodiments, R² is selected from: (2,2-difluorocyclopropyl)methyl, 1-(hydroxymethyl)cyclobutyl, 1-(hydroxymethyl)cyclopropyl, 1,1-difluoro-2-hydroxyethyl, 1-amino-2-methyl-1-oxopropan-2-yl, 1-ethoxy-2-methyl-1-oxopropan-2-yl, 1-fluoroethyl, 1-hydroxy-2-methylpropan-2-yl, 2-amino-2-oxoethyl, 2-aminoethyl, 2-hydroxyethyl, 3,3,3-trifluoropropyl, 3-amino-3-oxopropyl, 3-hydroxycyclobutyl, 3-hydroxypropyl, 3-methoxypropyl, 4,4,4-trifluorobutyl, azetidin-3-yl, benzyl, carboxymethyl, cyanomethyl, cyclobutyl, cyclobutylmethyl, cyclohexylmethyl, cyclopentyl, cyclopropyl, cyclopropylmethyl, ethyl, fluoromethyl, isobutyl, isopentyl, isopropyl, methoxymethyl, methyl, oxetan-3-yl, propan-1-yl, sec-butyl, and vinyl.

In some embodiments, R² is selected from: 1-(hydroxymethyl)cyclobutyl, 1-(hydroxymethyl)cyclopropyl, 1,1-difluoro-2-hydroxyethyl, 1-fluoroethyl, 1-hydroxy-2-methylpropan-2-yl, 2-amino-2-oxoethyl, 2-hydroxyethyl, 3-amino-3-oxopropyl, 3-hydroxypropyl, 3-methoxypropyl, cyclobutyl, cyclopropyl, cyclopropylmethyl, ethyl, isobutyl, isopropyl, methoxymethyl, methyl, and propan-1-yl.

The R^{3a}, R^{3b}, R^{3c}, and R^{3d} Groups

In some embodiments, R^{3a}, R^{3b}, R^{3c}, and R^{3d} are each independently H or halogen.

In some embodiments, R^{3a} is H or halogen; R^{3b} is H; R^{3c} is H or halogen; and R^{3d} is H.

In some embodiments, R^{3a} is halogen; R^{3b} is H; R^{3c} is H or halogen; and R^{3d} is H.

In some embodiments, R^{3a} is H; R^{3b} is H; R^{3c} is halogen; and R^{3d} is H.

In some embodiments, R^{3a}, R^{3b}, R^{3c}, and R^{3d} are each independently H or F.

In some embodiments, R^{3a} is H or F; R^{3b} is H; R^{3c} is H or F; and R^{3d} is H.

In some embodiments, R^{3a} is F; R^{3b} is H; R^{3c} is H; and R^{3d} is H.

In some embodiments, R^{3a} is H; R^{3b} is H; R^{3c} is F; and R^{3d} is H.

In some embodiments, R^{3a}, R^{3b}, R^{3c}, and R^{3d} are each H.

In some embodiments, R^{3a} is halogen.

In some embodiments, R^{3b} is halogen.

In some embodiments, R^{3c} is halogen.

In some embodiments, R^{3d} is halogen.

In some embodiments, R^{3a} is F.

In some embodiments, R^{3b} is F.

In some embodiments, R^{3c} is F.

In some embodiments, R^{3d} is F.

In some embodiments, R^{3a} is H.

In some embodiments, R^{3b} is H.

In some embodiments, R^{3c} is H.

In some embodiments, R^{3d} is H.

### Certain Combinations

One aspect of the present invention pertains to compounds of Formula (Ib) and pharmaceutically acceptable salts, solvates, and hydrates thereof:

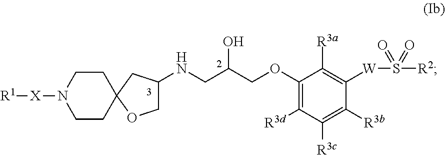

wherein: R¹ (as well as Y and Z that are both related to R¹), X, W, R², R^{3a}, R^{3b}, R^{3c}, and R^{3d} all have the same definitions as described herein, supra and infra.

One aspect of the present invention pertains to compounds of Formula (Ic) and pharmaceutically acceptable salts, solvates, and hydrates thereof:

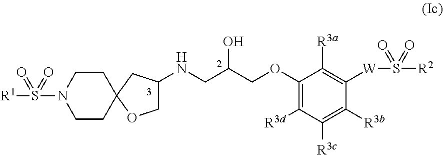

wherein:
W is absent or —CH₂—;
R¹ is aryl or heteroaryl, wherein each is optionally substituted with one or more substituents selected from: C₁-C₆ alkoxy, C₁-C₆ alkyl, amino, cyano, C₃-C₇ cycloalkyl, C₁-C₆ haloalkyl, halogen, hydroxyl, oxo, and sulfamoyl; and wherein said C₁-C₆ alkyl and C₃-C₇ cycloalkyl are each optionally substituted with one or more substituents selected from: amino, C₁-C₆ alkoxy, C₁-C₆ alkylcarboxamide, —NH—C₃-C₇-cycloalkyl, —NH—C₁-C₆-alkylene-NH₂, —NH—C₁-C₆-alkylene-O—C₁-C₆-alkyl, —NH—C₁-C₆-alkylene-NH—C₁-C₆-alkyl, C₁-C₆ alkylamino, C₁-C₆ haloalkylamino, and heterocyclyl;
R² is selected from: C₂-C₆ alkenyl, C₁-C₆ alkyl, C₃-C₇ cycloalkyl, heterocyclyl, and C₁-C₆ haloalkyl; each optionally substituted with one or more substituents selected from: C₁-C₆ alkoxy, C₁-C₆ alkylenehydroxyl, amino, aryl, C₃-C₇ cycloalkyl, cyano, C₃-C₇ halocycloalkyl, hydroxyl, and oxo; and
R^{3a}, R^{3b}, R^{3c}, and R^{3d} are each independently H or halogen.

One aspect of the present invention pertains to compounds of Formula (Ic) and pharmaceutically acceptable salts, solvates, and hydrates thereof:

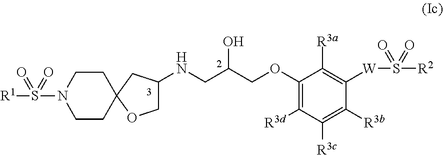

wherein:
W is absent or —CH₂—;
R¹ is selected from: (1H-pyrazolyl)phenyl, (1H-pyrazolyl)pyridinyl, (pyridinyl)phenyl, (pyrimidinyl)phenyl, 1,2,3,4-tetrahydropyrido[3,2-b]pyrazinyl, 1,2-dihydroquinolinyl, 1,4-dihydroquinolinyl, 1H-benzo[d]imidazolyl, 1H-indazolyl, 1H-indolyl, 1H-pyrazolo[4,3-b]pyridinyl, 1H-pyrazolyl, 1H-pyrrolo[2,3-b]pyridinyl, 1H-pyrrolo[3,2-b]pyridinyl, 2,3-dihydro-[1,4]dioxino[2,3-b]pyridinyl, 2,3-dihydro-1H-imidazo[4,5-b]pyridinyl, 2,3-dihydro-1H-pyrido[2,3-b][1,4]oxazinyl, 2,3-dihydro-1H-pyrrolo[2,3-b]pyridinyl, 2,3-dihydrobenzofuranyl, 3,4-dihydro-2H-benzo[b][1,4]oxazinyl, 3,4-dihydro-2H-pyrano[2,3-b]pyridinyl, 3,4-dihydro-2H-pyrido[3,2-b][1,4]oxazinyl, 3H-imidazo[4,5-b]pyridinyl, (phenyl)pyridinyl, 5,6,7,8-tetrahydronaphthalenyl, 5,6,7,8-tetrahydroquinolinyl, 6,7-dihydro-5H-pyrrolo[3,4-b]pyridin-3-yl, benzo[c][1,2,5]oxadiazolyl, benzofuranyl, biphenyl, chromanyl, isoquinolinyl, isoxazolyl, naphthalenyl, phenyl, phenylthiophenyl, pyridinyl, pyrrolo[1,2-a]pyrimidinyl, quinolinyl, and thiazolyl; wherein each is optionally substituted with one or more substituents selected from: 2-methylpropan-2-yl, amino, bromo, chloro, cyclopropyl, ethoxy, ethyl, fluoro, hydroxy, isopropoxy, methoxy, methyl, oxo, propan-2-yl, propan-1-yl, sulfamoyl, and trifluoromethyl; and wherein said 2-methylpropan-2-yl, cyclopropyl, ethyl, methyl, and propan-2-yl are each optionally substituted with one or more substituents selected from: 2,2,2-trifluoroethylamino, 2-aminoethylamino, 2-methoxyethylamino, 3-aminopropylamino, acetamido, amino, azetidin-1-yl, butylamino, cyclobutylamino, ethylamino, isobutylamino, isopropylamino, methoxy, methylamino, morpholino, propylamino, tert-butylamino, and tert-pentylamino;
R² is selected from: 1,1-difluoroethyl, 1-fluoroethyl, 2-methylpropan-2-yl, 3,3,3-trifluoropropyl, 4,4,4-trifluorobutyl, azetidin-3-yl, cyclobutyl, cyclopentyl, cyclopropyl, ethyl, fluoromethyl, isobutyl, isopentyl, isopropyl, methyl, oxetan-3-yl, propan-1-yl, sec-butyl, and vinyl; each optionally substituted with one or more substituents selected from: 2,2-difluorocyclopropyl, amino, cyano, cyclobutyl, cyclohexyl, cyclopropyl, ethoxy, hydroxy, hydroxymethyl, methoxy, oxo, and phenyl; and
R^{3a}, R^{3b}, R^{3c}, and R^{3d} are each independently H or F.

One aspect of the present invention pertains to compounds of Formula (Ic) and pharmaceutically acceptable salts, solvates, and hydrates thereof:

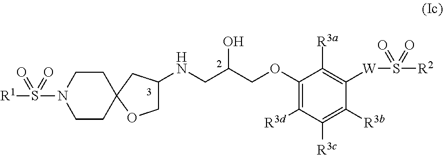

wherein:
W is absent or —CH₂—;
R¹ is selected from: (R)-1,3-dimethyl-2,3-dihydro-1H-pyrido[2,3-b][1,4]oxazin-7-yl, (S)-1,3-dimethyl-2,3-dihydro-1H-pyrido[2,3-b][1,4]oxazin-7-yl, 1-(2-methoxyethyl)-2,3-dihydro-1H-pyrido[2,3-b][1,4]oxazin-7-yl, 1,3,3-trimethyl-2,3-dihydro-1H-pyrido[2,3-b][1,4]oxazin-6-yl, 1,4-dimethyl-1,2,3,4-tetrahydropyrido[3,2-b]pyrazin-7-yl, 1,6-dimethyl-2,3-dihydro-1H-pyrido[2,3-b][1,4]oxazin-7-yl, 1,8-dimethyl-2,3-dihydro-1H-pyrido[2,3-b][1,4]oxazin-7-yl, 1-ethyl-2,3-dihydro-1H-pyrido[2,3-b][1,4]oxazin-7-yl, 1-ethyl-4-oxo-1,4-dihydroquinolin-3-yl, 1-ethyl-5-methyl-1H-pyrazol-4-yl, 1-ethyl-6-fluoro-4-oxo-1,4-dihydroquinolin-3-yl, 1-ethyl-6-methyl-4-oxo-1,4-dihydroquinolin-3-yl, 1-ethyl-7-fluoro-4-oxo-1,4-dihydroquinolin-3-yl, 1-ethyl-7-methyl-4-oxo-1,4-dihydroquinolin-3-yl, 1-ethyl-8-fluoro-4-oxo-1,4-dihydroquinolin-3-yl, 1-ethyl-8-methyl-4-oxo-1,4-dihydroquinolin-3-yl, 1H-benzo[d]imidazol-5-yl, 1H-indazol-5-yl, 1H-indol-2-yl, 1H-indol-3-yl, 1H-indol-5-yl, 1H-indol-6-yl, 1H-pyrazolo[4,3-b]pyridin-6-yl, 1H-pyrrolo[2,3-b]pyridin-3-yl, 1H-pyrrolo[3,2-b]pyridin-6-yl, 1-methyl-2,3-dihydro-1H-pyrido[2,3-b][1,4]oxazin-7-yl, 1-methyl-4-oxo-1,4-dihydroquinolin-3-yl, 2,3-dihydro-[1,4]dioxino[2,3-b]pyridin-7-yl, 2,3-dihydro-1H-pyrido[2,3-b][1,4]oxazin-7-yl, 2,3-dihydrobenzofuran-5-yl, 2-aminothiazol-4-yl, 2-oxo-2,3-dihydro-1H-imidazo[4,5-b]pyridin-6-yl, 2-oxo-2,3-dihydro-1H-pyrrolo[2,3-b]pyridin-5-yl, 3-(1-cyclopropyl-1H-pyrazol-4-yl)phenyl, 3-(1-ethyl-1H-pyrazol-4-yl)phenyl, 3-(1H-pyrazol-4-yl)phenyl, 3-(1-methyl-1H-pyrazol-4-yl)phenyl, 3-(1-propyl-1H-pyrazol-4-yl)phenyl, 3-(2-methylpyridin-4-yl)phenyl, 3-(3-fluoropyridin-2-yl)phenyl, 3-(4-methylpyridin-2-yl)phenyl, 3-(5-methylpyridin-2-yl)phenyl, 3-(6-(trifluoromethyl)pyridin-2-yl)phenyl, 3-(6-aminopyridin-3-yl)phenyl, 3-(6-fluoropyridin-2-yl)phenyl, 3-(6-methylpyridin-2-yl)phenyl, 3-(pyridin-2-yl)phenyl, 3-(pyridin-3-yl)phenyl, 3-(pyridin-4-yl)phenyl, 3-(pyrimidin-5-yl)phenyl, 3,4-dihydro-2H-pyrano[2,3-b]pyridin-6-yl, 3,5-dimethylisoxazol-4-yl, 3-methyl-3H-imidazo[4,5-b]pyridin-5-yl, 3-methyl-3H-imidazo[4,5-b]pyridin-6-yl, 3-oxo-3,4-dihydro-2H-benzo[b][1,4]oxazin-6-yl, 4-(pyridin-2-yl)phenyl, 4-(pyridin-3-yl)phenyl, 4-(pyridin-4-yl)phenyl, 4-hydroxy-6-methylquinolin-3-yl, 4-hydroxy-7-methylquinolin-3-yl, 4-hydroxy-8-methylquinolin-3-yl, 4-hydroxyquinolin-3-yl, 4-methoxyquinolin-3-yl, 4-methyl-2-oxo-1,2-dihydroquinolin-6-yl, 4-methyl-3,4-dihydro-2H-benzo[b][1,4]oxazin-6-yl, 4-methyl-3,4-dihydro-2H-benzo[b][1,4]oxazin-7-yl, 4-methyl-3,4-dihydro-2H-pyrido[3,2-b][1,4]oxazin-7-yl, 4-oxo-1,4-dihydroquinolin-3-yl, 5-(1-methyl-1H-pyrazol-4-yl)pyridin-3-yl, 5-(4-(aminomethyl)phenyl)pyridin-3-yl, 5,6,7,8-tetrahydroquinolin-3-yl, 5-bromo-6-chloropyridin-3-yl, 5-bromopyridin-3-yl, 5-oxo-6,7-dihydro-5H-pyrrolo[3,4-b]pyridin-3-yl, 5-phenylthiophen-2-yl, 6-fluoro-4-hydroxyquinolin-3-yl, 7-chlorobenzo[c][1,2,5]oxadiazol-4-yl, 7-fluoro-4-hydroxyquinolin-3-yl, 8-fluoro-4-hydroxyquinolin-3-yl, benzofuran-2-yl, benzofuran-5-yl, chroman-6-yl, chroman-7-yl, isoquinolin-5-yl, pyridin-2-yl, pyridin-3-yl, pyrrolo[1,2-a]pyrimidin-3-yl, quinolin-3-yl, quinolin-6-yl, and quinolin-7-yl;
R² is selected from: (2,2-difluorocyclopropyl)methyl, 1-(hydroxymethyl)cyclobutyl, 1-(hydroxymethyl)cyclopropyl, 1,1-difluoro-2-hydroxyethyl, 1-amino-2-methyl-1-oxopropan-2-yl, 1-ethoxy-2-methyl-1-oxopropan-2-yl, 1-fluoroethyl, 1-hydroxy-2-methylpropan-2-yl, 2-amino-2-oxoethyl, 2-aminoethyl, 2-hydroxyethyl, 3,3,3-trifluoropropyl, 3-amino-3-oxopropyl, 3-hydroxycyclobutyl, 3-hydroxypropyl, 3-methoxypropyl, 4,4,4-trifluorobutyl, azetidin-3-yl, benzyl, carboxymethyl, cyanomethyl, cyclobutyl, cyclobutylmethyl, cyclohexylmethyl, cyclopentyl, cyclopropyl, cyclopropylmethyl, ethyl, fluoromethyl, isobutyl, isopentyl, isopropyl, methoxymethyl, methyl, oxetan-3-yl, propan-1-yl, sec-butyl, and vinyl; and
R^{3a}, R^{3b}, R^{3c}, and R^{3d} are each independently H or F.

One aspect of the present invention pertains to compounds of Formula (Ie) and pharmaceutically acceptable salts, solvates, and hydrates thereof:

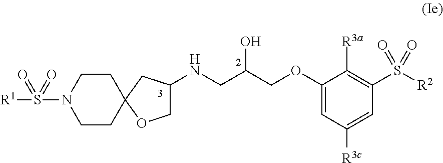

wherein:
R¹ is aryl or heteroaryl, wherein each is optionally substituted with one or more substituents selected from: C₁-C₆ alkoxy, C₁-C₆ alkyl, amino, cyano, C₃-C₇ cycloalkyl, C₁-C₆ haloalkyl, halogen, hydroxyl, oxo, and sulfamoyl; and wherein said C₁-C₆ alkyl and C₃-C₇ cycloalkyl are each optionally substituted with one or more substituents selected from: amino, C₁-C₆ alkoxy, C₁-C₆ alkylcarboxamide, —NH—C₃-C₇-cycloalkyl, —NH—C₁-C₆-alkylene-NH₂, —NH—C₁-C₆-alkylene-O—C₁-C₆-alkyl, —NH—C₁-C₆-alkylene-NH—C₁-C₆-alkyl, C₁-C₆ alkylamino, C₁-C₆ haloalkylamino, and heterocyclyl;
R² is selected from: C₂-C₆ alkenyl, C₁-C₆ alkyl, C₃-C₇ cycloalkyl, heterocyclyl, and C₁-C₆ haloalkyl; each optionally substituted with one or more substituents selected from: C₁-C₆ alkoxy, C₁-C₆ alkylenehydroxyl, amino, aryl, C₃-C₇ cycloalkyl, cyano, C₃-C₇ halocycloalkyl, hydroxyl, and oxo; and
R^{3a} and R^{3c} are each independently H or halogen.

One aspect of the present invention pertains to compounds of Formula (Ie) and pharmaceutically acceptable salts, solvates, and hydrates thereof:

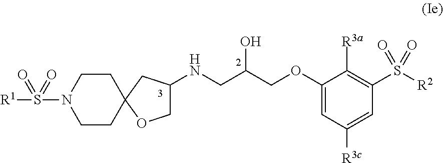

wherein:
R¹ is selected from: (1H-pyrazolyl)phenyl, (1H-pyrazolyl)pyridinyl, (pyridinyl)phenyl, (pyrimidinyl)phenyl, 1,2,3,4-tetrahydropyrido[3,2-b]pyrazinyl, 1,2-dihydroquinolinyl, 1,4-dihydroquinolinyl, 1H-benzo[d]imidazolyl, 1H-indazolyl, 1H-indolyl, 1H-pyrazolo[4,3-b]pyridinyl, 1H-pyrazolyl, 1H-pyrrolo[2,3-b]pyridinyl, 1H-pyrrolo[3,2-b]pyridinyl, 2,3-dihydro-[1,4]dioxino[2,3-b]pyridinyl, 2,3-dihydro-1H-imidazo[4,5-b]pyridinyl, 2,3-dihydro-1H-pyrido[2,3-b][1,4]oxazinyl, 2,3-dihydro-1H-pyrrolo[2,3-b]pyridinyl, 2,3-dihydrobenzofuranyl, 3,4-dihydro-2H-benzo[b][1,4]oxazinyl, 3,4-dihydro-2H-pyrano[2,3-b]pyridinyl, 3,4-dihydro-2H-pyrido[3,2-b][1,4]oxazinyl, 3H-imidazo[4,5-b]pyridinyl, (phenyl)pyridinyl, 5,6,7,8-tetrahydronaphthalenyl, 5,6,7,8-tetrahydroquinolinyl, 6,7-dihydro-5H-pyrrolo[3,4-b]pyridin-3-yl, benzo[c][1,2,5]oxadiazolyl, benzofuranyl, biphenyl, chromanyl, isoquinolinyl, isoxazolyl, naphthalenyl, phenyl, phenylthiophenyl, pyridinyl, pyrrolo[1,2-a]pyrimidinyl, quinolinyl, and thiazolyl; wherein each is optionally substituted with one or more substituents selected from: 2-methylpropan-2-yl, amino, bromo, chloro, cyclopropyl, ethoxy, ethyl, fluoro, hydroxy, isopropoxy, methoxy, methyl, oxo, propan-2-yl, propan-1-yl, sulfamoyl, and trifluoromethyl; and wherein said 2-methylpropan-2-yl, cyclopropyl, ethyl, methyl, and propan-2-yl are each optionally substituted with one or more substituents selected from: 2,2,2-trifluoroethylamino, 2-aminoethylamino, 2-methoxyethylamino, 3-aminopropylamino, acetamido, amino, azetidin-1-yl, butylamino, cyclobutylamino, ethylamino, isobutylamino, isopropylamino, methoxy, methylamino, morpholino, propylamino, tert-butylamino, and tert-pentylamino;
R² is selected from: 1,1-difluoroethyl, 1-fluoroethyl, 2-methylpropan-2-yl, 3,3,3-trifluoropropyl, 4,4,4-trifluorobutyl, azetidin-3-yl, cyclobutyl, cyclopentyl, cyclopropyl, ethyl, fluoromethyl, isobutyl, isopentyl, isopropyl, methyl, oxetan-3-yl, propan-1-yl, sec-butyl, and vinyl; each optionally substituted with one or more substituents selected from: 2,2-difluorocyclopropyl, amino, cyano, cyclobutyl, cyclohexyl, cyclopropyl, ethoxy, hydroxy, hydroxymethyl, methoxy, oxo, and phenyl; and
R^{3a} and R^{3c} are each independently H or F.

One aspect of the present invention pertains to compounds of Formula (Ie) and pharmaceutically acceptable salts, solvates, and hydrates thereof:

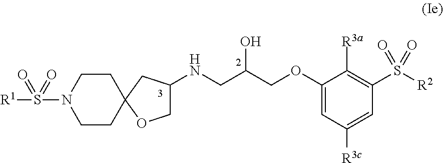

wherein:
R¹ is selected from: (R)-1,3-dimethyl-2,3-dihydro-1H-pyrido[2,3-b][1,4]oxazin-7-yl, (S)-1,3-dimethyl-2,3-dihydro-1H-pyrido[2,3-b][1,4]oxazin-7-yl, 1-(2-methoxyethyl)-2,3-dihydro-1H-pyrido[2,3-b][1,4]oxazin-7-yl, 1,3,3-trimethyl-2,3-dihydro-1H-pyrido[2,3-b][1,4]oxazin-6-yl, 1,4-dimethyl-1,2,3,4-tetrahydropyrido[3,2-b]pyrazin-7-yl, 1,6-dimethyl-2,3-dihydro-1H-pyrido[2,3-b][1,4]oxazin-7-yl, 1,8-dimethyl-2,3-dihydro-1H-pyrido[2,3-b][1,4]oxazin-7-yl, 1-ethyl-2,3-dihydro-1H-pyrido[2,3-b][1,4]oxazin-7-yl, 1-ethyl-4-oxo-1,4-dihydroquinolin-3-yl, 1-ethyl-5-methyl-1H-pyrazol-4-yl, 1-ethyl-6-fluoro-4-oxo-1,4-dihydroquinolin-3-yl, 1-ethyl-6-methyl-4-oxo-1,4-dihydroquinolin-3-yl, 1-ethyl-7-fluoro-4-oxo-1,4-dihydroquinolin-3-yl, 1-ethyl-7-methyl-4-oxo-1,4-dihydroquinolin-3-yl, 1-ethyl-8-fluoro-4-oxo-1,4-dihydroquinolin-3-yl, 1-ethyl-8-methyl-4-oxo-1,4-dihydroquinolin-3-yl, 1H-benzo[d]imidazol-5-yl, 1H-indazol-5-yl, 1H-indol-2-yl, 1H-indol-3-yl, 1H-indol-5-yl, 1H-indol-6-yl, 1H-pyrazolo[4,3-b]pyridin-6-yl, 1H-pyrrolo[2,3-b]pyridin-3-yl, 1H-pyrrolo[3,2-b]pyridin-6-yl, 1-methyl-2,3-dihydro-1H-pyrido[2,3-b][1,4]oxazin-7-yl, 1-methyl-4-oxo-1,4-dihydroquinolin-3-yl, 2,3-dihydro-[1,4]dioxino[2,3-b]pyridin-7-yl, 2,3-dihydro-1H-pyrido[2,3-b][1,4]oxazin-7-yl, 2,3-dihydrobenzofuran-5-yl, 2-aminothiazol-4-yl, 2-oxo-2,3-dihydro-1H-imidazo[4,5-b]pyridin-6-yl, 2-oxo-2,3-dihydro-1H-pyrrolo[2,3-b]pyridin-5-yl, 3-(1-cyclopropyl-1H-pyrazol-4-yl)phenyl, 3-(1-ethyl-1H-pyrazol-4-yl)phenyl, 3-(1H-pyrazol-4-yl)phenyl, 3-(1-methyl-1H-pyrazol-4-yl)phenyl, 3-(1-propyl-1H-pyrazol-4-yl)phenyl, 3-(2-methylpyridin-4-yl)phenyl, 3-(3-fluoropyridin-2-yl)phenyl, 3-(4-methylpyridin-2-yl)phenyl, 3-(5-methylpyridin-2-yl)phenyl, 3-(6-(trifluoromethyl)pyridin-2-yl)phenyl, 3-(6-aminopyridin-3-yl)phenyl, 3-(6-fluoropyridin-2-yl)phenyl, 3-(6-methylpyridin-2-yl)phenyl, 3-(pyridin-2-yl)phenyl, 3-(pyridin-3-yl)phenyl, 3-(pyridin-4-yl)phenyl, 3-(pyrimidin-5-yl)phenyl, 3,4-dihydro-2H-pyrano[2,3-b]pyridin-6-yl, 3,5-dimethylisoxazol-4-yl, 3-methyl-3H-imidazo[4,5-b]pyridin-5-yl, 3-methyl-3H-imidazo[4,5-b]pyridin-6-yl, 3-oxo-3,4-dihydro-2H-benzo[b][1,4]oxazin-6-yl, 4-(pyridin-2-yl)phenyl, 4-(pyridin-3-yl)phenyl, 4-(pyridin-4-yl)phenyl, 4-hydroxy-6-methylquinolin-3-yl, 4-hydroxy-7-methylquinolin-3-yl, 4-hydroxy-8-methylquinolin-3-yl, 4-hydroxyquinolin-3-yl, 4-methoxyquinolin-3-yl, 4-methyl-2-oxo-1,2-dihydroquinolin-6-yl, 4-methyl-3,4-dihydro-2H-benzo[b][1,4]oxazin-6-yl, 4-methyl-3,4-dihydro-2H-benzo[b][1,4]oxazin-7-yl, 4-methyl-3,4-dihydro-2H-pyrido[3,2-b][1,4]oxazin-7-yl, 4-oxo-1,4-dihydroquinolin-3-yl, 5-(1-methyl-1H-pyrazol-4-yl)pyridin-3-yl, 5-(4-(aminomethyl)phenyl)pyridin-3-yl, 5,6,7,8-tetrahydroquinolin-3-yl, 5-bromo-6-chloropyridin-3-yl, 5-bromopyridin-3-yl, 5-oxo-6,7-dihydro-5H-pyrrolo[3,4-b]pyridin-3-yl, 5-phenylthiophen-2-yl, 6-fluoro-4-hydroxyquinolin-3-yl, 7-chlorobenzo[c][1,2,5]oxadiazol-4-yl, 7-fluoro-4-hydroxyquinolin-3-yl, 8-fluoro-4-hydroxyquinolin-3-yl, benzofuran-2-yl, benzofuran-5-yl, chroman-6-yl, chroman-7-yl, isoquinolin-5-yl, pyridin-2-yl, pyridin-3-yl, pyrrolo[1,2-a]pyrimidin-3-yl, quinolin-3-yl, quinolin-6-yl, and quinolin-7-yl;
R² is selected from: (2,2-difluorocyclopropyl)methyl, 1-(hydroxymethyl)cyclobutyl, 1-(hydroxymethyl)cyclopropyl, 1,1-difluoro-2-hydroxyethyl, 1-amino-2-methyl-1-oxopropan-2-yl, 1-ethoxy-2-methyl-1-oxopropan-2-yl, 1-fluoroethyl, 1-hydroxy-2-methylpropan-2-yl, 2-amino-2-oxoethyl, 2-aminoethyl, 2-hydroxyethyl, 3,3,3-trifluoropropyl, 3-amino-3-oxopropyl, 3-hydroxycyclobutyl, 3-hydroxypropyl, 3-methoxypropyl, 4,4,4-trifluorobutyl, azetidin-3-yl, benzyl, carboxymethyl, cyanomethyl, cyclobutyl, cyclobutylmethyl, cyclohexylmethyl, cyclopentyl, cyclopropyl, cyclopropylmethyl, ethyl, fluoromethyl, isobutyl, isopentyl, isopropyl, methoxymethyl, methyl, oxetan-3-yl, propan-1-yl, sec-butyl, and vinyl; and
R^{3a} and R^{3c} are each independently H or F.

One aspect of the present invention pertains to compounds of Formula (Ig) and pharmaceutically acceptable salts, solvates, and hydrates thereof:

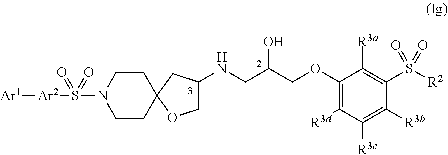

wherein:
Ar¹ and Ar² are independently 1H-pyrazolyl, phenyl, pyridinyl, pyrimidinyl, and thiophenyl, wherein each is optionally substituted with one or more substituents selected from: C₁-C₆ alkoxy, C₁-C₆ alkyl, amino, C₃-C₇ cycloalkyl, C₁-C₆ haloalkyl, halogen, and sulfamoyl; and wherein said C₁-C₆ alkyl and C₃-C₇ cycloalkyl are each optionally substituted with one or more substituents selected from: amino, C₁-C₆ alkylcarboxamide, —NH—C₃-C₇-cycloalkyl, —NH—C₁-C₆-alkylene-NH₂, —NH—C₁-C₆-alkylene-O—C₁-C₆-alkyl, C₁-C₆ alkylamino, C₁-C₆ haloalkylamino, and heterocyclyl;
R² is selected from: C₁-C₆ alkyl, C₃-C₇ cycloalkyl, and C₁-C₆ haloalkyl; each optionally substituted with one or more substituents selected from: C₁-C₆ alkoxy, C₁-C₆ alkylenehydroxyl, amino, hydroxyl, and oxo; and
R^{3a}, R^{3b}, R^{3c}, and R^{3d} are each independently H or halogen.

One aspect of the present invention pertains to compounds of Formula (Ig) and pharmaceutically acceptable salts, solvates, and hydrates thereof:

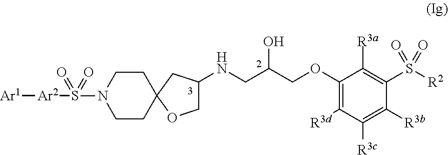

wherein:
Ar¹ and Ar² together form a group selected from: (1H-pyrazolyl)phenyl, (1H-pyrazolyl)pyridinyl, (phenyl)pyridinyl, (pyridinyl)phenyl, (pyrimidinyl)phenyl, biphenyl, and phenylthiophenyl, wherein each is optionally substituted with one or more substituents selected from: 2-methylpropan-2-yl, amino, cyclopropyl, ethoxy, ethyl, fluoro, isopropoxy, methoxy, methyl, n-propyl, propan-2-yl, sulfamoyl, and trifluoromethyl; and wherein said 2-methylpropan-2-yl, cyclopropyl, ethyl, methyl, and propan-2-yl are each optionally substituted with 2,2,2-trifluoroethylamino, 2-aminoethylamino, 2-methoxyethylamino, 3-aminopropylamino, acetamido, amino, azetidin-1-yl, butylamino, cyclobutylamino, ethylamino, isobutylamino, isopropylamino, isopropylamino, methylamino, morpholino, propylamino, tert-butylamino, and tert-pentylamino;
R² is selected from: 1,1-difluoroethyl, 2-methylpropan-2-yl, cyclopropyl, ethyl, isopropyl, and methyl; each optionally substituted with one or more substituents selected from: amino, hydroxy, hydroxymethyl, methoxy, and oxo; and
R^{3a}, R^{3b}, R^{3c}, and R^{3d} are each H.

One aspect of the present invention pertains to compounds of Formula (Ig) and pharmaceutically acceptable salts, solvates, and hydrates thereof:

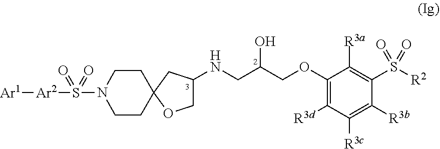

wherein:
Ar¹ and Ar² together form a group selected from: 3-(1H-pyrazol-4-yl)phenyl, 3-(pyridin-2-yl)phenyl, 3-(pyridin-3-yl)phenyl, 3-(pyridin-4-yl)phenyl, 3-(pyrimidin-5-yl)phenyl, 4-(pyridin-2-yl)phenyl, 4-(pyridin-3-yl)phenyl, 4-(pyridin-4-yl)phenyl, 5-(1H-pyrazol-4-yl)pyridin-3-yl, 5-(phenyl)pyridin-3-yl, 5-phenylthiophen-2-yl, biphenyl-3-yl, and biphenyl-4-yl, wherein each is optionally substituted with one or more substituents selected from: (2,2,2-trifluoroethylamino)methyl, (2-aminoethylamino)methyl, (2-methoxyethylamino)methyl, (3-aminopropylamino)methyl, (butylamino)methyl, (cyclobutylamino)methyl, (ethylamino)methyl, (isobutylamino)methyl, (isopropylamino)methyl, (methylamino)methyl, (propylamino)methyl, (tert-butylamino)methyl, (tert-pentylamino)methyl, 1-amino-2-methylpropan-2-yl, 1-aminocyclopropyl, 2-acetamidoethyl, 2-aminoethyl, 2-aminopropan-2-yl, amino, aminomethyl, azetidin-1-ylmethyl, cyclopropyl, ethoxy, ethyl, fluoro, isopropoxy, methoxy, methyl, morpholinomethyl, propyl, sulfamoyl, and trifluoromethyl;
R² is selected from: 1-(hydroxymethyl)cyclopropyl, 1,1-difluoro-2-hydroxyethyl, 1-hydroxy-2-methylpropan-2-yl, 2-amino-2-oxoethyl, 2-hydroxyethyl, cyclopropyl, ethyl, isopropyl, methoxymethyl, and methyl; and
R^{3a}, R^{3b}, R^{3c}, and R^{3d} are each H.

One aspect of the present invention pertains to compounds of Formula (Ig) and pharmaceutically acceptable salts, solvates, and hydrates thereof:

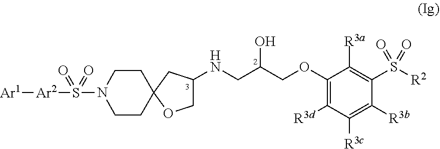

wherein:
Ar¹ and Ar² together form a group selected from: 3-(1-cyclopropyl-1H-pyrazol-4-yl)phenyl, 3-(1-ethyl-1H-pyrazol-4-yl)phenyl, 3-(1H-pyrazol-4-yl)phenyl, 3-(1-methyl-1H-pyrazol-4-yl)phenyl, 3-(1-propyl-1H-pyrazol-4-yl)phenyl, 3-(2-methylpyridin-4-yl)phenyl, 3-(3-fluoropyridin-2-yl)phenyl, 3-(4-methylpyridin-2-yl)phenyl, 3-(5-methylpyridin-2-yl)phenyl, 3-(6-(trifluoromethyl)pyridin-2-yl)phenyl, 3-(6-aminopyridin-3-yl)phenyl, 3-(6-fluoropyridin-2-yl)phenyl, 3-(6-methylpyridin-2-yl)phenyl, 3-(pyridin-2-yl)phenyl, 3-(pyridin-3-yl)phenyl, 3-(pyridin-4-yl)phenyl, 3-(pyrimidin-5-yl)phenyl, 4′-((2,2,2-trifluoroethylamino)methyl)biphenyl-3-yl, 4′-((2-aminoethylamino)methyl)biphenyl-3-yl, 4′-((2-methoxyethylamino)methyl)biphenyl-3-yl, 4′-((3-aminopropylamino)methyl)biphenyl-3-yl, 4′-((butylamino)methyl)biphenyl-3-yl, 4′-((cyclobutylamino)methyl)biphenyl-3-yl, 4′-((ethylamino)methyl)biphenyl-3-yl, 4′-((isobutylamino)methyl)biphenyl-3-yl, 4′-((isopropylamino)methyl)biphenyl-3-yl, 4′-((methylamino)methyl)biphenyl-3-yl, 4′-((propylamino)methyl)biphenyl-3-yl, 4′-((tert-butylamino)methyl)biphenyl-3-yl, 4′-((tert-pentylamino)methyl)biphenyl-3-yl, 4′-(1-amino-2-methylpropan-2-yl)-4-ethoxybiphenyl-3-yl, 4′-(1-amino-2-methylpropan-2-yl)biphenyl-3-yl, 4′-(1-aminocyclopropyl)-2-methylbiphenyl-3-yl, 4′-(1-aminocyclopropyl)-4-ethoxybiphenyl-3-yl, 4′-(1-aminocyclopropyl)-6-fluorobiphenyl-3-yl, 4′-(1-aminocyclopropyl)-6-methoxybiphenyl-3-yl, 4′-(1-aminocyclopropyl)biphenyl-3-yl, 4′-(2-acetamidoethyl)-4-ethoxy-biphenyl-3-yl, 4′-(2-acetamidoethyl)-biphenyl-3-yl, 4′-(2-aminoethyl)-4-ethoxybiphenyl-3-yl, 4′-(2-aminoethyl)-6-methoxybiphenyl-3-yl, 4′-(2-aminoethyl)biphenyl-3-yl, 4′-(2-aminopropan-2-yl)-4-ethoxybiphenyl-3-yl, 4′-(aminomethyl)-2-methoxybiphenyl-3-yl, 4′-(aminomethyl)-2-methylbiphenyl-3-yl, 4′-(aminomethyl)-3′-fluorobiphenyl-3-yl, 4′-(aminomethyl)-4-ethoxy-3′-fluorobiphenyl-3-yl, 4′-(aminomethyl)-4-ethoxybiphenyl-3-yl, 4′-(aminomethyl)-4-fluorobiphenyl-3-yl, 4′-(aminomethyl)-4-isopropoxybiphenyl-3-yl, 4′-(aminomethyl)-5-methoxybiphenyl-3-yl, 4′-(aminomethyl)-6-ethoxybiphenyl-3-yl, 4′-(aminomethyl)-6-fluorobiphenyl-3-yl, 4′-(aminomethyl)-6-methoxybiphenyl-3-yl, 4′-(aminomethyl)biphenyl-3-yl, 4′-(aminomethyl)biphenyl-4-yl, 4′-(azetidin-1-ylmethyl)biphenyl-3-yl, 4′-(morpholinomethyl)biphenyl-3-yl, 4-(pyridin-2-yl)phenyl, 4-(pyridin-3-yl)phenyl, 4-(pyridin-4-yl)phenyl, 4′-(sulfamoyl)biphenyl-3-yl, 4-ethoxy-4′-((isopropylamino)methyl)biphenyl-3-yl, 4′-methylbiphenyl-3-yl, 5-(1-methyl-1H-pyrazol-4-yl)pyridin-3-yl, 5-(4-(aminomethyl)phenyl)pyridin-3-yl, and 5-phenylthiophen-2-yl;
R² is selected from: 1-(hydroxymethyl)cyclopropyl, 1,1-difluoro-2-hydroxyethyl, 1-hydroxy-2-methylpropan-2-yl, 2-amino-2-oxoethyl, 2-hydroxyethyl, cyclopropyl, ethyl, isopropyl, methoxymethyl, and methyl; and
R^{3a}, R^{3b}, R^{3c}, and R^{3d} are each H.

One aspect of the present invention pertains to compounds of Formula (Ii) and pharmaceutically acceptable salts, solvates, and hydrates thereof:

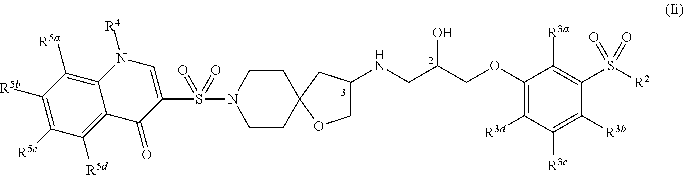

wherein:
R² is selected from: C₁-C₆ alkyl, C₃-C₇ cycloalkyl, and C₁-C₆ haloalkyl; each optionally substituted with one or more substituents selected from: C₁-C₆ alkoxy, C₁-C₆ alkylenehydroxyl, and hydroxyl;
R^{3a}, R^{3b}, R^{3c}, and R^{3d} are each independently H or halogen;
R⁴ is H or C₁-C₆ alkyl; and
R^{5a}, R^{5b}, R^{5c}, and R^{5d} are independently H, C₁-C₆ alkyl, and halogen.

One aspect of the present invention pertains to compounds of Formula (Ii) and pharmaceutically acceptable salts, solvates, and hydrates thereof:

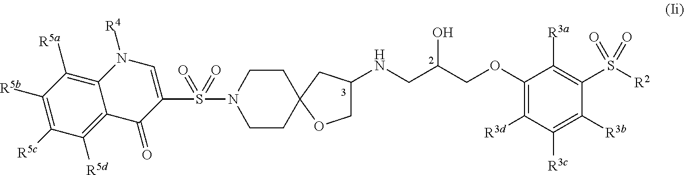

wherein:
R² is selected from: 1,1-difluoroethyl, 2-methylpropan-2-yl, cyclopropyl, ethyl, 1-fluoroethyl, isopropyl, and methyl; each optionally substituted with one or more substituents selected from: hydroxy, hydroxymethyl, and methoxy;
R^{3a}, R^{3b}, R^{3c}, and R^{3d} are each H;
R⁴ is selected from: H, methyl, and ethyl; and
R^{5a}, R^{5b}, R^{5c}, and R^{5d} are independently H, methyl, and fluoro.

One aspect of the present invention pertains to compounds of Formula (Ii) and pharmaceutically acceptable salts, solvates, and hydrates thereof:

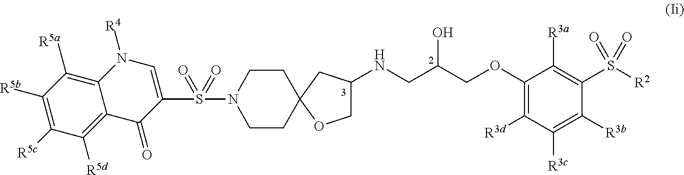

wherein:
R² is selected from: 1-(hydroxymethyl)cyclopropyl, 1,1-difluoro-2-hydroxyethyl, 1-fluoroethyl, 1-hydroxy-2-methylpropan-2-yl, cyclopropyl, isopropyl, methoxymethyl, and methyl;
R^{3a}, R^{3b}, R^{3c}, and R^{3d} are each H;
R⁴ is selected from: H, methyl, and ethyl;
R^{5a}, R^{5b}, and R^{5c} are independently H, methyl, and fluoro; and
R^{5d} is H.

Some embodiments of the present invention include every combination of one or more compounds and pharmaceutically acceptable salts, solvates, and hydrates thereof selected from the following group shown below.

| Cmpd | | |
|---|---|---|
| No. | Chemical Structure | Chemical Name |
| A5 | 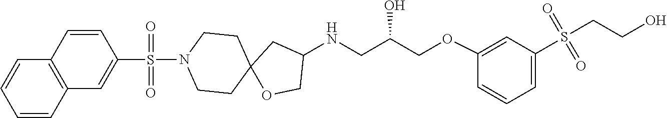 [SMILES: O=S(=O)(CCO)c1cccc(OC[C@@H](O)CNC2COC3(CCN(S(=O)(=O)c4ccc5ccccc5c4)CC3)C2)c1] | (2S)-1-(3-(2- hydroxyethylsulfonyl) phenoxy)-3-(8- (naphthalen-2- ylsulfonyl)-1-oxa-8- azaspiro[4.5]decan-3- ylamino)propan-2-ol |
| A88 | 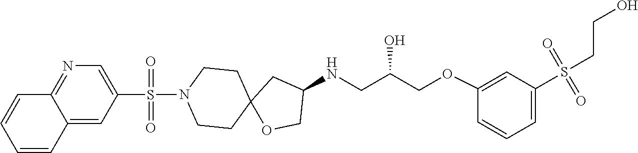 [SMILES: O=S(=O)(CCO)c1cccc(OC[C@@H](O)CN[C@H]2COC3(CCN(S(=O)(=O)c4cnc5ccccc5c4)CC3)C2)c1] | (S)-1-(3-(2- hydroxyethylsulfonyl) phenoxy)-3-((R)-8- (quinolin-3-ylsulfonyl)- 1-oxa-8- azaspiro[4.5]decan-3- ylamino)propan-2-ol |
| A123 | 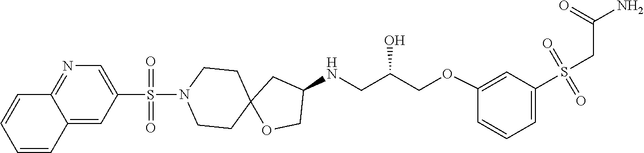 [SMILES: NC(=O)CS(=O)(=O)c1cccc(OC[C@@H](O)CN[C@H]2COC3(CCN(S(=O)(=O)c4cnc5ccccc5c4)CC3)C2)c1] | 2-(3-((S)-2-hydroxy-3- ((R)-8-(quinolin-3- ylsulfonyl)-1-oxa-8- azaspiro[4.5]decan-3- ylamino)propoxy) phenylsulfonyl)acetamide |
| A136 | 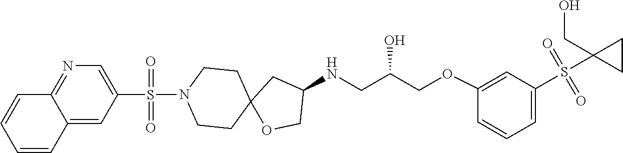 [SMILES: O=S(=O)(c1cnc2ccccc2c1)N1CCC2(CC1)C[C@@H](NC[C@H](O)COc1cccc(S(=O)(=O)C3(CO)CC3)c1)CO2] | (S)-1-(3-(1- (hydroxymethyl)cyclo- propylsulfonyl)phenoxy)- 3-((R)-8-(quinolin- 3-ylsulfonyl)-1-oxa-8- azaspiro[4.5]decan-3- ylamino)propan-2-ol |
| A154 | 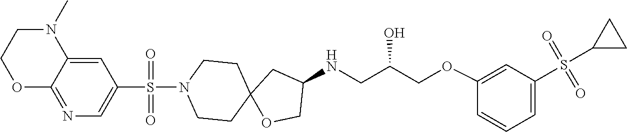 [SMILES: CN1CCOc2ncc(S(=O)(=O)N3CCC4(CC3)C[C@@H](NC[C@H](O)COc3cccc(S(=O)(=O)C5CC5)c3)CO4)cc21] | (S)-1-(3- (cyclopropylsulfonyl) phenoxy)-3-((R)-8-(1- methyl-2,3-dihydro- 1H-pyrido[2,3- b][1,4]oxazin-7- ylsulfonyl)-1-oxa-8- azaspiro[4.5]decan-3- ylamino)propan-2-ol |
| A161 | 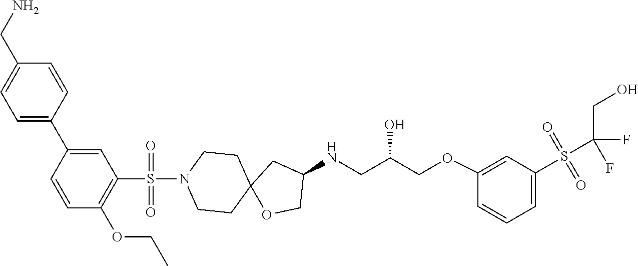 [SMILES: CCOc1ccc(-c2ccc(CN)cc2)cc1S(=O)(=O)N1CCC2(CC1)C[C@@H](NC[C@H](O)COc1cccc(S(=O)(=O)C(F)(F)CO)c1)CO2] | (S)-1-((R)-8-(4′- (aminomethyl)-4- ethoxybiphenyl-3- ylsulfonyl)-1-oxa-8- azaspiro[4.5]decan-3- ylamino)-3-(3-(1,1- difluoro-2- hydroxyethylsulfonyl) phenoxy)propan-2-ol |
| A163 | 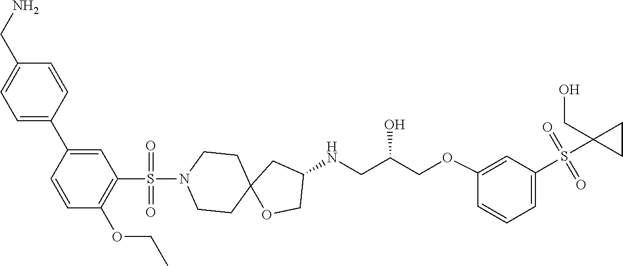 [SMILES: CCOc1ccc(-c2ccc(CN)cc2)cc1S(=O)(=O)N1CCC2(CC1)C[C@H](NC[C@H](O)COc1cccc(S(=O)(=O)C3(CO)CC3)c1)CO2] | (S)-1-((S)-8-(4′- (aminomethyl)-4- ethoxybiphenyl-3- ylsulfonyl)-1-oxa-8- azaspiro[4.5]decan-3- ylamino)-3-(3-(1- (hydroxymethyl)cyclo- propylsulfonyl)phenoxy) propan-2-ol |
| A169 | 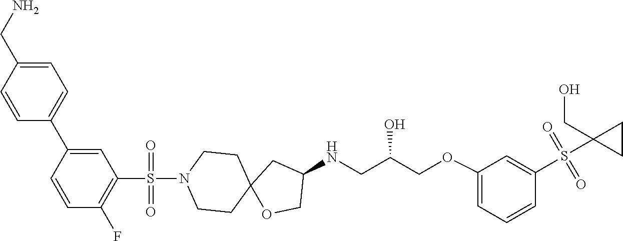 [SMILES: NCc1ccc(-c2ccc(F)c(S(=O)(=O)N3CCC4(CC3)C[C@@H](NC[C@H](O)COc3cccc(S(=O)(=O)C5(CO)CC5)c3)CO4)c2)cc1] | (S)-1-((R)-8-(4′- (aminomethyl)-4- fluorobiphenyl-3- ylsulfonyl)-1-oxa-8- azaspiro[4.5]decan-3- ylamino)-3-(3-(1- (hydroxymethyl)cyclo- propylsulfonyl)phenoxy) propan-2-ol |
| A199 | 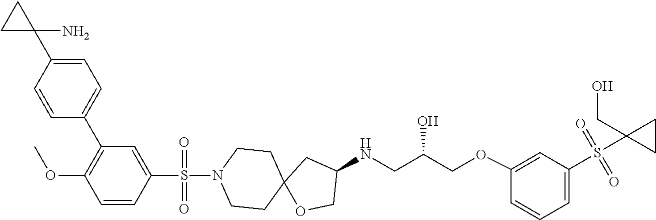 [SMILES: COc1ccc(S(=O)(=O)N2CCC3(CC2)C[C@@H](NC[C@H](O)COc2cccc(S(=O)(=O)C4(CO)CC4)c2)CO3)cc1-c1ccc(C2(N)CC2)cc1] | (S)-1-((R)-8-(4′-(1- aminocyclopropyl)-6- methoxybiphenyl-3- ylsulfonyl)-1-oxa-8- azaspiro[4.5]decan-3- ylamino)-3-(3-(1- (hydroxymethyl)cyclo- propylsulfonyl)phenoxy) propan-2-ol |
| A210 | 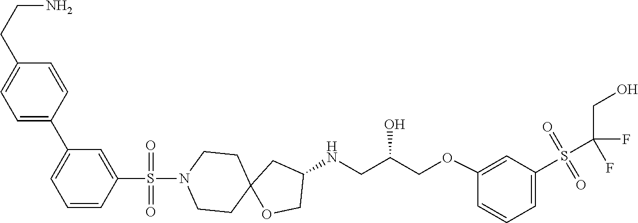 [SMILES: NCCc1ccc(-c2cccc(S(=O)(=O)N3CCC4(CC3)C[C@H](NC[C@H](O)COc3cccc(S(=O)(=O)C(F)(F)CO)c3)CO4)c2)cc1] | (S)-1-((S)-8-(4′-(2- aminoethyl)biphenyl- 3-ylsulfonyl)-1-oxa-8- azaspiro[4.5]decan-3- ylamino)-3-(3-(1,1- difluoro-2- hydroxyethylsulfonyl) phenoxy)propan-2-ol |
| A211 | 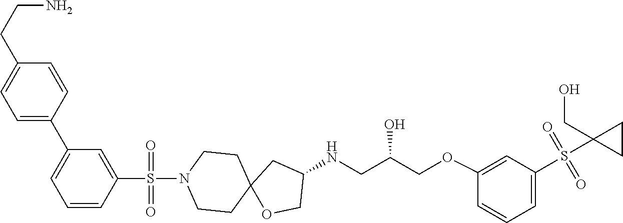 [SMILES: NCCc1ccc(-c2cccc(S(=O)(=O)N3CCC4(CC3)C[C@H](NC[C@H](O)COc3cccc(S(=O)(=O)C5(CO)CC5)c3)CO4)c2)cc1] | (S)-1-((S)-8-(4′-(2- aminoethyl)biphenyl- 3-ylsulfonyl)-1-oxa-8- azaspiro[4.5]decan-3- ylamino)-3-(3-(1- (hydroxymethyl)cyclo- propylsulfonyl)phenoxy) propan-2-ol |
| A217 | 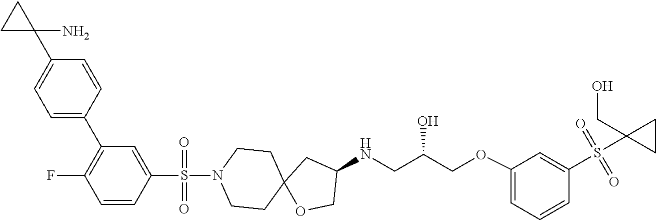 [SMILES: NC1(c2ccc(-c3cc(S(=O)(=O)N4CCC5(CC4)C[C@@H](NC[C@H](O)COc4cccc(S(=O)(=O)C6(CO)CC6)c4)CO5)ccc3F)cc2)CC1] | (S)-1-((R)-8-(4′-(1- aminocyclopropyl)-6- fluorobiphenyl-3- ylsulfonyl)-1-oxa-8- azaspiro[4.5]decan-3- ylamino)-3-(3-(1- (hydroxymethyl)cyclo- propylsulfonyl)phenoxy) propan-2-ol |
| A220 | 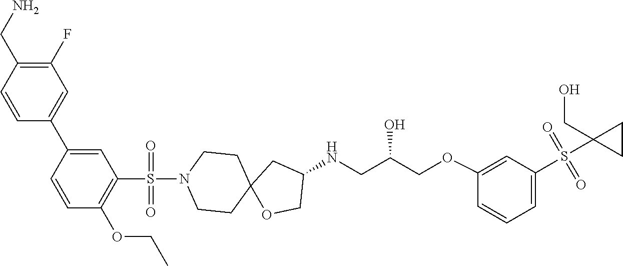 [SMILES: CCOc1ccc(-c2ccc(CN)c(F)c2)cc1S(=O)(=O)N1CCC2(CC1)C[C@H](NC[C@H](O)COc1cccc(S(=O)(=O)C3(CO)CC3)c1)CO2] | (S)-1-((S)-8-(4′- (aminomethyl)-4- ethoxy-3′- fluorobiphenyl-3- ylsulfonyl)-1-oxa-8- azaspiro[4.5]decan-3- ylamino)-3-(3-(1- (hydroxymethyl)cyclo- propylsulfonyl)phenoxy) propan-2-ol |
| A225 | 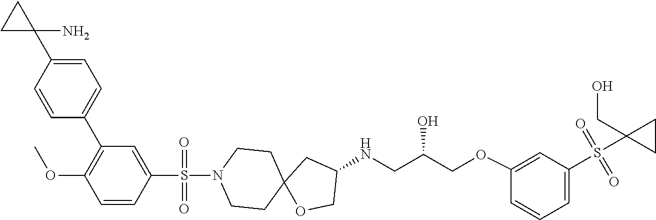 [SMILES: COc1ccc(S(=O)(=O)N2CCC3(CC2)C[C@H](NC[C@H](O)COc2cccc(S(=O)(=O)C4(CO)CC4)c2)CO3)cc1-c1ccc(C2(N)CC2)cc1] | (S)-1-((S)-8-(4′-(1- aminocyclopropyl)-6- methoxybiphenyl-3- ylsulfonyl)-1-oxa-8- azaspiro[4.5]decan-3- ylamino)-3-(3-(1- (hydroxymethyl)cyclo- propylsulfonyl)phenoxy) propan-2-ol |
| A227 | 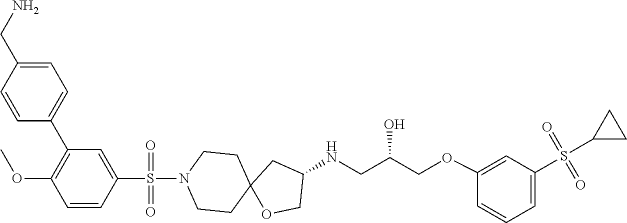 [SMILES: COc1ccc(S(=O)(=O)N2CCC3(CC2)C[C@H](NC[C@H](O)COc2cccc(S(=O)(=O)C4CC4)c2)CO3)cc1-c1ccc(CN)cc1] | (S)-1-((S)-8-(4′- (aminomethyl)-6- methoxybiphenyl-3- ylsulfonyl)-1-oxa-8- azaspiro[4.5]decan-3- ylamino)-3-(3- (cyclopropylsulfonyl) phenoxy)propan-2-ol |
| A229 | 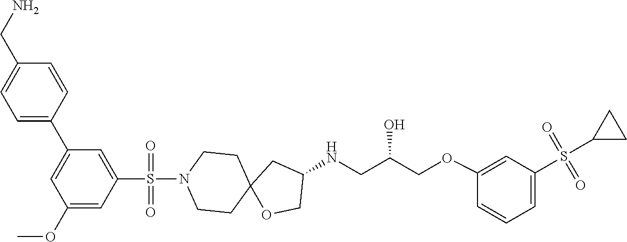 [SMILES: COc1cc(-c2ccc(CN)cc2)cc(S(=O)(=O)N2CCC3(CC2)C[C@H](NC[C@H](O)COc2cccc(S(=O)(=O)C4CC4)c2)CO3)c1] | (S)-1-((S)-8-(4′- (aminomethyl)-5- methoxybiphenyl-3- ylsulfonyl)-1-oxa-8- azaspiro[4.5]decan-3- ylamino)-3-(3- (cyclopropylsulfonyl) phenoxy)propan-2-ol |
| A230 | 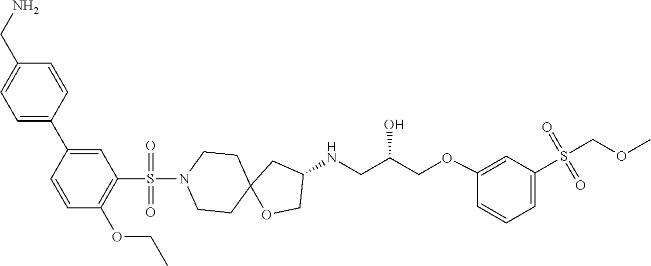 [SMILES: CCOc1ccc(-c2ccc(CN)cc2)cc1S(=O)(=O)N1CCC2(CC1)C[C@H](NC[C@H](O)COc1cccc(S(=O)(=O)COC)c1)CO2] | (S)-1-((S)-8-(4′- (aminomethyl)-4- ethoxybiphenyl-3- ylsulfonyl)-1-oxa-8- azaspiro[4.5]decan-3- ylamino)-3-(3- (methoxymethylsulfonyl) phenoxy)propan-2-ol |
| A232 | 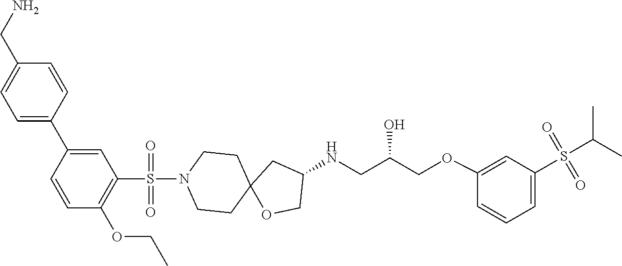 [SMILES: CCOc1ccc(-c2ccc(CN)cc2)cc1S(=O)(=O)N1CCC2(CC1)C[C@H](NC[C@H](O)COc1cccc(S(=O)(=O)C(C)C)c1)CO2] | (S)-1-((S)-8-(4′- (aminomethyl)-4- ethoxybiphenyl-3- ylsulfonyl)-1-oxa-8- azaspiro[4.5]decan-3- ylamino)-3-(3- (isopropylsulfonyl) phenoxy)propan-2-ol |
| A234 | 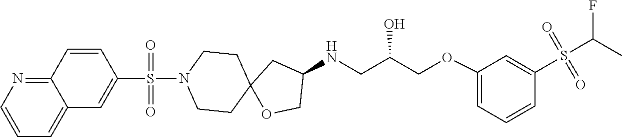 [SMILES: CC(F)S(=O)(=O)c1cccc(OC[C@@H](O)CN[C@H]2COC3(CCN(S(=O)(=O)c4ccc5ncccc5c4)CC3)C2)c1] | (2S)-1-(3-(1- fluoroethylsulfonyl) phenoxy)-3-((R)-8- (quinolin-6-ylsulfonyl)- 1-oxa-8- azaspiro[4.5]decan-3- ylamino)propan-2-ol |
| A240 | 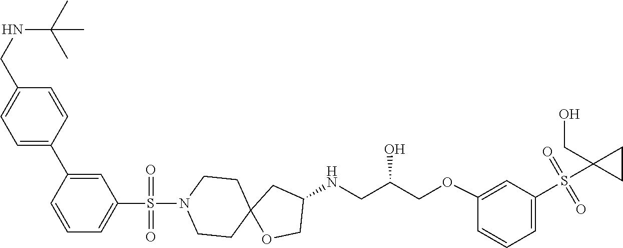 [SMILES: CC(C)(C)NCc1ccc(-c2cccc(S(=O)(=O)N3CCC4(CC3)C[C@H](NC[C@H](O)COc3cccc(S(=O)(=O)C5(CO)CC5)c3)CO4)c2)cc1] | (S)-1-((S)-8-(4′-((tert- butylamino)methyl) biphenyl-3-ylsulfonyl)-1- oxa-8- azaspiro[4.5]decan-3- ylamino)-3-(3-(1- (hydroxymethyl)cyclo- propylsulfonyl)phenoxy) propan-2-ol |
| A241 | 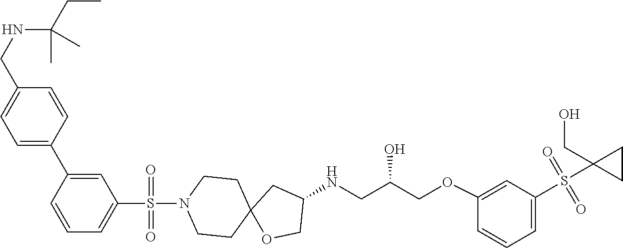 [SMILES: CCC(C)(C)NCc1ccc(-c2cccc(S(=O)(=O)N3CCC4(CC3)C[C@H](NC[C@H](O)COc3cccc(S(=O)(=O)C5(CO)CC5)c3)CO4)c2)cc1] | (S)-1-(3-(1- (hydroxymethyl)cyclo- propylsulfonyl)phenoxy)- 3-((S)-8-(4′-((tert- pentylamino)methyl) biphenyl-3-ylsulfonyl)-1- oxa-8- azaspiro[4.5]decan-3- ylamino)propan-2-ol |
| A243 | 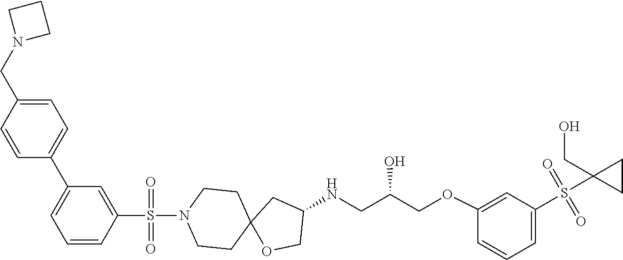 [SMILES: O=S(=O)(c1cccc(-c2ccc(CN3CCC3)cc2)c1)N1CCC2(CC1)C[C@H](NC[C@H](O)COc1cccc(S(=O)(=O)C3(CO)CC3)c1)CO2] | (S)-1-((S)-8-(4′- (azetidin-1- ylmethyl)biphenyl-3- ylsulfonyl)-1-oxa-8- azaspiro[4.5]decan-3- ylamino)-3-(3-(1- (hydroxymethyl)cyclo- propylsulfonyl)phenoxy) propan-2-ol |
| A244 | 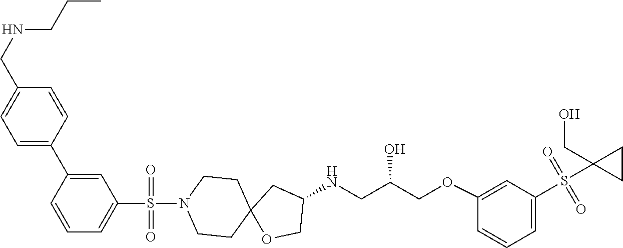 [SMILES: CCCNCc1ccc(-c2cccc(S(=O)(=O)N3CCC4(CC3)C[C@H](NC[C@H](O)COc3cccc(S(=O)(=O)C5(CO)CC5)c3)CO4)c2)cc1] | (S)-1-(3-(1- (hydroxymethyl)cyclo- propylsulfonyl)phenoxy)- 3-((S)-8-(4′- ((propylamino)methyl) biphenyl-3-ylsulfonyl)- 1-oxa-8- azaspiro[4.5]decan-3- ylamino)propan-2-ol |
| A245 | 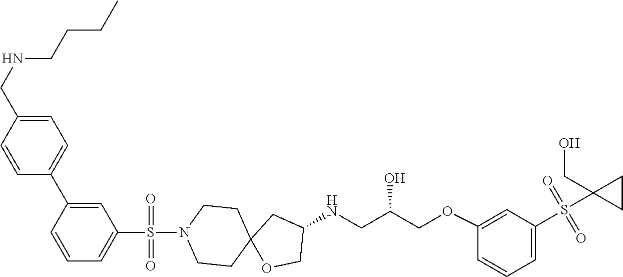 [SMILES: CCCCNCc1ccc(-c2cccc(S(=O)(=O)N3CCC4(CC3)C[C@H](NC[C@H](O)COc3cccc(S(=O)(=O)C5(CO)CC5)c3)CO4)c2)cc1] | (S)-1-((S)-8-(4′- ((butylamino)methyl) biphenyl-3-ylsulfonyl)-1- oxa-8- azaspiro[4.5]decan-3- ylamino)-3-(3-(1- (hydroxymethyl)cyclo- propylsulfonyl)phenoxy) propan-2-ol |
| A247 | 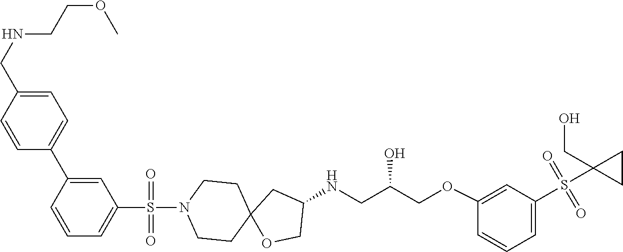 [SMILES: COCCNCc1ccc(-c2cccc(S(=O)(=O)N3CCC4(CC3)C[C@H](NC[C@H](O)COc3cccc(S(=O)(=O)C5(CO)CC5)c3)CO4)c2)cc1] | (S)-1-(3-(1- (hydroxymethyl)cyclo- propylsulfonyl)phenoxy)- 3-((S)-8-(4′-((2- methoxyethylamino) methyl)biphenyl-3- ylsulfonyl)-1-oxa-8- azaspiro[4.5]decan-3- ylamino)propan-2-ol |
| A296 | 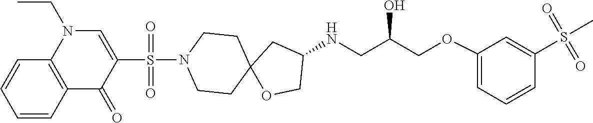 [SMILES: CCn1cc(S(=O)(=O)N2CCC3(CC2)C[C@H](NC[C@@H](O)COc2cccc(S(C)(=O)=O)c2)CO3)c(=O)c2ccccc21] | 1-ethyl-3-((S)-3-((R)- 2-hydroxy-3-(3- (methylsulfonyl) phenoxy)propylamino)- 1-oxa-8- azaspiro[4.5]decan-8- ylsulfonyl)quinolin- 4(1H)-one |
| A297 | 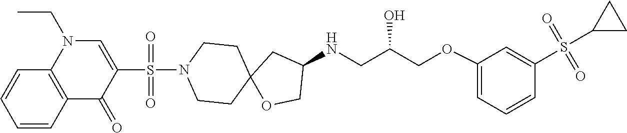 [SMILES: CCn1cc(S(=O)(=O)N2CCC3(CC2)C[C@@H](NC[C@H](O)COc2cccc(S(=O)(=O)C4CC4)c2)CO3)c(=O)c2ccccc21] | 3-((R)-3-((S)-3-(3- (cyclopropylsulfonyl) phenoxy)-2- hydroxypropylamino)- 1-oxa-8- azaspiro[4.5]decan-8- ylsulfonyl)-1- ethylquinolin-4(1H)- one |
| A300 | 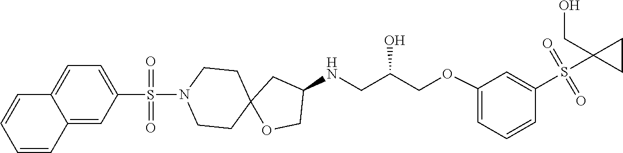 [SMILES: O=S(=O)(c1ccc2ccccc2c1)N1CCC2(CC1)C[C@@H](NC[C@H](O)COc1cccc(S(=O)(=O)C3(CO)CC3)c1)CO2] | (S)-1-(3-(1- (hydroxymethyl)cyclo- propylsulfonyl)phenoxy)- 3-((R)-8- (naphthalen-2- ylsulfonyl)-1-oxa-8- azaspiro[4.5]decan-3- ylamino)propan-2-ol |
| A309 | 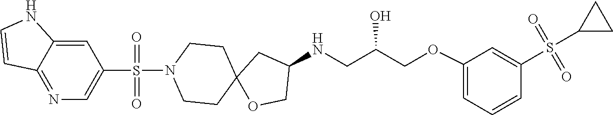 [SMILES: O=S(=O)(c1cccc(OC[C@@H](O)CN[C@H]2COC3(CCN(S(=O)(=O)c4cnc5cc[nH]c5c4)CC3)C2)c1)C1CC1] | (S)-1-((R)-8-(1H- pyrrolo[3,2-b]pyridin- 6-ylsulfonyl)-1-oxa-8- azaspiro[4.5]decan-3- ylamino)-3-(3- (cyclopropylsulfonyl) phenoxy)propan-2-ol |
| A310 | 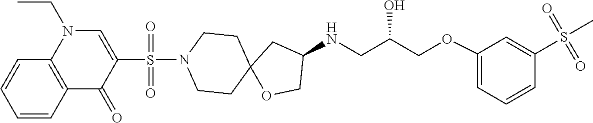 [SMILES: CCn1cc(S(=O)(=O)N2CCC3(CC2)C[C@@H](NC[C@H](O)COc2cccc(S(C)(=O)=O)c2)CO3)c(=O)c2ccccc21] | 1-ethyl-3-((R)-3-((S)- 2-hydroxy-3-(3- (methylsulfonyl) phenoxy)propylamino)- 1-oxa-8- azaspiro[4.5]decan-8- ylsulfonyl)quinolin- 4(1H)-one |
| A320 | 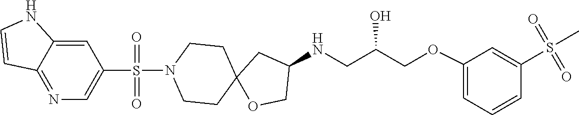 [SMILES: CS(=O)(=O)c1cccc(OC[C@@H](O)CN[C@H]2COC3(CCN(S(=O)(=O)c4cnc5cc[nH]c5c4)CC3)C2)c1] | (S)-1-((R)-8-(1H- pyrrolo[3,2-b]pyridin- 6-ylsulfonyl)-1-oxa-8- azaspiro[4.5]decan-3- ylamino)-3-(3- (methylsulfonyl) phenoxy)propan-2-ol |
| A321 | 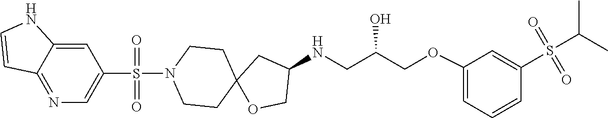 [SMILES: CC(C)S(=O)(=O)c1cccc(OC[C@@H](O)CN[C@H]2COC3(CCN(S(=O)(=O)c4cnc5cc[nH]c5c4)CC3)C2)c1] | (S)-1-((R)-8-(1H- pyrrolo[3,2-b]pyridin- 6-ylsulfonyl)-1-oxa-8- azaspiro[4.5]decan-3- ylamino)-3-(3- (isopropylsulfonyl) phenoxy)propan-2-ol |
| A322 | 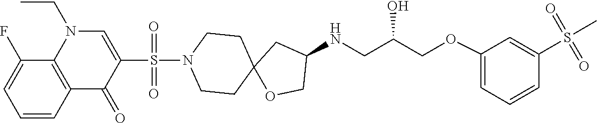 [SMILES: CCn1cc(S(=O)(=O)N2CCC3(CC2)C[C@@H](NC[C@H](O)COc2cccc(S(C)(=O)=O)c2)CO3)c(=O)c2cccc(F)c21] | 1-ethyl-8-fluoro-3- ((R)-3-((S)-2-hydroxy- 3-(3-(methylsulfonyl) phenoxy)propylamino)- 1-oxa-8- azaspiro[4.5]decan-8- ylsulfonyl)quinolin- 4(1H)-one |
| A326 | 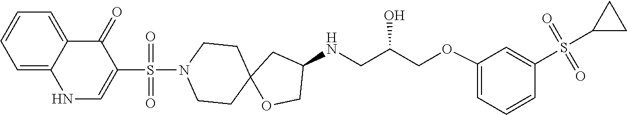 [SMILES: O=c1c(S(=O)(=O)N2CCC3(CC2)C[C@@H](NC[C@H](O)COc2cccc(S(=O)(=O)C4CC4)c2)CO3)c[nH]c2ccccc12] | 3-((R)-3-((S)-3-(3- (cyclopropylsulfonyl) phenoxy)-2- hydroxypropylamino)- 1-oxa-8- azaspiro[4.5]decan-8- ylsulfonyl)quinolin- 4(1H)-one |
| A327 | 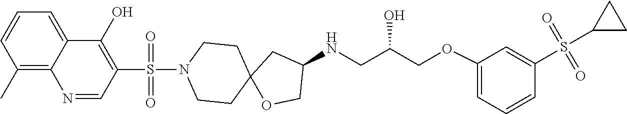 [SMILES: Cc1cccc2c(O)c(S(=O)(=O)N3CCC4(CC3)C[C@@H](NC[C@H](O)COc3cccc(S(=O)(=O)C5CC5)c3)CO4)cnc12] | 3-((R)-3-((S)-3-(3- (cyclopropylsulfonyl) phenoxy)-2- hydroxypropylamino)- 1-oxa-8- azaspiro[4.5]decan-8- ylsulfonyl)-8- methylquinolin-4-ol |
| A329 | 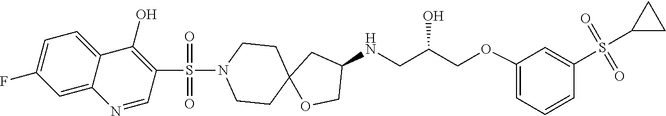 [SMILES: O=S(=O)(c1cccc(OC[C@@H](O)CN[C@H]2COC3(CCN(S(=O)(=O)c4cnc5cc(F)ccc5c4O)CC3)C2)c1)C1CC1] | 3-((R)-3-((S)-3-(3- (cyclopropylsulfonyl) phenoxy)-2- hydroxypropylamino)- 1-oxa-8- azaspiro[4.5]decan-8- ylsulfonyl)-7- fluoroquinolin-4-ol |
| A331 | 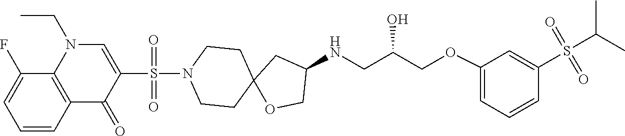 [SMILES: CCn1cc(S(=O)(=O)N2CCC3(CC2)C[C@@H](NC[C@H](O)COc2cccc(S(=O)(=O)C(C)C)c2)CO3)c(=O)c2cccc(F)c21] | 1-ethyl-8-fluoro-3- ((R)-3-((S)-2-hydroxy- 3-(3-(isopropylsulfonyl) phenoxy)propylamino)- 1-oxa-8- azaspiro[4.5]decan-8- ylsulfonyl)quinolin- 4(1H)-one |

One aspect of the present invention encompasses, inter alia, certain 1-oxa-8-azaspiro[4.5]decan-3-yl-aminopropanyl-ether derivatives selected from compounds of Formula (IIa) and pharmaceutically acceptable salts, solvates, hydrates, and N-oxides thereof:

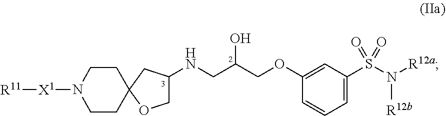

wherein: R¹¹ (as well as Y¹ and Z¹ that are both related to R¹¹), X¹, R^{12a}, and R^{12b} all have the same definitions as described herein, supra and intra.

In some embodiments, compounds of the present can have the following defined stereochemistry as shown in Formula (IIa-1):

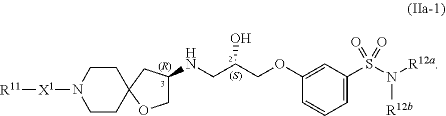

wherein: R¹¹, X¹, R^{12a}, and R^{12b}, have the same definitions as described herein, supra and infra, and wherein the carbon designated as C(3) of the oxa-azaspiro[4.5]decanyl group bonded to the nitrogen has the (R) stereochemistry and the carbon designated as C(2) of the propyl group bonded to the hydroxyl group has the (S) stereochemistry.

In some embodiments, compounds of the present can have the following defined stereochemistry as shown in Formula (IIa-2):

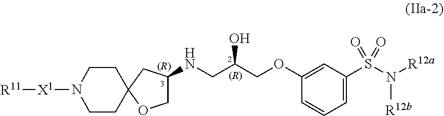

wherein: R¹¹, X¹, R^{12a}, and R^{12b}, have the same definitions as described herein, supra and infra, and wherein the carbon designated as C(3) of the oxa-azaspiro[4.5]decanyl group bonded to the nitrogen has the (R) stereochemistry and the carbon designated as C(2) of the propyl group bonded to the hydroxyl group has the (R) stereochemistry.

In some embodiments, compounds of the present can have the following defined stereochemistry as shown in Formula (IIa-3):

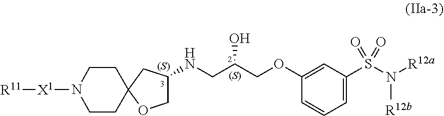

wherein: R¹¹, X¹, R^{12a}, and R^{12b}, have the same definitions as described herein, supra and infra, and wherein the carbon designated as C(3) of the oxa-azaspiro[4.5]decanyl group bonded to the nitrogen has the (S) stereochemistry and the carbon designated as C(2) of the propyl group bonded to the hydroxyl group has the (S) stereochemistry.

In some embodiments, compounds of the present can have the following defined stereochemistry as shown in Formula (IIa-4):

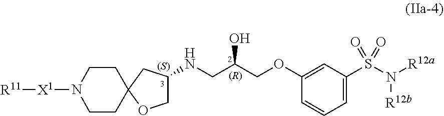

wherein: R¹¹, X¹, R^{12a}, and R^{12b}, have the same definitions as described herein, supra and infra, and wherein the carbon designated as C(3) of the oxa-azaspiro[4.5]decanyl group bonded to the nitrogen has the (S) stereochemistry and the carbon designated as C(2) of the propyl group bonded to the hydroxyl group has the (R) stereochemistry.

It is understood that any formulae described herein for which the stereochemistry is not specifically shown can be written to specifically show the stereochemistry as (R) and (S), (R) and (R), (S) and (S), or (S) and (R) for C(3) and C(2) respectively in a similar manner as Formulae (IIa-1), (IIa-2), (IIa-3), and IIa-4) shows the respective stereochemistry for Formula (IIa), supra. Similarly, any chemical name described herein for which the stereochemistry is not specifically shown can alternatively be defined using the language as described for Formulae (IIa-1), (IIa-2), (IIa-3), and IIa-4), supra, to define the stereochemistry for the chemical name as (R) and (S), (R) and (R), (S) and (S), and/or (S) and (R) respectively.

Accordingly, in some embodiments, the stereochemistry for the C(3) carbon of the oxa-azaspiro[4.5]decanyl group bonded to the nitrogen is (R). In some embodiments, the stereochemistry for the C(3) carbon of the oxa-azaspiro[4.5]decanyl group bonded to the nitrogen is (S). In some embodiments, the stereochemistry for the C(2) carbon of the propyl group bonded to the hydroxyl group is (S). In some embodiments, the stereochemistry for the C(2) carbon of the propyl group bonded to the hydroxyl group is (R). In some embodiments, the stereochemistry for the C(3) carbon of the oxa-azaspiro[4.5]decanyl group bonded to the nitrogen is (R) and the stereochemistry for the C(2) carbon of the propyl group bonded to the hydroxyl group is (S). In some embodiments, the stereochemistry for the C(3) carbon of the oxa-azaspiro[4.5]decanyl group bonded to the nitrogen is (R) and the stereochemistry for the C(2) carbon of the propyl group bonded to the hydroxyl group is (R). In some embodiments, the stereochemistry for the C(3) carbon of the oxa-azaspiro[4.5]decanyl group bonded to the nitrogen is (S) and the stereochemistry for the C(2) carbon of the propyl group bonded to the hydroxyl group is (S). In some embodiments, the stereochemistry for the C(3) carbon of the oxa-azaspiro[4.5]decanyl group bonded to the nitrogen is (S) and the stereochemistry for the C(2) carbon of the propyl group bonded to the hydroxyl group is (R).

It is understood that compounds of Formula (IIa) and the formulae used throughout this disclosure represent all individual enantiomers and mixtures thereof, unless specifically stated or shown otherwise.

### The X¹ Group

In some embodiments, X¹ is —SO₂— or absent.

In some embodiments, X¹ is —SO₂—.

In some embodiments, the present invention relates to compounds of Formula (IIb-1) and pharmaceutically acceptable salts, solvates, hydrates, and N-oxides thereof:

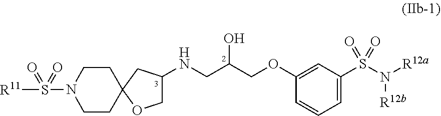

wherein: R¹¹, R^{12a}, and R^{12b} have the same definitions as described herein, supra and infra, and each can be selected independently from any of the embodiments as described herein, supra and infra.

In some embodiments, X¹ is absent.

In some embodiments, the present invention relates to compounds of Formula (IIb-2) and pharmaceutically acceptable salts, solvates, hydrates, and N-oxides thereof:

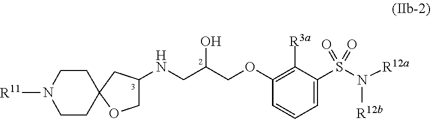

wherein: R¹¹, R^{12a}, and R^{12b} have the same definitions as described herein, supra and infra, and each can be selected independently from any of the embodiments as described herein, supra and infra.
The Y¹ and Z¹ Groups

The Y¹ and Z¹ groups are related to —Y¹-C₁-C₆-alkylene-Z¹ optionally substituted with oxo.

In some embodiments, Y¹ is selected from: —O— and —NH—; and Z¹ is selected from: C₁-C₆ alkoxy, amino, C₁-C₆ alkylamino, cyano, C₂-C₆ dialkylamino, hydroxyl, and phenyl.

In some embodiments, Y¹ is —NH—; and Z¹ is selected from: C₁-C₆ alkoxy, amino, cyano, C₂-C₆ dialkylamino, and hydroxyl.

In some embodiments, Y¹ is selected from: —O— and —NH—; and Z¹ is selected from: C₁-C₆ alkoxy, amino, cyano, C₂-C₆ dialkylamino, hydroxyl, and phenyl.

In some embodiments, Y¹ is —O—; and Z¹ is phenyl.

In some embodiments, Y¹ is selected from: —O— and —NH—.

In some embodiments, Y¹ is —O—.

In some embodiments, Y¹ is —NH—.

In some embodiments, Z¹ is selected from: C₁-C₆ alkoxy, amino, C₁-C₆ alkylamino, cyano, C₂-C₆ dialkylamino, hydroxyl, and phenyl. In some embodiments, Z¹ is C₁-C₆ alkoxy. In some embodiments, Z¹ is amino. In some embodiments, Z¹ is C₁-C₆ alkylamino. In some embodiments, Z¹ is cyano. In some embodiments, Z¹ is C₂-C₆ dialkylamino. In some embodiments, Z¹ is hydroxyl. In some embodiments, Z¹ is phenyl.

### The R¹¹ Group

In some embodiments, R¹¹ is selected from: C₁-C₆-alkylene-aryl, C₁-C₆-alkylene-heteroaryl, C₃-C₇ cycloalkyl, heterocyclyl, aryl, and heteroaryl; each optionally substituted with one or more substituents as described herein.

In some embodiments, R¹¹ is selected from: aryl, C₁-C₆-alkylene-aryl, C₁-C₆-alkylene-heteroaryl, C₃-C₇ cycloalkyl, heteroaryl, and heterocyclyl; each optionally substituted with one or more substituents selected from: C₁-C₆ alkoxy, C₁-C₆ alkoxycarbonyl, C₁-C₇ alkyl, C₁-C₆ alkylamino, C₁-C₆ alkylcarboxamide, C₁-C₆ alkylsulfonamido, C₁-C₆ alkylsulfonyl, amino, aryloxy, arylsulfonyl, carboxamide, carbamimidoyl, carboxy, cyano, C₃-C₇ cycloalkyl, C₂-C₈ dialkylamino, C₂-C₈ dialkylsulfamoyl, C₁-C₆ haloalkoxy, C₁-C₆ haloalkyl, halogen, heterocyclyl, hydroxycarbamimidoyl, hydroxyl, oxo, and sulfamoyl; and wherein said C₁-C₆ alkoxy, C₁-C₇ alkyl, C₁-C₆ alkylamino, aryloxy, C₃-C₇ cycloalkyl, and C₂-C₈ dialkylamino are each optionally substituted with one or more substituents selected from: amino, C₁-C₆ alkoxy, C₁-C₆ alkylamino, C₁-C₆ alkylcarboxamide, carboxy, —Y¹—C₁-C₆-alkylene-Z¹ optionally substituted with oxo, C₃-C₇ cycloalkyl, cyano, C₂-C₆ dialkylamino, C₁-C₆ haloalkyl, C₁-C₆ haloalkylamino, heterocyclyl, hydroxyl, oxo, and phenyl;
Y¹ is selected from: —O— and —NH—; and
Z¹ is selected from: C₁-C₆ alkoxy, amino, C₁-C₆ alkylamino, cyano, C₂-C₆ dialkylamino, hydroxyl, and phenyl.

In some embodiments, R¹¹ is selected from: aryl, C₁-C₆-alkylene-aryl, C₁-C₆-alkylene-heteroaryl, C₃-C₇ cycloalkyl, heteroaryl, and heterocyclyl; each optionally substituted with one or more substituents selected from: C₁-C₆ alkoxy, C₁-C₆ alkoxycarbonyl, C₁-C₇ alkyl, C₁-C₆ alkylamino, C₁-C₆ alkylcarboxamide, C₁-C₆ alkylsulfonamido, C₁-C₆ alkylsulfonyl, amino, aryloxy, arylsulfonyl, carboxamide, carbamimidoyl, cyano, C₃-C₇ cycloalkyl, C₂-C₈ dialkylamino, C₂-C₈ dialkylsulfamoyl, C₁-C₆ haloalkoxy, C₁-C₆ haloalkyl, halogen, heterocyclyl, hydroxycarbamimidoyl, hydroxyl, oxo, and sulfamoyl; and wherein said C₁-C₆ alkoxy, C₁-C₇ alkyl, C₁-C₆ alkylamino, aryloxy, C₃-C₇ cycloalkyl, and C₂-C₈ dialkylamino are each optionally substituted with one or more substituents selected from: amino, C₁-C₆ alkoxy, C₁-C₆ alkylamino, C₁-C₆ alkylcarboxamide, carboxy, —Y¹—C₁-C₆-alkylene-Z¹ optionally substituted with oxo, C₃-C₇ cycloalkyl, cyano, C₂-C₆ dialkylamino, C₁-C₆ haloalkyl, C₁-C₆ haloalkylamino, heterocyclyl, hydroxyl, oxo, and phenyl;
Y¹ is selected from: —O— and —NH—; and
Z¹ is selected from: C₁-C₆ alkoxy, amino, C₁-C₆ alkylamino, cyano, C₂-C₆ dialkylamino, hydroxyl, and phenyl.

In some embodiments, R¹¹ is selected from: C₁-C₆-alkylene-aryl, C₁-C₆-alkylene-heteroaryl, C₃-C₇ cycloalkyl, heterocyclyl, aryl, and heteroaryl; each optionally substituted with one or more substituents selected from: (2-ethyl)(methyl)amino, 4-(trifluoromethyl)phenoxy, acetamido, amino, bromo, carbamimidoyl, carboxamide, carboxy, chloro, cyano, cyclopropyl, dimethylamino, dimethylcarbamoyl, ethoxy, ethyl, ethylamino, fluoro, heptyl, hydroxycarbamimidoyl, hydroxyl, isobutyl, isopropoxy, isopropyl, isopropyl(methyl)amino, methoxy, methoxycarbonyl, methyl, methyl(propyl)amino, methylamino, methylsulfonamido, methylsulfonyl, morpholino, N,N-dimethylsulfamoyl, oxo, phenoxy, phenylsulfonyl, piperazinyl, piperidinyl, propoxy, propyl, sec-butyl, sulfamoyl, tert-butyl, tert-pentyl, trifluoromethoxy, and trifluoromethyl; and wherein (2-ethyl)(methyl)amino, cyclopropyl, ethoxy, ethyl, ethylamino, isopropyl(methyl)amino, methoxy, methyl, methyl(propyl)amino, phenoxy, and propoxy are each optionally substituted with one or more substituents selected from: 2-(dimethylamino)ethylamino, 2,2,2-trifluoroethylamino, 2,2-difluoroethylamino, 2-amino-2-oxoacetamido, 2-aminoacetamido, 2-fluoroethylamino, 2-hydroxyethylamino, 2-methoxyethylamino, acetamido, amino, amino, oxo, amino, benzyloxy, carboxy, cyano, cyanomethylamino, cyclopropyl, dimethylamino, ethylamino, hydroxyl, hydroxyl, oxo, isobutylamino, isopentylamino, isopropylamino, methoxy, methylamino, morpholino, oxo, phenyl, pyrrolidinyl, thiazolidinyl, and trifluoromethyl.

In some embodiments, R¹¹ is selected from: C₁-C₆-alkylene-aryl, C₁-C₆-alkylene-heteroaryl, C₃-C₇ cycloalkyl, heterocyclyl, aryl, and heteroaryl; each optionally substituted with one or more substituents selected from: (1-amino-3-hydroxy-1-oxopropan-2-yl)(methyl)amino, (2-(dimethylamino)ethylamino)methyl, (2,2,2-trifluoroethylamino)methyl, (2,2-difluoroethylamino)methyl, (2-acetamidoethyl)(methyl)amino, (2-amino-2-oxoacetamido)methyl, (2-aminoacetamido)methyl, (2-fluoroethylamino)methyl, (2-hydroxyethylamino)methyl, (2-methoxyethylamino)methyl, (cyanomethylamino)methyl, (dimethylamino)methyl, (ethylamino)methyl, (isobutylamino)methyl, (isopentylamino)methyl, (isopropylamino)methyl, (methylamino)methyl, 1-aminocyclopropyl, 2-(benzyloxy)ethyl, 2-(pyrrolidin-1-yl)ethoxy, 2-(trifluoromethyl)phenoxy, 2-aminoethylamino, 2-carboxy-N-methylacetamido, 2-hydroxyethyl, 2-hydroxyethylamino, 2-methoxyethyl, 2-methoxyethylamino, 2-morpholinoethylamino, 3-(dimethylamino)propoxy, 4-(trifluoromethyl)phenoxy, acetamido, acetyl, amino, aminomethyl, benzyl, bromo, carbamimidoyl, carboxamide, carboxy, carboxymethyl, chloro, cyano, cyanomethoxy, cyanomethyl, cyclopropylmethyl, dimethylamino, dimethylcarbamoyl, ethoxy, ethyl, fluoro, heptyl, hydroxycarbamimidoyl, hydroxyl, hydroxymethyl, isobutyl, isopropoxy, isopropyl, methoxy, methoxycarbonyl, methoxymethyl, methyl, methylamino, methylsulfonamido, methylsulfonyl, morpholino, N,N-dimethylsulfamoyl, oxo, phenoxy, phenylsulfonyl, piperazin-1-yl, piperidin-1-yl, propyl, sec-butyl, sulfamoyl, tert-butyl, tert-pentyl, thiazolidin-3-ylmethyl, trifluoromethoxy, and trifluoromethyl.

In some embodiments, R¹¹ is selected from: (1,2,3,4-tetrahydropyrimidin-5-yl)phenyl, (5-isoxazol-3-yl)thiophen-3-yl, (5-isoxazol-3-yl)thiophen-2-yl, (pyridin-2-yl)phenyl, [3,3′-bipyridin]yl, 1,2,3,4-tetrahydropyrido[2,3-b][1,4]oxazepinyl, 1′,2′-dihydrospiro[cyclopropane-1,3′-pyrido[2,3-b][1,4]oxazine]-7′-yl, 1,4-dihydropyridinyl, 1,4-dihydroquinolinyl, 1,5-naphthyridinyl, 1,8-naphthyridinyl, 1H-benzo[d]imidazolyl, 1H-imidazo[4,5-b]pyridinyl, 1H-imidazolyl, 1H-indazolyl, 1H-indolyl, (1H-pyrazol-5-yl)thiophen-3-yl, (1H-pyrazol-5-yl)thiophen-2-yl, 1H-pyrazolo[3,4-b]pyridinyl, 1H-pyrazolo[4,3-b]pyridinyl, 1H-pyrazolyl, 1H-pyrrolo[2,3-b]pyridinyl, 1H-pyrrolo[3,2-b]pyridinyl, 1-phenyl-1H-pyrazolyl, 2-(pyridin-4-yl)ethyl, 2,3-dihydro-[1,4]dioxino[2,3-b]pyridinyl, 2,3-dihydro-1H-benzo[d]imidazolyl, 2,3-dihydro-1H-imidazo[4,5-b]pyridinyl, 2,3-dihydro-1H-pyrido[2,3-b][1,4]oxazinyl, 2,3-dihydro-1H-pyrrolo[2,3-b]pyridinyl, 2,3-dihydrobenzo[b][1,4]dioxinyl, 2,3-dihydrobenzo[d]thiazolyl, 2,3-dihydrobenzofuranyl, 2,3-dihydrofuro[2,3-b]pyridinyl, 2-phenylthiazolyl, 3-(1H-pyrazol-3-yl)phenyl, 3-(1H-pyrazol-4-yl)phenyl, 3-(1H-pyrrol-3-yl)phenyl, 3-(2H-tetrazol-5-yl)phenyl, 3-(furan-2-yl)phenyl, 3-(isoxazol-4-yl)phenyl, 3-(pyridin-2-yl)phenyl, 3-(pyridin-3-yl)phenyl, 3-(pyridin-4-yl)phenyl, 3-(pyrimidin-5-yl)phenyl, 3-(thiazol-5-yl)phenyl, 3-(thiophen-2-yl)phenyl, 3-(thiophen-3-yl)phenyl, 3,4-dihydro-2H-benzo[b][1,4]oxazinyl, 3,4-dihydro-2H-pyrano[2,3-b]pyridinyl, 3,4-dihydro-2H-pyrido[3,2-b][1,4]oxazinyl, 3H-imidazo[4,5-b]pyridinyl, 4′-(1,2,4-oxadiazol-3-yl)biphenylyl, 4-(2H-tetrazol-5-yl)phenyl, 4-(phenyl)pyrimidinyl, 4-(pyridin-3-yl)phenyl, 4-(pyridin-4-yl)phenyl, 4-phenylpyrimidinyl, 5-(1H-pyrazol-4-yl)pyridinyl, 5-(phenyl)pyridinyl, 5,6,7,8-tetrahydro-1,6-naphthyridinyl, 5,6,7,8-tetrahydronaphthalenyl, 5,6,7,8-tetrahydroquinolinyl, 5-phenyl-2,3-dihydrobenzofuranyl, 5-phenylpyrimidinyl, 5-phenylthiophen-3-yl, 5-phenylthiophen-2-yl, 6,7-dihydro-5H-cyclopenta[b]pyridinyl, 6,7-dihydro-5H-pyrrolo[3,4-b]pyridinyl, 6-phenylpyrimidinyl, 7,8-dihydro-5H-pyrano[4,3-b]pyridinyl, benzo[c][1,2,5]oxadiazolyl, benzo[c][1,2,5]thiadiazolyl, benzo[d]isoxazolyl, benzofuranyl, benzyl, biphenylyl, chromanyl, cyclohexyl, furanyl, imidazo[1,2-a]pyridinyl, imidazo[2,1-b]thiazolyl, indolinyl, isoxazolyl, naphthalenyl, phenyl, pyrazinyl, pyridinyl, pyrimidinyl, quinolinyl, thiophen-3-yl, and thiophen-2-yl; each optionally substituted with one or more substituents selected from: C₁-C₆ alkoxy, C₁-C₆ alkoxycarbonyl, C₁-C₇ alkyl, C₁-C₆ alkylamino, C₁-C₆ alkylcarboxamide, C₁-C₆ alkylsulfonamido, C₁-C₆ alkylsulfonyl, amino, aryloxy, arylsulfonyl, carboxamide, carbamimidoyl, carboxy, cyano, C₃-C₇ cycloalkyl, C₂-C₈ dialkylamino, C₂-C₈ dialkylsulfamoyl, C₁-C₆ haloalkoxy, C₁-C₆ haloalkyl, halogen, heterocyclyl, hydroxycarbamimidoyl, hydroxyl, oxo, and sulfamoyl; and wherein said C₁-C₆ alkoxy, C₁-C₇ alkyl, C₁-C₆ alkylamino, aryloxy, C₃-C₇ cycloalkyl, and C₂-C₈ dialkylamino are each optionally substituted with one or more substituents selected from: amino, C₁-C₆ alkoxy, C₁-C₆ alkylamino, C₁-C₆ alkylcarboxamide, carboxy, —Y¹—C₁-C₆-alkylene-Z¹ optionally substituted with oxo, C₃-C₇ cycloalkyl, cyano, C₂-C₆ dialkylamino, C₁-C₆ haloalkyl, C₁-C₆ haloalkylamino, heterocyclyl, hydroxyl, oxo, and phenyl;
Y¹ is selected from: —O— and —NH—; and
Z¹ is selected from: C₁-C₆ alkoxy, amino, C₁-C₆ alkylamino, cyano, C₂-C₆ dialkylamino, hydroxyl, and phenyl.

In some embodiments, R¹ is selected from: (1,2,3,4-tetrahydropyrimidin-5-yl)phenyl, (5-isoxazol-3-yl)thiophen-3-yl, (5-isoxazol-3-yl)thiophen-2-yl, (pyridin-2-yl)phenyl, [3,3′-bipyridin]yl, 1,2,3,4-tetrahydropyrido[2,3-b][1,4]oxazepinyl, 1′,2′-dihydrospiro[cyclopropane-1,3′-pyrido[2,3-b][1,4]oxazine]-7′-yl, 1,4-dihydropyridinyl, 1,4-dihydroquinolinyl, 1,5-naphthyridinyl, 1,8-naphthyridinyl, 1H-benzo[d]imidazolyl, 1H-imidazo[4,5-b]pyridinyl, 1H-imidazolyl, 1H-indazolyl, 1H-indolyl, (1H-pyrazol-5-yl)thiophen-3-yl, (1H-pyrazol-5-yl)thiophen-2-yl, 1H-pyrazolo[3,4-b]pyridinyl, 1H-pyrazolo[4,3-b]pyridinyl, 1H-pyrazolyl, 1H-pyrrolo[2,3-b]pyridinyl, 1H-pyrrolo[3,2-b]pyridinyl, 1-phenyl-1H-pyrazolyl, 2-(pyridin-4-yl)ethyl, 2,3-dihydro-[1,4]dioxino[2,3-b]pyridinyl, 2,3-dihydro-1H-benzo[d]imidazolyl, 2,3-dihydro-1H-imidazo[4,5-b]pyridinyl, 2,3-dihydro-1H-pyrido[2,3-b][1,4]oxazinyl, 2,3-dihydro-1H-pyrrolo[2,3-b]pyridinyl, 2,3-dihydrobenzo[b][1,4]dioxinyl, 2,3-dihydrobenzo[d]thiazolyl, 2,3-dihydrobenzofuranyl, 2,3-dihydrofuro[2,3-b]pyridinyl, 2-phenylthiazolyl, 3-(1H-pyrazol-3-yl)phenyl, 3-(1H-pyrazol-4-yl)phenyl, 3-(1H-pyrrol-3-yl)phenyl, 3-(2H-tetrazol-5-yl)phenyl, 3-(furan-2-yl)phenyl, 3-(isoxazol-4-yl)phenyl, 3-(pyridin-2-yl)phenyl, 3-(pyridin-3-yl)phenyl, 3-(pyridin-4-yl)phenyl, 3-(pyrimidin-5-yl)phenyl, 3-(thiazol-5-yl)phenyl, 3-(thiophen-2-yl)phenyl, 3-(thiophen-3-yl)phenyl, 3,4-dihydro-2H-benzo[b][1,4]oxazinyl, 3,4-dihydro-2H-pyrano[2,3-b]pyridinyl, 3,4-dihydro-2H-pyrido[3,2-b][1,4]oxazinyl, 3H-imidazo[4,5-b]pyridinyl, 4′-(1,2,4-oxadiazol-3-yl)biphenylyl, 4-(2H-tetrazol-5-yl)phenyl, 4-(phenyl)pyrimidinyl, 4-(pyridin-3-yl)phenyl, 4-(pyridin-4-yl)phenyl, 4-phenylpyrimidinyl, 5-(1H-pyrazol-4-yl)pyridinyl, 5-(phenyl)pyridinyl, 5,6,7,8-tetrahydro-1,6-naphthyridinyl, 5,6,7,8-tetrahydronaphthalenyl, 5,6,7,8-tetrahydroquinolinyl, 5-phenyl-2,3-dihydrobenzofuranyl, 5-phenylpyrimidinyl, 5-phenylthiophen-3-yl, 5-phenylthiophen-2-yl, 6,7-dihydro-5H-cyclopenta[b]pyridinyl, 6,7-dihydro-5H-pyrrolo[3,4-b]pyridinyl, 6-phenylpyrimidinyl, 7,8-dihydro-5H-pyrano[4,3-b]pyridinyl, benzo[c][1,2,5]oxadiazolyl, benzo[c][1,2,5]thiadiazolyl, benzo[d]isoxazolyl, benzofuranyl, benzyl, biphenylyl, chromanyl, cyclohexyl, furanyl, imidazo[1,2-a]pyridinyl, imidazo[2,1-b]thiazolyl, indolinyl, isoxazolyl, naphthalenyl, phenyl, pyrazinyl, pyridinyl, pyrimidinyl, quinolinyl, thiophen-3-yl, and thiophen-2-yl; each optionally substituted with one or more substituents selected from: (2-ethyl)(methyl)amino, 4-(trifluoromethyl)phenoxy, acetamido, amino, bromo, carbamimidoyl, carboxamide, carboxy, chloro, cyano, cyclopropyl, dimethylamino, dimethylcarbamoyl, ethoxy, ethyl, ethylamino, fluoro, heptyl, hydroxycarbamimidoyl, hydroxyl, isobutyl, isopropoxy, isopropyl, isopropyl(methyl)amino, methoxy, methoxycarbonyl, methyl, methyl(propyl)amino, methylamino, methylsulfonamido, methylsulfonyl, morpholino, N,N-dimethylsulfamoyl, oxo, phenoxy, phenylsulfonyl, piperazinyl, piperidinyl, propoxy, propyl, sec-butyl, sulfamoyl, tert-butyl, tert-pentyl, trifluoromethoxy, and trifluoromethyl; and wherein (2-ethyl)(methyl)amino, cyclopropyl, ethoxy, ethyl, ethylamino, isopropyl(methyl)amino, methoxy, methyl, methyl(propyl)amino, phenoxy, and propoxy are each optionally substituted with one or more substituents selected from: 2-(dimethylamino)ethylamino, 2,2,2-trifluoroethylamino, 2,2-difluoroethylamino, 2-amino-2-oxoacetamido, 2-aminoacetamido, 2-fluoroethylamino, 2-hydroxyethylamino, 2-methoxyethylamino, acetamido, amino, amino, oxo, amino, benzyloxy, carboxy, cyano, cyanomethylamino, cyclopropyl, dimethylamino, ethylamino, hydroxyl, hydroxyl, oxo, isobutylamino, isopentylamino, isopropylamino, methoxy, methylamino, morpholino, oxo, phenyl, pyrrolidinyl, thiazolidinyl, and trifluoromethyl.

In some embodiments, R¹¹ is selected from: (1,2,3,4-tetrahydropyrimidin-5-yl)phenyl, (5-isoxazol-3-yl)thiophen-3-yl, (5-isoxazol-3-yl)thiophen-2-yl, (pyridin-2-yl)phenyl, [3,3′-bipyridin]yl, 1,2,3,4-tetrahydropyrido[2,3-b][1,4]oxazepinyl, 1′,2′-dihydrospiro[cyclopropane-1,3′-pyrido[2,3-b][1,4]oxazine]-7′-yl, 1,4-dihydropyridinyl, 1,4-dihydroquinolinyl, 1,5-naphthyridinyl, 1,8-naphthyridinyl, 1H-benzo[d]imidazolyl, 1H-imidazo[4,5-b]pyridinyl, 1H-imidazolyl, 1H-indazolyl, 1H-indolyl, (1H-pyrazol-5-yl)thiophen-3-yl, (1H-pyrazol-5-yl)thiophen-2-yl, 1H-pyrazolo[3,4-b]pyridinyl, 1H-pyrazolo[4,3-b]pyridinyl, 1H-pyrazolyl, 1H-pyrrolo[2,3-b]pyridinyl, 1H-pyrrolo[3,2-b]pyridinyl, 1-phenyl-1H-pyrazolyl, 2-(pyridin-4-yl)ethyl, 2,3-dihydro-[1,4]dioxino[2,3-b]pyridinyl, 2,3-dihydro-1H-benzo[d]imidazolyl, 2,3-dihydro-1H-imidazo[4,5-b]pyridinyl, 2,3-dihydro-1H-pyrido[2,3-b][1,4]oxazinyl, 2,3-dihydro-1H-pyrrolo[2,3-b]pyridinyl, 2,3-dihydrobenzo[b][1,4]dioxinyl, 2,3-dihydrobenzo[d]thiazolyl, 2,3-dihydrobenzofuranyl, 2,3-dihydrofuro[2,3-b]pyridinyl, 2-phenylthiazolyl, 3-(1H-pyrazol-3-yl)phenyl, 3-(1H-pyrazol-4-yl)phenyl, 3-(1H-pyrrol-3-yl)phenyl, 3-(2H-tetrazol-5-yl)phenyl, 3-(furan-2-yl)phenyl, 3-(isoxazol-4-yl)phenyl, 3-(pyridin-2-yl)phenyl, 3-(pyridin-3-yl)phenyl, 3-(pyridin-4-yl)phenyl, 3-(pyrimidin-5-yl)phenyl, 3-(thiazol-5-yl)phenyl, 3-(thiophen-2-yl)phenyl, 3-(thiophen-3-yl)phenyl, 3,4-dihydro-2H-benzo[b][1,4]oxazinyl, 3,4-dihydro-2H-pyrano[2,3-b]pyridinyl, 3,4-dihydro-2H-pyrido[3,2-b][1,4]oxazinyl, 3H-imidazo[4,5-b]pyridinyl, 4′-(1,2,4-oxadiazol-3-yl)biphenylyl, 4-(2H-tetrazol-5-yl)phenyl, 4-(phenyl)pyrimidinyl, 4-(pyridin-3-yl)phenyl, 4-(pyridin-4-yl)phenyl, 4-phenylpyrimidinyl, 5-(1H-pyrazol-4-yl)pyridinyl, 5-(phenyl)pyridinyl, 5,6,7,8-tetrahydro-1,6-naphthyridinyl, 5,6,7,8-tetrahydronaphthalenyl, 5,6,7,8-tetrahydroquinolinyl, 5-phenyl-2,3-dihydrobenzofuranyl, 5-phenylpyrimidinyl, 5-phenylthiophen-3-yl, 5-phenylthiophen-2-yl, 6,7-dihydro-5H-cyclopenta[b]pyridinyl, 6,7-dihydro-5H-pyrrolo[3,4-b]pyridinyl, 6-phenylpyrimidinyl, 7,8-dihydro-5H-pyrano[4,3-b]pyridinyl, benzo[c][1,2,5]oxadiazolyl, benzo[c][1,2,5]thiadiazolyl, benzo[d]isoxazolyl, benzofuranyl, benzyl, biphenylyl, chromanyl, cyclohexyl, furanyl, imidazo[1,2-a]pyridinyl, imidazo[2,1-b]thiazolyl, indolinyl, isoxazolyl, naphthalenyl, phenyl, pyrazinyl, pyridinyl, pyrimidinyl, quinolinyl, thiophen-3-yl, and thiophen-2-yl; each optionally substituted with one or more substituents selected from: (1-amino-3-hydroxy-1-oxopropan-2-yl)(methyl)amino, (2-(dimethylamino)ethylamino)methyl, (2,2,2-trifluoroethylamino)methyl, (2,2-difluoroethylamino)methyl, (2-acetamidoethyl)(methyl)amino, (2-amino-2-oxoacetamido)methyl, (2-aminoacetamido)methyl, (2-fluoroethylamino)methyl, (2-hydroxyethylamino)methyl, (2-methoxyethylamino)methyl, (cyanomethylamino)methyl, (dimethylamino)methyl, (ethylamino)methyl, (isobutylamino)methyl, (isopentylamino)methyl, (isopropylamino)methyl, (methylamino)methyl, 1-aminocyclopropyl, 2-(benzyloxy)ethyl, 2-(pyrrolidin-1-yl)ethoxy, 2-(trifluoromethyl)phenoxy, 2-aminoethylamino, 2-carboxy-N-methylacetamido, 2-hydroxyethyl, 2-hydroxyethylamino, 2-methoxyethyl, 2-methoxyethylamino, 2-morpholinoethylamino, 3-(dimethylamino)propoxy, 4-(trifluoromethyl)phenoxy, acetamido, acetyl, amino, aminomethyl, benzyl, bromo, carbamimidoyl, carboxamide, carboxy, carboxymethyl, chloro, cyano, cyanomethoxy, cyanomethyl, cyclopropylmethyl, dimethylamino, dimethylcarbamoyl, ethoxy, ethyl, fluoro, heptyl, hydroxycarbamimidoyl, hydroxyl, hydroxymethyl, isobutyl, isopropoxy, isopropyl, methoxy, methoxycarbonyl, methoxymethyl, methyl, methylamino, methylsulfonamido, methylsulfonyl, morpholino, N,N-dimethylsulfamoyl, oxo, phenoxy, phenylsulfonyl, piperazin-1-yl, piperidin-1-yl, propyl, sec-butyl, sulfamoyl, tert-butyl, tert-pentyl, thiazolidin-3-ylmethyl, trifluoromethoxy, and trifluoromethyl.

In some embodiments, R¹¹ is selected from: (1,2,3,4-tetrahydropyrimidin-5-yl)phenyl, (5-isoxazol-3-yl)thiophen-2-yl, (pyridin-2-yl)phenyl, [3,3′-bipyridin]-5-yl, 1,2,3,4-tetrahydropyrido[2,3-b][1,4]oxazepin-8-yl, 1′,2′-dihydrospiro[cyclopropane-1,3′-pyrido[2,3-b][1,4]oxazine]-7′-yl, 1,4-dihydropyridin-3-yl, 1,4-dihydroquinolin-3-yl, 1,5-naphthyridin-3-yl, 1,8-naphthyridin-3-yl, 1H-benzo[d]imidazol-5-yl, 1H-benzo[d]imidazol-6-yl, 1H-imidazo[4,5-b]pyridin-6-yl, 1H-imidazol-4-yl, 1H-indazol-5-yl, 1H-indazol-6-yl, 1H-indol-5-yl, 1H-pyrazol-3-yl, 1H-pyrazol-4-yl, (1H-pyrazol-5-yl)thiophen-2-yl, 1H-pyrazolo[3,4-b]pyridin-5-yl, 1H-pyrazolo[4,3-b]pyridin-6-yl, 1H-pyrrolo[2,3-b]pyridin-3-yl, 1H-pyrrolo[2,3-b]pyridin-5-yl, 1H-pyrrolo[3,2-b]pyridin-3-yl, 1H-pyrrolo[3,2-b]pyridin-6-yl, 1-phenyl-1H-pyrazol-4-yl, 2-(pyridin-4-yl)ethyl, 2,3-dihydro-[1,4]dioxino[2,3-b]pyridin-7-yl, 2,3-dihydro-1H-benzo[d]imidazol-5-yl, 2,3-dihydro-1H-imidazo[4,5-b]pyridin-6-yl, 2,3-dihydro-1H-pyrido[2,3-b][1,4]oxazin-6-yl, 2,3-dihydro-1H-pyrido[2,3-b][1,4]oxazin-7-yl, 2,3-dihydro-1H-pyrrolo[2,3-b]pyridin-5-yl, 2,3-dihydrobenzo[b][1,4]dioxin-6-yl, 2,3-dihydrobenzo[d]thiazol-6-yl, 2,3-dihydrobenzofuran-5-yl, 2,3-dihydrobenzofuran-7-yl, 2,3-dihydrofuro[2,3-b]pyridin-5-yl, 2-phenylthiazol-5-yl, 3-(1H-pyrazol-3-yl)phenyl, 3-(1H-pyrazol-4-yl)phenyl, 3-(1H-pyrrol-3-yl)phenyl, 3-(2H-tetrazol-5-yl)phenyl, 3-(furan-2-yl)phenyl, 3-(isoxazol-4-yl)phenyl, 3-(pyridin-2-yl)phenyl, 3-(pyridin-3-yl)phenyl, 3-(pyridin-4-yl)phenyl, 3-(pyrimidin-5-yl)phenyl, 3-(thiazol-5-yl)phenyl, 3-(thiophen-2-yl)phenyl, 3-(thiophen-3-yl)phenyl, 3,4-dihydro-2H-benzo[b][1,4]oxazin-6-yl, 3,4-dihydro-2H-pyrano[2,3-b]pyridin-6-yl, 3,4-dihydro-2H-pyrido[3,2-b][1,4]oxazin-7-yl, 3H-imidazo[4,5-b]pyridin-6-yl, 4′-(1,2,4-oxadiazol-3-yl)biphenyl-3-yl, 4-(2H-tetrazol-5-yl)phenyl, 4-(phenyl)pyrimidin-2-yl, 4-(pyridin-3-yl)phenyl, 4-(pyridin-4-yl)phenyl, 4-phenylpyrimidin-2-yl, 5-(1H-pyrazol-4-yl)pyridin-3-yl, 5-(phenyl)pyridin-3-yl, 5,6,7,8-tetrahydro-1,6-naphthyridin-3-yl, 5,6,7,8-tetrahydronaphthalen-1-yl, 5,6,7,8-tetrahydronaphthalen-2-yl, 5,6,7,8-tetrahydronaphthalenyl, 5,6,7,8-tetrahydroquinolin-3-yl, 5-phenyl-2,3-dihydrobenzofuran-7-yl, 5-phenylpyrimidin-2-yl, 5-phenylthiophen-2-yl, 6,7-dihydro-5H-cyclopenta[b]pyridin-3-yl, 6,7-dihydro-5H-pyrrolo[3,4-b]pyridin-3-yl, 6-phenylpyrimidin-2-yl, 7,8-dihydro-5H-pyrano[4,3-b]pyridin-3-yl, benzo[c][1,2,5]oxadiazol-4-yl, benzo[c][1,2,5]thiadiazol-4-yl, benzo[c][1,2,5]thiadiazol-5-yl, benzo[d]isoxazol-5-yl, benzofuran-2-yl, benzofuran-5-yl, benzyl, biphenyl-2-yl, biphenyl-3-yl, biphenyl-4-yl, chroman-6-yl, cyclohexyl, furan-3-yl, imidazo[1,2-a]pyridin-6-yl, imidazo[2,1-b]thiazol-5-yl, indolin-5-yl, isoxazol-4-yl, naphthalen-1-yl, naphthalen-2-yl, phenyl, pyrazin-2-yl, pyridin-2-yl, pyridin-3-yl, pyrimidin-2-yl, pyrimidin-4-yl, pyrimidin-5-yl, quinolin-3-yl, quinolin-6-yl, quinolin-8-yl, thiophen-2-yl, and thiophen-3-yl; each optionally substituted with one or more substituents selected from: C₁-C₆ alkoxy, C₁-C₆ alkoxycarbonyl, C₁-C₇ alkyl, C₁-C₆ alkylamino, C₁-C₆ alkylcarboxamide, C₁-C₆ alkylsulfonamido, C₁-C₆ alkylsulfonyl, amino, aryloxy, arylsulfonyl, carboxamide, carbamimidoyl, carboxy, cyano, C₃-C₇ cycloalkyl, C₂-C₈ dialkylamino, C₂-C₈ dialkylsulfamoyl, C₁-C₆ haloalkoxy, C₁-C₆ haloalkyl, halogen, heterocyclyl, hydroxycarbamimidoyl, hydroxyl, oxo, and sulfamoyl; and wherein said C₁-C₆ alkoxy, C₁-C₇ alkyl, C₁-C₆ alkylamino, aryloxy, C₃-C₇ cycloalkyl, and C₂-C₈ dialkylamino are each optionally substituted with one or more substituents selected from: amino, C₁-C₆ alkoxy, C₁-C₆ alkylamino, C₁-C₆ alkylcarboxamide, carboxy, —Y¹—C₁-C₆-alkylene-Z¹ optionally substituted with oxo, C₃-C₇ cycloalkyl, cyano, C₂-C₆ dialkylamino, C₁-C₆ haloalkyl, C₁-C₆ haloalkylamino, heterocyclyl, hydroxyl, oxo, and phenyl;
Y¹ is selected from: —O— and —NH—; and
Z¹ is selected from: C₁-C₆ alkoxy, amino, C₁-C₆ alkylamino, cyano, C₂-C₆ dialkylamino, hydroxyl, and phenyl.

In some embodiments, R¹¹ is selected from: (1,2,3,4-tetrahydropyrimidin-5-yl)phenyl, (5-isoxazol-3-yl)thiophen-2-yl, (pyridin-2-yl)phenyl, [3,3′-bipyridin]-5-yl, 1,2,3,4-tetrahydropyrido[2,3-b][1,4]oxazepin-8-yl, 1′,2′-dihydrospiro[cyclopropane-1,3′-pyrido[2,3-b][1,4]oxazine]-7′-yl, 1,4-dihydropyridin-3-yl, 1,4-dihydroquinolin-3-yl, 1,5-naphthyridin-3-yl, 1,8-naphthyridin-3-yl, 1H-benzo[d]imidazol-5-yl, 1H-benzo[d]imidazol-6-yl, 1H-imidazo[4,5-b]pyridin-6-yl, 1H-imidazol-4-yl, 1H-indazol-5-yl, 1H-indazol-6-yl, 1H-indol-5-yl, 1H-pyrazol-3-yl, 1H-pyrazol-4-yl, (1H-pyrazol-5-yl)thiophen-2-yl, 1H-pyrazolo[3,4-b]pyridin-5-yl, 1H-pyrazolo[4,3-b]pyridin-6-yl, 1H-pyrrolo[2,3-b]pyridin-3-yl, 1H-pyrrolo[2,3-b]pyridin-5-yl, 1H-pyrrolo[3,2-b]pyridin-3-yl, 1H-pyrrolo[3,2-b]pyridin-6-yl, 1-phenyl-1H-pyrazol-4-yl, 2-(pyridin-4-yl)ethyl, 2,3-dihydro-[1,4]dioxino[2,3-b]pyridin-7-yl, 2,3-dihydro-1H-benzo[d]imidazol-5-yl, 2,3-dihydro-1H-imidazo[4,5-b]pyridin-6-yl, 2,3-dihydro-1H-pyrido[2,3-b][1,4]oxazin-6-yl, 2,3-dihydro-1H-pyrido[2,3-b][1,4]oxazin-7-yl, 2,3-dihydro-1H-pyrrolo[2,3-b]pyridin-5-yl, 2,3-dihydrobenzo[b][1,4]dioxin-6-yl, 2,3-dihydrobenzo[d]thiazol-6-yl, 2,3-dihydrobenzofuran-5-yl, 2,3-dihydrobenzofuran-7-yl, 2,3-dihydrofuro[2,3-b]pyridin-5-yl, 2-phenylthiazol-5-yl, 3-(1H-pyrazol-3-yl)phenyl, 3-(1H-pyrazol-4-yl)phenyl, 3-(1H-pyrrol-3-yl)phenyl, 3-(2H-tetrazol-5-yl)phenyl, 3-(furan-2-yl)phenyl, 3-(isoxazol-4-yl)phenyl, 3-(pyridin-2-yl)phenyl, 3-(pyridin-3-yl)phenyl, 3-(pyridin-4-yl)phenyl, 3-(pyrimidin-5-yl)phenyl, 3-(thiazol-5-yl)phenyl, 3-(thiophen-2-yl)phenyl, 3-(thiophen-3-yl)phenyl, 3,4-dihydro-2H-benzo[b][1,4]oxazin-6-yl, 3,4-dihydro-2H-pyrano[2,3-b]pyridin-6-yl, 3,4-dihydro-2H-pyrido[3,2-b][1,4]oxazin-7-yl, 3H-imidazo[4,5-b]pyridin-6-yl, 4′-(1,2,4-oxadiazol-3-yl)biphenyl-3-yl, 4-(2H-tetrazol-5-yl)phenyl, 4-(phenyl)pyrimidin-2-yl, 4-(pyridin-3-yl)phenyl, 4-(pyridin-4-yl)phenyl, 4-phenylpyrimidin-2-yl, 5-(1H-pyrazol-4-yl)pyridin-3-yl, 5-(phenyl)pyridin-3-yl, 5,6,7,8-tetrahydro-1,6-naphthyridin-3-yl, 5,6,7,8-tetrahydronaphthalen-1-yl, 5,6,7,8-tetrahydronaphthalen-2-yl, 5,6,7,8-tetrahydronaphthalenyl, 5,6,7,8-tetrahydroquinolin-3-yl, 5-phenyl-2,3-dihydrobenzofuran-7-yl, 5-phenylpyrimidin-2-yl, 5-phenylthiophen-2-yl, 6,7-dihydro-5H-cyclopenta[b]pyridin-3-yl, 6,7-dihydro-5H-pyrrolo[3,4-b]pyridin-3-yl, 6-phenylpyrimidin-2-yl, 7,8-dihydro-5H-pyrano[4,3-b]pyridin-3-yl, benzo[c][1,2,5]oxadiazol-4-yl, benzo[c][1,2,5]thiadiazol-4-yl, benzo[c][1,2,5]thiadiazol-5-yl, benzo[d]isoxazol-5-yl, benzofuran-2-yl, benzofuran-5-yl, benzyl, biphenyl-2-yl, biphenyl-3-yl, biphenyl-4-yl, chroman-6-yl, cyclohexyl, furan-3-yl, imidazo[1,2-a]pyridin-6-yl, imidazo[2,1-b]thiazol-5-yl, indolin-5-yl, isoxazol-4-yl, naphthalen-1-yl, naphthalen-2-yl, phenyl, pyrazin-2-yl, pyridin-2-yl, pyridin-3-yl, pyrimidin-2-yl, pyrimidin-4-yl, pyrimidin-5-yl, quinolin-3-yl, quinolin-6-yl, quinolin-8-yl, thiophen-2-yl, and thiophen-3-yl; each optionally substituted with one or more substituents selected from: (2-ethyl)(methyl)amino, 4-(trifluoromethyl)phenoxy, acetamido, amino, bromo, carbamimidoyl, carboxamide, carboxy, chloro, cyano, cyclopropyl, dimethylamino, dimethylcarbamoyl, ethoxy, ethyl, ethylamino, fluoro, heptyl, hydroxycarbamimidoyl, hydroxyl, isobutyl, isopropoxy, isopropyl, isopropyl(methyl)amino, methoxy, methoxycarbonyl, methyl, methyl(propyl)amino, methylamino, methylsulfonamido, methylsulfonyl, morpholino, N,N-dimethylsulfamoyl, oxo, phenoxy, phenylsulfonyl, piperazinyl, piperidinyl, propoxy, propyl, sec-butyl, sulfamoyl, tert-butyl, tert-pentyl, trifluoromethoxy, and trifluoromethyl; and wherein (2-ethyl)(methyl)amino, cyclopropyl, ethoxy, ethyl, ethylamino, isopropyl(methyl)amino, methoxy, methyl, methyl(propyl)amino, phenoxy, and propoxy are each optionally substituted with one or more substituents selected from: 2-(dimethylamino)ethylamino, 2,2,2-trifluoroethylamino, 2,2-difluoroethylamino, 2-amino-2-oxoacetamido, 2-aminoacetamido, 2-fluoroethylamino, 2-hydroxyethylamino, 2-methoxyethylamino, acetamido, amino, amino, oxo, amino, benzyloxy, carboxy, cyano, cyanomethylamino, cyclopropyl, dimethylamino, ethylamino, hydroxyl, hydroxyl, oxo, isobutylamino, isopentylamino, isopropylamino, methoxy, methylamino, morpholino, oxo, phenyl, pyrrolidinyl, thiazolidinyl, and trifluoromethyl.

In some embodiments, R¹¹ is selected from: (1,2,3,4-tetrahydropyrimidin-5-yl)phenyl, (5-isoxazol-3-yl)thiophen-2-yl, (pyridin-2-yl)phenyl, [3,3′-bipyridin]-5-yl, 1,2,3,4-tetrahydropyrido[2,3-b][1,4]oxazepin-8-yl, 1′,2′-dihydrospiro[cyclopropane-1,3′-pyrido[2,3-b][1,4]oxazine]-7′-yl, 1,4-dihydropyridin-3-yl, 1,4-dihydroquinolin-3-yl, 1,5-naphthyridin-3-yl, 1,8-naphthyridin-3-yl, 1H-benzo[d]imidazol-5-yl, 1H-benzo[d]imidazol-6-yl, 1H-imidazo[4,5-b]pyridin-6-yl, 1H-imidazol-4-yl, 1H-indazol-5-yl, 1H-indazol-6-yl, 1H-indol-5-yl, 1H-pyrazol-3-yl, 1H-pyrazol-4-yl, (1H-pyrazol-5-yl)thiophen-2-yl, 1H-pyrazolo[3,4-b]pyridin-5-yl, 1H-pyrazolo[4,3-b]pyridin-6-yl, 1H-pyrrolo[2,3-b]pyridin-3-yl, 1H-pyrrolo[2,3-b]pyridin-5-yl, 1H-pyrrolo[3,2-b]pyridin-3-yl, 1H-pyrrolo[3,2-b]pyridin-6-yl, 1-phenyl-1H-pyrazol-4-yl, 2-(pyridin-4-yl)ethyl, 2,3-dihydro-[1,4]dioxino[2,3-b]pyridin-7-yl, 2,3-dihydro-1H-benzo[d]imidazol-5-yl, 2,3-dihydro-1H-imidazo[4,5-b]pyridin-6-yl, 2,3-dihydro-1H-pyrido[2,3-b][1,4]oxazin-6-yl, 2,3-dihydro-1H-pyrido[2,3-b][1,4]oxazin-7-yl, 2,3-dihydro-1H-pyrrolo[2,3-b]pyridin-5-yl, 2,3-dihydrobenzo[b][1,4]dioxin-6-yl, 2,3-dihydrobenzo[d]thiazol-6-yl, 2,3-dihydrobenzofuran-5-yl, 2,3-dihydrobenzofuran-7-yl, 2,3-dihydrofuro[2,3-b]pyridin-5-yl, 2-phenylthiazol-5-yl, 3-(1H-pyrazol-3-yl)phenyl, 3-(1H-pyrazol-4-yl)phenyl, 3-(1H-pyrrol-3-yl)phenyl, 3-(2H-tetrazol-5-yl)phenyl, 3-(furan-2-yl)phenyl, 3-(isoxazol-4-yl)phenyl, 3-(pyridin-2-yl)phenyl, 3-(pyridin-3-yl)phenyl, 3-(pyridin-4-yl)phenyl, 3-(pyrimidin-5-yl)phenyl, 3-(thiazol-5-yl)phenyl, 3-(thiophen-2-yl)phenyl, 3-(thiophen-3-yl)phenyl, 3,4-dihydro-2H-benzo[b][1,4]oxazin-6-yl, 3,4-dihydro-2H-pyrano[2,3-b]pyridin-6-yl, 3,4-dihydro-2H-pyrido[3,2-b][1,4]oxazin-7-yl, 3H-imidazo[4,5-b]pyridin-6-yl, 4′-(1,2,4-oxadiazol-3-yl)biphenyl-3-yl, 4-(2H-tetrazol-5-yl)phenyl, 4-(phenyl)pyrimidin-2-yl, 4-(pyridin-3-yl)phenyl, 4-(pyridin-4-yl)phenyl, 4-phenylpyrimidin-2-yl, 5-(1H-pyrazol-4-yl)pyridin-3-yl, 5-(phenyl)pyridin-3-yl, 5,6,7,8-tetrahydro-1,6-naphthyridin-3-yl, 5,6,7,8-tetrahydronaphthalen-1-yl, 5,6,7,8-tetrahydronaphthalen-2-yl, 5,6,7,8-tetrahydronaphthalenyl, 5,6,7,8-tetrahydroquinolin-3-yl, 5-phenyl-2,3-dihydrobenzofuran-7-yl, 5-phenylpyrimidin-2-yl, 5-phenylthiophen-2-yl, 6,7-dihydro-5H-cyclopenta[b]pyridin-3-yl, 6,7-dihydro-5H-pyrrolo[3,4-b]pyridin-3-yl, 6-phenylpyrimidin-2-yl, 7,8-dihydro-5H-pyrano[4,3-b]pyridin-3-yl, benzo[c][1,2,5]oxadiazol-4-yl, benzo[c][1,2,5]thiadiazol-4-yl, benzo[c][1,2,5]thiadiazol-5-yl, benzo[d]isoxazol-5-yl, benzofuran-2-yl, benzofuran-5-yl, benzyl, biphenyl-2-yl, biphenyl-3-yl, biphenyl-4-yl, chroman-6-yl, cyclohexyl, furan-3-yl, imidazo[1,2-a]pyridin-6-yl, imidazo[2,1-b]thiazol-5-yl, indolin-5-yl, isoxazol-4-yl, naphthalen-1-yl, naphthalen-2-yl, phenyl, pyrazin-2-yl, pyridin-2-yl, pyridin-3-yl, pyrimidin-2-yl, pyrimidin-4-yl, pyrimidin-5-yl, quinolin-3-yl, quinolin-6-yl, quinolin-8-yl, thiophen-2-yl, and thiophen-3-yl; each optionally substituted with one or more substituents selected from: (1-amino-3-hydroxy-1-oxopropan-2-yl)(methyl)amino, (2-(dimethylamino)ethylamino)methyl, (2,2,2-trifluoroethylamino)methyl, (2,2-difluoroethylamino)methyl, (2-acetamidoethyl)(methyl)amino, (2-amino-2-oxoacetamido)methyl, (2-aminoacetamido)methyl, (2-fluoroethylamino)methyl, (2-hydroxyethylamino)methyl, (2-methoxyethylamino)methyl, (cyanomethylamino)methyl, (dimethylamino)methyl, (ethylamino)methyl, (isobutylamino)methyl, (isopentylamino)methyl, (isopropylamino)methyl, (methylamino)methyl, 1-aminocyclopropyl, 2-(benzyloxy)ethyl, 2-(pyrrolidin-1-yl)ethoxy, 2-(trifluoromethyl)phenoxy, 2-aminoethylamino, 2-carboxy-N-methylacetamido, 2-hydroxyethyl, 2-hydroxyethylamino, 2-methoxyethyl, 2-methoxyethylamino, 2-morpholinoethylamino, 3-(dimethylamino)propoxy, 4-(trifluoromethyl)phenoxy, acetamido, acetyl, amino, aminomethyl, benzyl, bromo, carbamimidoyl, carboxamide, carboxy, carboxymethyl, chloro, cyano, cyanomethoxy, cyanomethyl, cyclopropylmethyl, dimethylamino, dimethylcarbamoyl, ethoxy, ethyl, fluoro, heptyl, hydroxycarbamimidoyl, hydroxyl, hydroxymethyl, isobutyl, isopropoxy, isopropyl, methoxy, methoxycarbonyl, methoxymethyl, methyl, methylamino, methylsulfonamido, methylsulfonyl, morpholino, N,N-dimethylsulfamoyl, oxo, phenoxy, phenylsulfonyl, piperazin-1-yl, piperidin-1-yl, propyl, sec-butyl, sulfamoyl, tert-butyl, tert-pentyl, thiazolidin-3-ylmethyl, trifluoromethoxy, and trifluoromethyl.

In some embodiments, R¹¹ is selected from: (1,2,3,4-tetrahydropyrimidin-5-yl)phenyl, (5-isoxazol-3-yl)thiophen-2-yl, (5-isoxazol-3-yl)thiophen-3-yl, (pyridin-2-yl)phenyl, [3,3′-bipyridin]yl, 1,2,3,4-tetrahydropyrido[2,3-b][1,4]oxazepinyl, 1′,2′-dihydrospiro[cyclopropane-1,3′-pyrido[2,3-b][1,4]oxazine]-7′-yl, 1,4-dihydropyridinyl, 1,4-dihydroquinolinyl, 1,5-naphthyridinyl, 1,8-naphthyridinyl, 1H-benzo[d]imidazolyl, 1H-imidazo[4,5-b]pyridinyl, 1H-imidazolyl, 1H-indazolyl, 1H-indolyl, (1H-pyrazol-5-yl)thiophen-2-yl, (1H-pyrazol-5-yl)thiophen-3-yl, 1H-pyrazolo[3,4-b]pyridinyl, 1H-pyrazolo[4,3-b]pyridinyl, 1H-pyrazolyl, 1H-pyrrolo[2,3-b]pyridinyl, 1H-pyrrolo[3,2-b]pyridinyl, 1-phenyl-1H-pyrazolyl, 2,3-dihydro-[1,4]dioxino[2,3-b]pyridinyl, 2,3-dihydro-1H-benzo[d]imidazolyl, 2,3-dihydro-1H-imidazo[4,5-b]pyridinyl, 2,3-dihydro-1H-pyrido[2,3-b][1,4]oxazinyl, 2,3-dihydro-1H-pyrrolo[2,3-b]pyridinyl, 2,3-dihydrobenzo[b][1,4]dioxinyl, 2,3-dihydrobenzo[d]thiazolyl, 2,3-dihydrobenzofuranyl, 2,3-dihydrofuro[2,3-b]pyridinyl, 2-phenylthiazolyl, 3-(1H-pyrazol-3-yl)phenyl, 3-(1H-pyrazol-4-yl)phenyl, 3-(1H-pyrrol-3-yl)phenyl, 3-(2H-tetrazol-5-yl)phenyl, 3-(furan-2-yl)phenyl, 3-(isoxazol-4-yl)phenyl, 3-(pyridin-2-yl)phenyl, 3-(pyridin-3-yl)phenyl, 3-(pyridin-4-yl)phenyl, 3-(pyrimidin-5-yl)phenyl, 3-(thiazol-5-yl)phenyl, 3-(thiophen-2-yl)phenyl, 3-(thiophen-3-yl)phenyl, 3,4-dihydro-2H-benzo[b][1,4]oxazinyl, 3,4-dihydro-2H-pyrano[2,3-b]pyridinyl, 3,4-dihydro-2H-pyrido[3,2-b][1,4]oxazinyl, 3H-imidazo[4,5-b]pyridinyl, 4′-(1,2,4-oxadiazol-3-yl)biphenylyl, 4-(2H-tetrazol-5-yl)phenyl, 4-(pyridin-3-yl)phenyl, 4-(pyridin-4-yl)phenyl, 5-(1H-pyrazol-4-yl)pyridinyl, 5-(phenyl)pyridinyl, 5,6,7,8-tetrahydro-1,6-naphthyridinyl, 5,6,7,8-tetrahydronaphthalenyl, 5,6,7,8-tetrahydroquinolinyl, 5-phenyl-2,3-dihydrobenzofuranyl, 5-phenylthiophen-2-yl, 5-phenylthiophen-3-yl, 6,7-dihydro-5H-cyclopenta[b]pyridinyl, 6,7-dihydro-5H-pyrrolo[3,4-b]pyridinyl, 7,8-dihydro-5H-pyrano[4,3-b]pyridinyl, benzo[c][1,2,5]oxadiazolyl, benzo[c][1,2,5]thiadiazolyl, benzo[d]isoxazolyl, benzofuranyl, biphenylyl, chromanyl, furanyl, imidazo[1,2-a]pyridinyl, imidazo[2,1-b]thiazolyl, indolinyl, isoxazolyl, naphthalenyl, phenyl, pyridinyl, pyrimidinyl, quinolinyl, thiophen-2-yl, and thiophen-3-yl; each optionally substituted with one or more substituents selected from: (1-amino-3-hydroxy-1-oxopropan-2-yl)(methyl)amino, (2-(dimethylamino)ethylamino)methyl, (2,2,2-trifluoroethylamino)methyl, (2,2-difluoroethylamino)methyl, (2-acetamidoethyl)(methyl)amino, (2-amino-2-oxoacetamido)methyl, (2-aminoacetamido)methyl, (2-fluoroethylamino)methyl, (2-hydroxyethylamino)methyl, (2-methoxyethylamino)methyl, (cyanomethylamino)methyl, (dimethylamino)methyl, (ethylamino)methyl, (isobutylamino)methyl, (isopentylamino)methyl, (isopropylamino)methyl, (methylamino)methyl, 1-aminocyclopropyl, 2-(benzyloxy)ethyl, 2-(pyrrolidin-1-yl)ethoxy, 2-(trifluoromethyl)phenoxy, 2-aminoethylamino, 2-carboxy-N-methylacetamido, 2-hydroxyethyl, 2-hydroxyethylamino, 2-methoxyethyl, 2-methoxyethylamino, 2-morpholinoethylamino, 3-(dimethylamino)propoxy, 4-(trifluoromethyl)phenoxy, acetamido, acetyl, amino, aminomethyl, benzyl, bromo, carbamimidoyl, carboxamide, carboxy, carboxymethyl, chloro, cyano, cyanomethoxy, cyanomethyl, cyclopropylmethyl, dimethylamino, dimethylcarbamoyl, ethoxy, ethyl, fluoro, hydroxycarbamimidoyl, hydroxyl, hydroxymethyl, isobutyl, isopropoxy, isopropyl, methoxy, methoxymethyl, methyl, methylamino, methylsulfonamido, methylsulfonyl, morpholino, N,N-dimethylsulfamoyl, oxo, phenoxy, phenylsulfonyl, piperazin-1-yl, piperidin-1-yl, propyl, sec-butyl, sulfamoyl, tert-butyl, tert-pentyl, thiazolidin-3-ylmethyl, trifluoromethoxy, and trifluoromethyl.

In some embodiments, R¹¹ is selected from: (1,2,3,4-tetrahydropyrimidin-5-yl)phenyl, (5-isoxazol-3-yl)thiophen-2-yl, (pyridin-2-yl)phenyl, [3,3′-bipyridin]-5-yl, 1,2,3,4-tetrahydropyrido[2,3-b][1,4]oxazepin-8-yl, 1′,2′-dihydrospiro[cyclopropane-1,3′-pyrido[2,3-b][1,4]oxazine]-7′-yl, 1,4-dihydropyridin-3-yl, 1,4-dihydroquinolin-3-yl, 1,5-naphthyridin-3-yl, 1,8-naphthyridin-3-yl, 1H-benzo[d]imidazol-5-yl, 1H-benzo[d]imidazol-6-yl, 1H-imidazo[4,5-b]pyridin-6-yl, 1H-imidazol-4-yl, 1H-indazol-5-yl, 1H-indazol-6-yl, 1H-indol-5-yl, 1H-pyrazol-3-yl, 1H-pyrazol-4-yl, (1H-pyrazol-5-yl)thiophen-2-yl, 1H-pyrazolo[3,4-b]pyridin-5-yl, 1H-pyrazolo[4,3-b]pyridin-6-yl, 1H-pyrrolo[2,3-b]pyridin-3-yl, 1H-pyrrolo[2,3-b]pyridin-5-yl, 1H-pyrrolo[3,2-b]pyridin-3-yl, 1H-pyrrolo[3,2-b]pyridin-6-yl, 1-phenyl-1H-pyrazol-4-yl, 2,3-dihydro-[1,4]dioxino[2,3-b]pyridin-7-yl, 2,3-dihydro-1H-benzo[d]imidazol-5-yl, 2,3-dihydro-1H-imidazo[4,5-b]pyridin-6-yl, 2,3-dihydro-1H-pyrido[2,3-b][1,4]oxazin-6-yl, 2,3-dihydro-1H-pyrido[2,3-b][1,4]oxazin-7-yl, 2,3-dihydro-1H-pyrrolo[2,3-b]pyridin-5-yl, 2,3-dihydrobenzo[b][1,4]dioxin-6-yl, 2,3-dihydrobenzo[d]thiazol-6-yl, 2,3-dihydrobenzofuran-5-yl, 2,3-dihydrobenzofuran-7-yl, 2,3-dihydrofuro[2,3-b]pyridin-5-yl, 2-phenylthiazol-5-yl, 3-(1H-pyrazol-3-yl)phenyl, 3-(1H-pyrazol-4-yl)phenyl, 3-(1H-pyrrol-3-yl)phenyl, 3-(2H-tetrazol-5-yl)phenyl, 3-(furan-2-yl)phenyl, 3-(isoxazol-4-yl)phenyl, 3-(pyridin-2-yl)phenyl, 3-(pyridin-3-yl)phenyl, 3-(pyridin-4-yl)phenyl, 3-(pyrimidin-5-yl)phenyl, 3-(thiazol-5-yl)phenyl, 3-(thiophen-2-yl)phenyl, 3-(thiophen-3-yl)phenyl, 3,4-dihydro-2H-benzo[b][1,4]oxazin-6-yl, 3,4-dihydro-2H-pyrano[2,3-b]pyridin-6-yl, 3,4-dihydro-2H-pyrido[3,2-b][1,4]oxazin-7-yl, 3H-imidazo[4,5-b]pyridin-6-yl, 4′-(1,2,4-oxadiazol-3-yl)biphenyl-3-yl, 4-(2H-tetrazol-5-yl)phenyl, 4-(pyridin-3-yl)phenyl, 4-(pyridin-4-yl)phenyl, 5-(1H-pyrazol-4-yl)pyridin-3-yl, 5-(phenyl)pyridin-3-yl, 5,6,7,8-tetrahydro-1,6-naphthyridin-3-yl, 5,6,7,8-tetrahydronaphthalen-1-yl, 5,6,7,8-tetrahydronaphthalen-2-yl, 5,6,7,8-tetrahydronaphthalenyl, 5,6,7,8-tetrahydroquinolin-3-yl, 5-phenyl-2,3-dihydrobenzofuran-7-yl, 5-phenylthiophen-2-yl, 6,7-dihydro-5H-cyclopenta[b]pyridin-3-yl, 6,7-dihydro-5H-pyrrolo[3,4-b]pyridin-3-yl, 7,8-dihydro-5H-pyrano[4,3-b]pyridin-3-yl, benzo[c][1,2,5]oxadiazol-4-yl, benzo[c][1,2,5]thiadiazol-4-yl, benzo[c][1,2,5]thiadiazol-5-yl, benzo[d]isoxazol-5-yl, benzofuran-2-yl, benzofuran-5-yl, biphenyl-2-yl, biphenyl-3-yl, biphenyl-4-yl, chroman-6-yl, furan-3-yl, imidazo[1,2-a]pyridin-6-yl, imidazo[2,1-b]thiazol-5-yl, indolin-5-yl, isoxazol-4-yl, naphthalen-1-yl, naphthalen-2-yl, phenyl, pyridin-2-yl, pyridin-3-yl, pyrimidin-5-yl, quinolin-3-yl, quinolin-6-yl, quinolin-8-yl, thiophen-2-yl, and thiophen-3-yl; each optionally substituted with one or more substituents selected from: (1-amino-3-hydroxy-1-oxopropan-2-yl)(methyl)amino, (2-(dimethylamino)ethylamino)methyl, (2,2,2-trifluoroethylamino)methyl, (2,2-difluoroethylamino)methyl, (2-acetamidoethyl)(methyl)amino, (2-amino-2-oxoacetamido)methyl, (2-aminoacetamido)methyl, (2-fluoroethylamino)methyl, (2-hydroxyethylamino)methyl, (2-methoxyethylamino)methyl, (cyanomethylamino)methyl, (dimethylamino)methyl, (ethylamino)methyl, (isobutylamino)methyl, (isopentylamino)methyl, (isopropylamino)methyl, (methylamino)methyl, 1-aminocyclopropyl, 2-(benzyloxy)ethyl, 2-(pyrrolidin-1-yl)ethoxy, 2-(trifluoromethyl)phenoxy, 2-aminoethylamino, 2-carboxy-N-methylacetamido, 2-hydroxyethyl, 2-hydroxyethylamino, 2-methoxyethyl, 2-methoxyethylamino, 2-morpholinoethylamino, 3-(dimethylamino)propoxy, 4-(trifluoromethyl)phenoxy, acetamido, acetyl, amino, aminomethyl, benzyl, bromo, carbamimidoyl, carboxamide, carboxy, carboxymethyl, chloro, cyano, cyanomethoxy, cyanomethyl, cyclopropylmethyl, dimethylamino, dimethylcarbamoyl, ethoxy, ethyl, fluoro, hydroxycarbamimidoyl, hydroxyl, hydroxymethyl, isobutyl, isopropoxy, isopropyl, methoxy, methoxymethyl, methyl, methylamino, methylsulfonamido, methylsulfonyl, morpholino, N,N-dimethylsulfamoyl, oxo, phenoxy, phenylsulfonyl, piperazin-1-yl, piperidin-1-yl, propyl, sec-butyl, sulfamoyl, tert-butyl, tert-pentyl, thiazolidin-3-ylmethyl, trifluoromethoxy, and trifluoromethyl.

In some embodiments, R¹¹ is selected from: (dimethylcarbamoyl)phenyl, 1-(2-(benzyloxy)ethyl)-2,3-dihydro-1H-pyrido[2,3-b][1,4]oxazin-7-yl, 1-(2-hydroxyethyl)-2,3-dihydro-1H-pyrido[2,3-b][1,4]oxazin-7-yl, 1-(2-methoxyethyl)-2,3-dihydro-1H-pyrido[2,3-b][1,4]oxazin-7-yl, 1-(carboxymethyl)-4-oxo-1,4-dihydroquinolin-3-yl, 1,2-dimethyl-1H-imidazol-4-yl, 1,3,3-trimethyl-2,3-dihydro-1H-pyrido[2,3-b][1,4]oxazin-7-yl, 1,3-dimethyl-2,3-dihydro-1H-pyrido[2,3-b][1,4]oxazin-7-yl, 1,5-naphthyridin-3-yl, 1,6-dimethyl-2,3-dihydro-1H-pyrido[2,3-b][1,4]oxazin-7-yl, 1,8-dimethyl-2,3-dihydro-1H-pyrido[2,3-b][1,4]oxazin-7-yl, 1-ethyl-2,3-dihydro-1H-pyrido[2,3-b][1,4]oxazin-7-yl, 1-ethyl-4-oxo-1,4-dihydropyridin-3-yl, 1-ethyl-4-oxo-1,4-dihydroquinolin-3-yl, 1-ethyl-5-methyl-1H-pyrazol-4-yl, 1-ethyl-6-fluoro-4-oxo-1,4-dihydroquinolin-3-yl, 1-ethyl-6-methyl-4-oxo-1,4-dihydroquinolin-3-yl, 1-ethyl-7-fluoro-4-oxo-1,4-dihydroquinolin-3-yl, 1-ethyl-7-methyl-4-oxo-1,4-dihydroquinolin-3-yl, 1-ethyl-8-fluoro-4-oxo-1,4-dihydroquinolin-3-yl, 1-ethyl-8-methyl-4-oxo-1,4-dihydroquinolin-3-yl, 1H-benzo[d]imidazol-5-yl, 1H-benzo[d]imidazol-6-yl, 1H-imidazo[4,5-b]pyridin-6-yl, 1H-indazol-5-yl, 1H-indazol-6-yl, 1H-pyrazol-4-yl, 1H-pyrazolo[3,4-b]pyridin-5-yl, 1H-pyrazolo[4,3-b]pyridin-6-yl, 1H-pyrrolo[2,3-b]pyridin-3-yl, 1H-pyrrolo[2,3-b]pyridin-5-yl, 1H-pyrrolo[3,2-b]pyridin-3-yl, 1H-pyrrolo[3,2-b]pyridin-6-yl, 1-isopropyl-2,3-dihydro-1H-pyrido[2,3-b][1,4]oxazin-7-yl, 1-methyl-1,2,3,4-tetrahydropyrido[2,3-b][1,4]oxazepin-8-yl, 1′-methyl-1′,2′-dihydrospiro[cyclopropane-1,3′-pyrido[2,3-b][1,4]oxazine]-7′-yl, 1-methyl-1H-imidazo[4,5-b]pyridin-6-yl, 1-methyl-1H-imidazol-4-yl, 1-methyl-1H-indol-5-yl, 1-methyl-1H-pyrazol-3-yl, 1-methyl-1H-pyrrolo[3,2-b]pyridin-6-yl, 1-methyl-2,3-dihydro-1H-pyrido[2,3-b][1,4]oxazin-7-yl, 1-methyl-2-oxo-2,3-dihydro-1H-imidazo[4,5-b]pyridin-6-yl, 1-methyl-3-(trifluoromethyl)-1H-pyrazol-4-yl, 1-methyl-4-oxo-1,4-dihydroquinolin-3-yl, 1-methyl-6-(methylamino)-2,3-dihydro-1H-pyrido[2,3-b][1,4]oxazin-7-yl, 1-phenyl-1H-pyrazol-4-yl, 1-propyl-2,3-dihydro-1H-pyrido[2,3-b][1,4]oxazin-7-yl, 2-(dimethylamino)pyridin-3-yl, 2-(methylsulfonyl)phenyl, 2-(pyridin-4-yl)ethyl, 2-(trifluoromethoxy)phenyl, 2-(trifluoromethyl)phenyl, 2,2-dimethylchroman-6-yl, 2,3-dichlorophenyl, 2,3-difluorophenyl, 2,3-dihydro-[1,4]dioxino[2,3-b]pyridin-7-yl, 2,3-dihydro-1H-pyrido[2,3-b][1,4]oxazin-7-yl, 2,3-dihydrobenzo[b][1,4]dioxin-6-yl, 2,3-dihydrobenzofuran-5-yl, 2,3-dihydrofuro[2,3-b]pyridin-5-yl, 2,3-dimethylphenyl, 2,3-dioxoindolin-5-yl, 2,4-dichlorophenyl, 2,4-difluorophenyl, 2,5-dichlorophenyl, 2,5-difluorophenyl, 2,5-dimethylphenyl, 2,6-difluorophenyl, 2-bromophenyl, 2-chloro-3-fluorophenyl, 2-chloro-4-cyanophenyl, 2-chloro-4-fluorophenyl, 2-chloro-5-(methylsulfonyl)phenyl, 2-chloro-5-(trifluoromethyl)phenyl, 2-chloro-5-fluorophenyl, 2-chlorophenyl, 2-cyano-5-methoxyphenyl, 2-cyano-5-methylphenyl, 2-cyanophenyl, 2-ethyl-3H-imidazo[4,5-b]pyridin-6-yl, 2-fluoro-3-methylphenyl, 2-fluoro-5-methoxyphenyl, 2-fluoro-5-methylphenyl, 2-fluorophenyl, 2-hydroxypyrimidin-5-yl, 2-methoxy-4-methylphenyl, 2-methoxy-5-methylphenyl, 2-methoxyphenyl, 2-methyl-1H-benzo[d]imidazol-5-yl, 2-methyl-1H-imidazo[4,5-b]pyridin-6-yl, 2-methyl-3H-imidazo[4,5-b]pyridin-6-yl, 2-morpholinopyridin-3-yl, 2-oxo-2,3-dihydro-1H-benzo[d]imidazol-5-yl, 2-oxo-2,3-dihydro-1H-imidazo[4,5-b]pyridin-6-yl, 2-oxo-2,3-dihydro-1H-pyrido[2,3-b][1,4]oxazin-6-yl, 2-oxo-2,3-dihydro-1H-pyrrolo[2,3-b]pyridin-5-yl, 2-oxo-2,3-dihydrobenzo[d]thiazol-6-yl, 2-oxoindolin-5-yl, 3′-((dimethylamino)methyl)biphenyl-3-yl, 3-(1-(2-hydroxyethyl)-1H-pyrazol-4-yl)phenyl, 3-(1-(cyclopropylmethyl)-1H-pyrazol-4-yl)phenyl, 3-(1,3-dimethyl-1H-pyrazol-4-yl)phenyl, 3-(1,3-dimethyl-2,4-dioxo-1,2,3,4-tetrahydropyrimidin-5-yl)phenyl, 3-(1-benzyl-1H-pyrazol-4-yl)phenyl, 3-(1-ethyl-1H-pyrazol-4-yl)phenyl, 3-(1H-pyrazol-3-yl)phenyl, 3-(1H-pyrazol-4-yl)phenyl, 3-(1H-pyrrol-3-yl)phenyl, 3-(1-isobutyl-1H-pyrazol-4-yl)phenyl, 3-(1-methyl-1H-pyrazol-4-yl)phenyl, 3-(1-methyl-1H-pyrrol-3-yl)phenyl, 3-(1-methyl-3-(trifluoromethyl)-1H-pyrazol-4-yl)phenyl, 3-(1-propyl-1H-pyrazol-4-yl)phenyl, 3-(2-(trifluoromethyl)phenoxy)phenyl, 3-(2,4-dimethylthiazol-5-yl)phenyl, 3-(2H-tetrazol-5-yl)phenyl, 3-(2-methoxypyrimidin-5-yl)phenyl, 3-(2-methylpyridin-4-yl)phenyl, 3-(3,5-dimethylisoxazol-4-yl)phenyl, 3-(3-methylthiophen-2-yl)phenyl, 3-(4-(trifluoromethyl)phenoxy)phenyl, 3-(4-methylthiophen-3-yl)phenyl, 3-(5-(aminomethyl)thiophen-2-yl)phenyl, 3-(5-cyanopyridin-3-yl)phenyl, 3-(5-methylpyridin-3-yl)phenyl, 3-(6-(2-morpholinoethylamino)pyridin-3-yl)phenyl, 3-(6-(3-(dimethylamino)propoxy)pyridin-3-yl)phenyl, 3-(6-(aminomethyl)pyridin-3-yl)phenyl, 3-(6-aminopyridin-3-yl)phenyl, 3-(6-methylpyridin-3-yl)phenyl, 3′-(aminomethyl)biphenyl-3-yl, 3-(aminomethyl)phenyl, 3′-(carboxy)biphenyl-3-yl, 3′-(dimethylamino)biphenyl-3-yl, 3-(furan-2-yl)phenyl, 3′-(hydroxymethyl)biphenyl-3-yl, 3-(hydroxymethyl)phenyl, 3′-(methoxymethyl)biphenyl-3-yl, 3′-(methylsulfonyl)biphenyl-3-yl, 3′-(N,N-dimethylsulfamoyl)biphenyl-3-yl, 3-(pyridin-2-yl)phenyl, 3-(pyridin-3-yl)phenyl, 3-(pyridin-4-yl)phenyl, 3-(pyrimidin-5-yl)phenyl, 3-(thiophen-3-yl)phenyl, 3-(trifluoromethyl)-1H-pyrrolo[2,3-b]pyridin-5-yl, 3-(trifluoromethyl)phenyl, 3,4-difluorophenyl, 3,4-dihydro-2H-benzo[b][1,4]oxazin-6-yl, 3,4-dihydro-2H-pyrano[2,3-b]pyridin-6-yl, 3,4-dihydro-2H-pyrido[3,2-b][1,4]oxazin-7-yl, 3,4-dimethoxyphenyl, 3,4-dimethylphenyl, 3,5-dichlorophenyl, 3,5-dimethyl-1-phenyl-1H-pyrazol-4-yl, 3,5-dimethylisoxazol-4-yl, 3,5-dimethylphenyl, 3-bromo-4-methylphenyl, 3-bromo-5-methylphenyl, 3-bromophenyl, 3-carboxyphenyl, 3-chloro-1H-pyrrolo[2,3-b]pyridin-5-yl, 3-chloro-1-methyl-1H-pyrrolo[2,3-b]pyridin-5-yl, 3-chloro-1-methyl-1H-pyrrolo[3,2-b]pyridin-6-yl, 3-chloro-2-fluorophenyl, 3-chloro-2-methyl-1H-pyrrolo[2,3-b]pyridin-5-yl, 3-chloro-2-methylphenyl, 3-chloro-4-cyanophenyl, 3-chloro-4-methoxyphenyl, 3-chlorophenyl, 3-cyano-1H-pyrrolo[2,3-b]pyridin-5-yl, 3-cyano-4-methylphenyl, 3′-cyanobiphenyl-3-yl, 3-cyanophenyl, 3-ethyl-3H-imidazo[4,5-b]pyridin-6-yl, 3-fluoro-4-methoxyphenyl, 3-fluoro-5-methylphenyl, 3-fluorophenyl, 3-methoxyphenyl, 3-methyl-1H-pyrazolo[3,4-b]pyridin-5-yl, 3-methyl-1H-pyrrolo[2,3-b]pyridin-5-yl, 3-methyl-2-oxo-2,3-dihydro-1H-imidazo[4,5-b]pyridin-6-yl, 3-methyl-3H-imidazo[4,5-b]pyridin-6-yl, 3-oxo-3,4-dihydro-2H-benzo[b][1,4]oxazin-6-yl, 3-phenoxyphenyl, 4′-((1-amino-3-hydroxy-1-oxopropan-2-yl)(methyl)amino)biphenyl-3-yl, 4′-((2-(dimethylamino)ethylamino)methyl)biphenyl-3-yl, 4′-((2,2,2-trifluoroethylamino)methyl)biphenyl-3-yl, 4′-((2,2-difluoroethylamino)methyl)biphenyl-3-yl, 4′-((2-acetamidoethyl)(methyl)amino)biphenyl-3-yl, 4′-((2-amino-2-oxoacetamido)methyl)biphenyl-3-yl, 4′-((2-aminoacetamido)methyl)biphenyl-3-yl, 4′-((2-fluoroethylamino)methyl)biphenyl-3-yl, 4′-((2-hydroxyethylamino)methyl)biphenyl-3-yl, 4′-((2-methoxyethylamino)methyl)biphenyl-3-yl, 4′-((cyanomethylamino)methyl)biphenyl-3-yl, 4′-((dimethylamino)methyl)biphenyl-4-yl, 4′-((ethylamino)methyl)biphenyl-3-yl, 4′-((isobutylamino)methyl)biphenyl-3-yl, 4′-((isopentylamino)methyl)biphenyl-3-yl, 4′-((isopropylamino)methyl)biphenyl-3-yl, 4′-((methylamino)methyl)biphenyl-3-yl, 4′-(1-aminocyclopropyl)-6-methoxybiphenyl-3-yl, 4′-(1-aminocyclopropyl)biphenyl-3-yl, 4′-(2-carboxy-N-methylacetamido)biphenyl-3-yl, 4-(2H-tetrazol-5-yl)phenyl, 4-(3-methoxyphenyl)pyrimidin-2-yl, 4-carboxypyrimidin-2-yl, 4′-(5-methyl-1,2,4-oxadiazol-3-yl)biphenyl-3-yl, 4′-(aminomethyl)-2-methoxybiphenyl-3-yl, 4′-(aminomethyl)-2-methylbiphenyl-3-yl, 4′-(aminomethyl)-3-(trifluoromethoxy)biphenyl-4-yl, 4′-(aminomethyl)-4-(trifluoromethoxy)biphenyl-3-yl, 4′-(aminomethyl)-4-chlorobiphenyl-3-yl, 4′-(aminomethyl)-4-ethoxybiphenyl-3-yl, 4′-(aminomethyl)-4-methoxybiphenyl-3-yl, 4′-(aminomethyl)-4-methylbiphenyl-3-yl, 4′-(aminomethyl)-5-(trifluoromethyl)biphenyl-3-yl, 4′-(aminomethyl)-5-methoxybiphenyl-3-yl, 4′-(aminomethyl)-5-methylbiphenyl-3-yl, 4′-(aminomethyl)-6-methoxybiphenyl-3-yl, 4′-(aminomethyl)-6-methylbiphenyl-3-yl, 4′-(aminomethyl)biphenyl-2-yl, 4′-(aminomethyl)biphenyl-3-yl, 4′-(aminomethyl)biphenyl-4-yl, 4-(aminomethyl)phenyl, 4′-(carboxymethyl)biphenyl-3-yl, 4′-(cyanomethoxy)biphenyl-3-yl, 4′-(cyanomethyl)biphenyl-3-yl, 4-(hydroxymethyl)phenyl, 4′-(methylsulfonamido)biphenyl-3-yl, 4-(methylsulfonyl)phenyl, 4′-(N′-hydroxycarbamimidoyl)-biphenyl-3-yl, 4-(phenylsulfonyl)thiophen-2-yl, 4-(pyridin-2-yl)phenyl, 4-(pyridin-3-yl)phenyl, 4-(pyridin-4-yl)phenyl, 4′-(sulfamoyl)biphenyl-3-yl, 4′-(thiazolidin-3-ylmethyl)biphenyl-3-yl, 4-(trifluoromethoxy)phenyl, 4′-(trifluoromethyl)biphenyl-4-yl, 4-(trifluoromethyl)phenyl, 4-(trifluoromethyl)pyrimidin-2-yl, 4,5-dichlorothiophen-2-yl, 4,6-dimethoxypyrimidin-2-yl, 4,6-dimethylpyrimidin-2-yl, 4-acetamidophenyl, 4-acetylphenyl, 4-aminopyrimidin-2-yl, 4-benzylpyrimidin-2-yl, 4-bromo-3-chlorophenyl, 4-bromo-3-methylphenyl, 4-bromophenyl, 4′-carbamimidoyl-biphenyl-3-yl, 4′-carbamoyl-biphenyl-3-yl, 4-carboxyphenyl, 4-chloro-3-methoxyphenyl, 4-chloro-3-methylphenyl, 4-chlorophenyl, 4-chloropyridin-2-yl, 4-cyanophenyl, 4-ethoxy-4′-((isopropylamino)methyl)biphenyl-3-yl, 4-ethoxyphenyl, 4-ethyl-3,4-dihydro-2H-benzo[b][1,4]oxazin-6-yl, 4-fluoro-3-methylphenyl, 4′-fluorobiphenyl-4-yl, 4-fluorophenyl, 4-hydroxy-6-methylquinolin-3-yl, 4-hydroxy-6-methylquinolin-8-yl, 4-hydroxy-7-methylquinolin-3-yl, 4-hydroxy-8-methylquinolin-3-yl, 4-hydroxyquinolin-3-yl, 4-isobutyl-3,4-dihydro-2H-benzo[b][1,4]oxazin-6-yl, 4-isopropoxyphenyl, 4-isopropyl-3,4-dihydro-2H-benzo[b][1,4]oxazin-6-yl, 4-methoxy-1H-indazol-5-yl, 4-methoxy-2,3-dimethylphenyl, 4-methoxy-2-methylphenyl, 4-methoxy-3-methylphenyl, 4-methoxy-5,6,7,8-tetrahydronaphthalen-1-yl, 4-methoxynaphthalen-1-yl, 4-methoxyphenyl, 4-methoxypyrimidin-2-yl, 4-methyl-2-phenylthiazol-5-yl, 4-methyl-3,4-dihydro-2H-benzo[b][1,4]oxazin-6-yl, 4-methyl-3,4-dihydro-2H-pyrido[3,2-b][1,4]oxazin-7-yl, 4-methyl-6-phenylpyrimidin-2-yl, 4-methylpyrimidin-2-yl, 4-oxo-1-propyl-1,4-dihydroquinolin-3-yl, 4-phenylpyrimidin-2-yl, 4-sec-butylphenyl, 4-tert-butylphenyl, 4-tert-pentylphenyl, 5-(1-methyl-1H-pyrazol-4-yl)pyridin-3-yl, 5-(1-methyl-3-(trifluoromethyl)-1H-pyrazol-5-yl)thiophen-2-yl, 5-(4-(aminomethyl)phenyl)-2,3-dihydrobenzofuran-7-yl, 5-(4-(aminomethyl)phenyl)pyridin-3-yl, 5-(5-(trifluoromethyl)isoxazol-3-yl)thiophen-2-yl, 5-(methoxycarbonyl)pyrimidin-2-yl, 5-(trifluoromethyl)pyrazin-2-yl, 5-(trifluoromethyl)pyrimidin-2-yl, 5,6,7,8-tetrahydro-1,6-naphthyridin-3-yl, 5,6,7,8-tetrahydronaphthalen-2-yl, 5,6,7,8-tetrahydroquinolin-3-yl, 5-benzylpyrimidin-2-yl, 5-bromo-2-(2-(pyrrolidin-1-yl)ethoxy)phenyl, 5-bromo-2,3-dihydrobenzofuran-7-yl, 5-bromo-2-chlorophenyl, 5-bromo-2-methoxyphenyl, 5-bromo-2-methylphenyl, 5-bromopyridin-3-yl, 5-chloro-1,3-dimethyl-1H-pyrazol-4-yl, 5-chloro-2-cyanophenyl, 5-chloro-2-fluorophenyl, 5-chloro-2-methoxyphenyl, 5-chloro-2-methylphenyl, 5-chlorobenzo[c][1,2,5]oxadiazol-4-yl, 5-chloronaphthalen-2-yl, 5-chlorothiophen-2-yl, 5-cyano-2-methylphenyl, 5-ethylpyrimidin-2-yl, 5-fluoro-2-methoxyphenyl, 5-fluoro-2-methylphenyl, 5-heptylpyrimidin-2-yl, 5-methoxy-2-methylpyridin-3-yl, 5-methoxypyridin-3-yl, 5-methyl-1-phenyl-1H-pyrazol-4-yl, 5-methyl-2-(trifluoromethyl)furan-3-yl, 5-methyl-3H-imidazo[4,5-b]pyridin-6-yl, 5-methylbenzo[c][1,2,5]oxadiazol-4-yl, 5-oxo-6,7-dihydro-5H-pyrrolo[3,4-b]pyridin-3-yl, 5-phenylpyrimidin-2-yl, 5-phenylthiophen-2-yl, 5-propylpyrimidin-2-yl, 6-(2-aminoethylamino)pyridin-3-yl, 6-(2-hydroxyethylamino)pyridin-3-yl, 6-(2-methoxyethylamino)pyridin-3-yl, 6′-(aminomethyl)-3,3′-bipyridin-5-yl, 6-(dimethylamino)-1-methyl-2,3-dihydro-1H-pyrido[2,3-b][1,4]oxazin-7-yl, 6-(dimethylamino)pyridin-3-yl, 6-(piperazin-1-yl)pyridin-3-yl, 6-(piperidin-1-yl)pyridin-3-yl, 6,7-dihydro-5H-cyclopenta[b]pyridin-3-yl, 6,7-dihydro-5H-pyrrolo[3,4-b]pyridin-3-yl, 6-amino-1-methyl-2,3-dihydro-1H-pyrido[2,3-b][1,4]oxazin-7-yl, 6-chloro-1-methyl-2,3-dihydro-1H-pyrido[2,3-b][1,4]oxazin-7-yl, 6-chloroimidazo[2,1-b]thiazol-5-yl, 6-chloronaphthalen-2-yl, 6-ethoxy-1-methyl-2,3-dihydro-1H-pyrido[2,3-b][1,4]oxazin-7-yl, 6-ethoxypyridin-3-yl, 6-fluoro-4-hydroxyquinolin-3-yl, 6-fluoro-4-oxo-1,4-dihydroquinolin-3-yl, 6-hydroxy-1-methyl-2,3-dihydro-1H-pyrido[2,3-b][1,4]oxazin-7-yl, 6-hydroxypyridin-3-yl, 6-methoxy-1-methyl-2,3-dihydro-1H-pyrido[2,3-b][1,4]oxazin-7-yl, 6-methoxynaphthalen-2-yl, 6-methoxypyridin-3-yl, 6-morpholinopyridin-3-yl, 6-phenoxypyridin-3-yl, 7,8-dihydro-5H-pyrano[4,3-b]pyridin-3-yl, 7-amino-1,8-naphthyridin-3-yl, 7-chlorobenzo[c][1,2,5]oxadiazol-4-yl, 7-fluoro-4-hydroxyquinolin-3-yl, 7-fluoro-4-oxo-1,4-dihydroquinolin-3-yl, 7-methoxybenzo[c][1,2,5]oxadiazol-4-yl, 7-methyl-3H-imidazo[4,5-b]pyridin-6-yl, 8-fluoro-4-hydroxyquinolin-3-yl, 8-fluoro-4-oxo-1,4-dihydroquinolin-3-yl, 8-methyl-4-oxo-1,4-dihydroquinolin-3-yl, benzo[c][1,2,5]thiadiazol-4-yl, benzo[c][1,2,5]thiadiazol-5-yl, benzo[d]isoxazol-5-yl, benzofuran-2-yl, benzofuran-5-yl, benzyl, biphenyl-2-yl, biphenyl-3-yl, biphenyl-4-yl, chroman-6-yl, cyclohexyl, furan-3-yl, imidazo[1,2-a]pyridin-6-yl, m-tolyl, naphthalen-1-yl, naphthalen-2-yl, phenyl, p-tolyl, pyrazin-2-yl, pyridin-2-yl, pyridin-3-yl, pyridin-3-yl N-oxide, pyrimidin-2-yl, pyrimidin-4-yl, quinolin-3-yl, quinolin-6-yl, and thiophen-3-yl.

### The R¹¹ Group (Aryl)

One aspect of the present invention relates to wherein R¹¹ is aryl optionally substituted with one or more substituents as described herein.

In some embodiments, R¹¹ is aryl optionally substituted with one or more substituents selected from: C₁-C₆ alkoxy, C₁-C₇ alkyl, C₁-C₆ alkylcarboxamide, C₁-C₆ alkylsulfonamido, C₁-C₆ alkylsulfonyl, aryloxy, carboxamide, carbamimidoyl, carboxy, cyano, C₃-C₇ cycloalkyl, C₂-C₈ dialkylamino, C₂-C₈ dialkylsulfamoyl, C₁-C₆ haloalkoxy, C₁-C₆ haloalkyl, halogen, hydroxycarbamimidoyl, and sulfamoyl; and wherein said C₁-C₆ alkoxy, C₁-C₇ alkyl, aryloxy, and C₂-C₈ dialkylamino are each optionally substituted with one or more substituents selected from: amino, C₁-C₆ alkoxy, C₁-C₆ alkylamino, C₁-C₆ alkylcarboxamide, carboxy, —Y¹—C₁-C₆-alkylene-Z¹ optionally substituted with oxo, cyano, C₂-C₆ dialkylamino, C₁-C₆ haloalkyl, C₁-C₆ haloalkylamino, heterocyclyl, hydroxyl, and oxo;
Y¹ is —NH—; and
Z¹ is selected from: C₁-C₆ alkoxy, amino, cyano, C₂-C₆ dialkylamino, and hydroxyl.

In some embodiments, R¹¹ is selected from: 5,6,7,8-tetrahydronaphthalenyl, biphenylyl, naphthalenyl, and phenyl; each optionally substituted with one or more substituents selected from: (2-ethyl)(methyl)amino, 4-(trifluoromethyl)phenoxy, acetamido, bromo, carbamimidoyl, carboxamide, carboxy, chloro, cyano, cyclopropyl, dimethylamino, dimethylcarbamoyl, ethoxy, ethyl, fluoro, hydroxycarbamimidoyl, isopropoxy, isopropyl(methyl)amino, methoxy, methyl, methyl(propyl)amino, methylsulfonamido, methylsulfonyl, N,N-dimethylsulfamoyl, phenoxy, sec-butyl, sulfamoyl, tert-butyl, tert-pentyl, trifluoromethoxy, and trifluoromethyl; and wherein (2-ethyl)(methyl)amino, cyclopropyl, ethoxy, ethyl, isopropyl(methyl)amino, methoxy, methyl, methyl(propyl)amino, and phenoxy; are each optionally substituted with one or more substituents selected from: 2-(dimethylamino)ethylamino, 2,2,2-trifluoroethylamino, 2,2-difluoroethylamino, 2-amino-2-oxoacetamido, 2-aminoacetamido, 2-fluoroethylamino, 2-hydroxyethylamino, 2-methoxyethylamino, acetamido, amino, carboxy, cyano, cyanomethylamino, dimethylamino, ethylamino, hydroxyl, isobutylamino, isopentylamino, isopropylamino, methoxy, methylamino, oxo, pyrrolidin-1-yl, thiazolidin-3-yl, and trifluoromethyl.

In some embodiments, R¹¹ is selected from: 5,6,7,8-tetrahydronaphthalen-1-yl, 5,6,7,8-tetrahydronaphthalen-2-yl, biphenyl-2-yl, biphenyl-3-yl, biphenyl-4-yl, naphthalen-1-yl, naphthalen-2-yl, and phenyl; each optionally substituted with one or more substituents selected from: (1-amino-3-hydroxy-1-oxopropan-2-yl)(methyl)amino, (2-(dimethylamino)ethylamino)methyl, (2,2,2-trifluoroethylamino)methyl, (2,2-difluoroethylamino)methyl, (2-acetamidoethyl)(methyl)amino, (2-amino-2-oxoacetamido)methyl, (2-aminoacetamido)methyl, (2-fluoroethylamino)methyl, (2-hydroxyethylamino)methyl, (2-methoxyethylamino)methyl, (cyanomethylamino)methyl, (dimethylamino)methyl, (ethylamino)methyl, (isobutylamino)methyl, (isopentylamino)methyl, (isopropylamino)methyl, (methylamino)methyl, 1-aminocyclopropyl, 2-(pyrrolidin-1-yl)ethoxy, 2-(trifluoromethyl)phenoxy, 2-carboxy-N-methylacetamido, 4-(trifluoromethyl)phenoxy, acetamido, acetyl, aminomethyl, bromo, carbamimidoyl, carboxamide, carboxy, carboxymethyl, chloro, cyano, cyanomethoxy, cyanomethyl, dimethylamino, dimethylcarbamoyl, ethoxy, fluoro, hydroxycarbamimidoyl, hydroxymethyl, isopropoxy, methoxy, methoxymethyl, methyl, methylsulfonamido, methylsulfonyl, N,N-dimethylsulfamoyl, phenoxy, sec-butyl, sulfamoyl, tert-butyl, tert-pentyl, thiazolidin-3-ylmethyl, trifluoromethoxy, and trifluoromethyl.

In some embodiments, R¹¹ is selected from: (dimethylcarbamoyl)phenyl, 2-(methylsulfonyl)phenyl, 2-(trifluoromethoxy)phenyl, 2-(trifluoromethyl)phenyl, 2,3-dichlorophenyl, 2,3-difluorophenyl, 2,3-dimethylphenyl, 2,4-dichlorophenyl, 2,4-difluorophenyl, 2,5-dichlorophenyl, 2,5-difluorophenyl, 2,5-dimethylphenyl, 2,6-difluorophenyl, 2-bromophenyl, 2-chloro-3-fluorophenyl, 2-chloro-4-cyanophenyl, 2-chloro-4-fluorophenyl, 2-chloro-5-(methylsulfonyl)phenyl, 2-chloro-5-(trifluoromethyl)phenyl, 2-chloro-5-fluorophenyl, 2-chlorophenyl, 2-cyano-5-methoxyphenyl, 2-cyano-5-methylphenyl, 2-cyanophenyl, 2-fluoro-3-methylphenyl, 2-fluoro-5-methoxyphenyl, 2-fluoro-5-methylphenyl, 2-fluorophenyl, 2-methoxy-4-methylphenyl, 2-methoxy-5-methylphenyl, 2-methoxyphenyl, 3′-((dimethylamino)methyl)biphenyl-3-yl, 3-(2-(trifluoromethyl)phenoxy)phenyl, 3-(4-(trifluoromethyl)phenoxy)phenyl, 3′-(aminomethyl)biphenyl-3-yl, 3-(aminomethyl)phenyl, 3′-(carboxy)biphenyl-3-yl, 3′-(dimethylamino)biphenyl-3-yl, 3′-(hydroxymethyl)biphenyl-3-yl, 3-(hydroxymethyl)phenyl, 3′-(methoxymethyl)biphenyl-3-yl, 3′-(methylsulfonyl)biphenyl-3-yl, 3′-(N,N-dimethylsulfamoyl)biphenyl-3-yl, 3-(trifluoromethyl)phenyl, 3,4-difluorophenyl, 3,4-dimethoxyphenyl, 3,4-dimethylphenyl, 3,5-dichlorophenyl, 3,5-dimethylphenyl, 3-bromo-4-methylphenyl, 3-bromo-5-methylphenyl, 3-bromophenyl, 3-carboxyphenyl, 3-chloro-2-fluorophenyl, 3-chloro-2-methylphenyl, 3-chloro-4-cyanophenyl, 3-chloro-4-methoxyphenyl, 3-chlorophenyl, 3-cyano-4-methylphenyl, 3′-cyanobiphenyl-3-yl, 3-cyanophenyl, 3-fluoro-4-methoxyphenyl, 3-fluoro-5-methylphenyl, 3-fluorophenyl, 3-methoxyphenyl, 3-phenoxyphenyl, 4′-((1-amino-3-hydroxy-1-oxopropan-2-yl)(methyl)amino)biphenyl-3-yl, 4′-((2-(dimethylamino)ethylamino)methyl)biphenyl-3-yl, 4′-((2,2,2-trifluoroethylamino)methyl)biphenyl-3-yl, 4′-((2,2-difluoroethylamino)methyl)biphenyl-3-yl, 4′-((2-acetamidoethyl)(methyl)amino)biphenyl-3-yl, 4′-((2-amino-2-oxoacetamido)methyl)biphenyl-3-yl, 4′-((2-aminoacetamido)methyl)biphenyl-3-yl, 4′-((2-fluoroethylamino)methyl)biphenyl-3-yl, 4′-((2-hydroxyethylamino)methyl)biphenyl-3-yl, 4′-((2-methoxyethylamino)methyl)biphenyl-3-yl, 4′-((cyanomethylamino)methyl)biphenyl-3-yl, 4′-((dimethylamino)methyl)biphenyl-4-yl, 4′-((ethylamino)methyl)biphenyl-3-yl, 4′-((isobutylamino)methyl)biphenyl-3-yl, 4′-((isopentylamino)methyl)biphenyl-3-yl, 4′-((isopropylamino)methyl)biphenyl-3-yl, 4′-((methylamino)methyl)biphenyl-3-yl, 4′-(1-aminocyclopropyl)-6-methoxybiphenyl-3-yl, 4′-(1-aminocyclopropyl)biphenyl-3-yl, 4′-(2-carboxy-N-methylacetamido)biphenyl-3-yl, 4′-(aminomethyl)-2-methoxybiphenyl-3-yl, 4′-(aminomethyl)-2-methylbiphenyl-3-yl, 4′-(aminomethyl)-3-(trifluoromethoxy)biphenyl-4-yl, 4′-(aminomethyl)-4-(trifluoromethoxy)biphenyl-3-yl, 4′-(aminomethyl)-4-chlorobiphenyl-3-yl, 4′-(aminomethyl)-4-ethoxybiphenyl-3-yl, 4′-(aminomethyl)-4-methoxybiphenyl-3-yl, 4′-(aminomethyl)-4-methylbiphenyl-3-yl, 4′-(aminomethyl)-5-(trifluoromethyl)biphenyl-3-yl, 4′-(aminomethyl)-5-methoxybiphenyl-3-yl, 4′-(aminomethyl)-5-methylbiphenyl-3-yl, 4′-(aminomethyl)-6-methoxybiphenyl-3-yl, 4′-(aminomethyl)-6-methylbiphenyl-3-yl, 4′-(aminomethyl)biphenyl-2-yl, 4′-(aminomethyl)biphenyl-3-yl, 4′-(aminomethyl)biphenyl-4-yl, 4-(aminomethyl)phenyl, 4′-(carboxymethyl)biphenyl-3-yl, 4′-(cyanomethoxy)biphenyl-3-yl, 4′-(cyanomethyl)biphenyl-3-yl, 4-(hydroxymethyl)phenyl, 4′-(methylsulfonamido)biphenyl-3-yl, 4-(methylsulfonyl)phenyl, 4′-(N′-hydroxycarbamimidoyl)-biphenyl-3-yl, 4′-(sulfamoyl)biphenyl-3-yl, 4′-(thiazolidin-3-ylmethyl)biphenyl-3-yl, 4-(trifluoromethoxy)phenyl, 4′-(trifluoromethyl)biphenyl-4-yl, 4-(trifluoromethyl)phenyl, 4-acetamidophenyl, 4-acetylphenyl, 4-bromo-3-chlorophenyl, 4-bromo-3-methylphenyl, 4-bromophenyl, 4′-carbamimidoyl-biphenyl-3-yl, 4′-carbamoyl-biphenyl-3-yl, 4-carboxyphenyl, 4-chloro-3-methoxyphenyl, 4-chloro-3-methylphenyl, 4-chlorophenyl, 4-cyanophenyl, 4-ethoxy-4′-((isopropylamino)methyl)biphenyl-3-yl, 4-ethoxyphenyl, 4-fluoro-3-methylphenyl, 4′-fluorobiphenyl-4-yl, 4-fluorophenyl, 4-isopropoxyphenyl, 4-methoxy-2,3-dimethylphenyl, 4-methoxy-2-methylphenyl, 4-methoxy-3-methylphenyl, 4-methoxy-5,6,7,8-tetrahydronaphthalen-1-yl, 4-methoxynaphthalen-1-yl, 4-methoxyphenyl, 4-sec-butylphenyl, 4-tert-butylphenyl, 4-tert-pentylphenyl, 5,6,7,8-tetrahydronaphthalen-2-yl, 5-bromo-2-(2-(pyrrolidin-1-yl)ethoxy)phenyl, 5-bromo-2-chlorophenyl, 5-bromo-2-methoxyphenyl, 5-bromo-2-methylphenyl, 5-chloro-2-cyanophenyl, 5-chloro-2-fluorophenyl, 5-chloro-2-methoxyphenyl, 5-chloro-2-methylphenyl, 5-chloronaphthalen-2-yl, 5-cyano-2-methylphenyl, 5-fluoro-2-methoxyphenyl, 5-fluoro-2-methylphenyl, 6-chloronaphthalen-2-yl, 6-methoxynaphthalen-2-yl, biphenyl-2-yl, biphenyl-3-yl, biphenyl-4-yl, m-tolyl, naphthalen-1-yl, naphthalen-2-yl, phenyl, and p-tolyl.

### The R¹¹ Group (Heteroaryl)

One aspect of the present invention relates to wherein R¹¹ is heteroaryl optionally substituted with one or more substituents as described herein.

In some embodiments, R¹¹ is heteroaryl optionally substituted with one or more substituents selected from: C₁-C₆ alkoxy, C₁-C₆ alkoxycarbonyl, C₁-C₇ alkyl, C₁-C₆ alkylamino, amino, aryloxy, arylsulfonyl, carboxy, cyano, C₂-C₈ dialkylamino, C₁-C₆ haloalkyl, halogen, heterocyclyl, hydroxyl, and oxo; and wherein said C₁-C₆ alkoxy, C₁-C₇ alkyl, and C₁-C₆ alkylamino are each optionally substituted with one or more substituents selected from: amino, C₁-C₆ alkoxy, carboxy, —Y¹—C₁-C₆-alkylene-Z¹ optionally substituted with oxo, C₃-C₇ cycloalkyl, C₂-C₆ dialkylamino, heterocyclyl, hydroxyl, and phenyl;
Y¹ is —O—; and
Z¹ is phenyl.

In some embodiments, R¹¹ is selected from: (1,2,3,4-tetrahydropyrimidin-5-yl)phenyl, (5-isoxazol-3-yl)thiophen-2-yl, (5-isoxazol-3-yl)thiophen-3-yl, (pyridin-2-yl)phenyl, [3,3′-bipyridin]yl, 1,2,3,4-tetrahydropyrido[2,3-b][1,4]oxazepinyl, 1′,2′-dihydrospiro[cyclopropane-1,3′-pyrido[2,3-b][1,4]oxazine]-7′-yl, 1,4-dihydroquinolinyl, 1,5-naphthyridinyl, 1,8-naphthyridinyl, 1H-benzo[d]imidazolyl, 1H-imidazo[4,5-b]pyridinyl, 1H-imidazolyl, 1H-indazolyl, 1H-indolyl, (1H-pyrazol-5-yl)thiophen-2-yl, (1H-pyrazol-5-yl)thiophen-3-yl, 1H-pyrazolo[3,4-b]pyridinyl, 1H-pyrazolo[4,3-b]pyridinyl, 1H-pyrazolyl, 1H-pyrrolo[2,3-b]pyridinyl, 1H-pyrrolo[3,2-b]pyridinyl, 1-phenyl-1H-pyrazolyl, 2,3-dihydro-[1,4]dioxino[2,3-b]pyridinyl, 2,3-dihydro-1H-benzo[d]imidazolyl, 2,3-dihydro-1H-imidazo[4,5-b]pyridinyl, 2,3-dihydro-1H-pyrido[2,3-b][1,4]oxazinyl, 2,3-dihydro-1H-pyrrolo[2,3-b]pyridinyl, 2,3-dihydrobenzo[b][1,4]dioxinyl, 2,3-dihydrobenzo[d]thiazolyl, 2,3-dihydrobenzofuranyl, 2,3-dihydrofuro[2,3-b]pyridinyl, 2-phenylthiazolyl, 3-(1H-pyrazol-3-yl)phenyl, 3-(1H-pyrazol-4-yl)phenyl, 3-(1H-pyrrol-3-yl)phenyl, 3-(2H-tetrazol-5-yl)phenyl, 3-(furan-2-yl)phenyl, 3-(isoxazol-4-yl)phenyl, 3-(pyridin-2-yl)phenyl, 3-(pyridin-3-yl)phenyl, 3-(pyridin-4-yl)phenyl, 3-(pyrimidin-5-yl)phenyl, 3-(thiazol-5-yl)phenyl, 3-(thiophen-2-yl)phenyl, 3-(thiophen-3-yl)phenyl, 3,4-dihydro-2H-benzo[b][1,4]oxazinyl, 3,4-dihydro-2H-pyrano[2,3-b]pyridinyl, 3,4-dihydro-2H-pyrido[3,2-b][1,4]oxazinyl, 3H-imidazo[4,5-b]pyridinyl, 4′-(1,2,4-oxadiazol-3-yl)biphenylyl, 4-(2H-tetrazol-5-yl)phenyl, 4-(phenyl)pyrimidinyl, 4-(pyridin-3-yl)phenyl, 4-(pyridin-4-yl)phenyl, 4-phenylpyrimidinyl, 5-(1H-pyrazol-4-yl)pyridinyl, 5-(phenyl)pyridinyl, 5,6,7,8-tetrahydro-1,6-naphthyridinyl, 5,6,7,8-tetrahydroquinolinyl, 5-phenyl-2,3-dihydrobenzofuranyl, 5-phenylpyrimidinyl, 5-phenylthiophen-2-yl, 5-phenylthiophen-3-yl, 6,7-dihydro-5H-cyclopenta[b]pyridinyl, 6,7-dihydro-5H-pyrrolo[3,4-b]pyridinyl, 6-phenylpyrimidinyl, 7,8-dihydro-5H-pyrano[4,3-b]pyridinyl, benzo[c][1,2,5]oxadiazolyl, benzo[c][1,2,5]thiadiazolyl, benzo[d]isoxazolyl, benzofuranyl, chromanyl, furanyl, imidazo[1,2-a]pyridinyl, imidazo[2,1-b]thiazolyl, indolinyl, isoxazolyl, pyrazinyl, pyridinyl, pyrimidinyl, quinolinyl, thiophen-2-yl, and thiophen-3-yl; each optionally substituted with one or more substituents selected from: amino, bromo, carboxy, chloro, cyano, dimethylamino, ethoxy, ethyl, ethylamino, fluoro, heptyl, hydroxyl, isobutyl, isopropyl, methoxy, methoxycarbonyl, methyl, methylamino, morpholino, oxo, phenoxy, phenylsulfonyl, piperazin-1-yl, piperidin-1-yl, propoxy, propyl, and trifluoromethyl; and wherein ethyl, ethylamino, methyl, and propoxy; are each optionally substituted with one or more substituents selected from: amino, benzyloxy, carboxy, cyclopropyl, dimethylamino, hydroxyl, methoxy, morpholino, and phenyl.

In some embodiments, R¹¹ is selected from: (1,2,3,4-tetrahydropyrimidin-5-yl)phenyl, (5-isoxazol-3-yl)thiophen-2-yl, (pyridin-2-yl)phenyl, [3,3′-bipyridin]-5-yl, 1,2,3,4-tetrahydropyrido[2,3-b][1,4]oxazepin-8-yl, 1′,2′-dihydrospiro[cyclopropane-1,3′-pyrido[2,3-b][1,4]oxazine]-7′-yl, 1,4-dihydroquinolin-3-yl, 1,5-naphthyridin-3-yl, 1,8-naphthyridin-3-yl, 1H-benzo[d]imidazol-5-yl, 1H-benzo[d]imidazol-6-yl, 1H-imidazo[4,5-b]pyridin-6-yl, 1H-imidazol-4-yl, 1H-indazol-5-yl, 1H-indazol-6-yl, 1H-indol-5-yl, 1H-pyrazol-3-yl, 1H-pyrazol-4-yl, 5-(1H-pyrazol-5-yl)thiophen-2-yl, 1H-pyrazolo[3,4-b]pyridin-5-yl, 1H-pyrazolo[4,3-b]pyridin-6-yl, 1H-pyrrolo[2,3-b]pyridin-3-yl, 1H-pyrrolo[2,3-b]pyridin-5-yl, 1H-pyrrolo[3,2-b]pyridin-3-yl, 1H-pyrrolo[3,2-b]pyridin-6-yl, 1-phenyl-1H-pyrazol-4-yl, 2,3-dihydro-[1,4]dioxino[2,3-b]pyridin-7-yl, 2,3-dihydro-1H-benzo[d]imidazol-5-yl, 2,3-dihydro-1H-imidazo[4,5-b]pyridin-6-yl, 2,3-dihydro-1H-pyrido[2,3-b][1,4]oxazin-6-yl, 2,3-dihydro-1H-pyrido[2,3-b][1,4]oxazin-7-yl, 2,3-dihydro-1H-pyrrolo[2,3-b]pyridin-5-yl, 2,3-dihydrobenzo[b][1,4]dioxin-6-yl, 2,3-dihydrobenzo[d]thiazol-6-yl, 2,3-dihydrobenzofuran-5-yl, 2,3-dihydrobenzofuran-7-yl, 2,3-dihydrofuro[2,3-b]pyridin-5-yl, 2-phenylthiazol-5-yl, 3-(1H-pyrazol-3-yl)phenyl, 3-(1H-pyrazol-4-yl)phenyl, 3-(1H-pyrrol-3-yl)phenyl, 3-(2H-tetrazol-5-yl)phenyl, 3-(furan-2-yl)phenyl, 3-(isoxazol-4-yl)phenyl, 3-(pyridin-2-yl)phenyl, 3-(pyridin-3-yl)phenyl, 3-(pyridin-4-yl)phenyl, 3-(pyrimidin-5-yl)phenyl, 3-(thiazol-5-yl)phenyl, 3-(thiophen-2-yl)phenyl, 3-(thiophen-3-yl)phenyl, 3,4-dihydro-2H-benzo[b][1,4]oxazin-6-yl, 3,4-dihydro-2H-pyrano[2,3-b]pyridin-6-yl, 3,4-dihydro-2H-pyrido[3,2-b][1,4]oxazin-7-yl, 3H-imidazo[4,5-b]pyridin-6-yl, 4′-(1,2,4-oxadiazol-3-yl)biphenyl-3-yl, 4-(2H-tetrazol-5-yl)phenyl, 4-(phenyl)pyrimidin-2-yl, 4-(pyridin-3-yl)phenyl, 4-(pyridin-4-yl)phenyl, 4-phenylpyrimidin-2-yl, 5-(1H-pyrazol-4-yl)pyridin-3-yl, 5-(phenyl)pyridin-3-yl, 5,6,7,8-tetrahydro-1,6-naphthyridin-3-yl, 5,6,7,8-tetrahydroquinolin-3-yl, 5-phenyl-2,3-dihydrobenzofuran-7-yl, 5-phenylpyrimidin-2-yl, 5-phenylthiophen-2-yl, 6,7-dihydro-5H-cyclopenta[b]pyridin-3-yl, 6,7-dihydro-5H-pyrrolo[3,4-b]pyridin-3-yl, 6-phenylpyrimidin-2-yl, 7,8-dihydro-5H-pyrano[4,3-b]pyridin-3-yl, benzo[c][1,2,5]oxadiazol-4-yl, benzo[c][1,2,5]thiadiazol-4-yl, benzo[c][1,2,5]thiadiazol-5-yl, benzo[d]isoxazol-5-yl, benzofuran-2-yl, benzofuran-5-yl, chroman-6-yl, furan-3-yl, imidazo[1,2-a]pyridin-6-yl, imidazo[2,1-b]thiazol-5-yl, indolin-5-yl, isoxazol-4-yl, pyrazin-2-yl, pyridin-2-yl, pyridin-3-yl, pyrimidin-2-yl, pyrimidin-4-yl, pyrimidin-5-yl, quinolin-3-yl, quinolin-6-yl, quinolin-8-yl, thiophen-2-yl, and thiophen-3-yl; each optionally substituted with one or more substituents selected from: 2-(benzyloxy)ethyl, 2-aminoethylamino, 2-hydroxyethyl, 2-hydroxyethylamino, 2-methoxyethyl, 2-methoxyethylamino, 2-morpholinoethylamino, 3-(dimethylamino)propoxy, amino, aminomethyl, benzyl, bromo, carboxy, carboxymethyl, chloro, cyano, cyclopropylmethyl, dimethylamino, ethoxy, ethyl, fluoro, heptyl, hydroxyl, isobutyl, isopropyl, methoxy, methoxycarbonyl, methyl, methylamino, morpholino, oxo, phenoxy, phenylsulfonyl, piperazin-1-yl, piperidin-1-yl, and propyl, trifluoromethyl.

In some embodiments, R¹¹ is selected from: 1-(2-(benzyloxy)ethyl)-2,3-dihydro-1H-pyrido[2,3-b][1,4]oxazin-7-yl, 1-(2-hydroxyethyl)-2,3-dihydro-1H-pyrido[2,3-b][1,4]oxazin-7-yl, 1-(2-methoxyethyl)-2,3-dihydro-1H-pyrido[2,3-b][1,4]oxazin-7-yl, 1-(carboxymethyl)-4-oxo-1,4-dihydroquinolin-3-yl, 1,2-dimethyl-1H-imidazol-4-yl, 1,3,3-trimethyl-2,3-dihydro-1H-pyrido[2,3-b][1,4]oxazin-7-yl, 1,3-dimethyl-2,3-dihydro-1H-pyrido[2,3-b][1,4]oxazin-7-yl, 1,5-naphthyridin-3-yl, 1,6-dimethyl-2,3-dihydro-1H-pyrido[2,3-b][1,4]oxazin-7-yl, 1,8-dimethyl-2,3-dihydro-1H-pyrido[2,3-b][1,4]oxazin-7-yl, 1-ethyl-2,3-dihydro-1H-pyrido[2,3-b][1,4]oxazin-7-yl, 1-ethyl-4-oxo-1,4-dihydroquinolin-3-yl, 1-ethyl-5-methyl-1H-pyrazol-4-yl, 1-ethyl-6-fluoro-4-oxo-1,4-dihydroquinolin-3-yl, 1-ethyl-6-methyl-4-oxo-1,4-dihydroquinolin-3-yl, 1-ethyl-7-fluoro-4-oxo-1,4-dihydroquinolin-3-yl, 1-ethyl-7-methyl-4-oxo-1,4-dihydroquinolin-3-yl, 1-ethyl-8-fluoro-4-oxo-1,4-dihydroquinolin-3-yl, 1-ethyl-8-methyl-4-oxo-1,4-dihydroquinolin-3-yl, 1H-benzo[d]imidazol-5-yl, 1H-benzo[d]imidazol-6-yl, 1H-imidazo[4,5-b]pyridin-6-yl, 1H-indazol-5-yl, 1H-indazol-6-yl, 1H-pyrazol-4-yl, 1H-pyrazolo[3,4-b]pyridin-5-yl, 1H-pyrazolo[4,3-b]pyridin-6-yl, 1H-pyrrolo[2,3-b]pyridin-3-yl, 1H-pyrrolo[2,3-b]pyridin-5-yl, 1H-pyrrolo[3,2-b]pyridin-3-yl, 1H-pyrrolo[3,2-b]pyridin-6-yl, 1-isopropyl-2,3-dihydro-1H-pyrido[2,3-b][1,4]oxazin-7-yl, 1-methyl-1,2,3,4-tetrahydropyrido[2,3-b][1,4]oxazepin-8-yl, 1′-methyl-1′,2′-dihydrospiro[cyclopropane-1,3′-pyrido[2,3-b][1,4]oxazine]-7′-yl, 1-methyl-1H-imidazo[4,5-b]pyridin-6-yl, 1-methyl-1H-imidazol-4-yl, 1-methyl-1H-indol-5-yl, 1-methyl-1H-pyrazol-3-yl, 1-methyl-1H-pyrrolo[3,2-b]pyridin-6-yl, 1-methyl-2,3-dihydro-1H-pyrido[2,3-b][1,4]oxazin-7-yl, 1-methyl-2-oxo-2,3-dihydro-1H-imidazo[4,5-b]pyridin-6-yl, 1-methyl-3-(trifluoromethyl)-1H-pyrazol-4-yl, 1-methyl-4-oxo-1,4-dihydroquinolin-3-yl, 1-methyl-6-(methylamino)-2,3-dihydro-1H-pyrido[2,3-b][1,4]oxazin-7-yl, 1-phenyl-1H-pyrazol-4-yl, 1-propyl-2,3-dihydro-1H-pyrido[2,3-b][1,4]oxazin-7-yl, 2-(dimethylamino)pyridin-3-yl, 2,2-dimethylchroman-6-yl, 2,3-dihydro-[1,4]dioxino[2,3-b]pyridin-7-yl, 2,3-dihydro-1H-pyrido[2,3-b][1,4]oxazin-7-yl, 2,3-dihydrobenzo[b][1,4]dioxin-6-yl, 2,3-dihydrobenzofuran-5-yl, 2,3-dihydrofuro[2,3-b]pyridin-5-yl, 2,3-dioxoindolin-5-yl, 2-ethyl-3H-imidazo[4,5-b]pyridin-6-yl, 2-hydroxypyrimidin-5-yl, 2-methyl-1H-benzo[d]imidazol-5-yl, 2-methyl-1H-imidazo[4,5-b]pyridin-6-yl, 2-methyl-3H-imidazo[4,5-b]pyridin-6-yl, 2-morpholinopyridin-3-yl, 2-oxo-2,3-dihydro-1H-benzo[d]imidazol-5-yl, 2-oxo-2,3-dihydro-1H-imidazo[4,5-b]pyridin-6-yl, 2-oxo-2,3-dihydro-1H-pyrido[2,3-b][1,4]oxazin-6-yl, 2-oxo-2,3-dihydro-1H-pyrrolo[2,3-b]pyridin-5-yl, 2-oxo-2,3-dihydrobenzo[d]thiazol-6-yl, 2-oxoindolin-5-yl, 3-(1-(2-hydroxyethyl)-1H-pyrazol-4-yl)phenyl, 3-(1-(cyclopropylmethyl)-1H-pyrazol-4-yl)phenyl, 3-(1,3-dimethyl-1H-pyrazol-4-yl)phenyl, 3-(1,3-dimethyl-2,4-dioxo-1,2,3,4-tetrahydropyrimidin-5-yl)phenyl, 3-(1-benzyl-1H-pyrazol-4-yl)phenyl, 3-(1-ethyl-1H-pyrazol-4-yl)phenyl, 3-(1H-pyrazol-3-yl)phenyl, 3-(1H-pyrazol-4-yl)phenyl, 3-(1H-pyrrol-3-yl)phenyl, 3-(1-isobutyl-1H-pyrazol-4-yl)phenyl, 3-(1-methyl-1H-pyrazol-4-yl)phenyl, 3-(1-methyl-1H-pyrrol-3-yl)phenyl, 3-(1-methyl-3-(trifluoromethyl)-1H-pyrazol-4-yl)phenyl, 3-(1-propyl-1H-pyrazol-4-yl)phenyl, 3-(2,4-dimethylthiazol-5-yl)phenyl, 3-(2H-tetrazol-5-yl)phenyl, 3-(2-methoxypyrimidin-5-yl)phenyl, 3-(2-methylpyridin-4-yl)phenyl, 3-(3,5-dimethylisoxazol-4-yl)phenyl, 3-(3-methylthiophen-2-yl)phenyl, 3-(4-methylthiophen-3-yl)phenyl, 3-(5-(aminomethyl)thiophen-2-yl)phenyl, 3-(5-cyanopyridin-3-yl)phenyl, 3-(5-methylpyridin-3-yl)phenyl, 3-(6-(2-morpholinoethylamino)pyridin-3-yl)phenyl, 3-(6-(3-(dimethylamino)propoxy)pyridin-3-yl)phenyl, 3-(6-(aminomethyl)pyridin-3-yl)phenyl, 3-(6-aminopyridin-3-yl)phenyl, 3-(6-methylpyridin-3-yl)phenyl, 3-(furan-2-yl)phenyl, 3-(pyridin-2-yl)phenyl, 3-(pyridin-3-yl)phenyl, 3-(pyridin-4-yl)phenyl, 3-(pyrimidin-5-yl)phenyl, 3-(thiophen-3-yl)phenyl, 3-(trifluoromethyl)-1H-pyrrolo[2,3-b]pyridin-5-yl, 3,4-dihydro-2H-benzo[b][1,4]oxazin-6-yl, 3,4-dihydro-2H-pyrano[2,3-b]pyridin-6-yl, 3,4-dihydro-2H-pyrido[3,2-b][1,4]oxazin-7-yl, 3,5-dimethyl-1-phenyl-1H-pyrazol-4-yl, 3,5-dimethylisoxazol-4-yl, 3-chloro-1H-pyrrolo[2,3-b]pyridin-5-yl, 3-chloro-1-methyl-1H-pyrrolo[2,3-b]pyridin-5-yl, 3-chloro-1-methyl-1H-pyrrolo[3,2-b]pyridin-6-yl, 3-chloro-2-methyl-1H-pyrrolo[2,3-b]pyridin-5-yl, 3-cyano-1H-pyrrolo[2,3-b]pyridin-5-yl, 3-ethyl-3H-imidazo[4,5-b]pyridin-6-yl, 3-methyl-1H-pyrazolo[3,4-b]pyridin-5-yl, 3-methyl-1H-pyrrolo[2,3-b]pyridin-5-yl, 3-methyl-2-oxo-2,3-dihydro-1H-imidazo[4,5-b]pyridin-6-yl, 3-methyl-3H-imidazo[4,5-b]pyridin-6-yl, 3-oxo-3,4-dihydro-2H-benzo[b][1,4]oxazin-6-yl, 4-(2H-tetrazol-5-yl)phenyl, 4-(3-methoxyphenyl)pyrimidin-2-yl, 4-carboxypyrimidin-2-yl, 4′-(5-methyl-1,2,4-oxadiazol-3-yl)biphenyl-3-yl, 4-(phenylsulfonyl)thiophen-2-yl, 4-(pyridin-2-yl)phenyl, 4-(pyridin-3-yl)phenyl, 4-(pyridin-4-yl)phenyl, 4-(trifluoromethyl)pyrimidin-2-yl, 4,5-dichlorothiophen-2-yl, 4,6-dimethoxypyrimidin-2-yl, 4,6-dimethylpyrimidin-2-yl, 4-aminopyrimidin-2-yl, 4-benzylpyrimidin-2-yl, 4-chloropyridin-2-yl, 4-ethyl-3,4-dihydro-2H-benzo[b][1,4]oxazin-6-yl, 4-hydroxy-6-methylquinolin-3-yl, 4-hydroxy-6-methylquinolin-8-yl, 4-hydroxy-7-methylquinolin-3-yl, 4-hydroxy-8-methylquinolin-3-yl, 4-hydroxyquinolin-3-yl, 4-isobutyl-3,4-dihydro-2H-benzo[b][1,4]oxazin-6-yl, 4-isopropyl-3,4-dihydro-2H-benzo[b][1,4]oxazin-6-yl, 4-methoxy-1H-indazol-5-yl, 4-methoxypyrimidin-2-yl, 4-methyl-2-phenylthiazol-5-yl, 4-methyl-3,4-dihydro-2H-benzo[b][1,4]oxazin-6-yl, 4-methyl-3,4-dihydro-2H-pyrido[3,2-b][1,4]oxazin-7-yl, 4-methyl-6-phenylpyrimidin-2-yl, 4-methylpyrimidin-2-yl, 4-oxo-1-propyl-1,4-dihydroquinolin-3-yl, 4-phenylpyrimidin-2-yl, 5-(1-methyl-1H-pyrazol-4-yl)pyridin-3-yl, 5-(1-methyl-3-(trifluoromethyl)-1H-pyrazol-5-yl)thiophen-2-yl, 5-(4-(aminomethyl)phenyl)-2,3-dihydrobenzofuran-7-yl, 5-(4-(aminomethyl)phenyl)pyridin-3-yl, 5-(5-(trifluoromethyl)isoxazol-3-yl)thiophen-2-yl, 5-(methoxycarbonyl)pyrimidin-2-yl, 5-(trifluoromethyl)pyrazin-2-yl, 5-(trifluoromethyl)pyrimidin-2-yl, 5,6,7,8-tetrahydro-1,6-naphthyridin-3-yl, 5,6,7,8-tetrahydroquinolin-3-yl, 5-benzylpyrimidin-2-yl, 5-bromo-2,3-dihydrobenzofuran-7-yl, 5-bromopyridin-3-yl, 5-chloro-1,3-dimethyl-1H-pyrazol-4-yl, 5-chlorobenzo[c][1,2,5]oxadiazol-4-yl, 5-chlorothiophen-2-yl, 5-ethylpyrimidin-2-yl, 5-heptylpyrimidin-2-yl, 5-methoxy-2-methylpyridin-3-yl, 5-methoxypyridin-3-yl, 5-methyl-1-phenyl-1H-pyrazol-4-yl, 5-methyl-2-(trifluoromethyl)furan-3-yl, 5-methyl-3H-imidazo[4,5-b]pyridin-6-yl, 5-methylbenzo[c][1,2,5]oxadiazol-4-yl, 5-oxo-6,7-dihydro-5H-pyrrolo[3,4-b]pyridin-3-yl, 5-phenylpyrimidin-2-yl, 5-phenylthiophen-2-yl, 5-propylpyrimidin-2-yl, 6-(2-aminoethylamino)pyridin-3-yl, 6-(2-hydroxyethylamino)pyridin-3-yl, 6-(2-methoxyethylamino)pyridin-3-yl, 6′-(aminomethyl)-3,3′-bipyridin-5-yl, 6-(dimethylamino)-1-methyl-2,3-dihydro-1H-pyrido[2,3-b][1,4]oxazin-7-yl, 6-(dimethylamino)pyridin-3-yl, 6-(piperazin-1-yl)pyridin-3-yl, 6-(piperidin-1-yl)pyridin-3-yl, 6,7-dihydro-5H-cyclopenta[b]pyridin-3-yl, 6,7-dihydro-5H-pyrrolo[3,4-b]pyridin-3-yl, 6-amino-1-methyl-2,3-dihydro-1H-pyrido[2,3-b][1,4]oxazin-7-yl, 6-chloro-1-methyl-2,3-dihydro-1H-pyrido[2,3-b][1,4]oxazin-7-yl, 6-chloroimidazo[2,1-b]thiazol-5-yl, 6-ethoxy-1-methyl-2,3-dihydro-1H-pyrido[2,3-b][1,4]oxazin-7-yl, 6-ethoxypyridin-3-yl, 6-fluoro-4-hydroxyquinolin-3-yl, 6-fluoro-4-oxo-1,4-dihydroquinolin-3-yl, 6-hydroxy-1-methyl-2,3-dihydro-1H-pyrido[2,3-b][1,4]oxazin-7-yl, 6-hydroxypyridin-3-yl, 6-methoxy-1-methyl-2,3-dihydro-1H-pyrido[2,3-b][1,4]oxazin-7-yl, 6-methoxypyridin-3-yl, 6-morpholinopyridin-3-yl, 6-phenoxypyridin-3-yl, 7,8-dihydro-5H-pyrano[4,3-b]pyridin-3-yl, 7-amino-1,8-naphthyridin-3-yl, 7-chlorobenzo[c][1,2,5]oxadiazol-4-yl, 7-fluoro-4-hydroxyquinolin-3-yl, 7-fluoro-4-oxo-1,4-dihydroquinolin-3-yl, 7-methoxybenzo[c][1,2,5]oxadiazol-4-yl, 7-methyl-3H-imidazo[4,5-b]pyridin-6-yl, 8-fluoro-4-hydroxyquinolin-3-yl, 8-fluoro-4-oxo-1,4-dihydroquinolin-3-yl, 8-methyl-4-oxo-1,4-dihydroquinolin-3-yl, benzo[c][1,2,5]thiadiazol-4-yl, benzo[c][1,2,5]thiadiazol-5-yl, benzo[d]isoxazol-5-yl, benzofuran-2-yl, benzofuran-5-yl, chroman-6-yl, furan-3-yl, imidazo[1,2-a]pyridin-6-yl, pyrazin-2-yl, pyridin-2-yl, pyridin-3-yl, pyridin-3-yl N-oxide, pyrimidin-2-yl, pyrimidin-4-yl, quinolin-3-yl, quinolin-6-yl, and thiophen-3-yl.

In some embodiments, R¹¹ is heteroaryl optionally substituted with one or more substituents selected from: C₁-C₇ alkyl, cyano, C₁-C₆ haloalkyl, halogen, hydroxyl, and oxo.

In some embodiments, R¹¹ is selected from: 1H-pyrrolo[3,2-b]pyridinyl, quinolinyl, 1,4-dihydroquinolinyl, 1H-pyrrolo[2,3-b]pyridinyl, and 2,3-dihydro-1H-pyrido[2,3-b][1,4]oxazinyl; each optionally substituted with one or more substituents selected from: ethyl, methyl, cyano, trifluoromethyl, fluoro, hydroxyl, and oxo.

In some embodiments, R¹¹ is selected from: 1H-pyrrolo[3,2-b]pyridin-6-yl, quinolin-3-yl, 1,4-dihydroquinolin-3-yl, 1H-pyrrolo[2,3-b]pyridin-5-yl, and 2,3-dihydro-1H-pyrido[2,3-b][1,4]oxazin-7-yl; each optionally substituted with one or more substituents selected from: ethyl, methyl, cyano, trifluoromethyl, fluoro, hydroxyl, and oxo.

In some embodiments, R¹¹ is selected from: 1H-pyrrolo[3,2-b]pyridin-6-yl, 4-hydroxyquinolin-3-yl, 1-ethyl-4-oxo-1,4-dihydroquinolin-3-yl, 3-cyano-1H-pyrrolo[2,3-b]pyridin-5-yl, 1,3-dimethyl-2,3-dihydro-1H-pyrido[2,3-b][1,4]oxazin-7-yl, and 3-(trifluoromethyl)-1H-pyrrolo[2,3-b]pyridin-5-yl.

The R¹² Groups (R^{12a} and R^{12b})

In some embodiments, R^{12a} is H or selected from: C₁-C₆ alkoxy, C₁-C₆ alkyl, C₃-C₇ cycloalkyl, and heterocyclyl; each optionally substituted with one or more substituents selected from: C₁-C₆ alkoxy, C₁-C₆ alkylamino, C₁-C₆ alkyl, C₁-C₆ alkylenehydroxyl, amino, C₃-C₇ cycloalkyl, cyano, C₂—C dialkylamino, heterocyclyl optionally substituted with one oxo group, halogen, hydroxyl, and oxo.

In some embodiments, R^{12a} is H or C₁-C₆ alkyl optionally substituted with one or more substituents selected from: C₁-C₆ alkoxy, C₁-C₆ alkylamino, C₁-C₆ alkyl, C₁-C₆ alkylenehydroxyl, amino, C₃-C₇ cycloalkyl, cyano, C₂—C dialkylamino, heterocyclyl optionally substituted with one oxo group, halogen, hydroxyl, and oxo.

In some embodiments, R^{12a} is H or selected from: C₁-C₆ alkoxy, C₁-C₆ alkyl, C₃-C₇ cycloalkyl, and heterocyclyl; each optionally substituted with one or more substituents selected from: amino, cyano, cyclopropyl, dimethylamino, ethoxy, ethyl, fluoro, hydroxyl, hydroxymethyl, methoxy, methylamino, oxo, oxopyrrolidinyl, and piperidinyl.

In some embodiments, R^{12a} is H or selected from: 2-methylpropanyl, butanyl, cyclobutyl, cyclohexyl, cyclopentyl, cyclopropyl, dimethylbutanyl, ethyl, ethylbutyl, isopentyl, isopropyl, methoxy, methyl, pentyl, piperidinyl, propanyl, propyl, sec-butyl, tert-butyl, and tetrahydro-2H-pyranyl; each optionally substituted with one or more substituents selected from: C₁-C₆ alkoxy, C₁-C₆ alkylamino, C₁-C₆ alkyl, C₁-C₆ alkylenehydroxyl, amino, C₃-C₇ cycloalkyl, cyano, C₂-C₈ dialkylamino, heterocyclyl optionally substituted with one oxo group, halogen, hydroxyl, and oxo.

In some embodiments, R^{12a} is H or selected from: 2-methylpropanyl, butanyl, cyclobutyl, cyclohexyl, cyclopentyl, cyclopropyl, dimethylbutanyl, ethyl, ethylbutyl, isopentyl, isopropyl, methoxy, methyl, pentyl, piperidinyl, propanyl, propyl, sec-butyl, tert-butyl, and tetrahydro-2H-pyranyl; each optionally substituted with one or more substituents selected from: amino, cyano, cyclopropyl, dimethylamino, ethoxy, ethyl, fluoro, hydroxyl, hydroxymethyl, methoxy, methylamino, oxo, oxopyrrolidinyl, and piperidinyl.

In some embodiments, R^{12a} is selected from: H, methyl, propyl, pentyl, (2,2,2-trifluoroethyl), isopropyl, cyclopropylmethyl, 2,2-difluoroethyl, sec-butyl, methoxy, 2-hydroxyethyl, 2-methoxyethyl, 2-hydroxypropyl, 2-ethoxyethyl, 1-hydroxypropan-2-yl, 1-hydroxy-2-methylpropan-2-yl, tetrahydro-2H-pyran-4-yl, 3-hydroxypropyl, cyclopropyl, 3-methoxypropyl, 3,3-difluorocyclobutyl, 2-aminoethyl, 3-hydroxy-1-(methylamino)-1-oxobutan-2-yl, 1-cyclopropylethyl, tert-butyl, 1,3-dihydroxypropan-2-yl, 2-ethylbutyl, isopentyl, 1-(hydroxymethyl)cyclopropyl, 3,3-dimethylbutan-2-yl, ethyl, 2-(2-oxopyrrolidin-1-yl)ethyl, 1-ethylpiperidin-4-yl, 2,3-dihydroxypropyl, 2-(dimethylamino)ethyl, piperidin-3-ylmethyl, 3-(dimethylamino)propyl, acetyl, 2-fluoroethyl, 2-hydroxycyclopentyl, 2-hydroxycyclohexyl, and cyanomethyl.

In some embodiments, R^{12a} is H or C₁-C₆ alkyl.

In some embodiments, R^{12a} is H, ethyl, or methyl.

In some embodiments, R^{12b} is H or C₁-C₆ alkyl.

In some embodiments, R^{12b} is selected from: H, ethyl, isopropyl, and methyl. In some embodiments, R^{12b} is selected from: H and methyl. In some embodiments, R^{12b} is H.

### Certain Combinations

One aspect of the present invention pertains to compounds of Formula (Ic) and pharmaceutically acceptable salts, solvates, and hydrates thereof:

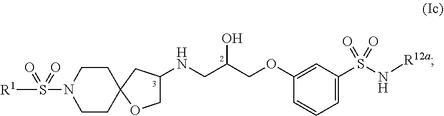

wherein: R¹¹ (as well as Y¹ and Z¹ that are both related to R¹¹), R^{12a}, and R^{12b} all have the same definitions as described herein, supra and infra.

One aspect of the present invention pertains to compounds of Formula (IIc) and pharmaceutically acceptable salts, solvates, and hydrates thereof:

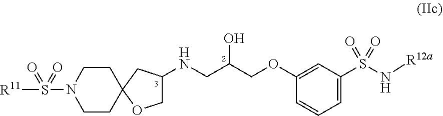

wherein:
R¹¹ is selected from: aryl, heteroaryl, and heterocyclyl; each optionally substituted with one or more substituents selected from: C₁-C₆ alkoxy, C₁-C₇ alkyl, C₁-C₆ alkylamino, C₁-C₆ alkylcarboxamide, C₁-C₆ alkylsulfonamido, C₁-C₆ alkylsulfonyl, amino, aryloxy, arylsulfonyl, carboxamide, carbamimidoyl, carboxy, cyano, C₃-C₇ cycloalkyl, C₂—C dialkylamino, C₂-C₈ dialkylsulfamoyl, C₁-C₆ haloalkoxy, C₁-C₆ haloalkyl, halogen, heterocyclyl, hydroxycarbamimidoyl, hydroxyl, oxo, and sulfamoyl; and wherein said C₁-C₆ alkoxy, C₁-C₇ alkyl, C₁-C₆ alkylamino, aryloxy, C₃-C₇ cycloalkyl, and C₂-C₈ dialkylamino are each optionally substituted with one or more substituents selected from: amino, C₁-C₆ alkoxy, C₁-C₆ alkylamino, C₁-C₆ alkylcarboxamide, carboxy, —Y¹—C₁-C₆-alkylene-Z¹ optionally substituted with oxo, C₃-C₇ cycloalkyl, cyano, C₂-C₆ dialkylamino, C₁-C₆ haloalkyl, C₁-C₆ haloalkylamino, heterocyclyl, hydroxyl, oxo, and phenyl;
Y¹ is selected from: —O— and —NH—;
Z¹ is selected from: C₁-C₆ alkoxy, amino, cyano, C₂-C₆ dialkylamino, hydroxyl, and phenyl; and
R^{12a} is H, ethyl, or methyl.

One aspect of the present invention pertains to compounds of Formula (Ic) and pharmaceutically acceptable salts, solvates, and hydrates thereof:

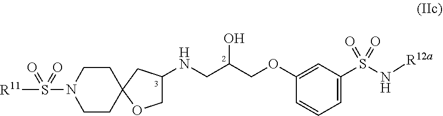

wherein:
R¹¹ is selected from: aryl, heteroaryl, and heterocyclyl; each optionally substituted with one or more substituents selected from: (2-ethyl)(methyl)amino, acetamido, amino, bromo, carbamimidoyl, carboxamide, carboxy, chloro, cyano, cyclopropyl, dimethylamino, dimethylcarbamoyl, ethoxy, ethyl, ethylamino, fluoro, hydroxycarbamimidoyl, hydroxyl, isobutyl, isopropoxy, isopropyl, isopropyl(methyl)amino, methoxy, methyl, methyl(propyl)amino, methylamino, methylsulfonamido, methylsulfonyl, morpholino, N,N-dimethylsulfamoyl, oxo, phenoxy, phenylsulfonyl, piperazin-1-yl, piperidin-1-yl, propoxy, propyl, sec-butyl, sulfamoyl, tert-butyl, tert-pentyl, trifluoromethoxy, and trifluoromethyl; and wherein (2-ethyl)(methyl)amino, cyclopropyl, ethoxy, ethyl, ethylamino, isopropyl(methyl)amino, methoxy, methyl, methyl(propyl)amino, phenoxy, and propoxy are each optionally substituted with one or more substituents selected from: 2-(dimethylamino)ethylamino, 2,2,2-trifluoroethylamino, 2,2-difluoroethylamino, 2-amino-2-oxoacetamido, 2-aminoacetamido, 2-fluoroethylamino, 2-hydroxyethylamino, 2-methoxyethylamino, acetamido, amino, benzyloxy, carboxy, cyano, cyanomethylamino, cyclopropyl, dimethylamino, ethylamino, hydroxyl, isobutylamino, isopentylamino, isopropylamino, methoxy, methylamino, morpholino, oxo, phenyl, pyrrolidin-1-yl, thiazolidin-3-yl, and trifluoromethyl; and
R^{12a} is H, ethyl, or methyl.

One aspect of the present invention pertains to compounds of Formula (IIc) and pharmaceutically acceptable salts, solvates, and hydrates thereof:

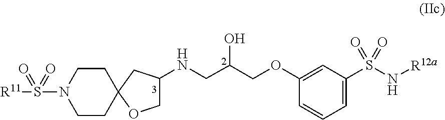

wherein:
R¹¹ is selected from: aryl, heteroaryl, and heterocyclyl; each optionally substituted with one or more substituents selected from: (1-amino-3-hydroxy-1-oxopropan-2-yl)(methyl)amino, (2-(dimethylamino)ethylamino)methyl, (2,2,2-trifluoroethylamino)methyl, (2,2-difluoroethylamino)methyl, (2-acetamidoethyl)(methyl)amino, (2-amino-2-oxoacetamido)methyl, (2-aminoacetamido)methyl, (2-fluoroethylamino)methyl, (2-hydroxyethylamino)methyl, (2-methoxyethylamino)methyl, (cyanomethylamino)methyl, (dimethylamino)methyl, (ethylamino)methyl, (isobutylamino)methyl, (isopentylamino)methyl, (isopropylamino)methyl, (methylamino)methyl, 1-aminocyclopropyl, 2-(benzyloxy)ethyl, 2-(pyrrolidin-1-yl)ethoxy, 2-(trifluoromethyl)phenoxy, 2-aminoethylamino, 2-carboxy-N-methylacetamido, 2-hydroxyethyl, 2-hydroxyethylamino, 2-methoxyethyl, 2-methoxyethylamino, 2-morpholinoethylamino, 3-(dimethylamino)propoxy, 4-(trifluoromethyl)phenoxy, acetamido, acetyl, amino, aminomethyl, benzyl, bromo, carbamimidoyl, carboxamide, carboxy, carboxymethyl, chloro, cyano, cyanomethoxy, cyanomethyl, cyclopropylmethyl, dimethylamino, dimethylcarbamoyl, ethoxy, ethyl, fluoro, hydroxycarbamimidoyl, hydroxyl, hydroxymethyl, isobutyl, isopropoxy, isopropyl, methoxy, methoxymethyl, methyl, methylamino, methylsulfonamido, methylsulfonyl, morpholino, N,N-dimethylsulfamoyl, oxo, phenoxy, phenylsulfonyl, piperazin-1-yl, piperidin-1-yl, propyl, sec-butyl, sulfamoyl, tert-butyl, tert-pentyl, thiazolidin-3-ylmethyl, trifluoromethoxy, and trifluoromethyl; and
R^{12a} is H, ethyl, or methyl.

One aspect of the present invention pertains to compounds of Formula (IIc) and pharmaceutically acceptable salts, solvates, and hydrates thereof:

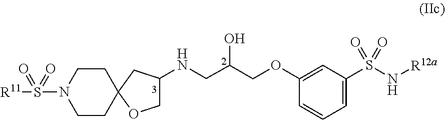

wherein:
R¹¹ is selected from: (1,2,3,4-tetrahydropyrimidin-5-yl)phenyl, (5-isoxazol-3-yl)thiophen-2-yl, (5-isoxazol-3-yl)thiophen-3-yl, (pyridin-2-yl)phenyl, [3,3′-bipyridin]yl, 1,2,3,4-tetrahydropyrido[2,3-b][1,4]oxazepinyl, 1′,2′-dihydrospiro[cyclopropane-1,3′-pyrido[2,3-b][1,4]oxazine]-7′-yl, 1,4-dihydropyridinyl, 1,4-dihydroquinolinyl, 1,5-naphthyridinyl, 1,8-naphthyridinyl, 1H-benzo[d]imidazolyl, 1H-imidazo[4,5-b]pyridinyl, 1H-imidazolyl, 1H-indazolyl, 1H-indolyl, (1H-pyrazol-5-yl)thiophen-2-yl, (1H-pyrazol-5-yl)thiophen-3-yl, 1H-pyrazolo[3,4-b]pyridinyl, 1H-pyrazolo[4,3-b]pyridinyl, 1H-pyrazolyl, 1H-pyrrolo[2,3-b]pyridinyl, 1H-pyrrolo[3,2-b]pyridinyl, 1-phenyl-1H-pyrazolyl, 2,3-dihydro-[1,4]dioxino[2,3-b]pyridinyl, 2,3-dihydro-1H-benzo[d]imidazolyl, 2,3-dihydro-1H-imidazo[4,5-b]pyridinyl, 2,3-dihydro-1H-pyrido[2,3-b][1,4]oxazinyl, 2,3-dihydro-1H-pyrrolo[2,3-b]pyridinyl, 2,3-dihydrobenzo[b][1,4]dioxinyl, 2,3-dihydrobenzo[d]thiazolyl, 2,3-dihydrobenzofuranyl, 2,3-dihydrofuro[2,3-b]pyridinyl, 2-phenylthiazolyl, 3-(1H-pyrazol-3-yl)phenyl, 3-(1H-pyrazol-4-yl)phenyl, 3-(1H-pyrrol-3-yl)phenyl, 3-(2H-tetrazol-5-yl)phenyl, 3-(furan-2-yl)phenyl, 3-(isoxazol-4-yl)phenyl, 3-(pyridin-2-yl)phenyl, 3-(pyridin-3-yl)phenyl, 3-(pyridin-4-yl)phenyl, 3-(pyrimidin-5-yl)phenyl, 3-(thiazol-5-yl)phenyl, 3-(thiophen-2-yl)phenyl, 3-(thiophen-3-yl)phenyl, 3,4-dihydro-2H-benzo[b][1,4]oxazinyl, 3,4-dihydro-2H-pyrano[2,3-b]pyridinyl, 3,4-dihydro-2H-pyrido[3,2-b][1,4]oxazinyl, 3H-imidazo[4,5-b]pyridinyl, 4′-(1,2,4-oxadiazol-3-yl)biphenylyl, 4-(2H-tetrazol-5-yl)phenyl, 4-(pyridin-3-yl)phenyl, 4-(pyridin-4-yl)phenyl, 5-(1H-pyrazol-4-yl)pyridinyl, 5-(phenyl)pyridinyl, 5,6,7,8-tetrahydro-1,6-naphthyridinyl, 5,6,7,8-tetrahydronaphthalenyl, 5,6,7,8-tetrahydroquinolinyl, 5-phenyl-2,3-dihydrobenzofuranyl, 5-phenylthiophen-2-yl, 5-phenylthiophen-3-yl, 6,7-dihydro-5H-cyclopenta[b]pyridinyl, 6,7-dihydro-5H-pyrrolo[3,4-b]pyridinyl, 7,8-dihydro-5H-pyrano[4,3-b]pyridinyl, benzo[c][1,2,5]oxadiazolyl, benzo[c][1,2,5]thiadiazolyl, benzo[d]isoxazolyl, benzofuranyl, biphenylyl, chromanyl, furanyl, imidazo[1,2-a]pyridinyl, imidazo[2,1-b]thiazolyl, indolinyl, isoxazolyl, naphthalenyl, phenyl, pyridinyl, pyrimidinyl, quinolinyl, thiophen-2-yl, and thiophen-3-yl; R¹ is selected from: aryl, heteroaryl, and heterocyclyl; each optionally substituted with one or more substituents selected from: (2-ethyl)(methyl)amino, acetamido, amino, bromo, carbamimidoyl, carboxamide, carboxy, chloro, cyano, cyclopropyl, dimethylamino, dimethylcarbamoyl, ethoxy, ethyl, ethylamino, fluoro, hydroxycarbamimidoyl, hydroxyl, isobutyl, isopropoxy, isopropyl, isopropyl(methyl)amino, methoxy, methyl, methyl(propyl)amino, methylamino, methylsulfonamido, methylsulfonyl, morpholino, N,N-dimethylsulfamoyl, oxo, phenoxy, phenylsulfonyl, piperazin-1-yl, piperidin-1-yl, propoxy, propyl, sec-butyl, sulfamoyl, tert-butyl, tert-pentyl, trifluoromethoxy, and trifluoromethyl; and wherein (2-ethyl)(methyl)amino, cyclopropyl, ethoxy, ethyl, ethylamino, isopropyl(methyl)amino, methoxy, methyl, methyl(propyl)amino, phenoxy, and propoxy are each optionally substituted with one or more substituents selected from: 2-(dimethylamino)ethylamino, 2,2,2-trifluoroethylamino, 2,2-difluoroethylamino, 2-amino-2-oxoacetamido, 2-aminoacetamido, 2-fluoroethylamino, 2-hydroxyethylamino, 2-methoxyethylamino, acetamido, amino, benzyloxy, carboxy, cyano, cyanomethylamino, cyclopropyl, dimethylamino, ethylamino, hydroxyl, isobutylamino, isopentylamino, isopropylamino, methoxy, methylamino, morpholino, oxo, phenyl, pyrrolidin-1-yl, thiazolidin-3-yl, and trifluoromethyl; and
R^{12a} is H, ethyl, or methyl.

One aspect of the present invention pertains to compounds of Formula (IIc) and pharmaceutically acceptable salts, solvates, and hydrates thereof:

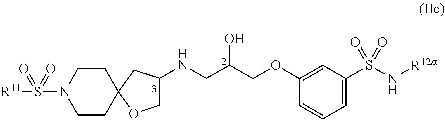

wherein:
R¹¹ is selected from: (1,2,3,4-tetrahydropyrimidin-5-yl)phenyl, (5-isoxazol-3-yl)thiophen-2-yl, (5-isoxazol-3-yl)thiophen-3-yl, (pyridin-2-yl)phenyl, [3,3′-bipyridin]yl, 1,2,3,4-tetrahydropyrido[2,3-b][1,4]oxazepinyl, 1′,2′-dihydrospiro[cyclopropane-1,3′-pyrido[2,3-b][1,4]oxazine]-7′-yl, 1,4-dihydropyridinyl, 1,4-dihydroquinolinyl, 1,5-naphthyridinyl, 1,8-naphthyridinyl, 1H-benzo[d]imidazolyl, 1H-imidazo[4,5-b]pyridinyl, 1H-imidazolyl, 1H-indazolyl, 1H-indolyl, (1H-pyrazol-5-yl)thiophen-2-yl, (1H-pyrazol-5-yl)thiophen-3-yl, 1H-pyrazolo[3,4-b]pyridinyl, 1H-pyrazolo[4,3-b]pyridinyl, 1H-pyrazolyl, 1H-pyrrolo[2,3-b]pyridinyl, 1H-pyrrolo[3,2-b]pyridinyl, 1-phenyl-1H-pyrazolyl, 2,3-dihydro-[1,4]dioxino[2,3-b]pyridinyl, 2,3-dihydro-1H-benzo[d]imidazolyl, 2,3-dihydro-1H-imidazo[4,5-b]pyridinyl, 2,3-dihydro-1H-pyrido[2,3-b][1,4]oxazinyl, 2,3-dihydro-1H-pyrrolo[2,3-b]pyridinyl, 2,3-dihydrobenzo[b][1,4]dioxinyl, 2,3-dihydrobenzo[d]thiazolyl, 2,3-dihydrobenzofuranyl, 2,3-dihydrofuro[2,3-b]pyridinyl, 2-phenylthiazolyl, 3-(1H-pyrazol-3-yl)phenyl, 3-(1H-pyrazol-4-yl)phenyl, 3-(1H-pyrrol-3-yl)phenyl, 3-(2H-tetrazol-5-yl)phenyl, 3-(furan-2-yl)phenyl, 3-(isoxazol-4-yl)phenyl, 3-(pyridin-2-yl)phenyl, 3-(pyridin-3-yl)phenyl, 3-(pyridin-4-yl)phenyl, 3-(pyrimidin-5-yl)phenyl, 3-(thiazol-5-yl)phenyl, 3-(thiophen-2-yl)phenyl, 3-(thiophen-3-yl)phenyl, 3,4-dihydro-2H-benzo[b][1,4]oxazinyl, 3,4-dihydro-2H-pyrano[2,3-b]pyridinyl, 3,4-dihydro-2H-pyrido[3,2-b][1,4]oxazinyl, 3H-imidazo[4,5-b]pyridinyl, 4′-(1,2,4-oxadiazol-3-yl)biphenylyl, 4-(2H-tetrazol-5-yl)phenyl, 4-(pyridin-3-yl)phenyl, 4-(pyridin-4-yl)phenyl, 5-(1H-pyrazol-4-yl)pyridinyl, 5-(phenyl)pyridinyl, 5,6,7,8-tetrahydro-1,6-naphthyridinyl, 5,6,7,8-tetrahydronaphthalenyl, 5,6,7,8-tetrahydroquinolinyl, 5-phenyl-2,3-dihydrobenzofuranyl, 5-phenylthiophen-2-yl, 5-phenylthiophen-3-yl, 6,7-dihydro-5H-cyclopenta[b]pyridinyl, 6,7-dihydro-5H-pyrrolo[3,4-b]pyridinyl, 7,8-dihydro-5H-pyrano[4,3-b]pyridinyl, benzo[c][1,2,5]oxadiazolyl, benzo[c][1,2,5]thiadiazolyl, benzo[d]isoxazolyl, benzofuranyl, biphenylyl, chromanyl, furanyl, imidazo[1,2-a]pyridinyl, imidazo[2,1-b]thiazolyl, indolinyl, isoxazolyl, naphthalenyl, phenyl, pyridinyl, pyrimidinyl, quinolinyl, thiophen-2-yl, and thiophen-3-yl; each optionally substituted with one or more substituents selected from: (1-amino-3-hydroxy-1-oxopropan-2-yl)(methyl)amino, (2-(dimethylamino)ethylamino)methyl, (2,2,2-trifluoroethylamino)methyl, (2,2-difluoroethylamino)methyl, (2-acetamidoethyl)(methyl)amino, (2-amino-2-oxoacetamido)methyl, (2-aminoacetamido)methyl, (2-fluoroethylamino)methyl, (2-hydroxyethylamino)methyl, (2-methoxyethylamino)methyl, (cyanomethylamino)methyl, (dimethylamino)methyl, (ethylamino)methyl, (isobutylamino)methyl, (isopentylamino)methyl, (isopropylamino)methyl, (methylamino)methyl, 1-aminocyclopropyl, 2-(benzyloxy)ethyl, 2-(pyrrolidin-1-yl)ethoxy, 2-(trifluoromethyl)phenoxy, 2-aminoethylamino, 2-carboxy-N-methylacetamido, 2-hydroxyethyl, 2-hydroxyethylamino, 2-methoxyethyl, 2-methoxyethylamino, 2-morpholinoethylamino, 3-(dimethylamino)propoxy, 4-(trifluoromethyl)phenoxy, acetamido, acetyl, amino, aminomethyl, benzyl, bromo, carbamimidoyl, carboxamide, carboxy, carboxymethyl, chloro, cyano, cyanomethoxy, cyanomethyl, cyclopropylmethyl, dimethylamino, dimethylcarbamoyl, ethoxy, ethyl, fluoro, hydroxycarbamimidoyl, hydroxyl, hydroxymethyl, isobutyl, isopropoxy, isopropyl, methoxy, methoxymethyl, methyl, methylamino, methylsulfonamido, methylsulfonyl, morpholino, N,N-dimethylsulfamoyl, oxo, phenoxy, phenylsulfonyl, piperazin-1-yl, piperidin-1-yl, propyl, sec-butyl, sulfamoyl, tert-butyl, tert-pentyl, thiazolidin-3-ylmethyl, trifluoromethoxy, and trifluoromethyl; and
R^{12a} is H, ethyl, or methyl.

One aspect of the present invention pertains to compounds of Formula (IIc) and pharmaceutically acceptable salts, solvates, and hydrates thereof:

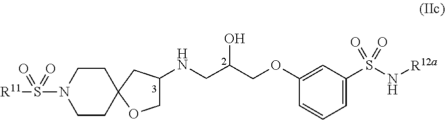

wherein:
R¹¹ is selected from: (1,2,3,4-tetrahydropyrimidin-5-yl)phenyl, (5-isoxazol-3-yl)thiophen-2-yl, (pyridin-2-yl)phenyl, [3,3′-bipyridin]-5-yl, 1,2,3,4-tetrahydropyrido[2,3-b][1,4]oxazepin-8-yl, 1′,2′-dihydrospiro[cyclopropane-1,3′-pyrido[2,3-b][1,4]oxazine]-7′-yl, 1,4-dihydropyridin-3-yl, 1,4-dihydroquinolin-3-yl, 1,5-naphthyridin-3-yl, 1,8-naphthyridin-3-yl, 1H-benzo[d]imidazol-5-yl, 1H-benzo[d]imidazol-6-yl, 1H-imidazo[4,5-b]pyridin-6-yl, 1H-imidazol-4-yl, 1H-indazol-5-yl, 1H-indazol-6-yl, 1H-indol-5-yl, 1H-pyrazol-3-yl, 1H-pyrazol-4-yl, (1H-pyrazol-5-yl)thiophen-2-yl, 1H-pyrazolo[3,4-b]pyridin-5-yl, 1H-pyrazolo[4,3-b]pyridin-6-yl, 1H-pyrrolo[2,3-b]pyridin-3-yl, 1H-pyrrolo[2,3-b]pyridin-5-yl, 1H-pyrrolo[3,2-b]pyridin-3-yl, 1H-pyrrolo[3,2-b]pyridin-6-yl, 1-phenyl-1H-pyrazol-4-yl, 2,3-dihydro-[1,4]dioxino[2,3-b]pyridin-7-yl, 2,3-dihydro-1H-benzo[d]imidazol-5-yl, 2,3-dihydro-1H-imidazo[4,5-b]pyridin-6-yl, 2,3-dihydro-1H-pyrido[2,3-b][1,4]oxazin-6-yl, 2,3-dihydro-1H-pyrido[2,3-b][1,4]oxazin-7-yl, 2,3-dihydro-1H-pyrrolo[2,3-b]pyridin-5-yl, 2,3-dihydrobenzo[b][1,4]dioxin-6-yl, 2,3-dihydrobenzo[d]thiazol-6-yl, 2,3-dihydrobenzofuran-5-yl, 2,3-dihydrobenzofuran-7-yl, 2,3-dihydrofuro[2,3-b]pyridin-5-yl, 2-phenylthiazol-5-yl, 3-(1H-pyrazol-3-yl)phenyl, 3-(1H-pyrazol-4-yl)phenyl, 3-(1H-pyrrol-3-yl)phenyl, 3-(2H-tetrazol-5-yl)phenyl, 3-(furan-2-yl)phenyl, 3-(isoxazol-4-yl)phenyl, 3-(pyridin-2-yl)phenyl, 3-(pyridin-3-yl)phenyl, 3-(pyridin-4-yl)phenyl, 3-(pyrimidin-5-yl)phenyl, 3-(thiazol-5-yl)phenyl, 3-(thiophen-2-yl)phenyl, 3-(thiophen-3-yl)phenyl, 3,4-dihydro-2H-benzo[b][1,4]oxazin-6-yl, 3,4-dihydro-2H-pyrano[2,3-b]pyridin-6-yl, 3,4-dihydro-2H-pyrido[3,2-b][1,4]oxazin-7-yl, 3H-imidazo[4,5-b]pyridin-6-yl, 4′-(1,2,4-oxadiazol-3-yl)biphenyl-3-yl, 4-(2H-tetrazol-5-yl)phenyl, 4-(pyridin-3-yl)phenyl, 4-(pyridin-4-yl)phenyl, 5-(1H-pyrazol-4-yl)pyridin-3-yl, 5-(phenyl)pyridin-3-yl, 5,6,7,8-tetrahydro-1,6-naphthyridin-3-yl, 5,6,7,8-tetrahydronaphthalen-1-yl, 5,6,7,8-tetrahydronaphthalen-2-yl, 5,6,7,8-tetrahydronaphthalenyl, 5,6,7,8-tetrahydroquinolin-3-yl, 5-phenyl-2,3-dihydrobenzofuran-7-yl, 5-phenylthiophen-2-yl, 6,7-dihydro-5H-cyclopenta[b]pyridin-3-yl, 6,7-dihydro-5H-pyrrolo[3,4-b]pyridin-3-yl, 7,8-dihydro-5H-pyrano[4,3-b]pyridin-3-yl, benzo[c][1,2,5]oxadiazol-4-yl, benzo[c][1,2,5]thiadiazol-4-yl, benzo[c][1,2,5]thiadiazol-5-yl, benzo[d]isoxazol-5-yl, benzofuran-2-yl, benzofuran-5-yl, biphenyl-2-yl, biphenyl-3-yl, biphenyl-4-yl, chroman-6-yl, furan-3-yl, imidazo[1,2-a]pyridin-6-yl, imidazo[2,1-b]thiazol-5-yl, indolin-5-yl, isoxazol-4-yl, naphthalen-1-yl, naphthalen-2-yl, phenyl, pyridin-2-yl, pyridin-3-yl, pyrimidin-5-yl, quinolin-3-yl, quinolin-6-yl, quinolin-8-yl, thiophen-2-yl, and thiophen-3-yl; each optionally substituted with one or more substituents selected from: (1-amino-3-hydroxy-1-oxopropan-2-yl)(methyl)amino, (2-(dimethylamino)ethylamino)methyl, (2,2,2-trifluoroethylamino)methyl, (2,2-difluoroethylamino)methyl, (2-acetamidoethyl)(methyl)amino, (2-amino-2-oxoacetamido)methyl, (2-aminoacetamido)methyl, (2-fluoroethylamino)methyl, (2-hydroxyethylamino)methyl, (2-methoxyethylamino)methyl, (cyanomethylamino)methyl, (dimethylamino)methyl, (ethylamino)methyl, (isobutylamino)methyl, (isopentylamino)methyl, (isopropylamino)methyl, (methylamino)methyl, 1-aminocyclopropyl, 2-(benzyloxy)ethyl, 2-(pyrrolidin-1-yl)ethoxy, 2-(trifluoromethyl)phenoxy, 2-aminoethylamino, 2-carboxy-N-methylacetamido, 2-hydroxyethyl, 2-hydroxyethylamino, 2-methoxyethyl, 2-methoxyethylamino, 2-morpholinoethylamino, 3-(dimethylamino)propoxy, 4-(trifluoromethyl)phenoxy, acetamido, acetyl, amino, aminomethyl, benzyl, bromo, carbamimidoyl, carboxamide, carboxy, carboxymethyl, chloro, cyano, cyanomethoxy, cyanomethyl, cyclopropylmethyl, dimethylamino, dimethylcarbamoyl, ethoxy, ethyl, fluoro, hydroxycarbamimidoyl, hydroxyl, hydroxymethyl, isobutyl, isopropoxy, isopropyl, methoxy, methoxymethyl, methyl, methylamino, methylsulfonamido, methylsulfonyl, morpholino, N,N-dimethylsulfamoyl, oxo, phenoxy, phenylsulfonyl, piperazin-1-yl, piperidin-1-yl, propyl, sec-butyl, sulfamoyl, tert-butyl, tert-pentyl, thiazolidin-3-ylmethyl, trifluoromethoxy, and trifluoromethyl; and
R^{12a} is H, ethyl, or methyl.

One aspect of the present invention pertains to compounds of Formula (IIc) and pharmaceutically acceptable salts, solvates, and hydrates thereof:

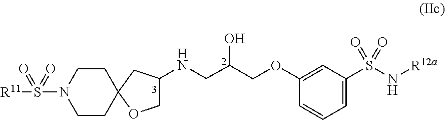

wherein:
R¹¹ is heteroaryl optionally substituted with one or more substituents selected from: C₁-C₇ alkyl, cyano, C₁-C₆ haloalkyl, halogen, hydroxyl, and oxo; and
R^{12a} is H or C₁-C₆ alkyl.

One aspect of the present invention pertains to compounds of Formula (IIc) and pharmaceutically acceptable salts, solvates, and hydrates thereof:

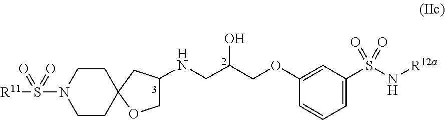

wherein:
R¹¹ is selected from: 1H-pyrrolo[3,2-b]pyridinyl, quinolinyl, 1,4-dihydroquinolinyl, 1H-pyrrolo[2,3-b]pyridinyl, and 2,3-dihydro-1H-pyrido[2,3-b][1,4]oxazinyl; each optionally substituted with one or more substituents selected from: ethyl, methyl, cyano, trifluoromethyl, fluoro, hydroxyl, and oxo; and
R^{12a} is H or C₁-C₆ alkyl.

One aspect of the present invention pertains to compounds of Formula (IIc) and pharmaceutically acceptable salts, solvates, and hydrates thereof:

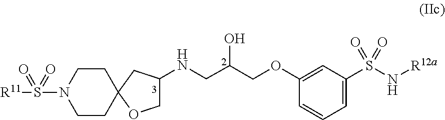

wherein:
R¹¹ is selected from: 1H-pyrrolo[3,2-b]pyridin-6-yl, quinolin-3-yl, 1,4-dihydroquinolin-3-yl, 1H-pyrrolo[2,3-b]pyridin-5-yl, and 2,3-dihydro-1H-pyrido[2,3-b][1,4]oxazin-7-yl; each optionally substituted with one or more substituents selected from: ethyl, methyl, cyano, trifluoromethyl, fluoro, hydroxyl, and oxo; and
R^{12a} is H, ethyl, or methyl.

One aspect of the present invention pertains to compounds of Formula (IIc) and pharmaceutically acceptable salts, solvates, and hydrates thereof:

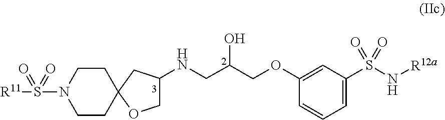

wherein:
R¹¹ is selected from: 1H-pyrrolo[3,2-b]pyridin-6-yl, 4-hydroxyquinolin-3-yl, 1-ethyl-4-oxo-1,4-dihydroquinolin-3-yl, 3-cyano-1H-pyrrolo[2,3-b]pyridin-5-yl, 1,3-dimethyl-2,3-dihydro-1H-pyrido[2,3-b][1,4]oxazin-7-yl, and 3-(trifluoromethyl)-1H-pyrrolo[2,3-b]pyridin-5-yl; and
R^{12a} is H or methyl.

Some embodiments of the present invention include every combination of one or more compounds and pharmaceutically acceptable salts, solvates, and hydrates thereof selected from the following group, wherein the Compound Number in bold directly preceding the chemical name is used elsewhere in this disclosure: Compound B1: 3-((2S)-2-hydroxy-3-(8-(naphthalen-2-ylsulfonyl)-1-oxa-8-azaspiro[4.5]decan-3-ylamino)propoxy)-N-methylbenzenesulfonamide; Compound B2: 3-((2S)-2-hydroxy-3-(8-(naphthalen-2-ylsulfonyl)-1-oxa-8-azaspiro[4.5]decan-3-ylamino)propoxy)-N-propylbenzenesulfonamide; Compound B3: 3-((2S)-2-hydroxy-3-(8-(naphthalen-2-ylsulfonyl)-1-oxa-8-azaspiro[4.5]decan-3-ylamino)propoxy)-N-pentylbenzenesulfonamide; Compound B4: 3-((2S)-2-hydroxy-3-(8-(naphthalen-2-ylsulfonyl)-1-oxa-8-azaspiro[4.5]decan-3-ylamino)propoxy)-N,N-dimethylbenzenesulfonamide; Compound B5: 3-((2S)-2-hydroxy-3-(8-(naphthalen-2-ylsulfonyl)-1-oxa-8-azaspiro[4.5]decan-3-ylamino)propoxy)-N-(2,2,2-trifluoroethyl)benzenesulfonamide; Compound B6: 3-((2S)-2-hydroxy-3-(8-(naphthalen-2-ylsulfonyl)-1-oxa-8-azaspiro[4.5]decan-3-ylamino)propoxy)-N-isopropylbenzenesulfonamide; Compound B7: N-ethyl-3-((2S)-2-hydroxy-3-(8-(naphthalen-2-ylsulfonyl)-1-oxa-8-azaspiro[4.5]decan-3-ylamino)propoxy)-N-isopropylbenzenesulfonamide; Compound B8: N-(cyclopropylmethyl)-3-((2S)-2-hydroxy-3-(8-(naphthalen-2-ylsulfonyl)-1-oxa-8-azaspiro[4.5]decan-3-ylamino)propoxy)benzenesulfonamide; Compound B9: N-(2,2-difluoroethyl)-3-((2S)-2-hydroxy-3-(8-(naphthalen-2-ylsulfonyl)-1-oxa-8-azaspiro[4.5]decan-3-ylamino)propoxy)benzenesulfonamide; Compound B10: N-sec-butyl-3-((2S)-2-hydroxy-3-(8-(naphthalen-2-ylsulfonyl)-1-oxa-8-azaspiro[4.5]decan-3-ylamino)propoxy)benzenesulfonamide; Compound B11: 3-((2S)-2-hydroxy-3-(8-(naphthalen-2-ylsulfonyl)-1-oxa-8-azaspiro[4.5]decan-3-ylamino)propoxy)-N-methoxybenzenesulfonamide; Compound B12: 3-((2S)-2-hydroxy-3-(8-(naphthalen-2-ylsulfonyl)-1-oxa-8-azaspiro[4.5]decan-3-ylamino)propoxy)-N-(2-hydroxyethyl)benzenesulfonamide; Compound B13: 3-((2S)-2-hydroxy-3-(8-(naphthalen-2-ylsulfonyl)-1-oxa-8-azaspiro[4.5]decan-3-ylamino)propoxy)-N-(2-methoxyethyl)benzenesulfonamide; Compound B14: 3-((2S)-2-hydroxy-3-(8-(phenylsulfonyl)-1-oxa-8-azaspiro[4.5]decan-3-ylamino)propoxy)-N-methylbenzenesulfonamide; Compound B15: 3-((2S)-3-(8-(3-chlorophenylsulfonyl)-1-oxa-8-azaspiro[4.5]decan-3-ylamino)-2-hydroxypropoxy)-N-methylbenzenesulfonamide; Compound B16: 3-((2S)-2-hydroxy-3-(8-(5,6,7,8-tetrahydronaphthalen-2-ylsulfonyl)-1-oxa-8-azaspiro[4.5]decan-3-ylamino)propoxy)-N-methylbenzenesulfonamide; Compound B17: 3-((2S)-3-(8-(4-chlorophenylsulfonyl)-1-oxa-8-azaspiro[4.5]decan-3-ylamino)-2-hydroxypropoxy)-N-methylbenzenesulfonamide; Compound B18: 3-((2S)-2-hydroxy-3-(8-(4-methoxyphenylsulfonyl)-1-oxa-8-azaspiro[4.5]decan-3-ylamino)propoxy)-N-methylbenzenesulfonamide; Compound B19: 3-((2S)-3-(8-(3,4-dimethylphenylsulfonyl)-1-oxa-8-azaspiro[4.5]decan-3-ylamino)-2-hydroxypropoxy)-N-methylbenzenesulfonamide; Compound B20: 3-((2S)-3-(8-(3-bromophenylsulfonyl)-1-oxa-8-azaspiro[4.5]decan-3-ylamino)-2-hydroxypropoxy)-N-methylbenzenesulfonamide; Compound B21: 3-((2S)-2-hydroxy-3-(8-(m-tolylsulfonyl)-1-oxa-8-azaspiro[4.5]decan-3-ylamino)propoxy)-N-methylbenzenesulfonamide; Compound B22: 3-((2S)-3-(8-(4-sec-butylphenylsulfonyl)-1-oxa-8-azaspiro[4.5]decan-3-ylamino)-2-hydroxypropoxy)-N-methylbenzenesulfonamide; Compound B23: 3-((2S)-3-(8-(3,5-dimethylphenylsulfonyl)-1-oxa-8-azaspiro[4.5]decan-3-ylamino)-2-hydroxypropoxy)-N-methylbenzenesulfonamide; Compound B24: 4-(3-((S)-2-hydroxy-3-(3-(N-methylsulfamoyl)phenoxy)propylamino)-1-oxa-8-azaspiro[4.5]decan-8-ylsulfonyl)-N,N-dimethylbenzamide; Compound B25: 3-((2S)-3-(8-(4-acetylphenylsulfonyl)-1-oxa-8-azaspiro[4.5]decan-3-ylamino)-2-hydroxypropoxy)-N-methylbenzenesulfonamide; Compound B26: 3-((2S)-3-(8-(4-fluorophenylsulfonyl)-1-oxa-8-azaspiro[4.5]decan-3-ylamino)-2-hydroxypropoxy)-N-methylbenzenesulfonamide; Compound B27: 3-((2S)-2-hydroxy-3-(8-(3-methoxyphenylsulfonyl)-1-oxa-8-azaspiro[4.5]decan-3-ylamino)propoxy)-N-methylbenzenesulfonamide; Compound B28: 3-((2S)-3-(8-(3-fluorophenylsulfonyl)-1-oxa-8-azaspiro[4.5]decan-3-ylamino)-2-hydroxypropoxy)-N-methylbenzenesulfonamide; Compound B29: 3-((S)-2-hydroxy-3-((S)-8-(naphthalen-2-ylsulfonyl)-1-oxa-8-azaspiro[4.5]decan-3-ylamino)propoxy)-N-methylbenzenesulfonamide; Compound B30: 3-((S)-2-hydroxy-3-((R)-8-(naphthalen-2-ylsulfonyl)-1-oxa-8-azaspiro[4.5]decan-3-ylamino)propoxy)-N-methylbenzenesulfonamide; Compound B31: 3-((2S)-3-(8-(biphenyl-3-ylsulfonyl)-1-oxa-8-azaspiro[4.5]decan-3-ylamino)-2-hydroxypropoxy)-N-methylbenzenesulfonamide; Compound B32: 3-((2S)-3-(8-(3-cyanophenylsulfonyl)-1-oxa-8-azaspiro[4.5]decan-3-ylamino)-2-hydroxypropoxy)-N-methylbenzenesulfonamide; Compound B33: 3-((2S)-2-hydroxy-3-(8-(2-(trifluoromethyl)phenylsulfonyl)-1-oxa-8-azaspiro[4.5]decan-3-ylamino)propoxy)-N-methylbenzenesulfonamide; Compound B34: 3-((2S)-3-(8-(2-fluorophenylsulfonyl)-1-oxa-8-azaspiro[4.5]decan-3-ylamino)-2-hydroxypropoxy)-N-methylbenzenesulfonamide; Compound B35: 3-((2S)-3-(8-(2-chlorophenylsulfonyl)-1-oxa-8-azaspiro[4.5]decan-3-ylamino)-2-hydroxypropoxy)-N-methylbenzenesulfonamide; Compound B36: 3-((2S)-3-(8-(4-tert-butylphenylsulfonyl)-1-oxa-8-azaspiro[4.5]decan-3-ylamino)-2-hydroxypropoxy)-N-methylbenzenesulfonamide; Compound B37: 3-((2S)-2-hydroxy-3-(8-(4-(methylsulfonyl)phenylsulfonyl)-1-oxa-8-azaspiro[4.5]decan-3-ylamino)propoxy)-N-methylbenzenesulfonamide; Compound B38: 3-((2S)-2-hydroxy-3-(8-(3-(trifluoromethyl)phenylsulfonyl)-1-oxa-8-azaspiro[4.5]decan-3-ylamino)propoxy)-N-methylbenzenesulfonamide; Compound B39: 3-((2S)-3-(8-(2-cyanophenylsulfonyl)-1-oxa-8-azaspiro[4.5]decan-3-ylamino)-2-hydroxypropoxy)-N-methylbenzenesulfonamide; Compound B40: 4-(3-((S)-2-hydroxy-3-(3-(N-methylsulfamoyl)phenoxy)propylamino)-1-oxa-8-azaspiro[4.5]decan-8-ylsulfonyl)benzoic acid; Compound B41: 3-((2S)-3-(8-(chroman-6-ylsulfonyl)-1-oxa-8-azaspiro[4.5]decan-3-ylamino)-2-hydroxypropoxy)-N-methylbenzenesulfonamide; Compound B42: N-(4-(3-((S)-2-hydroxy-3-(3-(N-methylsulfamoyl)phenoxy)propylamino)-1-oxa-8-azaspiro[4.5]decan-8-ylsulfonyl)phenyl)acetamide; Compound B43: 3-((2S)-2-hydroxy-3-(8-(2-(methylsulfonyl)phenylsulfonyl)-1-oxa-8-azaspiro[4.5]decan-3-ylamino)propoxy)-N-methylbenzenesulfonamide; Compound B44: 3-((2S)-3-(8-(4-cyanophenylsulfonyl)-1-oxa-8-azaspiro[4.5]decan-3-ylamino)-2-hydroxypropoxy)-N-methylbenzenesulfonamide; Compound B45: 3-((2S)-2-hydroxy-3-(8-(4-(trifluoromethyl)phenylsulfonyl)-1-oxa-8-azaspiro[4.5]decan-3-ylamino)propoxy)-N-methylbenzenesulfonamide; Compound B46: 3-((2S)-2-hydroxy-3-(8-(naphthalen-2-ylsulfonyl)-1-oxa-8-azaspiro[4.5]decan-3-ylamino)propoxy)-N-(2-hydroxypropyl)benzenesulfonamide; Compound B47: N-(2-ethoxyethyl)-3-((2S)-2-hydroxy-3-(8-(naphthalen-2-ylsulfonyl)-1-oxa-8-azaspiro[4.5]decan-3-ylamino)propoxy)benzenesulfonamide; Compound B48: 3-((2S)-2-hydroxy-3-(8-(naphthalen-2-ylsulfonyl)-1-oxa-8-azaspiro[4.5]decan-3-ylamino)propoxy)-N-((S)-1-hydroxypropan-2-yl)benzenesulfonamide; Compound B49: N-(1-hydroxy-2-methylpropan-2-yl)-3-((2S)-2-hydroxy-3-(8-(naphthalen-2-ylsulfonyl)-1-oxa-8-azaspiro[4.5]decan-3-ylamino)propoxy)benzenesulfonamide; Compound B50: 3-((2S)-2-hydroxy-3-(8-(naphthalen-2-ylsulfonyl)-1-oxa-8-azaspiro[4.5]decan-3-ylamino)propoxy)-N-(tetrahydro-2H-pyran-4-yl)benzenesulfonamide; Compound B51: 3-((2S)-2-hydroxy-3-(8-(naphthalen-2-ylsulfonyl)-1-oxa-8-azaspiro[4.5]decan-3-ylamino)propoxy)-N-(3-hydroxypropyl)benzenesulfonamide; Compound B52: 3-((2S)-3-(8-(4-bromophenylsulfonyl)-1-oxa-8-azaspiro[4.5]decan-3-ylamino)-2-hydroxypropoxy)-N-methylbenzenesulfonamide; Compound B53: 3-((2S)-2-hydroxy-3-(8-tosyl-1-oxa-8-azaspiro[4.5]decan-3-ylamino)propoxy)-N-methylbenzenesulfonamide; Compound B54: 3-((2S)-3-(8-(2-bromophenylsulfonyl)-1-oxa-8-azaspiro[4.5]decan-3-ylamino)-2-hydroxypropoxy)-N-methylbenzenesulfonamide; Compound B55: 3-((2S)-2-hydroxy-3-(8-(naphthalen-1-ylsulfonyl)-1-oxa-8-azaspiro[4.5]decan-3-ylamino)propoxy)-N-methylbenzenesulfonamide; Compound B56: 3-(3-((S)-2-hydroxy-3-(3-(N-methylsulfamoyl)phenoxy)propylamino)-1-oxa-8-azaspiro[4.5]decan-8-ylsulfonyl)benzoic acid; Compound B57: 3-((2S)-2-hydroxy-3-(8-(4-(trifluoromethoxy)phenylsulfonyl)-1-oxa-8-azaspiro[4.5]decan-3-ylamino)propoxy)-N-methylbenzenesulfonamide; Compound B58: 3-((2S)-2-hydroxy-3-(8-(2-oxo-2,3-dihydrobenzo[d]thiazol-6-ylsulfonyl)-1-oxa-8-azaspiro[4.5]decan-3-ylamino)propoxy)-N-methylbenzenesulfonamide; Compound B59: 3-((2S)-3-(8-(3-(3,5-dimethylisoxazol-4-yl)phenylsulfonyl)-1-oxa-8-azaspiro[4.5]decan-3-ylamino)-2-hydroxypropoxy)-N-methylbenzenesulfonamide; Compound B60: 3-((2S)-2-hydroxy-3-(8-(naphthalen-2-ylsulfonyl)-1-oxa-8-azaspiro[4.5]decan-3-ylamino)propoxy)-N-(2-methoxyethyl)-N-methylbenzenesulfonamide; Compound B61: 3-((2S)-2-hydroxy-3-(8-(3-(1-methyl-1H-pyrazol-4-yl)phenylsulfonyl)-1-oxa-8-azaspiro[4.5]decan-3-ylamino)propoxy)-N-methylbenzenesulfonamide; Compound B62: 3-((2S)-2-hydroxy-3-(8-(3-(1-methyl-3-(trifluoromethyl)-1H-pyrazol-4-yl)phenylsulfonyl)-1-oxa-8-azaspiro[4.5]decan-3-ylamino)propoxy)-N-methylbenzenesulfonamide; Compound B63: 3-((2S)-3-(8-(3-(1H-pyrrol-3-yl)phenylsulfonyl)-1-oxa-8-azaspiro[4.5]decan-3-ylamino)-2-hydroxypropoxy)-N-methylbenzenesulfonamide; Compound B64: 3-((2S)-3-(8-(3-(1H-pyrazol-4-yl)phenylsulfonyl)-1-oxa-8-azaspiro[4.5]decan-3-ylamino)-2-hydroxypropoxy)-N-methylbenzenesulfonamide: Compound B65: 3-((2S)-2-hydroxy-3-(8-(3′-(methoxymethyl)biphenyl-3-ylsulfonyl)-1-oxa-8-azaspiro[4.5]decan-3-ylamino)propoxy)-N-methylbenzenesulfonamide; Compound B66: 3-((2S)-2-hydroxy-3-(8-(3-(thiophen-3-yl)phenylsulfonyl)-1-oxa-8-azaspiro[4.5]decan-3-ylamino)propoxy)-N-methylbenzenesulfonamide; Compound B67: 3-((2S)-3-(8-(3′-(aminomethyl)biphenyl-3-ylsulfonyl)-1-oxa-8-azaspiro[4.5]decan-3-ylamino)-2-hydroxypropoxy)-N-methylbenzenesulfonamide; Compound B68: 3-((2S)-3-(8-(3-(1,3-dimethyl-1H-pyrazol-4-yl)phenylsulfonyl)-1-oxa-8-azaspiro[4.5]decan-3-ylamino)-2-hydroxypropoxy)-N-methylbenzenesulfonamide; Compound B69: 3-((2S)-3-(8-(3-(1-ethyl-1H-pyrazol-4-yl)phenylsulfonyl)-1-oxa-8-azaspiro[4.5]decan-3-ylamino)-2-hydroxypropoxy)-N-methylbenzenesulfonamide; Compound B70: 3-((2S)-2-hydroxy-3-(8-(3-(1-(2-hydroxyethyl)-1H-pyrazol-4-yl)phenylsulfonyl)-1-oxa-8-azaspiro[4.5]decan-3-ylamino)propoxy)-N-methylbenzenesulfonamide; Compound B71: 3-((2S)-2-hydroxy-3-(8-(3′-(methylsulfonyl)biphenyl-3-ylsulfonyl)-1-oxa-8-azaspiro[4.5]decan-3-ylamino)propoxy)-N-methylbenzenesulfonamide; Compound B72: 3-((2S)-3-(8-(3′-cyanobiphenyl-3-ylsulfonyl)-1-oxa-8-azaspiro[4.5]decan-3-ylamino)-2-hydroxypropoxy)-N-methylbenzenesulfonamide; Compound B73: 3-((2S)-3-(8-(3-(2,4-dimethylthiazol-5-yl)phenylsulfonyl)-1-oxa-8-azaspiro[4.5]decan-3-ylamino)-2-hydroxypropoxy)-N-methylbenzenesulfonamide; Compound B74: N-cyclopropyl-3-((2S)-2-hydroxy-3-(8-(naphthalen-2-ylsulfonyl)-1-oxa-8-azaspiro[4.5]decan-3-ylamino)propoxy)benzenesulfonamide; Compound B75: 3-((2S)-2-hydroxy-3-(8-(naphthalen-2-ylsulfonyl)-1-oxa-8-azaspiro[4.5]decan-3-ylamino)propoxy)-N-(3-methoxypropyl)benzenesulfonamide; Compound B76: 3-((2S)-2-hydroxy-3-(8-(3-(1-methyl-1H-pyrrol-3-yl)phenylsulfonyl)-1-oxa-8-azaspiro[4.5]decan-3-ylamino)propoxy)-N-methylbenzenesulfonamide; Compound B77: 3-((2S)-3-(8-(3-(1-benzyl-1H-pyrazol-4-yl)phenylsulfonyl)-1-oxa-8-azaspiro[4.5]decan-3-ylamino)-2-hydroxypropoxy)-N-methylbenzenesulfonamide; Compound B78: 3-((2S)-3-(8-(3-(furan-2-yl)phenylsulfonyl)-1-oxa-8-azaspiro[4.5]decan-3-ylamino)-2-hydroxypropoxy)-N-methylbenzenesulfonamide; Compound B79: 3-((2S)-3-(8-(4′-(aminomethyl)biphenyl-3-ylsulfonyl)-1-oxa-8-azaspiro[4.5]decan-3-ylamino)-2-hydroxypropoxy)-N-methylbenzenesulfonamide; Compound B80: 3-((2S)-2-hydroxy-3-(8-(3-(1-propyl-1H-pyrazol-4-yl)phenylsulfonyl)-1-oxa-8-azaspiro[4.5]decan-3-ylamino)propoxy)-N-methylbenzenesulfonamide; Compound B81: 3-((2S)-2-hydroxy-3-(8-(3-(1-isobutyl-1H-pyrazol-4-yl)phenylsulfonyl)-1-oxa-8-azaspiro[4.5]decan-3-ylamino)propoxy)-N-methylbenzenesulfonamide; Compound B82: 3-((2S)-3-(8-(3-(1-(cyclopropylmethyl)-1H-pyrazol-4-yl)phenylsulfonyl)-1-oxa-8-azaspiro[4.5]decan-3-ylamino)-2-hydroxypropoxy)-N-methylbenzenesulfonamide; Compound B83: 3-((2S)-3-(8-(3′-(dimethylamino)biphenyl-3-ylsulfonyl)-1-oxa-8-azaspiro[4.5]decan-3-ylamino)-2-hydroxypropoxy)-N-methylbenzenesulfonamide; Compound B84: 3-((2S)-3-(8-(3-(1H-pyrazol-3-yl)phenylsulfonyl)-1-oxa-8-azaspiro[4.5]decan-3-ylamino)-2-hydroxypropoxy)-N-methylbenzenesulfonamide; Compound B85: 3′-(3-((S)-2-hydroxy-3-(3-(N-methylsulfamoyl)phenoxy)propylamino)-1-oxa-8-azaspiro[4.5]decan-8-ylsulfonyl)biphenyl-4-sulfonamide; Compound B86: 3-((2S)-2-hydroxy-3-(8-(4′-(methylsulfonamido)biphenyl-3-ylsulfonyl)-1-oxa-8-azaspiro[4.5]decan-3-ylamino)propoxy)-N-methylbenzenesulfonamide; Compound B87: 2-(3′-(3-((S)-2-hydroxy-3-(3-(N-methylsulfamoyl)phenoxy)propylamino)-1-oxa-8-azaspiro[4.5]decan-8-ylsulfonyl)biphenyl-4-yl)acetic acid; Compound B88: 3-((2S)-2-hydroxy-3-(8-(3-(pyridin-3-yl)phenylsulfonyl)-1-oxa-8-azaspiro[4.5]decan-3-ylamino)propoxy)-N-methylbenzenesulfonamide; Compound B89: N-(3,3-difluorocyclobutyl)-3-((2S)-2-hydroxy-3-(8-(naphthalen-2-ylsulfonyl)-1-oxa-8-azaspiro[4.5]decan-3-ylamino)propoxy)benzenesulfonamide; Compound B90: N-cyclopropyl-3-((2S)-2-hydroxy-3-(8-(naphthalen-2-ylsulfonyl)-1-oxa-8-azaspiro[4.5]decan-3-ylamino)propoxy)-N-methylbenzenesulfonamide; Compound B91: N-(2-aminoethyl)-3-((2S)-2-hydroxy-3-(8-(naphthalen-2-ylsulfonyl)-1-oxa-8-azaspiro[4.5]decan-3-ylamino)propoxy)benzenesulfonamide; Compound B92: (2S,3R)-3-hydroxy-2-(3-((2S)-2-hydroxy-3-(8-(naphthalen-2-ylsulfonyl)-1-oxa-8-azaspiro[4.5]decan-3-ylamino)propoxy)phenylsulfonamido)-N-methylbutanamide; Compound B93: N-((R)-1-cyclopropylethyl)-3-((2S)-2-hydroxy-3-(8-(naphthalen-2-ylsulfonyl)-1-oxa-8-azaspiro[4.5]decan-3-ylamino)propoxy)benzenesulfonamide; Compound B94: N-tert-butyl-3-((2S)-2-hydroxy-3-(8-(naphthalen-2-ylsulfonyl)-1-oxa-8-azaspiro[4.5]decan-3-ylamino)propoxy)benzenesulfonamide; Compound B95: N-(1,3-dihydroxypropan-2-yl)-3-((2S)-2-hydroxy-3-(8-(naphthalen-2-ylsulfonyl)-1-oxa-8-azaspiro[4.5]decan-3-ylamino)propoxy)benzenesulfonamide; Compound B96: 3-((2S)-2-hydroxy-3-(8-(naphthalen-2-ylsulfonyl)-1-oxa-8-azaspiro[4.5]decan-3-ylamino)propoxy)-N-((R)-1-hydroxypropan-2-yl)benzenesulfonamide; Compound B97: N-(2-ethylbutyl)-3-((2S)-2-hydroxy-3-(8-(naphthalen-2-ylsulfonyl)-1-oxa-8-azaspiro[4.5]decan-3-ylamino)propoxy)benzenesulfonamide; Compound B98: 3-((2S)-2-hydroxy-3-(8-(naphthalen-2-ylsulfonyl)-1-oxa-8-azaspiro[4.5]decan-3-ylamino)propoxy)-N-isopentylbenzenesulfonamide; Compound B99: 3-((2S)-2-hydroxy-3-(8-(naphthalen-2-ylsulfonyl)-1-oxa-8-azaspiro[4.5]decan-3-ylamino)propoxy)-N-(1-(hydroxymethyl)cyclopropyl)benzenesulfonamide; Compound B100: N-((R)-3,3-dimethylbutan-2-yl)-3-((2S)-2-hydroxy-3-(8-(naphthalen-2-ylsulfonyl)-1-oxa-8-azaspiro[4.5]decan-3-ylamino)propoxy)benzenesulfonamide; Compound B101: N-ethyl-3-((2S)-2-hydroxy-3-(8-(naphthalen-2-ylsulfonyl)-1-oxa-8-azaspiro[4.5]decan-3-ylamino)propoxy)benzenesulfonamide; Compound B102: 3-((2S)-2-hydroxy-3-(8-(3-(pyridin-2-yl)phenylsulfonyl)-1-oxa-8-azaspiro[4.5]decan-3-ylamino)propoxy)-N-methylbenzenesulfonamide; Compound B103: 3-((2S)-3-(8-(3-(6-aminopyridin-3-yl)phenylsulfonyl)-1-oxa-8-azaspiro[4.5]decan-3-ylamino)-2-hydroxypropoxy)-N-methylbenzenesulfonamide; Compound B104: 3-((2S)-2-hydroxy-3-(8-(3-(pyrimidin-5-yl)phenylsulfonyl)-1-oxa-8-azaspiro[4.5]decan-3-ylamino)propoxy)-N-methylbenzenesulfonamide; Compound B105: 3-((2S)-3-(8-(3′-((dimethylamino)methyl)biphenyl-3-ylsulfonyl)-1-oxa-8-azaspiro[4.5]decan-3-ylamino)-2-hydroxypropoxy)-N-methylbenzenesulfonamide; Compound B106: 3-((2S)-2-hydroxy-3-(8-(3-(pyridin-4-yl)phenylsulfonyl)-1-oxa-8-azaspiro[4.5]decan-3-ylamino)propoxy)-N-methylbenzenesulfonamide; Compound B107: 3-((2S)-2-hydroxy-3-(8-(3-(2-methylpyridin-4-yl)phenylsulfonyl)-1-oxa-8-azaspiro[4.5]decan-3-ylamino)propoxy)-N-methylbenzenesulfonamide; Compound B108: 3-((2S)-2-hydroxy-3-(8-(3-(2-methoxypyrimidin-5-yl)phenylsulfonyl)-1-oxa-8-azaspiro[4.5]decan-3-ylamino)propoxy)-N-methylbenzenesulfonamide; Compound B109: 3-((2S)-2-hydroxy-3-(8-(3-(4-methylthiophen-3-yl)phenylsulfonyl)-1-oxa-8-azaspiro[4.5]decan-3-ylamino)propoxy)-N-methylbenzenesulfonamide; Compound B110: 3-((2S)-2-hydroxy-3-(8-(3-(5-methylpyridin-3-yl)phenylsulfonyl)-1-oxa-8-azaspiro[4.5]decan-3-ylamino)propoxy)-N-methylbenzenesulfonamide; Compound B111: 3-((2S)-2-hydroxy-3-(8-(3-(6-methylpyridin-3-yl)phenylsulfonyl)-1-oxa-8-azaspiro[4.5]decan-3-ylamino)propoxy)-N-methylbenzenesulfonamide; Compound B112: 3′-(3-((S)-2-hydroxy-3-(3-(N-methylsulfamoyl)phenoxy)propylamino)-1-oxa-8-azaspiro[4.5]decan-8-ylsulfonyl)biphenyl-3-carboxylic acid; Compound B113: 3-((2S)-3-(8-(3-(1,3-dimethyl-2,4-dioxo-1,2,3,4-tetrahydropyrimidin-5-yl)phenylsulfonyl)-1-oxa-8-azaspiro[4.5]decan-3-ylamino)-2-hydroxypropoxy)-N-methylbenzenesulfonamide; Compound B114: 3-((2S)-3-(8-(4′-(cyanomethoxy)biphenyl-3-ylsulfonyl)-1-oxa-8-azaspiro[4.5]decan-3-ylamino)-2-hydroxypropoxy)-N-methylbenzenesulfonamide; Compound B115: 3′-(3-((S)-2-hydroxy-3-(3-(N-methylsulfamoyl)phenoxy)propylamino)-1-oxa-8-azaspiro[4.5]decan-8-ylsulfonyl)-N,N-dimethylbiphenyl-3-sulfonamide; Compound B116: 3-((2S)-2-hydroxy-3-(8-(3-(3-methylthiophen-2-yl)phenylsulfonyl)-1-oxa-8-azaspiro[4.5]decan-3-ylamino)propoxy)-N-methylbenzenesulfonamide; Compound B117: 3-((2S)-3-(8-(4′-(cyanomethyl)biphenyl-3-ylsulfonyl)-1-oxa-8-azaspiro[4.5]decan-3-ylamino)-2-hydroxypropoxy)-N-methylbenzenesulfonamide; Compound B118: 3-((2S)-2-hydroxy-3-(8-(3′-(hydroxymethyl)biphenyl-3-ylsulfonyl)-1-oxa-8-azaspiro[4.5]decan-3-ylamino)propoxy)-N-methylbenzenesulfonamide; Compound B119: 3-((2S)-3-(8-(1,2-dimethyl-1H-imidazol-4-ylsulfonyl)-1-oxa-8-azaspiro[4.5]decan-3-ylamino)-2-hydroxypropoxy)-N-methylbenzenesulfonamide; Compound B120: 3-((2S)-2-hydroxy-3-(8-(4-tert-pentylphenylsulfonyl)-1-oxa-8-azaspiro[4.5]decan-3-ylamino)propoxy)-N-methylbenzenesulfonamide; Compound B121: 3-((2S)-2-hydroxy-3-(8-(4′-(trifluoromethyl)biphenyl-4-ylsulfonyl)-1-oxa-8-azaspiro[4.5]decan-3-ylamino)propoxy)-N-methylbenzenesulfonamide; Compound B122: 3-((2S)-3-(8-(4′-fluorobiphenyl-4-ylsulfonyl)-1-oxa-8-azaspiro[4.5]decan-3-ylamino)-2-hydroxypropoxy)-N-methylbenzenesulfonamide; Compound B123: 3-((2S)-3-(8-(biphenyl-4-ylsulfonyl)-1-oxa-8-azaspiro[4.5]decan-3-ylamino)-2-hydroxypropoxy)-N-methylbenzenesulfonamide; Compound B124: 3-((2S)-2-hydroxy-3-(8-(3-phenoxyphenylsulfonyl)-1-oxa-8-azaspiro[4.5]decan-3-ylamino)propoxy)-N-methylbenzenesulfonamide; Compound B125: 3-((2S)-3-(8-(cyclohexylsulfonyl)-1-oxa-8-azaspiro[4.5]decan-3-ylamino)-2-hydroxypropoxy)-N-methylbenzenesulfonamide; Compound B126: 3-((2S)-2-hydroxy-3-(8-(3-oxo-3,4-dihydro-2H-benzo[b][1,4]oxazin-6-ylsulfonyl)-1-oxa-8-azaspiro[4.5]decan-3-ylamino)propoxy)-N-methylbenzenesulfonamide; Compound B127: 3-((2S)-3-(8-(2,2-dimethylchroman-6-ylsulfonyl)-1-oxa-8-azaspiro[4.5]decan-3-ylamino)-2-hydroxypropoxy)-N-methylbenzenesulfonamide; Compound B128: 3-((2S)-3-(8-(benzo[c][1,2,5]thiadiazol-4-ylsulfonyl)-1-oxa-8-azaspiro[4.5]decan-3-ylamino)-2-hydroxypropoxy)-N-methylbenzenesulfonamide; Compound B129: 3-((2S)-3-(8-(6-chloroimidazo[2,1-b]thiazol-5-ylsulfonyl)-1-oxa-8-azaspiro[4.5]decan-3-ylamino)-2-hydroxypropoxy)-N-methylbenzenesulfonamide; Compound B130: 3-((2S)-2-hydroxy-3-(8-(6-phenoxypyridin-3-ylsulfonyl)-1-oxa-8-azaspiro[4.5]decan-3-ylamino)propoxy)-N-methylbenzenesulfonamide; Compound B131: 3-((2S)-3-(8-(1H-pyrazol-4-ylsulfonyl)-1-oxa-8-azaspiro[4.5]decan-3-ylamino)-2-hydroxypropoxy)-N-methylbenzenesulfonamide; Compound B132: 3-((2S)-2-hydroxy-3-(8-(5-methyl-1-phenyl-1H-pyrazol-4-ylsulfonyl)-1-oxa-8-azaspiro[4.5]decan-3-ylamino)propoxy)-N-methylbenzenesulfonamide; Compound B133: 3-((2S)-3-(8-(2-chloro-5-(trifluoromethyl)phenylsulfonyl)-1-oxa-8-azaspiro[4.5]decan-3-ylamino)-2-hydroxypropoxy)-N-methylbenzenesulfonamide; Compound B134: 3-((2S)-3-(8-(2,4-difluorophenylsulfonyl)-1-oxa-8-azaspiro[4.5]decan-3-ylamino)-2-hydroxypropoxy)-N-methylbenzenesulfonamide; Compound B135: 3-((2S)-2-hydroxy-3-(8-(2-methoxy-4-methylphenylsulfonyl)-1-oxa-8-azaspiro[4.5]decan-3-ylamino)propoxy)-N-methylbenzenesulfonamide; Compound B136: 3-((2S)-3-(8-(4-ethoxyphenylsulfonyl)-1-oxa-8-azaspiro[4.5]decan-3-ylamino)-2-hydroxypropoxy)-N-methylbenzenesulfonamide; Compound B137: 3-((2S)-2-hydroxy-3-(8-(4-isopropoxyphenylsulfonyl)-1-oxa-8-azaspiro[4.5]decan-3-ylamino)propoxy)-N-methylbenzenesulfonamide; Compound B138: 3-((2S)-3-(8-(3,5-dichlorophenylsulfonyl)-1-oxa-8-azaspiro[4.5]decan-3-ylamino)-2-hydroxypropoxy)-N-methylbenzenesulfonamide; Compound B139: 3-((2S)-3-(8-(3-chloro-2-fluorophenylsulfonyl)-1-oxa-8-azaspiro[4.5]decan-3-ylamino)-2-hydroxypropoxy)-N-methylbenzenesulfonamide; Compound B140: 3-((2S)-3-(8-(2,5-dichlorophenylsulfonyl)-1-oxa-8-azaspiro[4.5]decan-3-ylamino)-2-hydroxypropoxy)-N-methylbenzenesulfonamide; Compound B141: 3-((2S)-3-(8-(3,4-difluorophenylsulfonyl)-1-oxa-8-azaspiro[4.5]decan-3-ylamino)-2-hydroxypropoxy)-N-methylbenzenesulfonamide; Compound B142: 3-((2S)-3-(8-(2,3-dichlorophenylsulfonyl)-1-oxa-8-azaspiro[4.5]decan-3-ylamino)-2-hydroxypropoxy)-N-methylbenzenesulfonamide; Compound B143: 3-((2S)-3-(8-(3-chloro-2-methylphenylsulfonyl)-1-oxa-8-azaspiro[4.5]decan-3-ylamino)-2-hydroxypropoxy)-N-methylbenzenesulfonamide; Compound B144: 3-((2S)-3-(8-(5-chloro-2-fluorophenylsulfonyl)-1-oxa-8-azaspiro[4.5]decan-3-ylamino)-2-hydroxypropoxy)-N-methylbenzenesulfonamide; Compound B145: 3-((2S)-3-(8-(5-chloro-2-cyanophenylsulfonyl)-1-oxa-8-azaspiro[4.5]decan-3-ylamino)-2-hydroxypropoxy)-N-methylbenzenesulfonamide; Compound B146: 3-((2S)-3-(8-(1H-benzo[d]imidazol-6-ylsulfonyl)-1-oxa-8-azaspiro[4.5]decan-3-ylamino)-2-hydroxypropoxy)-N-methylbenzenesulfonamide; Compound B147: 3-((2S)-2-hydroxy-3-(8-(4-methoxy-3-methylphenylsulfonyl)-1-oxa-8-azaspiro[4.5]decan-3-ylamino)propoxy)-N-methylbenzenesulfonamide; Compound B148: 3-((2S)-3-(8-(5-chloro-2-methoxyphenylsulfonyl)-1-oxa-8-azaspiro[4.5]decan-3-ylamino)-2-hydroxypropoxy)-N-methylbenzenesulfonamide; Compound B149: 3-((2S)-3-(8-(4-fluoro-3-methylphenylsulfonyl)-1-oxa-8-azaspiro[4.5]decan-3-ylamino)-2-hydroxypropoxy)-N-methylbenzenesulfonamide; Compound B150: 3-((2S)-3-(8-(2-fluoro-5-methylphenylsulfonyl)-1-oxa-8-azaspiro[4.5]decan-3-ylamino)-2-hydroxypropoxy)-N-methylbenzenesulfonamide; Compound B151: 3-((2S)-3-(8-(4-chloro-3-methylphenylsulfonyl)-1-oxa-8-azaspiro[4.5]decan-3-ylamino)-2-hydroxypropoxy)-N-methylbenzenesulfonamide; Compound B152: 3-((2S)-2-hydroxy-3-(8-(1-methyl-3-(trifluoromethyl)-1H-pyrazol-4-ylsulfonyl)-1-oxa-8-azaspiro[4.5]decan-3-ylamino)propoxy)-N-methylbenzenesulfonamide; Compound B153: 3-((2S)-3-(8-(3,4-dimethoxyphenylsulfonyl)-1-oxa-8-azaspiro[4.5]decan-3-ylamino)-2-hydroxypropoxy)-N-methylbenzenesulfonamide; Compound B154: 3-((2S)-3-(8-(2,5-dimethylphenylsulfonyl)-1-oxa-8-azaspiro[4.5]decan-3-ylamino)-2-hydroxypropoxy)-N-methylbenzenesulfonamide; Compound B155: 3-((2S)-3-(8-(4-bromo-3-chlorophenylsulfonyl)-1-oxa-8-azaspiro[4.5]decan-3-ylamino)-2-hydroxypropoxy)-N-methylbenzenesulfonamide; Compound B156: 3-((2S)-3-(8-(2,6-difluorophenylsulfonyl)-1-oxa-8-azaspiro[4.5]decan-3-ylamino)-2-hydroxypropoxy)-N-methylbenzenesulfonamide; Compound B157: 3-((2S)-3-(8-(2-chloro-4-cyanophenylsulfonyl)-1-oxa-8-azaspiro[4.5]decan-3-ylamino)-2-hydroxypropoxy)-N-methylbenzenesulfonamide; Compound B158: 3-((2S)-3-(8-(2,4-dichlorophenylsulfonyl)-1-oxa-8-azaspiro[4.5]decan-3-ylamino)-2-hydroxypropoxy)-N-methylbenzenesulfonamide; Compound B159: 3-((2S)-3-(8-(4-bromo-3-methylphenylsulfonyl)-1-oxa-8-azaspiro[4.5]decan-3-ylamino)-2-hydroxypropoxy)-N-methylbenzenesulfonamide; Compound B160: 3-((2S)-3-(8-(2-fluoro-3-methylphenylsulfonyl)-1-oxa-8-azaspiro[4.5]decan-3-ylamino)-2-hydroxypropoxy)-N-methylbenzenesulfonamide; Compound B161: 3-((2S)-2-hydroxy-3-(8-(3-(4-(trifluoromethyl)phenoxy)phenylsulfonyl)-1-oxa-8-azaspiro[4.5]decan-3-ylamino)propoxy)-N-methylbenzenesulfonamide; Compound B162: 3-((2S)-2-hydroxy-3-(8-(5-methyl-2-(trifluoromethyl)furan-3-ylsulfonyl)-1-oxa-8-azaspiro[4.5]decan-3-ylamino)propoxy)-N-methylbenzenesulfonamide; Compound B163: 3-((2S)-3-(8-(5-chloro-1,3-dimethyl-1H-pyrazol-4-ylsulfonyl)-1-oxa-8-azaspiro[4.5]decan-3-ylamino)-2-hydroxypropoxy)-N-methylbenzenesulfonamide; Compound B164: 3-((2S)-2-hydroxy-3-(8-(naphthalen-2-ylsulfonyl)-1-oxa-8-azaspiro[4.5]decan-3-ylamino)propoxy)-N-(2-(2-oxopyrrolidin-1-yl)ethyl)benzenesulfonamide; Compound B165: N-(1-ethylpiperidin-4-yl)-3-((2S)-2-hydroxy-3-(8-(naphthalen-2-ylsulfonyl)-1-oxa-8-azaspiro[4.5]decan-3-ylamino)propoxy)benzenesulfonamide; Compound B166: N-((S)-2,3-dihydroxypropyl)-3-((2S)-2-hydroxy-3-(8-(naphthalen-2-ylsulfonyl)-1-oxa-8-azaspiro[4.5]decan-3-ylamino)propoxy)benzenesulfonamide; Compound B167: N-(2-(dimethylamino)ethyl)-3-((2S)-2-hydroxy-3-(8-(naphthalen-2-ylsulfonyl)-1-oxa-8-azaspiro[4.5]decan-3-ylamino)propoxy)benzenesulfonamide; Compound B168: 3-((2S)-2-hydroxy-3-(8-(naphthalen-2-ylsulfonyl)-1-oxa-8-azaspiro[4.5]decan-3-ylamino)propoxy)-N-(piperidin-3-ylmethyl)benzenesulfonamide; Compound B169: 3-((2S)-2-hydroxy-3-(8-(6-methoxynaphthalen-2-ylsulfonyl)-1-oxa-8-azaspiro[4.5]decan-3-ylamino)propoxy)-N-methylbenzenesulfonamide; Compound B170: 3-((2S)-2-hydroxy-3-(8-(3-(2-(trifluoromethyl)phenoxy)phenylsulfonyl)-1-oxa-8-azaspiro[4.5]decan-3-ylamino)propoxy)-N-methylbenzenesulfonamide; Compound B171: 3-((2S)-2-hydroxy-3-(8-(2-(trifluoromethoxy)phenylsulfonyl)-1-oxa-8-azaspiro[4.5]decan-3-ylamino)propoxy)-N-methylbenzenesulfonamide; Compound B172: 3-((2S)-3-(8-(benzylsulfonyl)-1-oxa-8-azaspiro[4.5]decan-3-ylamino)-2-hydroxypropoxy)-N-methylbenzenesulfonamide; Compound B173: 3-((2S)-2-hydroxy-3-(8-(7-methoxybenzo[c][1,2,5]oxadiazol-4-ylsulfonyl)-1-oxa-8-azaspiro[4.5]decan-3-ylamino)propoxy)-N-methylbenzenesulfonamide; Compound B174: 3-((2S)-3-(8-(biphenyl-2-ylsulfonyl)-1-oxa-8-azaspiro[4.5]decan-3-ylamino)-2-hydroxypropoxy)-N-methylbenzenesulfonamide; Compound B175: 3-((2S)-2-hydroxy-3-(8-(5-methylbenzo[c][1,2,5]oxadiazol-4-ylsulfonyl)-1-oxa-8-azaspiro[4.5]decan-3-ylamino)propoxy)-N-methylbenzenesulfonamide; Compound B176: 3-((2S)-2-hydroxy-3-(8-(2-methoxy-5-methylphenylsulfonyl)-1-oxa-8-azaspiro[4.5]decan-3-ylamino)propoxy)-N-methylbenzenesulfonamide; Compound B177: 3-((2S)-3-(8-(benzo[c][1,2,5]thiadiazol-5-ylsulfonyl)-1-oxa-8-azaspiro[4.5]decan-3-ylamino)-2-hydroxypropoxy)-N-methylbenzenesulfonamide; Compound B178: 3-((2S)-3-(8-(4,5-dichlorothiophen-2-ylsulfonyl)-1-oxa-8-azaspiro[4.5]decan-3-ylamino)-2-hydroxypropoxy)-N-methylbenzenesulfonamide; Compound B179: 3-((2S)-3-(8-(3-fluoro-5-methylphenylsulfonyl)-1-oxa-8-azaspiro[4.5]decan-3-ylamino)-2-hydroxypropoxy)-N-methylbenzenesulfonamide; Compound B180: 3-((2S)-3-(8-(2-cyano-5-methylphenylsulfonyl)-1-oxa-8-azaspiro[4.5]decan-3-ylamino)-2-hydroxypropoxy)-N-methylbenzenesulfonamide; Compound B181: 3-((2S)-3-(8-(5-chlorothiophen-2-ylsulfonyl)-1-oxa-8-azaspiro[4.5]decan-3-ylamino)-2-hydroxypropoxy)-N-methylbenzenesulfonamide; Compound B182: 3-((2S)-2-hydroxy-3-(8-(2-oxoindolin-5-ylsulfonyl)-1-oxa-8-azaspiro[4.5]decan-3-ylamino)propoxy)-N-methylbenzenesulfonamide; Compound B183: 3-((S)-3-((S)-8-(chroman-6-ylsulfonyl)-1-oxa-8-azaspiro[4.5]decan-3-ylamino)-2-hydroxypropoxy)-N-methylbenzenesulfonamide; Compound B184: 3-((S)-3-((R)-8-(chroman-6-ylsulfonyl)-1-oxa-8-azaspiro[4.5]decan-3-ylamino)-2-hydroxypropoxy)-N-methylbenzenesulfonamide; Compound B185: 3-((2S)-3-(8-(7-chlorobenzo[c][1,2,5]oxadiazol-4-ylsulfonyl)-1-oxa-8-azaspiro[4.5]decan-3-ylamino)-2-hydroxypropoxy)-N-methylbenzenesulfonamide; Compound B186: 3-((2S)-2-hydroxy-3-(8-(4-methyl-2-phenylthiazol-5-ylsulfonyl)-1-oxa-8-azaspiro[4.5]decan-3-ylamino)propoxy)-N-methylbenzenesulfonamide; Compound B187: 3-((2S)-2-hydroxy-3-(8-(5-phenylthiophen-2-ylsulfonyl)-1-oxa-8-azaspiro[4.5]decan-3-ylamino)propoxy)-N-methylbenzenesulfonamide; Compound B188: 3-((2S)-3-(8-(3,5-dimethylisoxazol-4-ylsulfonyl)-1-oxa-8-azaspiro[4.5]decan-3-ylamino)-2-hydroxypropoxy)-N-methylbenzenesulfonamide; Compound B189: 3-((2S)-2-hydroxy-3-(8-(4-methyl-3,4-dihydro-2H-benzo[b][1,4]oxazin-6-ylsulfonyl)-1-oxa-8-azaspiro[4.5]decan-3-ylamino)propoxy)-N-methylbenzenesulfonamide; Compound B190: 3-((2S)-2-hydroxy-3-(8-(4-(phenylsulfonyl)thiophen-2-ylsulfonyl)-1-oxa-8-azaspiro[4.5]decan-3-ylamino)propoxy)-N-methylbenzenesulfonamide; Compound B191: 3-((2S)-2-hydroxy-3-(8-(5-(1-methyl-3-(trifluoromethyl)-1H-pyrazol-5-yl)thiophen-2-ylsulfonyl)-1-oxa-8-azaspiro[4.5]decan-3-ylamino)propoxy)-N-methylbenzenesulfonamide; Compound B192: 3-((2S)-3-(8-(furan-3-ylsulfonyl)-1-oxa-8-azaspiro[4.5]decan-3-ylamino)-2-hydroxypropoxy)-N-methylbenzenesulfonamide; Compound B193: 3-((2S)-2-hydroxy-3-(8-(1-methyl-1H-pyrazol-3-ylsulfonyl)-1-oxa-8-azaspiro[4.5]decan-3-ylamino)propoxy)-N-methylbenzenesulfonamide; Compound B194: 3-((2S)-2-hydroxy-3-(8-(1-methyl-1H-imidazol-4-ylsulfonyl)-1-oxa-8-azaspiro[4.5]decan-3-ylamino)propoxy)-N-methylbenzenesulfonamide; Compound B195: 3-((2S)-3-(8-(3-fluoro-4-methoxyphenylsulfonyl)-1-oxa-8-azaspiro[4.5]decan-3-ylamino)-2-hydroxypropoxy)-N-methylbenzenesulfonamide; Compound B196: 3-((2S)-2-hydroxy-3-(8-(thiophen-3-ylsulfonyl)-1-oxa-8-azaspiro[4.5]decan-3-ylamino)propoxy)-N-methylbenzenesulfonamide; Compound B197: 3-((2S)-3-(8-(5-chlorobenzo[c][1,2,5]thiadiazol-4-ylsulfonyl)-1-oxa-8-azaspiro[4.5]decan-3-ylamino)-2-hydroxypropoxy)-N-methylbenzenesulfonamide; Compound B198: 3-((2S)-3-(8-(3-cyano-4-methylphenylsulfonyl)-1-oxa-8-azaspiro[4.5]decan-3-ylamino)-2-hydroxypropoxy)-N-methylbenzenesulfonamide; Compound B199: 3-((2S)-3-(8-(2-chloro-3-fluorophenylsulfonyl)-1-oxa-8-azaspiro[4.5]decan-3-ylamino)-2-hydroxypropoxy)-N-methylbenzenesulfonamide; Compound B200: 3-((2S)-3-(8-(2-chloro-4-fluorophenylsulfonyl)-1-oxa-8-azaspiro[4.5]decan-3-ylamino)-2-hydroxypropoxy)-N-methylbenzenesulfonamide; Compound B201: 3-((2S)-3-(8-(2,3-difluorophenylsulfonyl)-1-oxa-8-azaspiro[4.5]decan-3-ylamino)-2-hydroxypropoxy)-N-methylbenzenesulfonamide; Compound B202: 3-((2S)-3-(8-(2-chloro-5-fluorophenylsulfonyl)-1-oxa-8-azaspiro[4.5]decan-3-ylamino)-2-hydroxypropoxy)-N-methylbenzenesulfonamide; Compound B203: 3-((2S)-3-(8-(2-chloro-5-(methylsulfonyl)phenylsulfonyl)-1-oxa-8-azaspiro[4.5]decan-3-ylamino)-2-hydroxypropoxy)-N-methylbenzenesulfonamide; Compound B204: 3-((2S)-3-(8-(5-fluoro-2-methylphenylsulfonyl)-1-oxa-8-azaspiro[4.5]decan-3-ylamino)-2-hydroxypropoxy)-N-methylbenzenesulfonamide; Compound B205: 3-((2S)-3-(8-(2,5-difluorophenylsulfonyl)-1-oxa-8-azaspiro[4.5]decan-3-ylamino)-2-hydroxypropoxy)-N-methylbenzenesulfonamide; Compound B206: 3-((2S)-3-(8-(4-chloro-3-methoxyphenylsulfonyl)-1-oxa-8-azaspiro[4.5]decan-3-ylamino)-2-hydroxypropoxy)-N-methylbenzenesulfonamide; Compound B207: 3-((2S)-3-(8-(3-chloro-4-methoxyphenylsulfonyl)-1-oxa-8-azaspiro[4.5]decan-3-ylamino)-2-hydroxypropoxy)-N-methylbenzenesulfonamide; Compound B208: 3-((2S)-2-hydroxy-3-(8-(5-(5-(trifluoromethyl)isoxazol-3-yl)thiophen-2-ylsulfonyl)-1-oxa-8-azaspiro[4.5]decan-3-ylamino)propoxy)-N-methylbenzenesulfonamide; Compound B209: N-ethyl-3-((2S)-3-(8-(3-(1-ethyl-1H-pyrazol-4-yl)phenylsulfonyl)-1-oxa-8-azaspiro[4.5]decan-3-ylamino)-2-hydroxypropoxy)benzenesulfonamide; Compound B210: 3-((2S)-3-(8-(4′-(aminomethyl)biphenyl-3-ylsulfonyl)-1-oxa-8-azaspiro[4.5]decan-3-ylamino)-2-hydroxypropoxy)-N-ethylbenzenesulfonamide; Compound B211: 3′-(3-((S)-3-(3-(N-ethylsulfamoyl)phenoxy)-2-hydroxypropylamino)-1-oxa-8-azaspiro[4.5]decan-8-ylsulfonyl)biphenyl-4-sulfonamide; Compound B212: 3-((2S)-3-(8-(3-(6-aminopyridin-3-yl)phenylsulfonyl)-1-oxa-8-azaspiro[4.5]decan-3-ylamino)-2-hydroxypropoxy)-N-ethylbenzenesulfonamide; Compound B213: 3-((2S)-3-(8-(4′-(cyanomethoxy)biphenyl-3-ylsulfonyl)-1-oxa-8-azaspiro[4.5]decan-3-ylamino)-2-hydroxypropoxy)-N-ethylbenzenesulfonamide; Compound B214: 3-((2S)-3-(8-(4′-(cyanomethyl)biphenyl-3-ylsulfonyl)-1-oxa-8-azaspiro[4.5]decan-3-ylamino)-2-hydroxypropoxy)-N-ethylbenzenesulfonamide; Compound B215: 3-((S)-2-hydroxy-3-((R)-8-(3-(1-methyl-1H-pyrazol-4-yl)phenylsulfonyl)-1-oxa-8-azaspiro[4.5]decan-3-ylamino)propoxy)-N-methylbenzenesulfonamide; Compound B216: 3-((S)-2-hydroxy-3-((S)-8-(3-(1-methyl-1H-pyrazol-4-yl)phenylsulfonyl)-1-oxa-8-azaspiro[4.5]decan-3-ylamino)propoxy)-N-methylbenzenesulfonamide; Compound B217: 3-((2S)-3-(8-(5-cyano-2-methylphenylsulfonyl)-1-oxa-8-azaspiro[4.5]decan-3-ylamino)-2-hydroxypropoxy)-N-methylbenzenesulfonamide; Compound B218: 3-((2S)-3-(8-(3-bromo-4-methylphenylsulfonyl)-1-oxa-8-azaspiro[4.5]decan-3-ylamino)-2-hydroxypropoxy)-N-methylbenzenesulfonamide; Compound B219: 3-((2S)-3-(8-(3-bromo-5-methylphenylsulfonyl)-1-oxa-8-azaspiro[4.5]decan-3-ylamino)-2-hydroxypropoxy)-N-methylbenzenesulfonamide; Compound B220: 3-((2S)-3-(8-(5-bromo-2-methoxyphenylsulfonyl)-1-oxa-8-azaspiro[4.5]decan-3-ylamino)-2-hydroxypropoxy)-N-methylbenzenesulfonamide; Compound B221: 3-((2S)-2-hydroxy-3-(8-(1-methyl-1H-indol-5-ylsulfonyl)-1-oxa-8-azaspiro[4.5]decan-3-ylamino)propoxy)-N-methylbenzenesulfonamide; Compound B222: 3-((2S)-2-hydroxy-3-(8-(4-methoxy-5,6,7,8-tetrahydronaphthalen-1-ylsulfonyl)-1-oxa-8-azaspiro[4.5]decan-3-ylamino)propoxy)-N-methylbenzenesulfonamide; Compound B223: 3-((2S)-3-(8-(5-chloronaphthalen-2-ylsulfonyl)-1-oxa-8-azaspiro[4.5]decan-3-ylamino)-2-hydroxypropoxy)-N-methylbenzenesulfonamide; Compound B224: 3-((2S)-3-(8-(3-chlorophenylsulfonyl)-1-oxa-8-azaspiro[4.5]decan-3-ylamino)-2-hydroxypropoxy)-N-ethylbenzenesulfonamide; Compound B225: N-ethyl-3-((2S)-2-hydroxy-3-(8-(4-methoxy-3-methylphenylsulfonyl)-1-oxa-8-azaspiro[4.5]decan-3-ylamino)propoxy)benzenesulfonamide; Compound B226: N-ethyl-3-((2S)-3-(8-(2-fluoro-5-methylphenylsulfonyl)-1-oxa-8-azaspiro[4.5]decan-3-ylamino)-2-hydroxypropoxy)benzenesulfonamide; Compound B227: 3-((2S)-3-(8-(4-bromo-3-methylphenylsulfonyl)-1-oxa-8-azaspiro[4.5]decan-3-ylamino)-2-hydroxypropoxy)-N-ethylbenzenesulfonamide; Compound B228: N-ethyl-3-((2S)-2-hydroxy-3-(8-(3-oxo-3,4-dihydro-2H-benzo[b][1,4]oxazin-6-ylsulfonyl)-1-oxa-8-azaspiro[4.5]decan-3-ylamino)propoxy)benzenesulfonamide; Compound B229: 3-((2S)-3-(8-(benzo[c][1,2,5]thiadiazol-5-ylsulfonyl)-1-oxa-8-azaspiro[4.5]decan-3-ylamino)-2-hydroxypropoxy)-N-ethylbenzenesulfonamide; Compound B230: N-ethyl-3-((2S)-2-hydroxy-3-(8-(5-methyl-1-phenyl-1H-pyrazol-4-ylsulfonyl)-1-oxa-8-azaspiro[4.5]decan-3-ylamino)propoxy)benzenesulfonamide; Compound B231: N-ethyl-3-((2S)-2-hydroxy-3-(8-(5,6,7,8-tetrahydronaphthalen-2-ylsulfonyl)-1-oxa-8-azaspiro[4.5]decan-3-ylamino)propoxy)benzenesulfonamide; Compound B232: 3-((2S)-3-(8-(3,5-dimethylisoxazol-4-ylsulfonyl)-1-oxa-8-azaspiro[4.5]decan-3-ylamino)-2-hydroxypropoxy)-N-ethylbenzenesulfonamide; Compound B233: N-ethyl-3-((2S)-2-hydroxy-3-(8-(5-phenylthiophen-2-ylsulfonyl)-1-oxa-8-azaspiro[4.5]decan-3-ylamino)propoxy)benzenesulfonamide; Compound B234: N-ethyl-3-((2S)-2-hydroxy-3-(8-(4-methyl-3,4-dihydro-2H-benzo[b][1,4]oxazin-6-ylsulfonyl)-1-oxa-8-azaspiro[4.5]decan-3-ylamino)propoxy)benzenesulfonamide; Compound B235: 3-((2S)-3-(8-(7-chlorobenzo[c][1,2,5]oxadiazol-4-ylsulfonyl)-1-oxa-8-azaspiro[4.5]decan-3-ylamino)-2-hydroxypropoxy)-N-ethylbenzenesulfonamide; Compound B236: 3-((2S)-2-hydroxy-3-(8-(4-methoxy-2-methylphenylsulfonyl)-1-oxa-8-azaspiro[4.5]decan-3-ylamino)propoxy)-N-methylbenzenesulfonamide; Compound B237: 3-((2S)-3-(8-(5-bromo-2-methylphenylsulfonyl)-1-oxa-8-azaspiro[4.5]decan-3-ylamino)-2-hydroxypropoxy)-N-methylbenzenesulfonamide; Compound B238: 3-((2S)-3-(8-(5-fluoro-2-methoxyphenylsulfonyl)-1-oxa-8-azaspiro[4.5]decan-3-ylamino)-2-hydroxypropoxy)-N-methylbenzenesulfonamide; Compound B239: 3-((2S)-3-(8-(3-chloro-4-cyanophenylsulfonyl)-1-oxa-8-azaspiro[4.5]decan-3-ylamino)-2-hydroxypropoxy)-N-methylbenzenesulfonamide; Compound B240: 3-((2S)-3-(8-(5-chloro-2-methylphenylsulfonyl)-1-oxa-8-azaspiro[4.5]decan-3-ylamino)-2-hydroxypropoxy)-N-methylbenzenesulfonamide; Compound B241: 3-((2S)-3-(8-(2-fluoro-5-methoxyphenylsulfonyl)-1-oxa-8-azaspiro[4.5]decan-3-ylamino)-2-hydroxypropoxy)-N-methylbenzenesulfonamide; Compound B242: 3-((2S)-2-hydroxy-3-(8-(4-methoxy-2,3-dimethylphenylsulfonyl)-1-oxa-8-azaspiro[4.5]decan-3-ylamino)propoxy)-N-methylbenzenesulfonamide; Compound B243: 3-((2S)-2-hydroxy-3-(8-(1-phenyl-1H-pyrazol-4-ylsulfonyl)-1-oxa-8-azaspiro[4.5]decan-3-ylamino)propoxy)-N-methylbenzenesulfonamide; Compound B244: 3-((S)-3-((S)-8-(benzo[c][1,2,5]thiadiazol-4-ylsulfonyl)-1-oxa-8-azaspiro[4.5]decan-3-ylamino)-2-hydroxypropoxy)-N-methylbenzenesulfonamide; Compound B245: 3-((S)-3-((R)-8-(benzo[c][1,2,5]thiadiazol-4-ylsulfonyl)-1-oxa-8-azaspiro[4.5]decan-3-ylamino)-2-hydroxypropoxy)-N-methylbenzenesulfonamide; Compound B246: 3-((2S)-3-(8-(3-chlorophenylsulfonyl)-1-oxa-8-azaspiro[4.5]decan-3-ylamino)-2-hydroxypropoxy)-N,N-dimethylbenzenesulfonamide; Compound B247: 3-((2S)-2-hydroxy-3-(8-(4-methoxy-3-methylphenylsulfonyl)-1-oxa-8-azaspiro[4.5]decan-3-ylamino)propoxy)-N,N-dimethylbenzenesulfonamide; Compound B248: 3-((2S)-3-(8-(2-fluoro-5-methylphenylsulfonyl)-1-oxa-8-azaspiro[4.5]decan-3-ylamino)-2-hydroxypropoxy)-N,N-dimethylbenzenesulfonamide; Compound B249: 3-((2S)-3-(8-(4-bromo-3-methylphenylsulfonyl)-1-oxa-8-azaspiro[4.5]decan-3-ylamino)-2-hydroxypropoxy)-N,N-dimethylbenzenesulfonamide; Compound B250: 3-((2S)-2-hydroxy-3-(8-(3-oxo-3,4-dihydro-2H-benzo[b][1,4]oxazin-6-ylsulfonyl)-1-oxa-8-azaspiro[4.5]decan-3-ylamino)propoxy)-N,N-dimethylbenzenesulfonamide; Compound B251: 3-((2S)-3-(8-(benzo[c][1,2,5]thiadiazol-5-ylsulfonyl)-1-oxa-8-azaspiro[4.5]decan-3-ylamino)-2-hydroxypropoxy)-N,N-dimethylbenzenesulfonamide; Compound B252: 3-((2S)-2-hydroxy-3-(8-(5-methyl-1-phenyl-1H-pyrazol-4-ylsulfonyl)-1-oxa-8-azaspiro[4.5]decan-3-ylamino)propoxy)-N,N-dimethylbenzenesulfonamide; Compound B253: 3-((2S)-2-hydroxy-3-(8-(5,6,7,8-tetrahydronaphthalen-2-ylsulfonyl)-1-oxa-8-azaspiro[4.5]decan-3-ylamino)propoxy)-N,N-dimethylbenzenesulfonamide; Compound B254: 3-((2S)-3-(8-(3,5-dimethylisoxazol-4-ylsulfonyl)-1-oxa-8-azaspiro[4.5]decan-3-ylamino)-2-hydroxypropoxy)-N,N-dimethylbenzenesulfonamide; Compound B255: 3-((2S)-2-hydroxy-3-(8-(5-phenylthiophen-2-ylsulfonyl)-1-oxa-8-azaspiro[4.5]decan-3-ylamino)propoxy)-N,N-dimethylbenzenesulfonamide; Compound B256: 3-((2S)-2-hydroxy-3-(8-(4-methyl-3,4-dihydro-2H-benzo[b][1,4]oxazin-6-ylsulfonyl)-1-oxa-8-azaspiro[4.5]decan-3-ylamino)propoxy)-N,N-dimethylbenzenesulfonamide; Compound B257: 3-((2S)-3-(8-(7-chlorobenzo[c][1,2,5]oxadiazol-4-ylsulfonyl)-1-oxa-8-azaspiro[4.5]decan-3-ylamino)-2-hydroxypropoxy)-N,N-dimethylbenzenesulfonamide; Compound B258: 3-((2S)-2-hydroxy-3-(8-(4-methoxynaphthalen-1-ylsulfonyl)-1-oxa-8-azaspiro[4.5]decan-3-ylamino)propoxy)-N-methylbenzenesulfonamide; Compound B259: 3-((2S)-3-(8-(5-bromo-2-chlorophenylsulfonyl)-1-oxa-8-azaspiro[4.5]decan-3-ylamino)-2-hydroxypropoxy)-N-methylbenzenesulfonamide; Compound B260: 3-((2S)-3-(8-(5-bromo-2,3-dihydrobenzofuran-7-ylsulfonyl)-1-oxa-8-azaspiro[4.5]decan-3-ylamino)-2-hydroxypropoxy)-N-methylbenzenesulfonamide; Compound B261: 3-((2S)-3-(8-(3,5-dimethyl-1-phenyl-1H-pyrazol-4-ylsulfonyl)-1-oxa-8-azaspiro[4.5]decan-3-ylamino)-2-hydroxypropoxy)-N-methylbenzenesulfonamide; Compound B262: 3-((2S)-3-(8-(5-chlorobenzo[c][1,2,5]oxadiazol-4-ylsulfonyl)-1-oxa-8-azaspiro[4.5]decan-3-ylamino)-2-hydroxypropoxy)-N-methylbenzenesulfonamide; Compound B263: 3-((2S)-3-(8-(2-cyano-5-methoxyphenylsulfonyl)-1-oxa-8-azaspiro[4.5]decan-3-ylamino)-2-hydroxypropoxy)-N-methylbenzenesulfonamide; Compound B264: 3-((2S)-3-(8-(6-chloronaphthalen-2-ylsulfonyl)-1-oxa-8-azaspiro[4.5]decan-3-ylamino)-2-hydroxypropoxy)-N-methylbenzenesulfonamide; Compound B265: 3-((2S)-3-(8-(2,3-dimethylphenylsulfonyl)-1-oxa-8-azaspiro[4.5]decan-3-ylamino)-2-hydroxypropoxy)-N-methylbenzenesulfonamide; Compound B266: 3-((S)-2-hydroxy-3-((S)-8-(4-methoxy-3-methylphenylsulfonyl)-1-oxa-8-azaspiro[4.5]decan-3-ylamino)propoxy)-N-methylbenzenesulfonamide; Compound B267: 3-((S)-2-hydroxy-3-((R)-8-(4-methoxy-3-methylphenylsulfonyl)-1-oxa-8-azaspiro[4.5]decan-3-ylamino)propoxy)-N-methylbenzenesulfonamide; Compound B268: 3-((S)-3-((S)-8-(2-fluoro-5-methylphenylsulfonyl)-1-oxa-8-azaspiro[4.5]decan-3-ylamino)-2-hydroxypropoxy)-N-methylbenzenesulfonamide; Compound B269: 3-((S)-3-((R)-8-(2-fluoro-5-methylphenylsulfonyl)-1-oxa-8-azaspiro[4.5]decan-3-ylamino)-2-hydroxypropoxy)-N-methylbenzenesulfonamide; Compound B270: 3-((S)-3-((S)-8-(4-bromo-3-methylphenylsulfonyl)-1-oxa-8-azaspiro[4.5]decan-3-ylamino)-2-hydroxypropoxy)-N-methylbenzenesulfonamide; Compound B271: 3-((S)-3-((R)-8-(4-bromo-3-methylphenylsulfonyl)-1-oxa-8-azaspiro[4.5]decan-3-ylamino)-2-hydroxypropoxy)-N-methylbenzenesulfonamide; Compound B272: 3-((2S)-3-(8-(3-(1-ethyl-1H-pyrazol-4-yl)phenylsulfonyl)-1-oxa-8-azaspiro[4.5]decan-3-ylamino)-2-hydroxypropoxy)-N,N-dimethylbenzenesulfonamide; Compound B273: 3-((2S)-3-(8-(4′-(aminomethyl)biphenyl-3-ylsulfonyl)-1-oxa-8-azaspiro[4.5]decan-3-ylamino)-2-hydroxypropoxy)-N,N-dimethylbenzenesulfonamide; Compound B274: 3′-(3-((S)-3-(3-(N,N-dimethylsulfamoyl)phenoxy)-2-hydroxypropylamino)-1-oxa-8-azaspiro[4.5]decan-8-ylsulfonyl)biphenyl-4-sulfonamide; Compound B275: 3-((2S)-3-(8-(3-(6-aminopyridin-3-yl)phenylsulfonyl)-1-oxa-8-azaspiro[4.5]decan-3-ylamino)-2-hydroxypropoxy)-N,N-dimethylbenzenesulfonamide; Compound B276: 3-((2S)-3-(8-(4′-(cyanomethoxy)biphenyl-3-ylsulfonyl)-1-oxa-8-azaspiro[4.5]decan-3-ylamino)-2-hydroxypropoxy)-N,N-dimethylbenzenesulfonamide; Compound B277: 3-((2S)-3-(8-(4′-(cyanomethyl)biphenyl-3-ylsulfonyl)-1-oxa-8-azaspiro[4.5]decan-3-ylamino)-2-hydroxypropoxy)-N,N-dimethylbenzenesulfonamide; Compound B278: 3-((S)-2-hydroxy-3-((S)-8-(3-oxo-3,4-dihydro-2H-benzo[b][1,4]oxazin-6-ylsulfonyl)-1-oxa-8-azaspiro[4.5]decan-3-ylamino)propoxy)-N-methylbenzenesulfonamide; Compound B279: 3-((S)-2-hydroxy-3-((R)-8-(3-oxo-3,4-dihydro-2H-benzo[b][1,4]oxazin-6-ylsulfonyl)-1-oxa-8-azaspiro[4.5]decan-3-ylamino)propoxy)-N-methylbenzenesulfonamide; Compound B280: 3-((S)-2-hydroxy-3-((S)-8-(5-methyl-1-phenyl-1H-pyrazol-4-ylsulfonyl)-1-oxa-8-azaspiro[4.5]decan-3-ylamino)propoxy)-N-methylbenzenesulfonamide; Compound B281: 3-((S)-2-hydroxy-3-((R)-8-(5-methyl-1-phenyl-1H-pyrazol-4-ylsulfonyl)-1-oxa-8-azaspiro[4.5]decan-3-ylamino)propoxy)-N-methylbenzenesulfonamide; Compound B282: 3-((S)-3-((S)-8-(3-(1-ethyl-1H-pyrazol-4-yl)phenylsulfonyl)-1-oxa-8-azaspiro[4.5]decan-3-ylamino)-2-hydroxypropoxy)-N-methylbenzenesulfonamide; Compound B283: 3-((S)-3-((R)-8-(3-(1-ethyl-1H-pyrazol-4-yl)phenylsulfonyl)-1-oxa-8-azaspiro[4.5]decan-3-ylamino)-2-hydroxypropoxy)-N-methylbenzenesulfonamide; Compound B284: 3-((S)-2-hydroxy-3-((S)-8-(3-(pyridin-3-yl)phenylsulfonyl)-1-oxa-8-azaspiro[4.5]decan-3-ylamino)propoxy)-N-methylbenzenesulfonamide; Compound B285: 3-((S)-2-hydroxy-3-((R)-8-(3-(pyridin-3-yl)phenylsulfonyl)-1-oxa-8-azaspiro[4.5]decan-3-ylamino)propoxy)-N-methylbenzenesulfonamide; Compound B286: 3-((S)-3-((S)-8-(3-(6-aminopyridin-3-yl)phenylsulfonyl)-1-oxa-8-azaspiro[4.5]decan-3-ylamino)-2-hydroxypropoxy)-N-methylbenzenesulfonamide; Compound B287: 3-((S)-3-((R)-8-(3-(6-aminopyridin-3-yl)phenylsulfonyl)-1-oxa-8-azaspiro[4.5]decan-3-ylamino)-2-hydroxypropoxy)-N-methylbenzenesulfonamide; Compound B288: 3-((S)-3-((S)-8-(4′-(aminomethyl)biphenyl-3-ylsulfonyl)-1-oxa-8-azaspiro[4.5]decan-3-ylamino)-2-hydroxypropoxy)-N-methylbenzenesulfonamide; Compound B289: 3-((S)-3-((R)-8-(4′-(aminomethyl)biphenyl-3-ylsulfonyl)-1-oxa-8-azaspiro[4.5]decan-3-ylamino)-2-hydroxypropoxy)-N-methylbenzenesulfonamide; Compound B290: 3-((2S)-3-(8-(4-(aminomethyl)phenylsulfonyl)-1-oxa-8-azaspiro[4.5]decan-3-ylamino)-2-hydroxypropoxy)-N,N-dimethylbenzenesulfonamide; Compound B291: 3′-((S)-3-((S)-2-hydroxy-3-(3-(N-methylsulfamoyl)phenoxy)propylamino)-1-oxa-8-azaspiro[4.5]decan-8-ylsulfonyl)biphenyl-4-sulfonamide; Compound B292: 3′-((R)-3-((S)-2-hydroxy-3-(3-(N-methylsulfamoyl)phenoxy)propylamino)-1-oxa-8-azaspiro[4.5]decan-8-ylsulfonyl)biphenyl-4-sulfonamide; Compound B293: 3-((S)-3-((S)-8-(4′-(cyanomethyl)biphenyl-3-ylsulfonyl)-1-oxa-8-azaspiro[4.5]decan-3-ylamino)-2-hydroxypropoxy)-N-methylbenzenesulfonamide; Compound B294: 3-((S)-3-((R)-8-(4′-(cyanomethyl)biphenyl-3-ylsulfonyl)-1-oxa-8-azaspiro[4.5]decan-3-ylamino)-2-hydroxypropoxy)-N-methylbenzenesulfonamide; Compound B295: 3-((S)-3-((S)-8-(3-(1H-pyrazol-3-yl)phenylsulfonyl)-1-oxa-8-azaspiro[4.5]decan-3-ylamino)-2-hydroxypropoxy)-N-methylbenzenesulfonamide; Compound B296: 3-((S)-3-((R)-8-(3-(1H-pyrazol-3-yl)phenylsulfonyl)-1-oxa-8-azaspiro[4.5]decan-3-ylamino)-2-hydroxypropoxy)-N-methylbenzenesulfonamide; Compound B297: 3-((S)-3-((S)-8-(4′-(cyanomethoxy)biphenyl-3-ylsulfonyl)-1-oxa-8-azaspiro[4.5]decan-3-ylamino)-2-hydroxypropoxy)-N-methylbenzenesulfonamide; Compound B298: 3-((S)-3-((R)-8-(4′-(cyanomethoxy)biphenyl-3-ylsulfonyl)-1-oxa-8-azaspiro[4.5]decan-3-ylamino)-2-hydroxypropoxy)-N-methylbenzenesulfonamide; Compound B299: 3-((S)-2-hydroxy-3-((S)-8-(4-methyl-3,4-dihydro-2H-benzo[b][1,4]oxazin-6-ylsulfonyl)-1-oxa-8-azaspiro[4.5]decan-3-ylamino)propoxy)-N-methylbenzenesulfonamide; Compound B300: 3-((S)-2-hydroxy-3-((R)-8-(4-methyl-3,4-dihydro-2H-benzo[b][1,4]oxazin-6-ylsulfonyl)-1-oxa-8-azaspiro[4.5]decan-3-ylamino)propoxy)-N-methylbenzenesulfonamide; Compound B301: 3-((2S)-2-hydroxy-3-(8-(4′-((methylamino)methyl)biphenyl-3-ylsulfonyl)-1-oxa-8-azaspiro[4.5]decan-3-ylamino)propoxy)-N-methylbenzenesulfonamide; Compound B302: 3-((2S)-3-(8-(4′-((ethylamino)methyl)biphenyl-3-ylsulfonyl)-1-oxa-8-azaspiro[4.5]decan-3-ylamino)-2-hydroxypropoxy)-N-methylbenzenesulfonamide; Compound B303: 3-((2S)-2-hydroxy-3-(8-(4′-((isopropylamino)methyl)biphenyl-3-ylsulfonyl)-1-oxa-8-azaspiro[4.5]decan-3-ylamino)propoxy)-N-methylbenzenesulfonamide; Compound B304: 3-((2S)-2-hydroxy-3-(8-(4′-((2-hydroxyethylamino)methyl)biphenyl-3-ylsulfonyl)-1-oxa-8-azaspiro[4.5]decan-3-ylamino)propoxy)-N-methylbenzenesulfonamide; Compound B305: 3-((2S)-3-(8-(4′-((2-(dimethylamino)ethylamino)methyl)biphenyl-3-ylsulfonyl)-1-oxa-8-azaspiro[4.5]decan-3-ylamino)-2-hydroxypropoxy)-N-methylbenzenesulfonamide; Compound B306: 3-((2S)-2-hydroxy-3-(8-(naphthalen-2-ylsulfonyl)-1-oxa-8-azaspiro[4.5]decan-3-ylamino)propoxy)benzenesulfonamide; Compound B307: 3-((2S)-3-(8-(3-chlorophenylsulfonyl)-1-oxa-8-azaspiro[4.5]decan-3-ylamino)-2-hydroxypropoxy)-N-(3-(dimethylamino)propyl)benzenesulfonamide; Compound B308: N-(3-(dimethylamino)propyl)-3-((2S)-2-hydroxy-3-(8-(4-methoxy-3-methylphenylsulfonyl)-1-oxa-8-azaspiro[4.5]decan-3-ylamino)propoxy)benzenesulfonamide; Compound B309: N-(3-(dimethylamino)propyl)-3-((2S)-3-(8-(2-fluoro-5-methylphenylsulfonyl)-1-oxa-8-azaspiro[4.5]decan-3-ylamino)-2-hydroxypropoxy)benzenesulfonamide; Compound B310: 3-((2S)-3-(8-(4-bromo-3-methylphenylsulfonyl)-1-oxa-8-azaspiro[4.5]decan-3-ylamino)-2-hydroxypropoxy)-N-(3-(dimethylamino)propyl)benzenesulfonamide; Compound B311: N-(3-(dimethylamino)propyl)-3-((2S)-2-hydroxy-3-(8-(3-oxo-3,4-dihydro-2H-benzo[b][1,4]oxazin-6-ylsulfonyl)-1-oxa-8-azaspiro[4.5]decan-3-ylamino)propoxy)benzenesulfonamide; Compound B312: 3-((2S)-3-(8-(benzo[c][1,2,5]thiadiazol-5-ylsulfonyl)-1-oxa-8-azaspiro[4.5]decan-3-ylamino)-2-hydroxypropoxy)-N-(3-(dimethylamino)propyl)benzenesulfonamide; Compound B313: N-(3-(dimethylamino)propyl)-3-((2S)-2-hydroxy-3-(8-(5-methyl-1-phenyl-1H-pyrazol-4-ylsulfonyl)-1-oxa-8-azaspiro[4.5]decan-3-ylamino)propoxy)benzenesulfonamide; Compound B314: N-(3-(dimethylamino)propyl)-3-((2S)-2-hydroxy-3-(8-(5,6,7,8-tetrahydronaphthalen-2-ylsulfonyl)-1-oxa-8-azaspiro[4.5]decan-3-ylamino)propoxy)benzenesulfonamide; Compound B315: N-(3-(dimethylamino)propyl)-3-((2S)-2-hydroxy-3-(8-(5-phenylthiophen-2-ylsulfonyl)-1-oxa-8-azaspiro[4.5]decan-3-ylamino)propoxy)benzenesulfonamide; Compound B316: N-(3-(dimethylamino)propyl)-3-((2S)-2-hydroxy-3-(8-(4-methyl-3,4-dihydro-2H-benzo[b][1,4]oxazin-6-ylsulfonyl)-1-oxa-8-azaspiro[4.5]decan-3-ylamino)propoxy)benzenesulfonamide; Compound B317: N-(3-(dimethylamino)propyl)-3-((2S)-2-hydroxy-3-(8-(naphthalen-2-ylsulfonyl)-1-oxa-8-azaspiro[4.5]decan-3-ylamino)propoxy)benzenesulfonamide; Compound B318: N-(3-((2S)-2-hydroxy-3-(8-(naphthalen-2-ylsulfonyl)-1-oxa-8-azaspiro[4.5]decan-3-ylamino)propoxy)phenylsulfonyl)acetamide; Compound B319: 3-((2S)-2-hydroxy-3-(8-(4′-((isobutylamino)methyl)biphenyl-3-ylsulfonyl)-1-oxa-8-azaspiro[4.5]decan-3-ylamino)propoxy)-N-methylbenzenesulfonamide; Compound B320: 3-((2S)-2-hydroxy-3-(8-(4′-((isopentylamino)methyl)biphenyl-3-ylsulfonyl)-1-oxa-8-azaspiro[4.5]decan-3-ylamino)propoxy)-N-methylbenzenesulfonamide; Compound B321: 3-((2S)-2-hydroxy-3-(8-(4′-((2,2,2-trifluoroethylamino)methyl)biphenyl-3-ylsulfonyl)-1-oxa-8-azaspiro[4.5]decan-3-ylamino)propoxy)-N-methylbenzenesulfonamide; Compound B322: 3-((2S)-2-hydroxy-3-(8-(pyridin-2-ylsulfonyl)-1-oxa-8-azaspiro[4.5]decan-3-ylamino)propoxy)-N-methylbenzenesulfonamide; Compound B323: 3-((2S)-2-hydroxy-3-(8-(quinolin-6-ylsulfonyl)-1-oxa-8-azaspiro[4.5]decan-3-ylamino)propoxy)-N-methylbenzenesulfonamide; Compound B324: 3-((2S)-2-hydroxy-3-(8-(pyridin-3-ylsulfonyl)-1-oxa-8-azaspiro[4.5]decan-3-ylamino)propoxy)-N-methylbenzenesulfonamide; Compound B325: 3-((2S)-3-(8-(1H-indazol-5-ylsulfonyl)-1-oxa-8-azaspiro[4.5]decan-3-ylamino)-2-hydroxypropoxy)-N-methylbenzenesulfonamide; Compound B326: 3-((2S)-3-(8-(benzofuran-2-ylsulfonyl)-1-oxa-8-azaspiro[4.5]decan-3-ylamino)-2-hydroxypropoxy)-N-methylbenzenesulfonamide; Compound B327: 3-((2S)-3-(8-(benzo[d]isoxazol-5-ylsulfonyl)-1-oxa-8-azaspiro[4.5]decan-3-ylamino)-2-hydroxypropoxy)-N-methylbenzenesulfonamide; Compound B328: 3-((2S)-3-(8-(1H-indazol-6-ylsulfonyl)-1-oxa-8-azaspiro[4.5]decan-3-ylamino)-2-hydroxypropoxy)-N-methylbenzenesulfonamide; Compound B329: 3-((2S)-3-(8-(1-ethyl-5-methyl-1H-pyrazol-4-ylsulfonyl)-1-oxa-8-azaspiro[4.5]decan-3-ylamino)-2-hydroxypropoxy)-N-methylbenzenesulfonamide; Compound B330: 3-((2S)-3-(8-(2,3-dihydrobenzofuran-5-ylsulfonyl)-1-oxa-8-azaspiro[4.5]decan-3-ylamino)-2-hydroxypropoxy)-N-methylbenzenesulfonamide; Compound B331: 3-((2S)-3-(8-(benzofuran-5-ylsulfonyl)-1-oxa-8-azaspiro[4.5]decan-3-ylamino)-2-hydroxypropoxy)-N-methylbenzenesulfonamide; Compound B332: 3-((2S)-3-(8-(2,3-dihydrobenzo[b][1,4]dioxin-6-ylsulfonyl)-1-oxa-8-azaspiro[4.5]decan-3-ylamino)-2-hydroxypropoxy)-N-methylbenzenesulfonamide; Compound B333: 3-((2S)-3-(8-(4-ethyl-3,4-dihydro-2H-benzo[b][1,4]oxazin-6-ylsulfonyl)-1-oxa-8-azaspiro[4.5]decan-3-ylamino)-2-hydroxypropoxy)-N-methylbenzenesulfonamide; Compound B334: 3-((2S)-2-hydroxy-3-(8-(4-isopropyl-3,4-dihydro-2H-benzo[b][1,4]oxazin-6-ylsulfonyl)-1-oxa-8-azaspiro[4.5]decan-3-ylamino)propoxy)-N-methylbenzenesulfonamide; Compound B335: 3-((S)-2-hydroxy-3-((S)-8-(quinolin-3-ylsulfonyl)-1-oxa-8-azaspiro[4.5]decan-3-ylamino)propoxy)-N-methylbenzenesulfonamide; Compound B336: 3-((S)-2-hydroxy-3-((R)-8-(quinolin-3-ylsulfonyl)-1-oxa-8-azaspiro[4.5]decan-3-ylamino)propoxy)-N-methylbenzenesulfonamide; Compound B337: 3-((2S)-3-(8-(5-bromopyridin-3-ylsulfonyl)-1-oxa-8-azaspiro[4.5]decan-3-ylamino)-2-hydroxypropoxy)-N-methylbenzenesulfonamide; Compound B338: 3-((2S)-3-(8-(4′-((cyanomethylamino)methyl)biphenyl-3-ylsulfonyl)-1-oxa-8-azaspiro[4.5]decan-3-ylamino)-2-hydroxypropoxy)-N-methylbenzenesulfonamide; Compound B339: 3-((2S)-3-(8-(4′-((2,2-difluoroethylamino)methyl)biphenyl-3-ylsulfonyl)-1-oxa-8-azaspiro[4.5]decan-3-ylamino)-2-hydroxypropoxy)-N-methylbenzenesulfonamide; Compound B340: 3-((2S)-2-hydroxy-3-(8-(4′-((2-methoxyethylamino)methyl)biphenyl-3-ylsulfonyl)-1-oxa-8-azaspiro[4.5]decan-3-ylamino)propoxy)-N-methylbenzenesulfonamide; Compound B341: 3-((2S)-3-(8-(5-(4-(aminomethyl)phenyl)pyridin-3-ylsulfonyl)-1-oxa-8-azaspiro[4.5]decan-3-ylamino)-2-hydroxypropoxy)-N-methylbenzenesulfonamide; Compound B342: 3-((2S)-2-hydroxy-3-(8-(5-(1-methyl-1H-pyrazol-4-yl)pyridin-3-ylsulfonyl)-1-oxa-8-azaspiro[4.5]decan-3-ylamino)propoxy)-N-methylbenzenesulfonamide; Compound B343: 3-((S)-2-hydroxy-3-((S)-8-(quinolin-6-ylsulfonyl)-1-oxa-8-azaspiro[4.5]decan-3-ylamino)propoxy)-N-methylbenzenesulfonamide; Compound B344: 3-((S)-2-hydroxy-3-((R)-8-(quinolin-6-ylsulfonyl)-1-oxa-8-azaspiro[4.5]decan-3-ylamino)propoxy)-N-methylbenzenesulfonamide; Compound B345: (2S)-3-hydroxy-2-((3′-(3-((S)-2-hydroxy-3-(3-(N-methylsulfamoyl)phenoxy)propylamino)-1-oxa-8-azaspiro[4.5]decan-8-ylsulfonyl)biphenyl-4-yl)methylamino)propanamide; Compound B346: 3-((2S)-2-hydroxy-3-(8-(4′-(thiazolidin-3-ylmethyl)biphenyl-3-ylsulfonyl)-1-oxa-8-azaspiro[4.5]decan-3-ylamino)propoxy)-N-methylbenzenesulfonamide; Compound B347: 3-((2S)-3-(8-(4′-((2-fluoroethylamino)methyl)biphenyl-3-ylsulfonyl)-1-oxa-8-azaspiro[4.5]decan-3-ylamino)-2-hydroxypropoxy)-N-methylbenzenesulfonamide; Compound B348: N-(2-((3′-(3-((S)-2-hydroxy-3-(3-(N-methylsulfamoyl)phenoxy)propylamino)-1-oxa-8-azaspiro[4.5]decan-8-ylsulfonyl)biphenyl-4-yl)methylamino)ethyl)acetamide; Compound B349: 3-((2S)-2-hydroxy-3-(8-(4-isobutyl-3,4-dihydro-2H-benzo[b][1,4]oxazin-6-ylsulfonyl)-1-oxa-8-azaspiro[4.5]decan-3-ylamino)propoxy)-N-methylbenzenesulfonamide; Compound B350: 3-((S)-3-((S)-8-(4-ethyl-3,4-dihydro-2H-benzo[b][1,4]oxazin-6-ylsulfonyl)-1-oxa-8-azaspiro[4.5]decan-3-ylamino)-2-hydroxypropoxy)-N-methylbenzenesulfonamide; Compound B351: 3-((S)-3-((R)-8-(4-ethyl-3,4-dihydro-2H-benzo[b][1,4]oxazin-6-ylsulfonyl)-1-oxa-8-azaspiro[4.5]decan-3-ylamino)-2-hydroxypropoxy)-N-methylbenzenesulfonamide; Compound B352: 3-((3′-(3-((S)-2-hydroxy-3-(3-(N-methylsulfamoyl)phenoxy)propylamino)-1-oxa-8-azaspiro[4.5]decan-8-ylsulfonyl)biphenyl-4-yl)methylamino)-3-oxopropanoic acid; Compound B353: 2-amino-N-((3′-(3-((S)-2-hydroxy-3-(3-(N-methylsulfamoyl)phenoxy)propylamino)-1-oxa-8-azaspiro[4.5]decan-8-ylsulfonyl)biphenyl-4-yl)methyl)acetamide; Compound B354: N¹-((3′-(3-((S)-2-hydroxy-3-(3-(N-methylsulfamoyl)phenoxy)propylamino)-1-oxa-8-azaspiro[4.5]decan-8-ylsulfonyl)biphenyl-4-yl)methyl)oxalamide; Compound B355: 3-((2S)-3-(8-(3,4-dihydro-2H-benzo[b][1,4]oxazin-6-ylsulfonyl)-1-oxa-8-azaspiro[4.5]decan-3-ylamino)-2-hydroxypropoxy)-N-methylbenzenesulfonamide; Compound B356: N-(2-fluoroethyl)-3-((2S)-2-hydroxy-3-(8-(naphthalen-2-ylsulfonyl)-1-oxa-8-azaspiro[4.5]decan-3-ylamino)propoxy)benzenesulfonamide; Compound B357: 3-((2S)-3-(8-(4′-(aminomethyl)-4-methoxybiphenyl-3-ylsulfonyl)-1-oxa-8-azaspiro[4.5]decan-3-ylamino)-2-hydroxypropoxy)-N-methylbenzenesulfonamide; Compound B358: 3-((2S)-3-(8-(5-(4-(aminomethyl)phenyl)-2,3-dihydrobenzofuran-7-ylsulfonyl)-1-oxa-8-azaspiro[4.5]decan-3-ylamino)-2-hydroxypropoxy)-N-methylbenzenesulfonamide; Compound B359: 3-((S)-2-hydroxy-3-((S)-8-(4′-((isopropylamino)methyl)biphenyl-3-ylsulfonyl)-1-oxa-8-azaspiro[4.5]decan-3-ylamino)propoxy)-N-methylbenzenesulfonamide; Compound B360: 3-((S)-2-hydroxy-3-((S)-8-(4′-((isobutylamino)methyl)biphenyl-3-ylsulfonyl)-1-oxa-8-azaspiro[4.5]decan-3-ylamino)propoxy)-N-methylbenzenesulfonamide; Compound B361: 3-((S)-2-hydroxy-3-((S)-8-(4′-((isopentylamino)methyl)biphenyl-3-ylsulfonyl)-1-oxa-8-azaspiro[4.5]decan-3-ylamino)propoxy)-N-methylbenzenesulfonamide; Compound B362: 3-((S)-2-hydroxy-3-((R)-8-(4′-((isopropylamino)methyl)biphenyl-3-ylsulfonyl)-1-oxa-8-azaspiro[4.5]decan-3-ylamino)propoxy)-N-methylbenzenesulfonamide; Compound B363: 3-((S)-2-hydroxy-3-((R)-8-(4′-((isobutylamino)methyl)biphenyl-3-ylsulfonyl)-1-oxa-8-azaspiro[4.5]decan-3-ylamino)propoxy)-N-methylbenzenesulfonamide; Compound B364: 3-((S)-2-hydroxy-3-((R)-8-(4′-((isopentylamino)methyl)biphenyl-3-ylsulfonyl)-1-oxa-8-azaspiro[4.5]decan-3-ylamino)propoxy)-N-methylbenzenesulfonamide; Compound B365: 3-((2S)-3-(8-(4′-(aminomethyl)-4-methylbiphenyl-3-ylsulfonyl)-1-oxa-8-azaspiro[4.5]decan-3-ylamino)-2-hydroxypropoxy)-N-methylbenzenesulfonamide; Compound B366: 3-((2S)-3-(8-(4′-(aminomethyl)-6-methoxybiphenyl-3-ylsulfonyl)-1-oxa-8-azaspiro[4.5]decan-3-ylamino)-2-hydroxypropoxy)-N-methylbenzenesulfonamide; Compound B367: 3-((2S)-3-(8-(4′-(aminomethyl)-5-(trifluoromethyl)biphenyl-3-ylsulfonyl)-1-oxa-8-azaspiro[4.5]decan-3-ylamino)-2-hydroxypropoxy)-N-methylbenzenesulfonamide; Compound B368: 3′-(3-((S)-2-hydroxy-3-(3-(N-methylsulfamoyl)phenoxy)propylamino)-1-oxa-8-azaspiro[4.5]decan-8-ylsulfonyl)biphenyl-4-carboxamide; Compound B369: 3-((2S)-3-(8-(3-(5-(aminomethyl)thiophen-2-yl)phenylsulfonyl)-1-oxa-8-azaspiro[4.5]decan-3-ylamino)-2-hydroxypropoxy)-N-methylbenzenesulfonamide; Compound B370: 3-((2S)-3-(8-(3-(5-cyanopyridin-3-yl)phenylsulfonyl)-1-oxa-8-azaspiro[4.5]decan-3-ylamino)-2-hydroxypropoxy)-N-methylbenzenesulfonamide; Compound B371: 3-((2S)-2-hydroxy-3-(8-(3-(6-(2-morpholinoethylamino)pyridin-3-yl)phenylsulfonyl)-1-oxa-8-azaspiro[4.5]decan-3-ylamino)propoxy)-N-methylbenzenesulfonamide; Compound B372: 3-((2S)-3-(8-(3-(6-(3-(dimethylamino)propoxy)pyridin-3-yl)phenylsulfonyl)-1-oxa-8-azaspiro[4.5]decan-3-ylamino)-2-hydroxypropoxy)-N-methylbenzenesulfonamide; Compound B373: 3-((2S)-3-(8-(4′-(aminomethyl)-5-methylbiphenyl-3-ylsulfonyl)-1-oxa-8-azaspiro[4.5]decan-3-ylamino)-2-hydroxypropoxy)-N-methylbenzenesulfonamide; Compound B374: 3-((S)-3-((S)-8-(5-(4-(aminomethyl)phenyl)pyridin-3-ylsulfonyl)-1-oxa-8-azaspiro[4.5]decan-3-ylamino)-2-hydroxypropoxy)-N-methylbenzenesulfonamide; Compound B375: 3-((S)-3-((R)-8-(5-(4-(aminomethyl)phenyl)pyridin-3-ylsulfonyl)-1-oxa-8-azaspiro[4.5]decan-3-ylamino)-2-hydroxypropoxy)-N-methylbenzenesulfonamide; Compound B376: 3-((2S)-3-(8-(4′-(aminomethyl)-6-methylbiphenyl-3-ylsulfonyl)-1-oxa-8-azaspiro[4.5]decan-3-ylamino)-2-hydroxypropoxy)-N-methylbenzenesulfonamide; Compound B377: 3-((2S)-3-(8-(4′-(aminomethyl)-4-chlorobiphenyl-3-ylsulfonyl)-1-oxa-8-azaspiro[4.5]decan-3-ylamino)-2-hydroxypropoxy)-N-methylbenzenesulfonamide; Compound B378: 3-((2S)-2-hydroxy-3-(8-(2-(pyridin-4-yl)ethylsulfonyl)-1-oxa-8-azaspiro[4.5]decan-3-ylamino)propoxy)-N-methylbenzenesulfonamide; Compound B379: (Z)-N′-hydroxy-3′-(3-((S)-2-hydroxy-3-(3-(N-methylsulfamoyl)phenoxy)propylamino)-1-oxa-8-azaspiro[4.5]decan-8-ylsulfonyl)biphenyl-4-carboximidamide; Compound B380: 3-((2S)-2-hydroxy-3-(8-(4′-(5-methyl-1,2,4-oxadiazol-3-yl)biphenyl-3-ylsulfonyl)-1-oxa-8-azaspiro[4.5]decan-3-ylamino)propoxy)-N-methylbenzenesulfonamide; Compound B381: 3-((2S)-3-(8-(4′-(aminomethyl)biphenyl-2-ylsulfonyl)-1-oxa-8-azaspiro[4.5]decan-3-ylamino)-2-hydroxypropoxy)-N-methylbenzenesulfonamide; Compound B382: 3-((2S)-3-(8-(4′-(aminomethyl)biphenyl-4-ylsulfonyl)-1-oxa-8-azaspiro[4.5]decan-3-ylamino)-2-hydroxypropoxy)-N-methylbenzenesulfonamide; Compound B383: 3′-(3-((S)-2-hydroxy-3-(3-(N-methylsulfamoyl)phenoxy)propylamino)-1-oxa-8-azaspiro[4.5]decan-8-ylsulfonyl)biphenyl-4-carboximidamide; Compound B384: 3-((S)-3-((S)-8-(4′-(aminomethyl)-4-methoxybiphenyl-3-ylsulfonyl)-1-oxa-8-azaspiro[4.5]decan-3-ylamino)-2-hydroxypropoxy)-N-methylbenzenesulfonamide; Compound B385: 3-((S)-3-((R)-8-(4′-(aminomethyl)-4-methoxybiphenyl-3-ylsulfonyl)-1-oxa-8-azaspiro[4.5]decan-3-ylamino)-2-hydroxypropoxy)-N-methylbenzenesulfonamide; Compound B386: 3-((2S)-2-hydroxy-3-(8-(5-methoxypyridin-3-ylsulfonyl)-1-oxa-8-azaspiro[4.5]decan-3-ylamino)propoxy)-N-methylbenzenesulfonamide; Compound B387: 3-((2S)-3-(8-(3-(6-(aminomethyl)pyridin-3-yl)phenylsulfonyl)-1-oxa-8-azaspiro[4.5]decan-3-ylamino)-2-hydroxypropoxy)-N-methylbenzenesulfonamide; Compound B388: 3-((2S)-3-(8-(6′-(aminomethyl)-3,3′-bipyridin-5-ylsulfonyl)-1-oxa-8-azaspiro[4.5]decan-3-ylamino)-2-hydroxypropoxy)-N-methylbenzenesulfonamide; Compound B389: 3-((2S)-3-(8-(3-(aminomethyl)phenylsulfonyl)-1-oxa-8-azaspiro[4.5]decan-3-ylamino)-2-hydroxypropoxy)-N-methylbenzenesulfonamide; Compound B390: 3-((2S)-2-hydroxy-3-(8-(3-(hydroxymethyl)phenylsulfonyl)-1-oxa-8-azaspiro[4.5]decan-3-ylamino)propoxy)-N-methylbenzenesulfonamide; Compound B391: 3-((2S)-3-(8-(4-(aminomethyl)phenylsulfonyl)-1-oxa-8-azaspiro[4.5]decan-3-ylamino)-2-hydroxypropoxy)-N-methylbenzenesulfonamide; Compound B 392: 3-((2S)-2-hydroxy-3-(8-(4-(hydroxymethyl)phenylsulfonyl)-1-oxa-8-azaspiro[4.5]decan-3-ylamino)propoxy)-N-methylbenzenesulfonamide; Compound B393: N-(2-fluoroethyl)-3-((S)-2-hydroxy-3-((R)-8-(quinolin-6-ylsulfonyl)-1-oxa-8-azaspiro[4.5]decan-3-ylamino)propoxy)benzenesulfonamide; Compound B394: N-(2-fluoroethyl)-3-((S)-2-hydroxy-3-((R)-8-(quinolin-3-ylsulfonyl)-1-oxa-8-azaspiro[4.5]decan-3-ylamino)propoxy)benzenesulfonamide; Compound B395: 3-((2S)-2-hydroxy-3-(8-(6-hydroxypyridin-3-ylsulfonyl)-1-oxa-8-azaspiro[4.5]decan-3-ylamino)propoxy)-N-methylbenzenesulfonamide; Compound B396: 3-((2S)-2-hydroxy-3-(8-(6-methoxypyridin-3-ylsulfonyl)-1-oxa-8-azaspiro[4.5]decan-3-ylamino)propoxy)-N-methylbenzenesulfonamide; Compound B397: 3-((2S)-2-hydroxy-3-(8-(5-methoxy-2-methylpyridin-3-ylsulfonyl)-1-oxa-8-azaspiro[4.5]decan-3-ylamino)propoxy)-N-methylbenzenesulfonamide; Compound B398: 3-((2S)-2-hydroxy-3-(8-(2-methoxyphenylsulfonyl)-1-oxa-8-azaspiro[4.5]decan-3-ylamino)propoxy)-N-methylbenzenesulfonamide; Compound B399: 3-((2S)-2-hydroxy-3-(8-(1-methyl-2,3-dihydro-1H-pyrido[2,3-b][1,4]oxazin-7-ylsulfonyl)-1-oxa-8-azaspiro[4.5]decan-3-ylamino)propoxy)-N-methylbenzenesulfonamide; Compound B400: 3-((2S)-3-(8-(6-chloro-1-methyl-2,3-dihydro-1H-pyrido[2,3-b][1,4]oxazin-7-ylsulfonyl)-1-oxa-8-azaspiro[4.5]decan-3-ylamino)-2-hydroxypropoxy)-N-methylbenzenesulfonamide; Compound 401: 3-((2S)-3-(8-(6-ethoxy-1-methyl-2,3-dihydro-1H-pyrido[2,3-b][1,4]oxazin-7-ylsulfonyl)-1-oxa-8-azaspiro[4.5]decan-3-ylamino)-2-hydroxypropoxy)-N-methylbenzenesulfonamide; Compound 402: 3-((2S)-2-hydroxy-3-(8-(4-(pyridin-2-yl)phenylsulfonyl)-1-oxa-8-azaspiro[4.5]decan-3-ylamino)propoxy)-N-methylbenzenesulfonamide; Compound B403: 3-((2S)-3-(8-(4′-(aminomethyl)-3-(trifluoromethoxy)biphenyl-4-ylsulfonyl)-1-oxa-8-azaspiro[4.5]decan-3-ylamino)-2-hydroxypropoxy)-N-methylbenzenesulfonamide; Compound B404: 3-((2S)-3-(8-(4′-(aminomethyl)-4-ethoxybiphenyl-3-ylsulfonyl)-1-oxa-8-azaspiro[4.5]decan-3-ylamino)-2-hydroxypropoxy)-N-methylbenzenesulfonamide; Compound B405: 3-((S)-3-((R)-8-(4′-((dimethylamino)methyl)biphenyl-4-ylsulfonyl)-1-oxa-8-azaspiro[4.5]decan-3-ylamino)-2-hydroxypropoxy)-N-methylbenzenesulfonamide; Compound B406: 3-((2S)-2-hydroxy-3-(8-(6-morpholinopyridin-3-ylsulfonyl)-1-oxa-8-azaspiro[4.5]decan-3-ylamino)propoxy)-N-methylbenzenesulfonamide; Compound B407: 3-((2S)-2-hydroxy-3-(8-(4-(pyridin-3-yl)phenylsulfonyl)-1-oxa-8-azaspiro[4.5]decan-3-ylamino)propoxy)-N-methylbenzenesulfonamide; Compound B408: 3-((2S)-2-hydroxy-3-(8-(4-(pyridin-4-yl)phenylsulfonyl)-1-oxa-8-azaspiro[4.5]decan-3-ylamino)propoxy)-N-methylbenzenesulfonamide; Compound B409: 3-((2S)-3-(8-(3-(2H-tetrazol-5-yl)phenylsulfonyl)-1-oxa-8-azaspiro[4.5]decan-3-ylamino)-2-hydroxypropoxy)-N-methylbenzenesulfonamide; Compound B410: 3-((2S)-3-(8-(4-(2H-tetrazol-5-yl)phenylsulfonyl)-1-oxa-8-azaspiro[4.5]decan-3-ylamino)-2-hydroxypropoxy)-N-methylbenzenesulfonamide; Compound B411: 3-((2S)-3-(8-(4′-(aminomethyl)-4-(trifluoromethoxy)biphenyl-3-ylsulfonyl)-1-oxa-8-azaspiro[4.5]decan-3-ylamino)-2-hydroxypropoxy)-N-methylbenzenesulfonamide; Compound B412: 3-((2S)-3-(8-(5-bromo-2-(2-(pyrrolidin-1-yl)ethoxy)phenylsulfonyl)-1-oxa-8-azaspiro[4.5]decan-3-ylamino)-2-hydroxypropoxy)-N-methylbenzenesulfonamide; Compound B413: 3-((S)-2-hydroxy-3-((S)-8-(1-methyl-2,3-dihydro-1H-pyrido[2,3-b][1,4]oxazin-7-ylsulfonyl)-1-oxa-8-azaspiro[4.5]decan-3-ylamino)propoxy)-N-methylbenzenesulfonamide; Compound B414: 3-((S)-2-hydroxy-3-((R)-8-(1-methyl-2,3-dihydro-1H-pyrido[2,3-b][1,4]oxazin-7-ylsulfonyl)-1-oxa-8-azaspiro[4.5]decan-3-ylamino)propoxy)-N-methylbenzenesulfonamide; Compound B415: 3-((S)-3-((S)-8-(4′-(aminomethyl)-4-(trifluoromethoxy)biphenyl-3-ylsulfonyl)-1-oxa-8-azaspiro[4.5]decan-3-ylamino)-2-hydroxypropoxy)-N-methylbenzenesulfonamide; Compound B416: 3-((S)-3-((R)-8-(4′-(aminomethyl)-4-(trifluoromethoxy)biphenyl-3-ylsulfonyl)-1-oxa-8-azaspiro[4.5]decan-3-ylamino)-2-hydroxypropoxy)-N-methylbenzenesulfonamide; Compound B417: 3-((S)-2-hydroxy-3-((R)-8-(1-methyl-2,3-dihydro-1H-pyrido[2,3-b][1,4]oxazin-7-ylsulfonyl)-1-oxa-8-azaspiro[4.5]decan-3-ylamino)propoxy)-N-(2-hydroxyethyl)benzenesulfonamide; Compound B418: 3-((S)-2-hydroxy-3-((R)-8-(1-methyl-2,3-dihydro-1H-pyrido[2,3-b][1,4]oxazin-7-ylsulfonyl)-1-oxa-8-azaspiro[4.5]decan-3-ylamino)propoxy)-N-((R)-1-hydroxypropan-2-yl)benzenesulfonamide; Compound B419: 3-((S)-3-((S)-8-(4′-(aminomethyl)-4-ethoxybiphenyl-3-ylsulfonyl)-1-oxa-8-azaspiro[4.5]decan-3-ylamino)-2-hydroxypropoxy)-N-methylbenzenesulfonamide; Compound B420: 3-((S)-3-((R)-8-(4′-(aminomethyl)-4-ethoxybiphenyl-3-ylsulfonyl)-1-oxa-8-azaspiro[4.5]decan-3-ylamino)-2-hydroxypropoxy)-N-methylbenzenesulfonamide; Compound B421: 3-((2S)-2-hydroxy-3-(8-(6-(piperidin-1-yl)pyridin-3-ylsulfonyl)-1-oxa-8-azaspiro[4.5]decan-3-ylamino)propoxy)-N-methylbenzenesulfonamide; Compound B422: 3-((2S)-3-(8-(6-(dimethylamino)pyridin-3-ylsulfonyl)-1-oxa-8-azaspiro[4.5]decan-3-ylamino)-2-hydroxypropoxy)-N-methylbenzenesulfonamide; Compound B423: 3-((2S)-3-(8-(1-ethyl-2,3-dihydro-1H-pyrido[2,3-b][1,4]oxazin-7-ylsulfonyl)-1-oxa-8-azaspiro[4.5]decan-3-ylamino)-2-hydroxypropoxy)-N-methylbenzenesulfonamide; Compound B424: 3-((S)-3-((S)-8-(4′-(aminomethyl)biphenyl-3-ylsulfonyl)-1-oxa-8-azaspiro[4.5]decan-3-ylamino)-2-hydroxypropoxy)-N-((R)-1-hydroxypropan-2-yl)benzenesulfonamide; Compound B425: 3-((2S)-2-hydroxy-3-(8-(2-hydroxypyrimidin-5-ylsulfonyl)-1-oxa-8-azaspiro[4.5]decan-3-ylamino)propoxy)-N-methylbenzenesulfonamide; Compound B426: 3-((2S)-2-hydroxy-3-(8-(6-(2-methoxyethylamino)pyridin-3-ylsulfonyl)-1-oxa-8-azaspiro[4.5]decan-3-ylamino)propoxy)-N-methylbenzenesulfonamide; Compound B427: 3-((S)-2-hydroxy-3-((R)-8-(1-methyl-2,3-dihydro-1H-pyrido[2,3-b][1,4]oxazin-7-ylsulfonyl)-1-oxa-8-azaspiro[4.5]decan-3-ylamino)propoxy)-N-((1 S,2S)-2-hydroxycyclopentyl)benzenesulfonamide; Compound B428: 3-((2S)-2-hydroxy-3-(8-(6-(2-hydroxyethylamino)pyridin-3-ylsulfonyl)-1-oxa-8-azaspiro[4.5]decan-3-ylamino)propoxy)-N-methylbenzenesulfonamide; Compound B429: 3-((2S)-3-(8-(6-(2-aminoethylamino)pyridin-3-ylsulfonyl)-1-oxa-8-azaspiro[4.5]decan-3-ylamino)-2-hydroxypropoxy)-N-methylbenzenesulfonamide; Compound B430: 3-((2S)-2-hydroxy-3-(8-(6-(piperazin-1-yl)pyridin-3-ylsulfonyl)-1-oxa-8-azaspiro[4.5]decan-3-ylamino)propoxy)-N-methylbenzenesulfonamide; Compound B431: 3-((S)-2-hydroxy-3-((R)-8-(1-methyl-2,3-dihydro-1H-pyrido[2,3-b][1,4]oxazin-7-ylsulfonyl)-1-oxa-8-azaspiro[4.5]decan-3-ylamino)propoxy)-N-((1 S,2S)-2-hydroxycyclohexyl)benzenesulfonamide; Compound B432: 3-((S)-2-hydroxy-3-((R)-8-(1-methyl-2,3-dihydro-1H-pyrido[2,3-b][1,4]oxazin-7-ylsulfonyl)-1-oxa-8-azaspiro[4.5]decan-3-ylamino)propoxy)-N-((S)-1-hydroxypropan-2-yl)benzenesulfonamide; Compound B433: 3-((2S)-3-(8-(4-chloropyridin-2-yl)-1-oxa-8-azaspiro[4.5]decan-3-ylamino)-2-hydroxypropoxy)-N-methylbenzenesulfonamide; Compound B434: 3-((S)-2-hydroxy-3-((R)-8-(1-methyl-2,3-dihydro-1H-pyrido[2,3-b][1,4]oxazin-7-ylsulfonyl)-1-oxa-8-azaspiro[4.5]decan-3-ylamino)propoxy)-N-((S)-2-hydroxypropyl)benzenesulfonamide; Compound B435: 3-((S)-2-hydroxy-3-((R)-8-(1-methyl-2,3-dihydro-1H-pyrido[2,3-b][1,4]oxazin-7-ylsulfonyl)-1-oxa-8-azaspiro[4.5]decan-3-ylamino)propoxy)-N-((1R,2S)-2-hydroxycyclohexyl)benzenesulfonamide; Compound B436: 3-((S)-2-hydroxy-3-((R)-8-(1-methyl-2,3-dihydro-1H-pyrido[2,3-b][1,4]oxazin-7-ylsulfonyl)-1-oxa-8-azaspiro[4.5]decan-3-ylamino)propoxy)-N-((R)-2-hydroxypropyl)benzenesulfonamide; Compound B437: 3-((S)-2-hydroxy-3-((R)-8-(1-methyl-2,3-dihydro-1H-pyrido[2,3-b][1,4]oxazin-7-ylsulfonyl)-1-oxa-8-azaspiro[4.5]decan-3-ylamino)propoxy)-N-((1R,2S)-2-hydroxycyclopentyl)benzenesulfonamide; Compound B438: 3-((S)-3-((S)-8-(4-ethoxy-4′-((isopropylamino)methyl)biphenyl-3-ylsulfonyl)-1-oxa-8-azaspiro[4.5]decan-3-ylamino)-2-hydroxypropoxy)-N-methylbenzenesulfonamide; Compound B439: 3-((S)-3-((R)-8-(4-ethoxy-4′-((isopropylamino)methyl)biphenyl-3-ylsulfonyl)-1-oxa-8-azaspiro[4.5]decan-3-ylamino)-2-hydroxypropoxy)-N-methylbenzenesulfonamide; Compound B440: 3-((S)-2-hydroxy-3-((R)-8-(4′-((isopropylamino)methyl)biphenyl-3-ylsulfonyl)-1-oxa-8-azaspiro[4.5]decan-3-ylamino)propoxy)benzenesulfonamide; Compound B441: 3-((2S)-3-(8-(2-(dimethylamino)pyridin-3-ylsulfonyl)-1-oxa-8-azaspiro[4.5]decan-3-ylamino)-2-hydroxypropoxy)-N-methylbenzenesulfonamide; Compound B442: 3-((2S)-2-hydroxy-3-(8-(2-morpholinopyridin-3-ylsulfonyl)-1-oxa-8-azaspiro[4.5]decan-3-ylamino)propoxy)-N-methylbenzenesulfonamide; Compound B443: 3-((S)-2-hydroxy-3-((R)-8-(2-oxo-2,3-dihydro-1H-pyrido[2,3-b][1,4]oxazin-6-ylsulfonyl)-1-oxa-8-azaspiro[4.5]decan-3-ylamino)propoxy)-N-methylbenzenesulfonamide; Compound B444: 3-((S)-3-((R)-8-(2,3-dihydro-1H-pyrido[2,3-b][1,4]oxazin-7-ylsulfonyl)-1-oxa-8-azaspiro[4.5]decan-3-ylamino)-2-hydroxypropoxy)-N-methylbenzenesulfonamide; Compound B445: 3-((2S)-3-(8-(6-ethoxypyridin-3-ylsulfonyl)-1-oxa-8-azaspiro[4.5]decan-3-ylamino)-2-hydroxypropoxy)-N-methylbenzenesulfonamide; Compound B446: 3-((S)-3-((S)-8-(4′-(1-aminocyclopropyl)biphenyl-3-ylsulfonyl)-1-oxa-8-azaspiro[4.5]decan-3-ylamino)-2-hydroxypropoxy)-N-methylbenzenesulfonamide; Compound B447: 3-((S)-3-((S)-8-(4′-(aminomethyl)-2-methylbiphenyl-3-ylsulfonyl)-1-oxa-8-azaspiro[4.5]decan-3-ylamino)-2-hydroxypropoxy)-N-methylbenzenesulfonamide; Compound B448: 3-((S)-3-((S)-8-(4′-(aminomethyl)-2-methoxybiphenyl-3-ylsulfonyl)-1-oxa-8-azaspiro[4.5]decan-3-ylamino)-2-hydroxypropoxy)-N-methylbenzenesulfonamide; Compound B449: 3-((S)-3-((S)-8-(4′-(aminomethyl)-5-methoxybiphenyl-3-ylsulfonyl)-1-oxa-8-azaspiro[4.5]decan-3-ylamino)-2-hydroxypropoxy)-N-methylbenzenesulfonamide; Compound B450: 3-((S)-3-((S)-8-(6-ethoxy-1-methyl-2,3-dihydro-1H-pyrido[2,3-b][1,4]oxazin-7-ylsulfonyl)-1-oxa-8-azaspiro[4.5]decan-3-ylamino)-2-hydroxypropoxy)-N-methylbenzenesulfonamide; Compound B451: 3-((S)-2-hydroxy-3-((R)-8-(1-methyl-2,3-dihydro-1H-pyrido[2,3-b][1,4]oxazin-7-ylsulfonyl)-1-oxa-8-azaspiro[4.5]decan-3-ylamino)propoxy)-N-methoxybenzenesulfonamide; Compound B452: 3-((S)-2-hydroxy-3-((R)-8-(1-methyl-6-(methylamino)-2,3-dihydro-1H-pyrido[2,3-b][1,4]oxazin-7-ylsulfonyl)-1-oxa-8-azaspiro[4.5]decan-3-ylamino)propoxy)-N-methylbenzenesulfonamide; Compound B453: 3-((S)-3-((R)-8-(6-(dimethylamino)-1-methyl-2,3-dihydro-1H-pyrido[2,3-b][1,4]oxazin-7-ylsulfonyl)-1-oxa-8-azaspiro[4.5]decan-3-ylamino)-2-hydroxypropoxy)-N-methylbenzenesulfonamide; Compound B454: 3-((S)-2-hydroxy-3-((R)-8-(6-hydroxy-1-methyl-2,3-dihydro-1H-pyrido[2,3-b][1,4]oxazin-7-ylsulfonyl)-1-oxa-8-azaspiro[4.5]decan-3-ylamino)propoxy)-N-methylbenzenesulfonamide; Compound B455: 3-((S)-3-((R)-8-(6-amino-1-methyl-2,3-dihydro-1H-pyrido[2,3-b][1,4]oxazin-7-ylsulfonyl)-1-oxa-8-azaspiro[4.5]decan-3-ylamino)-2-hydroxypropoxy)-N-methylbenzenesulfonamide; Compound B456: N-ethyl-3-((S)-2-hydroxy-3-((R)-8-(1-methyl-2,3-dihydro-1H-pyrido[2,3-b][1,4]oxazin-7-ylsulfonyl)-1-oxa-8-azaspiro[4.5]decan-3-ylamino)propoxy)benzenesulfonamide; Compound B457: N-(2-fluoroethyl)-3-((S)-2-hydroxy-3-((R)-8-(1-methyl-2,3-dihydro-1H-pyrido[2,3-b][1,4]oxazin-7-ylsulfonyl)-1-oxa-8-azaspiro[4.5]decan-3-ylamino)propoxy)benzenesulfonamide; Compound B458: 3-((S)-2-hydroxy-3-((R)-8-(1-methyl-2,3-dihydro-1H-pyrido[2,3-b][1,4]oxazin-7-ylsulfonyl)-1-oxa-8-azaspiro[4.5]decan-3-ylamino)propoxy)benzenesulfonamide; Compound B459: 3-((S)-2-hydroxy-3-((R)-8-(1-methyl-2,3-dihydro-1H-pyrido[2,3-b][1,4]oxazin-7-ylsulfonyl)-1-oxa-8-azaspiro[4.5]decan-3-ylamino)propoxy)-N-(2-methoxyethyl)benzenesulfonamide; Compound B460: N-(cyanomethyl)-3-((S)-2-hydroxy-3-((R)-8-(1-methyl-2,3-dihydro-1H-pyrido[2,3-b][1,4]oxazin-7-ylsulfonyl)-1-oxa-8-azaspiro[4.5]decan-3-ylamino)propoxy)benzenesulfonamide; Compound B461: N-(2,2-difluoroethyl)-3-((S)-2-hydroxy-3-((R)-8-(1-methyl-2,3-dihydro-1H-pyrido[2,3-b][1,4]oxazin-7-ylsulfonyl)-1-oxa-8-azaspiro[4.5]decan-3-ylamino)propoxy)benzenesulfonamide; Compound B462: 3-((S)-3-((R)-8-(1-(2-(benzyloxy)ethyl)-2,3-dihydro-1H-pyrido[2,3-b][1,4]oxazin-7-ylsulfonyl)-1-oxa-8-azaspiro[4.5]decan-3-ylamino)-2-hydroxypropoxy)-N-methylbenzenesulfonamide; Compound B463: 3-((S)-3-((S)-8-(4′-(1-aminocyclopropyl)-6-methoxybiphenyl-3-ylsulfonyl)-1-oxa-8-azaspiro[4.5]decan-3-ylamino)-2-hydroxypropoxy)-N-methylbenzenesulfonamide; Compound B464: 3-((S)-3-((R)-8-(4′-(1-aminocyclopropyl)-6-methoxybiphenyl-3-ylsulfonyl)-1-oxa-8-azaspiro[4.5]decan-3-ylamino)-2-hydroxypropoxy)-N-methylbenzenesulfonamide; Compound B465: 3-((S)-2-hydroxy-3-((R)-8-(1-(2-hydroxyethyl)-2,3-dihydro-1H-pyrido[2,3-b][1,4]oxazin-7-ylsulfonyl)-1-oxa-8-azaspiro[4.5]decan-3-ylamino)propoxy)-N-methylbenzenesulfonamide; Compound B466: 3-((S)-3-((R)-8-(1,6-dimethyl-2,3-dihydro-1H-pyrido[2,3-b][1,4]oxazin-7-ylsulfonyl)-1-oxa-8-azaspiro[4.5]decan-3-ylamino)-2-hydroxypropoxy)-N-methylbenzenesulfonamide; Compound B467: 3-((S)-3-((R)-8-(1,8-dimethyl-2,3-dihydro-1H-pyrido[2,3-b][1,4]oxazin-7-ylsulfonyl)-1-oxa-8-azaspiro[4.5]decan-3-ylamino)-2-hydroxypropoxy)-N-methylbenzenesulfonamide; Compound B468: 3-((S)-3-((R)-8-((R)-1,3-dimethyl-2,3-dihydro-1H-pyrido[2,3-b][1,4]oxazin-7-ylsulfonyl)-1-oxa-8-azaspiro[4.5]decan-3-ylamino)-2-hydroxypropoxy)-N-methylbenzenesulfonamide; Compound B469: 3-((S)-2-hydroxy-3-((R)-8-(1-isopropyl-2,3-dihydro-1H-pyrido[2,3-b][1,4]oxazin-7-ylsulfonyl)-1-oxa-8-azaspiro[4.5]decan-3-ylamino)propoxy)-N-methylbenzenesulfonamide; Compound B470: 3-((S)-2-hydroxy-3-((R)-8-(1-propyl-2,3-dihydro-1H-pyrido[2,3-b][1,4]oxazin-7-ylsulfonyl)-1-oxa-8-azaspiro[4.5]decan-3-ylamino)propoxy)-N-methylbenzenesulfonamide; Compound B471: 3-((S)-2-hydroxy-3-((R)-8-(1-(2-methoxyethyl)-2,3-dihydro-1H-pyrido[2,3-b][1,4]oxazin-7-ylsulfonyl)-1-oxa-8-azaspiro[4.5]decan-3-ylamino)propoxy)-N-methylbenzenesulfonamide; Compound B472: 3-((S)-3-((R)-8-((S)-1,3-dimethyl-2,3-dihydro-1H-pyrido[2,3-b][1,4]oxazin-7-ylsulfonyl)-1-oxa-8-azaspiro[4.5]decan-3-ylamino)-2-hydroxypropoxy)-N-methylbenzenesulfonamide; Compound B473: 3-((S)-2-hydroxy-3-((R)-8-(1,3,3-trimethyl-2,3-dihydro-1H-pyrido[2,3-b][1,4]oxazin-7-ylsulfonyl)-1-oxa-8-azaspiro[4.5]decan-3-ylamino)propoxy)-N-methylbenzenesulfonamide; Compound B474: 3-((S)-2-hydroxy-3-((R)-8-(1′-methyl-1′,2′-dihydrospiro[cyclopropane-1,3′-pyrido[2,3-b][1,4]oxazine]-7′-ylsulfonyl)-1-oxa-8-azaspiro[4.5]decan-3-ylamino)propoxy)-N-methylbenzenesulfonamide; Compound B475: 3-((S)-2-hydroxy-3-((R)-8-(3-methyl-3H-imidazo[4,5-b]pyridin-6-ylsulfonyl)-1-oxa-8-azaspiro[4.5]decan-3-ylamino)propoxy)-N-methylbenzenesulfonamide; Compound B476: 3-((S)-2-hydroxy-3-((R)-8-(6-methoxy-1-methyl-2,3-dihydro-1H-pyrido[2,3-b][1,4]oxazin-7-ylsulfonyl)-1-oxa-8-azaspiro[4.5]decan-3-ylamino)propoxy)-N-methylbenzenesulfonamide; Compound B477: 3-((S)-2-hydroxy-3-((R)-8-(2-oxo-2,3-dihydro-1H-imidazo[4,5-b]pyridin-6-ylsulfonyl)-1-oxa-8-azaspiro[4.5]decan-3-ylamino)propoxy)-N-methylbenzenesulfonamide; Compound B478: 3-((S)-2-hydroxy-3-((R)-8-(5-oxo-6,7-dihydro-5H-pyrrolo[3,4-b]pyridin-3-ylsulfonyl)-1-oxa-8-azaspiro[4.5]decan-3-ylamino)propoxy)-N-methylbenzenesulfonamide; Compound B479: 3-((S)-2-hydroxy-3-((R)-8-(5,6,7,8-tetrahydroquinolin-3-ylsulfonyl)-1-oxa-8-azaspiro[4.5]decan-3-ylamino)propoxy)-N-methylbenzenesulfonamide; Compound B480: 3-((S)-3-((R)-8-(2,3-dihydro-[1,4]dioxino[2,3-b]pyridin-7-ylsulfonyl)-1-oxa-8-azaspiro[4.5]decan-3-ylamino)-2-hydroxypropoxy)-N-methylbenzenesulfonamide; Compound B481: 3-((S)-2-hydroxy-3-((R)-8-(4-methyl-3,4-dihydro-2H-pyrido[3,2-b][1,4]oxazin-7-ylsulfonyl)-1-oxa-8-azaspiro[4.5]decan-3-ylamino)propoxy)-N-methylbenzenesulfonamide; Compound B482: 3-((S)-3-((R)-8-(6,7-dihydro-5H-pyrrolo[3,4-b]pyridin-3-ylsulfonyl)-1-oxa-8-azaspiro[4.5]decan-3-ylamino)-2-hydroxypropoxy)-N-methylbenzenesulfonamide; Compound B483: 3-((S)-3-((R)-8-(3,4-dihydro-2H-pyrano[2,3-b]pyridin-6-ylsulfonyl)-1-oxa-8-azaspiro[4.5]decan-3-ylamino)-2-hydroxypropoxy)-N-methylbenzenesulfonamide; Compound B484: 3-((S)-2-hydroxy-3-((R)-8-(5,6,7,8-tetrahydro-1,6-naphthyridin-3-ylsulfonyl)-1-oxa-8-azaspiro[4.5]decan-3-ylamino)propoxy)-N-methylbenzenesulfonamide; Compound B485: 3-((S)-3-((R)-8-(1,5-naphthyridin-3-ylsulfonyl)-1-oxa-8-azaspiro[4.5]decan-3-ylamino)-2-hydroxypropoxy)-N-methylbenzenesulfonamide; Compound B486: 3-((S)-2-hydroxy-3-((R)-8-(2-methyl-1H-imidazo[4,5-b]pyridin-6-ylsulfonyl)-1-oxa-8-azaspiro[4.5]decan-3-ylamino)propoxy)-N-methylbenzenesulfonamide; Compound B487: 3-((S)-2-hydroxy-3-((R)-8-(1-methyl-1,2,3,4-tetrahydropyrido[2,3-b][1,4]oxazepin-8-ylsulfonyl)-1-oxa-8-azaspiro[4.5]decan-3-ylamino)propoxy)-N-methylbenzenesulfonamide; Compound B488: 3-((S)-3-((R)-8-(7,8-dihydro-5H-pyrano[4,3-b]pyridin-3-ylsulfonyl)-1-oxa-8-azaspiro[4.5]decan-3-ylamino)-2-hydroxypropoxy)-N-methylbenzenesulfonamide; Compound B489: 3-((S)-3-((R)-8-(7-amino-1,8-naphthyridin-3-ylsulfonyl)-1-oxa-8-azaspiro[4.5]decan-3-ylamino)-2-hydroxypropoxy)-N-methylbenzenesulfonamide; Compound B490: 3-((S)-3-((R)-8-(1H-pyrazolo[3,4-b]pyridin-5-ylsulfonyl)-1-oxa-8-azaspiro[4.5]decan-3-ylamino)-2-hydroxypropoxy)-N-methylbenzenesulfonamide; Compound B491: 3-((S)-3-((R)-8-(6,7-dihydro-5H-cyclopenta[b]pyridin-3-ylsulfonyl)-1-oxa-8-azaspiro[4.5]decan-3-ylamino)-2-hydroxypropoxy)-N-methylbenzenesulfonamide; Compound B492: 3-((S)-3-((R)-8-(1-ethyl-4-oxo-1,4-dihydroquinolin-3-ylsulfonyl)-1-oxa-8-azaspiro[4.5]decan-3-ylamino)-2-hydroxypropoxy)-N-methylbenzenesulfonamide; Compound B493: 3-((S)-3-((R)-8-(3-cyano-1H-pyrrolo[2,3-b]pyridin-5-ylsulfonyl)-1-oxa-8-azaspiro[4.5]decan-3-ylamino)-2-hydroxypropoxy)-N-methylbenzenesulfonamide; Compound B494: 3-((S)-2-hydroxy-3-((R)-8-(1-methyl-4-oxo-1,4-dihydroquinolin-3-ylsulfonyl)-1-oxa-8-azaspiro[4.5]decan-3-ylamino)propoxy)-N-methylbenzenesulfonamide; Compound B495: 3-((S)-3-((R)-8-(1H-imidazo[4,5-b]pyridin-6-ylsulfonyl)-1-oxa-8-azaspiro[4.5]decan-3-ylamino)-2-hydroxypropoxy)-N-methylbenzenesulfonamide; Compound B496: 3-((R)-3-((S)-2-hydroxy-3-(3-(N-methylsulfamoyl)phenoxy)propylamino)-1-oxa-8-azaspiro[4.5]decan-8-ylsulfonyl)pyridine 1-oxide; Compound B497: 3-((S)-2-hydroxy-3-((R)-8-(3-methyl-1H-pyrazolo[3,4-b]pyridin-5-ylsulfonyl)-1-oxa-8-azaspiro[4.5]decan-3-ylamino)propoxy)-N-methylbenzenesulfonamide; Compound B498: 3-((S)-3-((R)-8-(2,3-dioxoindolin-5-ylsulfonyl)-1-oxa-8-azaspiro[4.5]decan-3-ylamino)-2-hydroxypropoxy)-N-methylbenzenesulfonamide; Compound B499: 3-((S)-2-hydroxy-3-((R)-8-(2-oxo-2,3-dihydro-1H-benzo[d]imidazol-5-ylsulfonyl)-1-oxa-8-azaspiro[4.5]decan-3-ylamino)propoxy)-N-methylbenzenesulfonamide; Compound B500: 3-((S)-3-((R)-8-(3,4-dihydro-2H-pyrido[3,2-b][1,4]oxazin-7-ylsulfonyl)-1-oxa-8-azaspiro[4.5]decan-3-ylamino)-2-hydroxypropoxy)-N-methylbenzenesulfonamide; Compound B501: 3-((S)-2-hydroxy-3-((R)-8-(2-methyl-1H-benzo[d]imidazol-5-ylsulfonyl)-1-oxa-8-azaspiro[4.5]decan-3-ylamino)propoxy)-N-methylbenzenesulfonamide; Compound B502: 3-((S)-3-((R)-8-(1H-pyrazolo[4,3-b]pyridin-6-ylsulfonyl)-1-oxa-8-azaspiro[4.5]decan-3-ylamino)-2-hydroxypropoxy)-N-methylbenzenesulfonamide; Compound B503: 3-((S)-3-((R)-8-(1H-benzo[d]imidazol-5-ylsulfonyl)-1-oxa-8-azaspiro[4.5]decan-3-ylamino)-2-hydroxypropoxy)-N-methylbenzenesulfonamide; Compound B504: 3-((S)-3-((R)-8-(2,3-dihydrofuro[2,3-b]pyridin-5-ylsulfonyl)-1-oxa-8-azaspiro[4.5]decan-3-ylamino)-2-hydroxypropoxy)-N-methylbenzenesulfonamide; Compound B505: 3-((S)-3-((R)-8-(1H-pyrrolo[3,2-b]pyridin-6-ylsulfonyl)-1-oxa-8-azaspiro[4.5]decan-3-ylamino)-2-hydroxypropoxy)-N-methylbenzenesulfonamide; Compound B506: 3-((S)-3-((R)-8-(1H-pyrrolo[2,3-b]pyridin-5-ylsulfonyl)-1-oxa-8-azaspiro[4.5]decan-3-ylamino)-2-hydroxypropoxy)-N-methylbenzenesulfonamide; Compound B507: 3-((S)-3-((R)-8-(1H-pyrrolo[2,3-b]pyridin-3-ylsulfonyl)-1-oxa-8-azaspiro[4.5]decan-3-ylamino)-2-hydroxypropoxy)-N-methylbenzenesulfonamide; Compound B508: 3-((S)-2-hydroxy-3-((R)-8-(4-methoxy-1H-indazol-5-ylsulfonyl)-1-oxa-8-azaspiro[4.5]decan-3-ylamino)propoxy)-N-methylbenzenesulfonamide; Compound B509: 3-((S)-2-hydroxy-3-((R)-8-(3-(trifluoromethyl)-1H-pyrrolo[2,3-b]pyridin-5-ylsulfonyl)-1-oxa-8-azaspiro[4.5]decan-3-ylamino)propoxy)-N-methylbenzenesulfonamide; Compound B510: 3-((S)-3-((R)-8-(3-chloro-1H-pyrrolo[2,3-b]pyridin-5-ylsulfonyl)-1-oxa-8-azaspiro[4.5]decan-3-ylamino)-2-hydroxypropoxy)-N-methylbenzenesulfonamide; Compound B511: 3-((S)-3-((R)-8-(3-ethyl-3H-imidazo[4,5-b]pyridin-6-ylsulfonyl)-1-oxa-8-azaspiro[4.5]decan-3-ylamino)-2-hydroxypropoxy)-N-methylbenzenesulfonamide; Compound B512: 3-((S)-2-hydroxy-3-((R)-8-(3-methyl-1H-pyrrolo[2,3-b]pyridin-5-ylsulfonyl)-1-oxa-8-azaspiro[4.5]decan-3-ylamino)propoxy)-N-methylbenzenesulfonamide; Compound B513: 3-((S)-3-((R)-8-(3-chloro-1-methyl-1H-pyrrolo[2,3-b]pyridin-5-ylsulfonyl)-1-oxa-8-azaspiro[4.5]decan-3-ylamino)-2-hydroxypropoxy)-N-methylbenzenesulfonamide; Compound B514: 3-((S)-2-hydroxy-3-((R)-8-(2-oxo-2,3-dihydro-1H-pyrrolo[2,3-b]pyridin-5-ylsulfonyl)-1-oxa-8-azaspiro[4.5]decan-3-ylamino)propoxy)-N-methylbenzenesulfonamide; Compound B515: 3-((S)-3-((R)-8-(2-ethyl-3H-imidazo[4,5-b]pyridin-6-ylsulfonyl)-1-oxa-8-azaspiro[4.5]decan-3-ylamino)-2-hydroxypropoxy)-N-methylbenzenesulfonamide; Compound B516: 3-((S)-2-hydroxy-3-((R)-8-(5-methyl-3H-imidazo[4,5-b]pyridin-6-ylsulfonyl)-1-oxa-8-azaspiro[4.5]decan-3-ylamino)propoxy)-N-methylbenzenesulfonamide; Compound B517: 3-((S)-3-((R)-8-(3-chloro-2-methyl-1H-pyrrolo[2,3-b]pyridin-5-ylsulfonyl)-1-oxa-8-azaspiro[4.5]decan-3-ylamino)-2-hydroxypropoxy)-N-methylbenzenesulfonamide; Compound B518: 3-((S)-2-hydroxy-3-((R)-8-(3-methyl-2-oxo-2,3-dihydro-1H-imidazo[4,5-b]pyridin-6-ylsulfonyl)-1-oxa-8-azaspiro[4.5]decan-3-ylamino)propoxy)-N-methylbenzenesulfonamide; Compound B519: 3-((S)-2-hydroxy-3-((R)-8-(7-methyl-3H-imidazo[4,5-b]pyridin-6-ylsulfonyl)-1-oxa-8-azaspiro[4.5]decan-3-ylamino)propoxy)-N-methylbenzenesulfonamide; Compound B520: 3-((S)-3-((R)-8-(1H-pyrrolo[3,2-b]pyridin-6-ylsulfonyl)-1-oxa-8-azaspiro[4.5]decan-3-ylamino)-2-hydroxypropoxy)-N-ethylbenzenesulfonamide; Compound B521: 3-((S)-3-((R)-8-(1H-pyrrolo[3,2-b]pyridin-6-ylsulfonyl)-1-oxa-8-azaspiro[4.5]decan-3-ylamino)-2-hydroxypropoxy)-N-(2-hydroxyethyl)benzenesulfonamide; Compound B522: 3-((S)-2-hydroxy-3-((R)-8-(1-methyl-2-oxo-2,3-dihydro-1H-imidazo[4,5-b]pyridin-6-ylsulfonyl)-1-oxa-8-azaspiro[4.5]decan-3-ylamino)propoxy)-N-methylbenzenesulfonamide; Compound B523: 3-((S)-2-hydroxy-3-((R)-8-(1-methyl-1H-imidazo[4,5-b]pyridin-6-ylsulfonyl)-1-oxa-8-azaspiro[4.5]decan-3-ylamino)propoxy)-N-methylbenzenesulfonamide; Compound B524: 3-((S)-2-hydroxy-3-((R)-8-(4-oxo-1-propyl-1,4-dihydroquinolin-3-ylsulfonyl)-1-oxa-8-azaspiro[4.5]decan-3-ylamino)propoxy)-N-methylbenzenesulfonamide; Compound B525: 3-((S)-3-((R)-8-(1-ethyl-8-fluoro-4-oxo-1,4-dihydroquinolin-3-ylsulfonyl)-1-oxa-8-azaspiro[4.5]decan-3-ylamino)-2-hydroxypropoxy)-N-methylbenzenesulfonamide; Compound B526: 3-((S)-2-hydroxy-3-((R)-8-(1-methyl-1H-pyrrolo[3,2-b]pyridin-6-ylsulfonyl)-1-oxa-8-azaspiro[4.5]decan-3-ylamino)propoxy)-N-methylbenzenesulfonamide; Compound B527: 3-((S)-3-((R)-8-(3-chloro-1-methyl-1H-pyrrolo[3,2-b]pyridin-6-ylsulfonyl)-1-oxa-8-azaspiro[4.5]decan-3-ylamino)-2-hydroxypropoxy)-N-methylbenzenesulfonamide; Compound B528: 3-((S)-2-hydroxy-3-((R)-8-(4-hydroxy-8-methylquinolin-3-ylsulfonyl)-1-oxa-8-azaspiro[4.5]decan-3-ylamino)propoxy)-N-methylbenzenesulfonamide; Compound B529: 3-((S)-3-((R)-8-(1H-pyrrolo[3,2-b]pyridin-3-ylsulfonyl)-1-oxa-8-azaspiro[4.5]decan-3-ylamino)-2-hydroxypropoxy)-N-methylbenzenesulfonamide; Compound B530: 3-((S)-3-((R)-8-(1H-pyrrolo[3,2-b]pyridin-6-ylsulfonyl)-1-oxa-8-azaspiro[4.5]decan-3-ylamino)-2-hydroxypropoxy)-N-(2-methoxyethyl)benzenesulfonamide; Compound B531: 3-((S)-3-((R)-8-(1-ethyl-7-methyl-4-oxo-1,4-dihydroquinolin-3-ylsulfonyl)-1-oxa-8-azaspiro[4.5]decan-3-ylamino)-2-hydroxypropoxy)-N-methylbenzenesulfonamide; Compound B532: 3-((S)-3-((R)-8-(1H-pyrrolo[3,2-b]pyridin-6-ylsulfonyl)-1-oxa-8-azaspiro[4.5]decan-3-ylamino)-2-hydroxypropoxy)benzenesulfonamide; Compound B533: 3-((S)-2-hydroxy-3-((R)-8-(4-hydroxy-6-methylquinolin-3-ylsulfonyl)-1-oxa-8-azaspiro[4.5]decan-3-ylamino)propoxy)-N-methylbenzenesulfonamide; Compound B534: 3-((S)-3-((R)-8-(6-fluoro-4-hydroxyquinolin-3-ylsulfonyl)-1-oxa-8-azaspiro[4.5]decan-3-ylamino)-2-hydroxypropoxy)-N-methylbenzenesulfonamide; Compound B535: 3-((S)-3-((R)-8-(1-ethyl-6-methyl-4-oxo-1,4-dihydroquinolin-3-ylsulfonyl)-1-oxa-8-azaspiro[4.5]decan-3-ylamino)-2-hydroxypropoxy)-N-methylbenzenesulfonamide; Compound B536: 3-((S)-3-((R)-8-(1-ethyl-6-fluoro-4-oxo-1,4-dihydroquinolin-3-ylsulfonyl)-1-oxa-8-azaspiro[4.5]decan-3-ylamino)-2-hydroxypropoxy)-N-methylbenzenesulfonamide; Compound B537: 3-((S)-2-hydroxy-3-((R)-8-(imidazo[1,2-a]pyridin-6-ylsulfonyl)-1-oxa-8-azaspiro[4.5]decan-3-ylamino)propoxy)-N-methylbenzenesulfonamide; Compound B538: 3-((S)-2-hydroxy-3-((R)-8-(4-hydroxy-7-methylquinolin-3-ylsulfonyl)-1-oxa-8-azaspiro[4.5]decan-3-ylamino)propoxy)-N-methylbenzenesulfonamide; Compound B539: 3-((S)-3-((R)-8-(7-fluoro-4-hydroxyquinolin-3-ylsulfonyl)-1-oxa-8-azaspiro[4.5]decan-3-ylamino)-2-hydroxypropoxy)-N-methylbenzenesulfonamide; Compound B540: 3-((S)-3-((R)-8-(8-fluoro-4-hydroxyquinolin-3-ylsulfonyl)-1-oxa-8-azaspiro[4.5]decan-3-ylamino)-2-hydroxypropoxy)-N-methylbenzenesulfonamide; Compound B541: 3-((S)-2-hydroxy-3-((R)-8-(4-hydroxyquinolin-3-ylsulfonyl)-1-oxa-8-azaspiro[4.5]decan-3-ylamino)propoxy)-N-methylbenzenesulfonamide; Compound B542: 3-((S)-2-hydroxy-3-((R)-8-(4-hydroxy-7-methylquinolin-3-ylsulfonyl)-1-oxa-8-azaspiro[4.5]decan-3-ylamino)propoxy)benzenesulfonamide; Compound B543: 3-((S)-3-((R)-8-(1-ethyl-7-fluoro-4-oxo-1,4-dihydroquinolin-3-ylsulfonyl)-1-oxa-8-azaspiro[4.5]decan-3-ylamino)-2-hydroxypropoxy)benzenesulfonamide; Compound B544: 2-(3-((R)-3-((S)-2-hydroxy-3-(3-(N-methylsulfamoyl)phenoxy)propylamino)-1-oxa-8-azaspiro[4.5]decan-8-ylsulfonyl)-4-oxoquinolin-1(4H)-yl)acetic acid; Compound B545: 3-((S)-3-((R)-8-(1-ethyl-8-methyl-4-oxo-1,4-dihydroquinolin-3-ylsulfonyl)-1-oxa-8-azaspiro[4.5]decan-3-ylamino)-2-hydroxypropoxy)-N-methylbenzenesulfonamide; Compound B546: 3-((S)-3-((R)-8-(1-ethyl-7-fluoro-4-oxo-1,4-dihydroquinolin-3-ylsulfonyl)-1-oxa-8-azaspiro[4.5]decan-3-ylamino)-2-hydroxypropoxy)-N-methylbenzenesulfonamide; Compound B547: 3-((S)-2-hydroxy-3-((R)-8-(4-hydroxy-6-methylquinolin-8-ylsulfonyl)-1-oxa-8-azaspiro[4.5]decan-3-ylamino)propoxy)-N-methylbenzenesulfonamide; Compound B548: 3-((S)-2-hydroxy-3-((R)-8-(4-hydroxyquinolin-3-ylsulfonyl)-1-oxa-8-azaspiro[4.5]decan-3-ylamino)propoxy)benzenesulfonamide; Compound B549: 3-((S)-2-hydroxy-3-((R)-8-(1-methyl-4-oxo-1,4-dihydroquinolin-3-ylsulfonyl)-1-oxa-8-azaspiro[4.5]decan-3-ylamino)propoxy)benzenesulfonamide; Compound B550: 3-((S)-3-((R)-8-(1-ethyl-4-oxo-1,4-dihydroquinolin-3-ylsulfonyl)-1-oxa-8-azaspiro[4.5]decan-3-ylamino)-2-hydroxypropoxy)benzenesulfonamide; Compound B551: 3-((S)-2-hydroxy-3-((R)-8-(4-oxo-1-propyl-1,4-dihydroquinolin-3-ylsulfonyl)-1-oxa-8-azaspiro[4.5]decan-3-ylamino)propoxy)benzenesulfonamide; Compound B552: 3-((S)-3-((R)-8-(6-fluoro-4-oxo-1,4-dihydroquinolin-3-ylsulfonyl)-1-oxa-8-azaspiro[4.5]decan-3-ylamino)-2-hydroxypropoxy)benzenesulfonamide; Compound B553: 3-((S)-2-hydroxy-3-((R)-8-(8-methyl-4-oxo-1,4-dihydroquinolin-3-ylsulfonyl)-1-oxa-8-azaspiro[4.5]decan-3-ylamino)propoxy)benzenesulfonamide; Compound B554: 3-((S)-3-((R)-8-(8-fluoro-4-oxo-1,4-dihydroquinolin-3-ylsulfonyl)-1-oxa-8-azaspiro[4.5]decan-3-ylamino)-2-hydroxypropoxy)benzenesulfonamide; Compound B555: 3-((S)-3-((R)-8-(7-fluoro-4-oxo-1,4-dihydroquinolin-3-ylsulfonyl)-1-oxa-8-azaspiro[4.5]decan-3-ylamino)-2-hydroxypropoxy)benzenesulfonamide; Compound B556: 3-((S)-2-hydroxy-3-((R)-8-(5,6,7,8-tetrahydronaphthalen-2-ylsulfonyl)-1-oxa-8-azaspiro[4.5]decan-3-ylamino)propoxy)benzenesulfonamide; Compound B557: 3-((S)-3-((R)-8-(1-ethyl-7-methyl-4-oxo-1,4-dihydroquinolin-3-ylsulfonyl)-1-oxa-8-azaspiro[4.5]decan-3-ylamino)-2-hydroxypropoxy)benzenesulfonamide; Compound B558: 3-((S)-2-hydroxy-3-((R)-8-(3-methyl-1H-pyrrolo[2,3-b]pyridin-5-ylsulfonyl)-1-oxa-8-azaspiro[4.5]decan-3-ylamino)propoxy)benzenesulfonamide; Compound B559: 3-((S)-2-hydroxy-3-((R)-8-(2-methyl-3H-imidazo[4,5-b]pyridin-6-ylsulfonyl)-1-oxa-8-azaspiro[4.5]decan-3-ylamino)propoxy)benzenesulfonamide; Compound B560: 3-((S)-2-hydroxy-3-((R)-8-(naphthalen-2-ylsulfonyl)-1-oxa-8-azaspiro[4.5]decan-3-ylamino)propoxy)benzenesulfonamide; Compound 561: 3-((S)-2-hydroxy-3-((R)-8-(pyridin-2-yl)-1-oxa-8-azaspiro[4.5]decan-3-ylamino)propoxy)-N-methylbenzenesulfonamide; Compound B562: 3-((S)-2-hydroxy-3-((R)-8-(quinolin-3-ylsulfonyl)-1-oxa-8-azaspiro[4.5]decan-3-ylamino)propoxy)benzenesulfonamide; Compound B563: 3-((S)-3-((S)-8-(1H-pyrrolo[3,2-b]pyridin-6-ylsulfonyl)-1-oxa-8-azaspiro[4.5]decan-3-ylamino)-2-hydroxypropoxy)benzenesulfonamide; Compound B564: 3-((S)-2-hydroxy-3-((R)-8-(3-methyl-1H-pyrazolo[3,4-b]pyridin-5-ylsulfonyl)-1-oxa-8-azaspiro[4.5]decan-3-ylamino)propoxy)benzenesulfonamide; Compound B565: 3-((S)-3-((R)-8-(chroman-6-ylsulfonyl)-1-oxa-8-azaspiro[4.5]decan-3-ylamino)-2-hydroxypropoxy)benzenesulfonamide; Compound B566: 3-((S)-2-hydroxy-3-((R)-8-(quinolin-6-ylsulfonyl)-1-oxa-8-azaspiro[4.5]decan-3-ylamino)propoxy)benzenesulfonamide; Compound B567: 3-((S)-3-((R)-8-(4′-(aminomethyl)-4-ethoxybiphenyl-3-ylsulfonyl)-1-oxa-8-azaspiro[4.5]decan-3-ylamino)-2-hydroxypropoxy)benzenesulfonamide; Compound B568: 3-((S)-2-hydroxy-3-((R)-8-(4-methoxypyrimidin-2-yl)-1-oxa-8-azaspiro[4.5]decan-3-ylamino)propoxy)-N-methylbenzenesulfonamide; Compound B569: 3-((S)-3-((R)-8-(4-aminopyrimidin-2-yl)-1-oxa-8-azaspiro[4.5]decan-3-ylamino)-2-hydroxypropoxy)-N-methylbenzenesulfonamide; Compound B570: 3-((S)-3-((R)-8-(5-benzylpyrimidin-2-yl)-1-oxa-8-azaspiro[4.5]decan-3-ylamino)-2-hydroxypropoxy)-N-methylbenzenesulfonamide; Compound B571: 3-((S)-3-((R)-8-(5-ethylpyrimidin-2-yl)-1-oxa-8-azaspiro[4.5]decan-3-ylamino)-2-hydroxypropoxy)-N-methylbenzenesulfonamide; Compound B572: 3-((S)-2-hydroxy-3-((R)-8-(4-phenylpyrimidin-2-yl)-1-oxa-8-azaspiro[4.5]decan-3-ylamino)propoxy)-N-methylbenzenesulfonamide; Compound B573: 3-((S)-3-((R)-8-(4-benzylpyrimidin-2-yl)-1-oxa-8-azaspiro[4.5]decan-3-ylamino)-2-hydroxypropoxy)-N-methylbenzenesulfonamide; Compound B574: 3-((S)-2-hydroxy-3-((R)-8-(4-(trifluoromethyl)pyrimidin-2-yl)-1-oxa-8-azaspiro[4.5]decan-3-ylamino)propoxy)-N-methylbenzenesulfonamide; Compound B575: 3-((S)-2-hydroxy-3-((R)-8-(4-methylpyrimidin-2-yl)-1-oxa-8-azaspiro[4.5]decan-3-ylamino)propoxy)-N-methylbenzenesulfonamide; Compound B576: 3-((S)-2-hydroxy-3-((R)-8-(pyrimidin-2-yl)-1-oxa-8-azaspiro[4.5]decan-3-ylamino)propoxy)-N-methylbenzenesulfonamide; Compound B577: 3-((S)-3-((R)-8-(4,6-dimethylpyrimidin-2-yl)-1-oxa-8-azaspiro[4.5]decan-3-ylamino)-2-hydroxypropoxy)-N-methylbenzenesulfonamide; Compound B578: 3-((S)-3-((R)-8-(5-heptylpyrimidin-2-yl)-1-oxa-8-azaspiro[4.5]decan-3-ylamino)-2-hydroxypropoxy)-N-methylbenzenesulfonamide; Compound B579: 3-((S)-2-hydroxy-3-((R)-8-(5-propylpyrimidin-2-yl)-1-oxa-8-azaspiro[4.5]decan-3-ylamino)propoxy)-N-methylbenzenesulfonamide; Compound B580: 3-((S)-2-hydroxy-3-((R)-8-(5-phenylpyrimidin-2-yl)-1-oxa-8-azaspiro[4.5]decan-3-ylamino)propoxy)-N-methylbenzenesulfonamide; Compound B581: methyl 2-((R)-3-((S)-2-hydroxy-3-(3-(N-methylsulfamoyl)phenoxy)propylamino)-1-oxa-8-azaspiro[4.5]decan-8-yl)pyrimidine-5-carboxylate; Compound B582: 2-((R)-3-((S)-2-hydroxy-3-(3-(N-methylsulfamoyl)phenoxy)propylamino)-1-oxa-8-azaspiro[4.5]decan-8-yl)pyrimidine-4-carboxylic acid; Compound B583: 3-((S)-2-hydroxy-3-((R)-8-(5-(trifluoromethyl)pyrimidin-2-yl)-1-oxa-8-azaspiro[4.5]decan-3-ylamino)propoxy)-N-methylbenzenesulfonamide; Compound B584: 3-((S)-3-((R)-8-(4,6-dimethoxypyrimidin-2-yl)-1-oxa-8-azaspiro[4.5]decan-3-ylamino)-2-hydroxypropoxy)-N-methylbenzenesulfonamide; Compound B585: 3-((S)-2-hydroxy-3-((R)-8-(5-(trifluoromethyl)pyrazin-2-yl)-1-oxa-8-azaspiro[4.5]decan-3-ylamino)propoxy)-N-methylbenzenesulfonamide; Compound B586: 3-((S)-2-hydroxy-3-((R)-8-(pyrazin-2-yl)-1-oxa-8-azaspiro[4.5]decan-3-ylamino)propoxy)-N-methylbenzenesulfonamide; Compound B587: 3-((S)-3-((R)-8-(1-ethyl-4-oxo-1,4-dihydropyridin-3-ylsulfonyl)-1-oxa-8-azaspiro[4.5]decan-3-ylamino)-2-hydroxypropoxy)benzenesulfonamide; Compound B588: 3-((S)-3-((R)-8-(1-ethyl-4-oxo-1,4-dihydropyridin-3-ylsulfonyl)-1-oxa-8-azaspiro[4.5]decan-3-ylamino)-2-hydroxypropoxy)-N-methylbenzenesulfonamide; Compound B589: 3-((S)-2-hydroxy-3-((R)-8-(pyrimidin-4-yl)-1-oxa-8-azaspiro[4.5]decan-3-ylamino)propoxy)-N-methylbenzenesulfonamide; Compound B590: 3-((S)-3-((R)-8-(4-bromo-3-methylphenylsulfonyl)-1-oxa-8-azaspiro[4.5]decan-3-ylamino)-2-hydroxypropoxy)benzenesulfonamide; Compound B591: 3-((S)-3-((R)-8-(3-chlorophenylsulfonyl)-1-oxa-8-azaspiro[4.5]decan-3-ylamino)-2-hydroxypropoxy)benzenesulfonamide; Compound B592: 3-((S)-3-((R)-8-(3-chlorophenylsulfonyl)-1-oxa-8-azaspiro[4.5]decan-3-ylamino)-2-hydroxypropoxy)-N-methylbenzenesulfonamide; Compound B593: 3-((S)-2-hydroxy-3-((R)-8-(4-(3-methoxyphenyl)pyrimidin-2-yl)-1-oxa-8-azaspiro[4.5]decan-3-ylamino)propoxy)-N-methylbenzenesulfonamide; and Compound B594: 3-((S)-2-hydroxy-3-((R)-8-(4-methyl-6-phenylpyrimidin-2-yl)-1-oxa-8-azaspiro[4.5]decan-3-ylamino)propoxy)-N-methylbenzenesulfonamide.

As used herein, “substituted” indicates that at least one hydrogen atom of the chemical group is replaced by a non-hydrogen substituent or group, the non-hydrogen substituent or group can be monovalent or divalent. When the substituent or group is divalent, then it is understood that this group is further substituted with another substituent or group. When a chemical group herein is “substituted” it may have up to the full valance of substitution; for example, a methyl group can be substituted by 1, 2, or 3 substituents, a methylene group can be substituted by 1 or 2 substituents, a phenyl group can be substituted by 1, 2, 3, 4, or 5 substituents, a naphthyl group can be substituted by 1, 2, 3, 4, 5, 6, or 7 substituents, and the like. Likewise, “substituted with one or more substituents” refers to the substitution of a group substituted with one substituent up to the total number of substituents physically allowed by the group. Further, when a group is substituted with more than one group they can be identical or they can be different.

Compounds of the invention can also include tautomeric forms, such as keto-enol tautomers and the like. Tautomeric forms can be in equilibrium or sterically locked into one form by appropriate substitution. It is understood that the various tautomeric forms are within the scope of the compounds of the present invention. One example relates to compounds containing the group described herein as 4-oxo-1,4-dihydroquinolin-3-yl, such as Compound A326. Even though one tautomer is shown for a compound, it is understood that the compound embraces all such tautomers; below are two representative tautomers of 4-oxo-1,4-dihydroquinolin-3-yl:

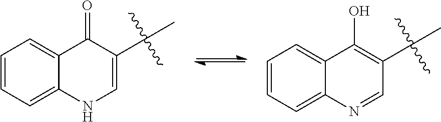 [SMILES: *c1c[nH]c2ccccc2c1=O ; *c1cnc2ccccc2c1O]

It is understood and appreciated that compounds of Formula (Ia) and formulae related thereto may have one or more chiral centers and therefore can exist as enantiomers and/or diastereoisomers. The invention is understood to extend to and embrace all such enantiomers, diastereoisomers, and mixtures thereof, including but not limited to racemates.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables contained within the generic chemical formulae described herein are specifically embraced by the present invention just as if each and every combination was individually and explicitly recited, to the extent that such combinations embrace compounds that result in stable compounds (i.e., compounds that can be isolated, characterized, and tested for biological activity). In addition, all subcombinations of the chemical groups listed in the embodiments describing such variables, as well as all subcombinations of uses and medical indications described herein, are also specifically embraced by the present invention just as if each and every subcombination of chemical groups and subcombination of uses and medical indications was individually and explicitly recited herein.

Additionally, chemical genera of the present invention and individual compounds, for example those compounds found in the above lists and tables below, including diastereoisomers and enantiomers thereof, encompass all pharmaceutically acceptable salts, solvates, hydrates, and N-oxides thereof.

The compounds of the present invention may be prepared according to relevant published literature procedures that are used by one skilled in the art. See, e.g., U.S. Pat. No. 10,479,797 and U.S. Patent Publication No. 2020-0385395, each of which is incorporated herein in its entirety.

It is understood that the present invention embraces each isomer, each diastereoisomer, each enantiomer and mixtures thereof of each compound and generic formulae disclosed herein just as if they were each individually disclosed with the specific stereochemical designation for each chiral carbon. Individual isomers and enantiomers can be prepared by selective synthesis, such as, by enantiomeric selective syntheses; or they can be obtained using separation techniques which are well known to practitioners in the art, such as, by HPLC (including, normal phase, reverse phase, and chiral), recrystallization (i.e., diastereoisomeric mixtures) and the like techniques.

### Disorders and Methods of Treatment

The compounds disclosed herein are useful in the treatment or prevention of several diseases, disorders, conditions, and/or indications (which are cumulatively referred to herein as “disorders”). One of skill in the art will recognize that when a disorder, or a method of treatment or prevention, is disclosed herein, such disclosure encompasses second medical uses (e.g., a compound for use in the treatment of the disorder, use of a compound for the treatment of the disorder, and use of a compound in the manufacture of a medicament for the treatment of the disorder).

In some embodiments, the compounds disclosed herein are useful for the treatment or prevention of a disorder. In some embodiments, the compounds disclosed herein are useful for the treatment or prevention of a subtype of a disorder. In some embodiments, the compounds disclosed herein are useful for the treatment or prevention of a symptom of a disorder.

Provided herein are methods for treating or preventing a beta-3 adrenergic receptor-mediated disorder. In some embodiments, the compounds disclosed herein are useful for the prevention of a beta-3 adrenergic receptor-mediated disorder. In some embodiments, the compounds disclosed herein are useful for the treatment or prevention of a beta-3 adrenergic receptor-mediated disorder.

More specifically, provide are methods of treating renal cystic disease and/or cardiorenal syndrome by administrating certain compounds that modulate the activity of the beta-3 adrenergic receptor.

In some embodiments, the renal cystic disease is chosen from polycystic kidney disease, unilateral renal cystic disease (localized cystic disease), renal simple cysts, multicystic dysplastic kidney, pluricystic kidney of the multiple malformation syndromes, juvenile nephronophthisis and medullary cystic disease, medullary sponge kidney, primary glomerulocystic kidney disease, glomerulocystic kidney associated with several systemic disorders mainly of genetic or chromosomal etiology, cystic kidney in tuberous sclerosis, and in von Hippel-Lindau syndrome, cystic nephroma, cystic variant of congenital mesoblastic nephroma, mixed epithelial stromal tumor of the kidney, renal lymphangioma, pyelocalyceal cyst, peripylic cyst and perinephric pseudocyst, acquired renal cystic disease of long-term dialysis, and cystic renal cell carcinoma and sarcoma.

In some embodiments, the renal cystic disease is polycystic kidney disease. In some embodiments, the polycystic kidney disease is autosomal-dominant polycystic kidney disease.

In some embodiments, the cardiorenal disease is cardiorenal syndrome type 1. In some embodiments, the cardiorenal disease is cardiorenal syndrome type 2. In some embodiments, the cardiorenal disease is cardiorenal syndrome type 3. In some embodiments, the cardiorenal disease is cardiorenal syndrome type 4.

### Polymorphs and Pseudopolymorphs

Polymorphism is the ability of a substance to exist as two or more crystalline phases that have different arrangements and/or conformations of the molecules in the crystal lattice. Polymorphs show the same properties in the liquid or gaseous state but they behave differently in the solid state.

Besides single-component polymorphs, drugs can also exist as salts and other multicomponent crystalline phases. For example, solvates and hydrates may contain an API host and either solvent or water molecules, respectively, as guests. Analogously, when the guest compound is a solid at room temperature, the resulting form is often called a cocrystal. Salts, solvates, hydrates, and cocrystals may show polymorphism as well. Crystalline phases that share the same API host, but differ with respect to their guests, may be referred to as pseudopolymorphs of one another.

Solvates contain molecules of the solvent of crystallization in a definite crystal lattice. Solvates, in which the solvent of crystallization is water, are termed hydrates. Because water is a constituent of the atmosphere, hydrates of drugs may be formed rather easily.

By way of example, Stahly published a polymorph screen of 245 compounds consisting of a “wide variety of structural types” that revealed about 90% of them exhibited multiple solid forms. Overall, approximately half of the compounds were polymorphic, often having one to three forms. About one-third of the compounds formed hydrates, and about one-third formed solvates. Data from cocrystal screens of 64 compounds showed that 60% formed cocrystals other than hydrates or solvates. (G. P. Stahly, Crystal Growth & Design (2007), 7(6), 1007-1026).

### Isotopes

The present disclosure includes all isotopes of atoms occurring in the compounds provided herein. Isotopes include those atoms having the same atomic number but different mass numbers. It is appreciated that certain features of the invention(s) include every combination of one or more atoms in the compounds provided herein that is replaced with an atom having the same atomic number but a different mass number. One such example is the replacement of an atom that is the most naturally abundant isotope, such as ¹H or ¹²C, found in one of the compounds provided herein with a different atom that is not the most naturally abundant isotope, such as ²H or ³H (replacing ¹H), or ¹¹C, ¹³C, or ¹⁴C (replacing ¹²C). A compound wherein such a replacement has taken place is commonly referred to as being isotopically-labeled. Isotopic-labeling of the present compounds can be accomplished using any one of a variety of different synthetic methods know to those of ordinary skill in the art and they are readily credited with understanding the synthetic methods and available reagents needed to conduct such isotopic-labeling. By way of general example, and without limitation, isotopes of hydrogen include ²H (deuterium) and ³H (tritium). Isotopes of carbon include ¹¹C, ¹³C, and ¹⁴C. Isotopes of nitrogen include ¹³N and ¹⁵N. Isotopes of oxygen include ¹⁵O, ¹⁷O, and ¹⁸O. An isotope of fluorine includes ¹⁸F. An isotope of sulfur includes ³⁵S. An isotope of chlorine includes ³⁶Cl. Isotopes of bromine include ⁷⁵Br, ⁷⁶Br, ⁷⁷Br, and ⁸²Br. Isotopes of iodine include ¹²³I, ¹²⁴I, ¹²⁵I, and ¹³¹I. Also provided are compositions, such as, those prepared during synthesis, preformulation, and the like, and pharmaceutical compositions, such as, those prepared with the intent of using in a mammal for the treatment of one or more of the disorders described herein, comprising one or more of the present compounds, wherein the naturally occurring distribution of the isotopes in the composition is perturbed. Also provided herein are compositions and pharmaceutical compositions comprising compounds of the invention as described herein, wherein the salt is enriched at one or more positions with an isotope other than the most naturally abundant isotope. Methods are readily available to measure such isotope perturbations or enrichments, such as, mass spectrometry, and for isotopes that are radio-isotopes additional methods are available, such as, radio-detectors used in connection with HPLC or GC.

One challenge in drug development is improving absorption, distribution, metabolism, excretion, and toxicity (ADMET) properties while maintaining a desired pharmacological profile. Structural changes to improve ADMET properties often alter the pharmacology of a lead compound. While the effects of deuterium substitution on ADMET properties are unpredictable, in select cases deuterium can improve a compound's ADMET properties with minimal perturbation of its pharmacology. Two examples where deuterium has enabled improvements in therapeutic entities are: CTP-347 and CTP-354. CTP-347 is a deuterated version of paroxetine with a reduced liability for mechanism-based inactivation of CYP2D6 that is observed clinically with paroxetine. CTP-354 is a deuterated version of a promising preclinical gamma-aminobutyric acid A receptor (GABAA) modulator (L-838417) that was not developed due to poor pharmacokinetic (PK) properties. In both cases, deuterium substitution resulted in improved ADMET profiles that provide the potential for improved safety, efficacy, and/or tolerability without significantly altering the biochemical potency and selectivity versus the all-hydrogen compounds. Provided are deuterium substituted compounds of the present invention with improved ADMET profiles and substantially similar biochemical potency and selectivity versus the corresponding all-hydrogen compounds.

### Other Utilities

Another object of the present invention relates to radio-labeled compounds of the present invention that would be useful not only in radio-imaging but also in assays, both in vitro and in vivo, for localizing and quantitating beta-3 adrenergic receptors in tissue samples, including human and for identifying beta-3 adrenergic receptor ligands by inhibition binding of a radio-labeled compound. It is a further object of this invention to develop novel beta-3 adrenergic receptor assays of which comprise such radio-labeled compounds.

The present disclosure includes all isotopes of atoms occurring in the present compounds, intermediates, salts and crystalline forms thereof. Isotopes include those atoms having the same atomic number but different mass numbers. One aspect of the present invention includes every combination of one or more atoms in the present compounds, intermediates, salts, and crystalline forms thereof that is replaced with an atom having the same atomic number but a different mass number. One such example is the replacement of an atom that is the most naturally abundant isotope, such as ¹H or ¹²C, found in one the present compounds, intermediates, salts, and crystalline forms thereof, with a different atom that is not the most naturally abundant isotope, such as ²H or ³H (replacing ¹H), or ¹¹C, ¹³C, or ¹⁴C (replacing ¹²C). A compound wherein such a replacement has taken place is commonly referred to as being an isotopically-labeled compound. Isotopic-labeling of the present compounds, intermediates, salts, and crystalline forms thereof can be accomplished using any one of a variety of different synthetic methods know to those of ordinary skill in the art and they are readily credited with understanding the synthetic methods and available reagents needed to conduct such isotopic-labeling. By way of general example, and without limitation, isotopes of hydrogen include ²H (deuterium) and ³H (tritium). Isotopes of carbon include ¹¹C, ¹³C, and ¹⁴C. Isotopes of nitrogen include ¹³N and ¹⁵N. Isotopes of oxygen include ¹⁵O, ¹⁷O, and ¹⁸O. An isotope of fluorine includes ¹⁸F. An isotope of sulfur includes ³⁵S. An isotope of chlorine includes ³⁶Cl. Isotopes of bromine include ⁷⁵Br, ⁷⁶Br, ⁷⁷Br, and ⁸²Br. Isotopes of iodine include ¹²³I, ¹²⁴I, ¹²⁵I, and ¹³¹I. Another aspect of the present invention includes compositions, such as, those prepared during synthesis, preformulation, and the like, and pharmaceutical compositions, such as, those prepared with the intent of using in a mammal for the treatment of one or more of the disorders described herein, comprising one or more of the present compounds, intermediates, salts, and crystalline forms thereof, wherein the naturally occurring distribution of the isotopes in the composition is perturbed. Another aspect of the present invention includes compositions and pharmaceutical compositions comprising compounds as described herein wherein the compound is enriched at one or more positions with an isotope other than the most naturally abundant isotope. Methods are readily available to measure such isotope perturbations or enrichments, such as, mass spectrometry, and for isotopes that are radio-isotopes additional methods are available, such as, radio-detectors used in connection with HPLC or GC.

Certain isotopically-labeled compounds of the present invention are useful in compound and/or substrate tissue distribution assays. In some embodiments the radionuclide ³H and/or ¹⁴C isotopes are useful in these studies. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the present invention can generally be prepared by following procedures analogous to those disclosed in the Drawings and Examples infra, by substituting an isotopically labeled reagent for a non-isotopically labeled reagent. Other synthetic methods that are useful are discussed infra. Moreover, it should be understood that all of the atoms represented in the compounds of the invention can be either the most commonly occurring isotope of such atoms or the scarcer radio-isotope or nonradioactive isotope.

Synthetic methods for incorporating radio-isotopes into organic compounds are applicable to compounds of the invention and are well known in the art. Representative synthetic methods for incorporating activity levels of tritium into target molecules include, for example:
A. Catalytic Reduction with Tritium Gas: This procedure normally yields high specific activity products and requires halogenated or unsaturated precursors.
B. Reduction with Sodium Borohydride [³H]: This procedure is rather inexpensive and requires precursors containing reducible functional groups such as aldehydes, ketones, lactones, esters and the like.
C. Reduction with Lithium Aluminum Hydride [³H]: This procedure offers products at almost theoretical specific activities. It also requires precursors containing reducible functional groups such as aldehydes, ketones, lactones, esters and the like.
D. Tritium Gas Exposure Labeling: This procedure involves exposing precursors containing exchangeable protons to tritium gas in the presence of a suitable catalyst.
E. N-Methylation using Methyl Iodide [³H]: This procedure is usually employed to prepare O-methyl or N-methyl (³H) products by treating appropriate precursors with high specific activity methyl iodide (³H). This method in general allows for higher specific activity, such as for example, about 70-90 Ci/mmol.

Synthetic methods for incorporating activity levels of ¹²⁵I into target molecules include:
A. Sandmeyer and like reactions: This procedure transforms an aryl amine or a heteroaryl amine into a diazonium salt, such as a diazonium tetrafluoroborate salt and subsequently to ¹²⁵I labeled compound using Na¹²⁵I. A represented procedure was reported by Zhu, G-D. and co-workers in J. Org. Chem., 2002, 67, 943-948.
B. Ortho ¹²⁵Iodination of phenols: This procedure allows for the incorporation of ¹²⁵I at the ortho position of a phenol as reported by Collier, T. L. and co-workers in J. Labelled Compd. Radiopharm., 1999, 42, S264-S266.
C. Aryl and heteroaryl bromide exchange with ¹²⁵I: This method is generally a two step process. The first step is the conversion of the aryl or heteroaryl bromide to the corresponding tri-alkyltin intermediate using for example, a Pd catalyzed reaction [i.e. Pd(Ph₃P)₄] or through an aryl or heteroaryl lithium, in the presence of a tri-alkyltinhalide or hexaalkylditin [e.g., (CH₃)₃SnSn(CH₃)₃]. A representative procedure was reported by Le Bas, M.-D. and co-workers in J. Labelled Compd. Radiopharm. 2001, 44, S280-S282.

A radiolabeled beta-3 adrenergic receptor compound of Formula (Ia) can be used in a screening assay to identify/evaluate compounds. In general terms, a newly synthesized or identified compound (i.e., test compound) can be evaluated for its ability to reduce binding of the “radiolabeled compound of Formula (Ia)” to a beta-3 adrenergic receptor. Accordingly, the ability of a test compound to compete with the “radiolabeled compound of Formula (Ia)” for the binding to a beta-3 adrenergic receptor directly correlates to its binding affinity.

Certain labeled compounds of the present invention bind to certain beta-3 adrenergic receptors. In one embodiment the labeled compound has an IC₅₀ less than about 500 μM, in another embodiment the labeled compound has an IC₅₀ less than about 100 μM, in yet another embodiment the labeled compound has an IC₅₀ less than about 10 μM, in yet another embodiment the labeled compound has an IC₅₀ less than about 1 μM and in still yet another embodiment the labeled compound has an IC₅₀ less than about 0.1 μM.

### Compositions and Formulations

Formulations may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tabletting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the compound provided herein in a suitable liquid vehicle and filter sterilizing the solution before filling and sealing an appropriate vial or ampule. These are just a few examples of the many appropriate methods well known in the art for preparing dosage forms.

A compound of the present invention can be formulated into pharmaceutical compositions using techniques well known to those in the art. Suitable pharmaceutically-acceptable carriers, outside those mentioned herein, are known in the art; for example, see Remington, The Science and Practice of Pharmacy, 20th Edition, 2000, Lippincott Williams & Wilkins, (Editors: Gennaro et. al.).

While it is possible that, for use in the prophylaxis or treatment, a compound provided herein may, in an alternative use, be administered as a raw or pure chemical, it is preferable however to present the compound or active ingredient as a pharmaceutical formulation or composition further comprising a pharmaceutically acceptable carrier.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation, insufflation or by a transdermal patch. Transdermal patches dispense a drug at a controlled rate by presenting the drug for absorption in an efficient manner with minimal degradation of the drug. Typically, transdermal patches comprise an impermeable backing layer, a single pressure sensitive adhesive and a removable protective layer with a release liner. One of ordinary skill in the art will understand and appreciate the techniques appropriate for manufacturing a desired efficacious transdermal patch based upon the needs of the artisan.

The compounds provided herein, together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof may comprise conventional ingredients in conventional proportions, with or without additional active compounds or principles and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

Compounds provided herein or a salt, solvate, or hydrate thereof can be used as active ingredients in pharmaceutical compositions, specifically as beta-3 adrenergic receptor modulators. The term “active ingredient”, defined in the context of a “pharmaceutical composition”,” refers to a component of a pharmaceutical composition that provides the primary pharmacological effect, as opposed to an “inactive ingredient” which would generally be recognized as providing no pharmaceutical benefit.

The dose when using the compounds provided herein can vary within wide limits and as is customary and is known to the physician or other clinician, it is to be tailored to the individual conditions in each individual case. It depends, for example, on the nature and severity of the illness to be treated, on the condition of the patient, on the compound employed or on whether an acute or chronic disease state is treated, or prophylaxis conducted, or on whether further active compounds are administered in addition to the compounds provided herein. Representative doses include, but are not limited to, about 0.001 mg to about 5000 mg, about 0.001 mg to about 2500 mg, about 0.001 mg to about 1000 mg, about 0.001 mg to about 500 mg, about 0.001 mg to about 250 mg, about 0.001 mg to 100 mg, about 0.001 mg to about 50 mg and about 0.001 mg to about 25 mg. Multiple doses may be administered during the day, especially when relatively large amounts are deemed to be needed, for example 2, 3, or 4 doses. Depending on the individual and as deemed appropriate from the healthcare provider it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated, or prophylaxis conducted, or on whether further active compounds are administered in addition to the compounds provided herein and as part of a drug combination. The dosage regimen for treating a disease condition with the compounds and/or compositions provided herein is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods provided herein.

The desired dose may conveniently be presented in a single dose or as divided doses administered at appropriate intervals, for example, as two, three, four, or more sub-doses per day. The sub-dose itself may be further divided, e.g., into a number of discrete loosely spaced administrations. The daily dose can be divided, especially when relatively large amounts are administered as deemed appropriate, into several, for example two, three, or four-part administrations. If appropriate, depending on individual behavior, it may be necessary to deviate upward or downward from the daily dose indicated.

The compounds provided herein can be administrated in a wide variety of oral and parenteral dosage forms. It will be obvious to those skilled in the art that the dosage forms may comprise, as the active component, either a compound provided herein or a pharmaceutically acceptable salt, hydrate, or solvate of a compound provided herein.

For preparing pharmaceutical compositions from the compounds provided herein, the selection of a suitable pharmaceutically acceptable carrier can be either solid, liquid or a mixture of both. Solid form preparations include powders, tablets, pills, capsules, cachets, suppositories and dispersible granules. A solid carrier can be one or more substances which may also act as diluents, flavoring agents, solubilizers, lubricants, suspending agents, binders, preservatives, tablet disintegrating agents, or an encapsulating material.

In powders, the carrier is a finely divided solid which is in a mixture with the finely divided active component.

In tablets, the active component is admixed with the carrier having the necessary binding capacity in suitable proportions and compacted to the desire shape and size.

The powders and tablets may contain varying percentage amounts of the active compound. A representative amount in a powder or tablet may contain from 0.5 to about 90 percent of the active compound; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethyl cellulose, a low melting wax, cocoa butter and the like.

The term “preparation” refers to the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool and thereby to solidify.

Formulations suitable for vaginal administration may be presented as pessaries, tampons, creams, gels, pastes, foams, or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents. The sterile injectable preparation may also be a sterile injectable solution or suspension in a nontoxic parenterally acceptable diluent or solvent, for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds provided herein may thus be formulated for parenteral administration (e.g. by injection, for example bolus injection or continuous infusion) and may be presented in unit dose form in ampoules, pre-filled syringes, small volume infusion or in multi-dose containers with an added preservative. The pharmaceutical compositions may take such forms as suspensions, solutions, or emulsions in oily or aqueous vehicles and may contain formulatory agents such as suspending, stabilizing and/or dispersing agents. Alternatively, the active ingredient may be in powder form, obtained by aseptic isolation of sterile solid or by lyophilization from solution, for constitution with a suitable vehicle, e.g. sterile, pyrogen-free water, before use.

Aqueous formulations suitable for oral use can be prepared by dissolving or suspending the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethyl cellulose, or other well-known suspending agents.

Also included are solid form preparations which are intended to be converted, shortly before use, to liquid form preparations for oral administration. Such liquid forms include solutions, suspensions and emulsions. These preparations may contain, in addition to the active component, colorants, flavors, stabilizers, buffers, artificial and natural sweeteners, dispersants, thickeners, solubilizing agents and the like.

For topical administration to the epidermis the compounds provided herein may be formulated as ointments, creams or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations may be provided in single or multi-dose form. In the latter case of a dropper or pipette, this may be achieved by the patient administering an appropriate, predetermined volume of the solution or suspension. In the case of a spray, this may be achieved for example by means of a metering atomizing spray pump.

Administration to the respiratory tract may also be achieved by means of an aerosol formulation in which the active ingredient is provided in a pressurized pack with a suitable propellant. If the compounds provided herein or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds provided herein as an aerosol can be prepared by processes well known to the person skilled in the art. For their preparation, for example, solutions or dispersions of the compounds provided herein in water, water/alcohol mixtures or suitable saline solutions can be employed using customary additives, for example benzyl alcohol or other suitable preservatives, absorption enhancers for increasing the bioavailability, solubilizers, dispersants and others and, if appropriate, customary propellants, for example include carbon dioxide, CFCs, such as, dichlorodifluoromethane, trichlorofluoromethane, or dichlorotetrafluoroethane; and the like. The aerosol may conveniently also contain a surfactant such as lecithin. The dose of drug may be controlled by provision of a metered valve.

In formulations intended for administration to the respiratory tract, including intranasal formulations, the compound will generally have a small particle size for example of the order of 10 microns or less. Such a particle size may be obtained by means known in the art, for example by micronization. When desired, formulations adapted to give sustained release of the active ingredient may be employed.

Alternatively the active ingredients may be provided in the form of a dry powder, for example, a powder mix of the compound in a suitable powder base such as lactose, starch, starch derivatives such as hydroxypropylmethyl cellulose and polyvinylpyrrolidone (PVP).

Conveniently the powder carrier will form a gel in the nasal cavity. The powder composition may be presented in unit dose form for example in capsules or cartridges of, e.g., gelatin, or blister packs from which the powder may be administered by means of an inhaler.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

The compounds provided herein may optionally exist as pharmaceutically acceptable salts including pharmaceutically acceptable acid addition salts prepared from pharmaceutically acceptable non-toxic acids including inorganic and organic acids. Representative acids include, but are not limited to, acetic, benzenesulfonic, benzoic, camphorsulfonic, citric, ethenesulfonic, dichloroacetic, formic, fumaric, gluconic, glutamic, hippuric, hydrobromic, hydrochloric, isethionic, lactic, maleic, malic, mandelic, methanesulfonic, mucic, nitric, oxalic, pamoic, pantothenic, phosphoric, succinic, sulfiric, tartaric, oxalic, p-toluenesulfonic and the like. Certain compounds provided herein which contain a carboxylic acid functional group may optionally exist as pharmaceutically acceptable salts containing non-toxic, pharmaceutically acceptable metal cations and cations derived from organic bases. Representative metals include, but are not limited to, aluminium, calcium, lithium, magnesium, potassium, sodium, zinc and the like. In some embodiments the pharmaceutically acceptable metal is sodium. Representative organic bases include, but are not limited to, benzathine (N¹,N²-dibenzylethane-1,2-diamine), chloroprocaine (2-(diethylamino)ethyl 4-(chloroamino)benzoate), choline, diethanolamine, ethylenediamine, meglumine ((2R,3R,4R,5S)-6-(methylamino)hexane-1,2,3,4,5-pentaol), procaine (2-(diethylamino)ethyl 4-aminobenzoate), and the like. Certain pharmaceutically acceptable salts are listed in Berge, et. al., Journal of Pharmaceutical Sciences, 66:1-19 (1977).

The acid addition salts may be obtained as the direct products of compound synthesis. In the alternative, the free base may be dissolved in a suitable solvent containing the appropriate acid and the salt isolated by evaporating the solvent or otherwise separating the salt and solvent. The compounds provided herein may form solvates with standard low molecular weight solvents using methods known to the skilled artisan.

Compounds provided herein can be converted to “pro-drugs.” The term “pro-drugs” refers to compounds that have been modified with specific chemical groups known in the art and when administered into an individual these groups undergo biotransformation to give the parent compound. Pro-drugs can thus be viewed as compounds provided herein containing one or more specialized non-toxic protective groups used in a transient manner to alter or to eliminate a property of the compound. In one general aspect, the “pro-drug” approach is utilized to facilitate oral absorption. A thorough discussion is provided in T. Higuchi and V. Stella, Pro-drugs as Novel Delivery Systems Vol. 14 of the A.C.S. Symposium Series; and in Bioreversible Carriers in Drug Design, ed. Edward B. Roche, American Pharmaceutical Association and Pergamon Press, 1987.

Some embodiments include a method of producing a pharmaceutical composition for “combination-therapy” comprising admixing at least one compound according to any of the compound embodiments disclosed herein, together with at least one known pharmaceutical agent and a pharmaceutically acceptable carrier.

It is noted that when the beta-3 adrenergic receptor modulators are utilized as active ingredients in pharmaceutical compositions, these are not intended for use in humans only, but in non-human mammals as well. Recent advances in the area of animal health-care mandate that consideration be given for the use of active agents, such as beta-3 adrenergic receptor modulators, for the treatment of a beta-3 adrenergic receptor-associated disease or disorder in companionship animals (e.g., cats, dogs, etc.) and in livestock animals (e.g., horses, cows, etc.) Those of ordinary skill in the art are readily credited with understanding the utility of such compounds in such settings.

Other uses of the disclosed receptors and methods will become apparent to those skilled in the art based upon, inter alia, a review of this disclosure.

As will be recognized, the steps of the methods of the present invention need not be performed any particular number of times or in any particular sequence. Additional objects, advantages and novel features of this invention will become apparent to those skilled in the art upon examination of the following examples thereof, which are intended to be illustrative and not intended to be limiting.

### EXAMPLES

### Example 1: IC50 Determinations in Homogeneous Time-Resolved Fluorescence (HTRF®) cAMP Antagonist Assays

HTRF cAMP assays were performed according to manufacturer's instructions (Cisbio, cAMP Dynamic 2 Assay Kit; #62AM4PEJ). CHO-K1 cells stably expressing recombinant receptor were harvested and suspended in warm PBS to make a 300,000 cells/mL stock. This cell suspension was dispensed into 384 well assay plates (PerkinElmer ProxiPlate #6008280) at 5 μL per well (1500 cells/well) along with a cAMP standard curve.

Compounds were dissolved and serially diluted (5-fold) in DMSO to generate a 10-point dose response stock. The stock was then diluted 100-fold in assay buffer (PBS containing 1 mM IBMX) before a volume of 2.5 μL was added to the cells (the final, top concentration of compound in the dose-response is typically 10 or 100 μM). After a brief incubation, 2.5 μL of isoproterenol stock, prepared at a concentration 4 times its EC₉₀ at the receptor of interest, was added to the wells. The EC₉₀ for isoproterenol, a beta-adrenergic agonist, was determined in separate experiments using standard methods to measure agonist potencies.

Following a 1-hour incubation at room temperature, 5 μL of cAMP-D2 Reagent diluted in Lysis Buffer was added to each well followed by 5 μL of Cryptate Reagent. Plates were further incubated at room temperature for 1 hour prior to reading. Time resolved fluorescence measurements were collected on a suitable, HTRF-capable plate reader.

Counts from the plate reader were fit to the cAMP standard curve on the assay plate in order to determine cAMP concentrations in each well, and these values were used to construct dose-response curves to obtain IC₅₀ values.

Beta-3 Adrenergic Receptor IC₅₀ Values

| Cmpd No. | IC₅₀ | |
|---|---|---|
| A5 | 33.31 | nM |
| A88 | 25.12 | nM |
| A123 | 53.17 | nM |
| A136 | 18.83 | nM |
| A154 | 17.5 | nM |
| A161 | 21.14 | nM |
| A163 | 17.31 | nM |
| A169 | 17.47 | nM |
| A199 | 25.01 | nM |
| A210 | 22.43 | nM |
| A211 | 26.18 | nM |
| A217 | 28.43 | nM |
| A225 | 33.1 | nM |
| A227 | 17.9 | nM |
| A229 | 11.58 | nM |
| A230 | 14.99 | nM |
| A232 | 16.63 | nM |
| A234 | 10.19 | nM |
| A240 | 31.69 | nM |
| A241 | 21.06 | nM |
| A243 | 35.01 | nM |
| A244 | 27.83 | nM |
| A245 | 18.38 | nM |
| A247 | 33.47 | nM |
| A296 | 107.3 | nM |
| A297 | 26.5 | nM |
| A300 | 20.39 | nM |
| A309 | 7.003 | nM |
| A310 | 37.65 | nM |
| A320 | 11.18 | nM |
| A321 | 17.83 | nM |
| A322 | 21.77 | nM |
| A326 | 17.26 | nM |
| A327 | 28.92 | nM |
| A329 | 29.49 | nM |
| A331 | 30.04 | nM |

Each of the compounds specifically described herein was observed to have a beta-3 adrenergic receptor IC₅₀ value in the range of about 3.0 nM to about 2.0 μM.

Specific IC₅₀ values for certain compounds are provided below, where the number directly preceeding the IC₅₀ value refers to the compound number (e.g., 1: 4.42 nM refers to Compound 1 with an IC₅₀ value of 4.42 nM): A1: 4.42 nM; A2: 44.79 nM; A3: 5.47 nM; A4: 4.45 nM; A6: 16.46 nM; A7: 34.24 nM; A8: 12.08 nM; A9: 24.25 nM; A10: 25.05 nM; A11: 35.52 nM; A12: 130.50 nM; A13: 97.85 nM; A14: 56.71 nM; A15: 5.70 nM; A16: 132.70 nM; A17: 38.76 nM; A18: 48.93 nM; A19: 66.74 nM; A20: 76.21 nM; A21: 252.30 nM; A22: 302.00 nM; A23: 242.00 nM; A24: 23.63 nM; A25: 54.56 nM; A26: 5.25 nM; A27: 19.33 nM; A28: 18.52 nM; A29: 16.09 nM; A30: 34.28 nM; A31: 49.26 nM; A32: 21.06 nM; A33: 189.20 nM; A34: 31.24 nM; A35: 20.26 nM; A36: 21.51 nM; A37: 9.37 nM; A38: 3.99 nM; A39: 70.81 nM; A40: 20.33 nM; A41: 13.68 nM; A42: 18.68 nM; A43: 32.42 nM; A44: 21.51 nM; A45: 23.64 nM; A46: 87.03 nM; A47: 82.09 nM; A48: 46.11 nM; A49: 33.13 nM; A50: 42.23 nM; A51: 17.36 nM; A52: 61.20 nM; A53: 23.93 nM; A54: 45.94 nM; A55: 30.55 nM; A56: 1988.00 nM; A57: 486.90 nM; A58: 109.30 nM; A59: 12.30 nM; A60: 17.37 nM; A61: 29.81 nM; A62: 24.46 nM; A63: 17.20 nM; A64: 921.70 nM; A65: 710.50 nM; A66: 981.60 nM; A67: 821.90 nM; A68: 493.60 nM; A69: 21.25 nM; A70: 24.43 nM; A71: 38.95 nM; A72: 328.70 nM; A73: 23.95 nM; A74: 37.97 nM; A75: 26.70 nM; A76: 35.91 nM; A77: 35.08 nM; A78: 41.93 nM; A79: 42.20 nM; A80: 38.10 nM; A81: 27.79 nM; A82: 43.23 nM; A83: 53.47 nM; A84: 54.69 nM; A85: 17.84 nM; A86: 9.10 nM; A87: 41.12 nM; A89: 110.10 nM; A90: 354.30 nM; A91: 37.14 nM; A92: 26.93 nM; A93: 75.04 nM; A94: 113.50 nM; A95: 21.09 nM; A96: 410.60 nM; A97: 212.10 nM; A98: 89.00 nM; A99: 287.30 nM; A100: 92.16 nM; A101: 466.20 nM; A102: 113.80 nM; A103: 1195.00 nM; A104: 36.50 nM; A105: 277.40 nM; A106: 36.24 nM; A107: 25.73 nM; A108: 325.30 nM; A109: 144.60 nM; A110: 68.65 nM; A111: 31.07 nM; A112: 39.47 nM; A113: 26.79 nM; A114: 51.96 nM; A115: 120.50 nM; A116: 319.70 nM; A117: 171.60 nM; A118: 10.44 nM; A119: 11.64 nM; A120: 177.30 nM; A121: 562.40 nM; A122: 7.81 nM; A124: 78.20 nM; A125: 48.81 nM; A126: 126.50 nM; A127: 156.60 nM; A128: 108.50 nM; A129: 485.00 nM; A130: 12.00 nM; A131: 23.72 nM; A132: 739.30 nM; A133: 443.00 nM; A134: 55.13 nM; A135: 88.77 nM; A137: 349.50 nM; A138: 20.41 nM; A139: 36.18 nM; A140: 45.89 nM; A141: 11.63 nM; A142: 106.00 nM; A143: 13.23 nM; A144: 7.77 nM; A145: 81.89 nM; A146: 103.60 nM; A147: 53.32 nM; A148: 21.76 nM; A149: 97.26 nM; A150: 226.60 nM; A151: 40.85 nM; A152: 24.68 nM; A153: 18.80 nM; A155: 48.24 nM; A156: 24.78 nM; A157: 25.73 nM; A158: 27.29 nM; A159: 177.90 nM; A160: 11.58 nM; A162: 29.51 nM; A164: 27.73 nM; A165: 64.38 nM; A166: 51.18 nM; A167: 15.79 nM; A168: 15.38 nM; A170: 416.40 nM; A171: 25.03 nM; A172: 50.34 nM; A173: 31.25 nM; A174: 699.30 nM; A175: 715.60 nM; A176: 58.42 nM; A177: 50.59 nM; A178: 84.61 nM; A179: 49.11 nM; A180: 30.67 nM; A181: 179.70 nM; A182: 24.74 nM; A183: 1600.00 nM; A184: 26.72 nM; A185: 257.80 nM; A186: 78.60 nM; A187: 33.40 nM; A188: 35.02 nM; A189: 10.31 nM; A190: 31.27 nM; A191: 439.90 nM; A192: 298.30 nM; A193: 26.90 nM; A194: 13.65 nM; A195: 1530.00 nM; A196: 34.61 nM; A197: 84.99 nM; A198: 62.23 nM; A200: 72.38 nM; A201: 41.80 nM; A202: 345.90 nM; A203: 1111.00 nM; A204: 503.80 nM; A205: 92.19 nM; A206: 402.80 nM; A207: 51.11 nM; A208: 34.76 nM; A209: 8.79 nM; A212: 25.96 nM; A213: 18.62 nM; A214: 36.66 nM; A215: 38.42 nM; A216: 26.42 nM; A218: 29.32 nM; A219: 21.59 nM; A220: 27.50 nM; A221: 20.57 nM; A222: 47.57 nM; A223: 83.59 nM; A224: 73.16 nM; A226: 12.46 nM; A228: 29.07 nM; A231: 19.61 nM; A233: 9.72 nM; A235: 14.97 nM; A236: 368.60 nM; A237: 81.97 nM; A238: 22.84 nM; A239: 30.94 nM; A242: 35.50 nM; A246: 48.29 nM; A248: 72.39 nM; A249: 224.40 nM; A250: 335.70 nM; A251: 51.62 nM; A252: 29.26 nM; A253: 29.15 nM; A254: 41.77 nM; A255: 27.94 nM; A256: 32.64 nM; A257: 37.84 nM; A258: 27.73 nM; A259: 158.30 nM; A260: 177.90 nM; A261: 38.77 nM; A262: 981.90 nM; A263: 754.60 nM; A264: 979.70 nM; A265: 374.30 nM; A266: 1291.00 nM; A267: 442.30 nM; A268: 138.30 nM; A269: 399.70 nM; A270: 92.31 nM; A271: 205.50 nM; A272: 566.10 nM; A273: 29.35 nM; A274: 38.43 nM; A275: 62.29 nM; A276: 1808.00 nM; A277: 16.46 nM; A278: 585.90 nM; A279: 39.75 nM; A280: 96.39 nM; A281: 57.87 nM; A282: 23.74 nM; A283: 68.93 nM; A284: 106.40 nM; A285: 303.30 nM; A286: 56.94 nM; A287: 150.50 nM; A288: 176.50 nM; A289: 65.82 nM; A290: 34.18 nM; A291: 69.80 nM; A292: 90.25 nM; A293: 130.20 nM; A294: 109.80 nM; A295: 143.20 nM; A298: 82.52 nM; A299: 44.31 nM; A301: 357.00 nM; A302: 696.00 nM; A303: 17.11 nM; A304: 69.02 nM; A305: 17.11 nM; A306: 62.90 nM; A307: 59.18 nM; A308: 34.50 nM; A311: 43.50 nM; A312: 31.55 nM; A313: 141.50 nM; A314: 269.70 nM; A315: 23.94 nM; A316: 63.76 nM; A317: 104.00 nM; A318: 95.17 nM; A319: 16.51 nM; A323: 49.56 nM; A324: 22.46 nM; A325: 34.05 nM; A328: 35.37 nM; A330: 70.08 nM; A332: 42.26 nM; A333: 16.28 nM; A334: 79.94 nM; A335: 14.45 nM; A336: 101.70 nM; A337: 1689.00 nM; and A338: 32.15 nM.

Beta-3 Adrenergic Receptor IC₅₀ Values

| Cmpd | | |
|---|---|---|
| No. | IC₅₀ | |
| B1 | 5.4 | nM |
| B2 | 21.1 | nM |
| B3 | 133 | nM |
| B4 | 11.2 | nM |
| B5 | 220 | nM |
| B6 | 24.3 | nM |
| B7 | 285 | nM |
| B8 | 38.7 | nM |
| B9 | 107 | nM |
| B10 | 49.2 | nM |
| B11 | 15.1 | nM |
| B12 | 31.7 | nM |
| B13 | 28.7 | nM |
| B14 | 30.1 | nM |
| B15 | 12.4 | nM |
| B16 | 7.47 | nM |
| B17 | 15.3 | nM |
| B18 | 20.8 | nM |
| B19 | 10 | nM |
| B20 | 13.2 | nM |
| B21 | 13.4 | nM |
| B22 | 15 | nM |
| B23 | 13.8 | nM |
| B24 | 106 | nM |
| B25 | 26 | nM |
| B26 | 34.1 | nM |
| B27 | 13.8 | nM |
| B28 | 16.2 | nM |
| B29 | 4.53 | nM |
| B30 | 4.41 | nM |
| B31 | 21 | nM |
| B32 | 16.6 | nM |
| B33 | 17.6 | nM |
| B34 | 24.2 | nM |
| B35 | 18.9 | nM |
| B36 | 13.2 | nM |
| B37 | 49.3 | nM |
| B38 | 18.7 | nM |
| B39 | 30.7 | nM |
| B40 | 577 | nM |
| B41 | 6.82 | nM |
| B42 | 31.9 | nM |
| B43 | 74 | nM |
| B44 | 34.1 | nM |
| B45 | 19.4 | nM |
| B46 | 54.1 | nM |
| B47 | 155 | nM |
| B48 | 133 | nM |
| B49 | 107 | nM |
| B50 | 87.2 | nM |
| B51 | 58.5 | nM |
| B52 | 14.1 | nM |
| B53 | 21.8 | nM |
| B54 | 17.4 | nM |
| B55 | 18 | nM |
| B56 | 387 | nM |
| B57 | 31.7 | nM |
| B58 | 43 | nM |
| B59 | 14.5 | nM |
| B60 | 58.8 | nM |
| B61 | 13.5 | nM |
| B62 | 44.9 | nM |
| B63 | 19.3 | nM |
| B64 | 11.1 | nM |
| B65 | 85.6 | nM |
| B66 | 22.7 | nM |
| B67 | 23.1 | nM |
| B68 | 36.9 | nM |
| B69 | 21.1 | nM |
| B70 | 25.7 | nM |
| B71 | 31.2 | nM |
| B72 | 28.7 | nM |
| B73 | 22.1 | nM |
| B74 | 236 | nM |
| B75 | 39.8 | nM |
| B76 | 26.5 | nM |
| B77 | 17.5 | nM |
| B78 | 20.7 | nM |
| B79 | 7.15 | nM |
| B80 | 14.3 | nM |
| B81 | 16.6 | nM |
| B82 | 15.9 | nM |
| B83 | 99.9 | nM |
| B84 | 9.09 | nM |
| B85 | 9.73 | nM |
| B86 | 9.36 | nM |
| B87 | 66.1 | nM |
| B88 | 9.82 | nM |
| B89 | 429 | nM |
| B90 | 49.2 | nM |
| B91 | 1.12 | μM |
| B92 | 1.99 | μM |
| B93 | 112 | nM |
| B94 | 59.8 | nM |
| B95 | 159 | nM |
| B96 | 42.1 | nM |
| B97 | 127 | nM |
| B98 | 71.1 | nM |
| B99 | 121 | nM |
| B100 | 256 | nM |
| B101 | 9.62 | nM |
| B102 | 26 | nM |
| B103 | 10.5 | nM |
| B104 | 17.3 | nM |
| B105 | 31.2 | nM |
| B106 | 13.3 | nM |
| B107 | 10.9 | nM |
| B108 | 13.4 | nM |
| B109 | 13.7 | nM |
| B110 | 19 | nM |
| B111 | 26.3 | nM |
| B112 | 239 | nM |
| B113 | 213 | nM |
| B114 | 10.3 | nM |
| B115 | 33.5 | nM |
| B116 | 23.5 | nM |
| B117 | 14.5 | nM |
| B118 | 24.1 | nM |
| B119 | 203 | nM |
| B120 | 36.4 | nM |
| B121 | 67 | nM |
| B122 | 34.1 | nM |
| B123 | 40.5 | nM |
| B124 | 36.2 | nM |
| B125 | 78.1 | nM |
| B126 | 13 | nM |
| B127 | 14.9 | nM |
| B128 | 11.4 | nM |
| B129 | 24.9 | nM |
| B130 | 8.32 | nM |
| B131 | 84.7 | nM |
| B132 | 10.8 | nM |
| B133 | 65.4 | nM |
| B134 | 37.9 | nM |
| B135 | 33.3 | nM |
| B136 | 23.5 | nM |
| B137 | 23.8 | nM |
| B138 | 14.2 | nM |
| B139 | 10.2 | nM |
| B140 | 11.3 | nM |
| B141 | 35.1 | nM |
| B142 | 16.8 | nM |
| B143 | 17.1 | nM |
| B144 | 12.2 | nM |
| B145 | 21.5 | nM |
| B146 | 36.2 | nM |
| B147 | 13 | nM |
| B148 | 11 | nM |
| B149 | 20.6 | nM |
| B150 | 13.5 | nM |
| B151 | 5.38 | nM |
| B152 | 90.7 | nM |
| B153 | 43.6 | nM |
| B154 | 34.4 | nM |
| B155 | 4.36 | nM |
| B156 | 25.4 | nM |
| B157 | 26.4 | nM |
| B158 | 25.6 | nM |
| B159 | 4.78 | nM |
| B160 | 14.6 | nM |
| B161 | 83.7 | nM |
| B162 | 42.6 | nM |
| B163 | 64.1 | nM |
| B164 | 84.9 | nM |
| B165 | 311 | nM |
| B166 | 143 | nM |
| B167 | 2.15 | μM |
| B168 | 1.47 | μM |
| B169 | 11.6 | nM |
| B170 | 50.6 | nM |
| B171 | 30.7 | nM |
| B172 | 78.9 | nM |
| B173 | 29 | nM |
| B174 | 125 | nM |
| B175 | 23.8 | nM |
| B176 | 23.3 | nM |
| B177 | 11.9 | nM |
| B178 | 20.8 | nM |
| B179 | 8.15 | nM |
| B180 | 29.8 | nM |
| B181 | 24.3 | nM |
| B182 | 105 | nM |
| B183 | 16.4 | nM |
| B184 | 11.9 | nM |
| B185 | 7.25 | nM |
| B186 | 15.7 | nM |
| B187 | 7.72 | nM |
| B188 | 18.1 | nM |
| B189 | 8.51 | nM |
| B190 | 18 | nM |
| B191 | 80.9 | nM |
| B192 | 28.3 | nM |
| B193 | 54.8 | nM |
| B194 | 97.6 | nM |
| B195 | 23.4 | nM |
| B196 | 39.7 | nM |
| B197 | 12.8 | nM |
| B198 | 8.65 | nM |
| B199 | 15 | nM |
| B200 | 22.2 | nM |
| B201 | 17.1 | nM |
| B202 | 10.8 | nM |
| B203 | 14.9 | nM |
| B204 | 12.1 | nM |
| B205 | 14 | nM |
| B206 | 9.62 | nM |
| B207 | 8.79 | nM |
| B208 | 64.6 | nM |
| B209 | 41 | nM |
| B210 | 20.4 | nM |
| B211 | 16.5 | nM |
| B212 | 13.6 | nM |
| B213 | 49.1 | nM |
| B214 | 31.4 | nM |
| B215 | 7.41 | nM |
| B216 | 57.7 | nM |
| B217 | 35.1 | nM |
| B218 | 39.3 | nM |
| B219 | 25.3 | nM |
| B220 | 19.1 | nM |
| B221 | 9.58 | nM |
| B222 | 109 | nM |
| B223 | 13.6 | nM |
| B224 | 33.1 | nM |
| B225 | 186 | nM |
| B226 | 155 | nM |
| B227 | 31.3 | nM |
| B228 | 24.2 | nM |
| B229 | 272 | nM |
| B230 | 85.4 | nM |
| B231 | 72.6 | nM |
| B232 | 188 | nM |
| B233 | 156 | nM |
| B234 | 52.6 | nM |
| B235 | 108 | nM |
| B236 | 54.9 | nM |
| B237 | 42.8 | nM |
| B238 | 38.4 | nM |
| B239 | 22.3 | nM |
| B240 | 28.4 | nM |
| B241 | 27.8 | nM |
| B242 | 420 | nM |
| B243 | 26.2 | nM |
| B244 | 76.1 | nM |
| B245 | 15.5 | nM |
| B246 | 38.6 | nM |
| B247 | 27.2 | nM |
| B248 | 40.5 | nM |
| B249 | 29.5 | nM |
| B250 | 24.2 | nM |
| B251 | 32.9 | nM |
| B252 | 29.3 | nM |
| B253 | 22.6 | nM |
| B254 | 97.3 | nM |
| B255 | 55.1 | nM |
| B256 | 21.1 | nM |
| B257 | 37 | nM |
| B258 | 67.9 | nM |
| B259 | 23.1 | nM |
| B260 | 20.3 | nM |
| B261 | 47.5 | nM |
| B262 | 54.3 | nM |
| B263 | 30.8 | nM |
| B264 | 39 | nM |
| B265 | 28.8 | nM |
| B266 | 24.7 | nM |
| B267 | 9.43 | nM |
| B268 | 12.3 | nM |
| B269 | 24.7 | nM |
| B270 | 16.7 | nM |
| B271 | 7.53 | nM |
| B272 | 107 | nM |
| B273 | 15.5 | nM |
| B274 | 21.1 | nM |
| B275 | 21.3 | nM |
| B276 | 200 | nM |
| B277 | 46.7 | nM |
| B278 | 12.8 | nM |
| B279 | 31.6 | nM |
| B280 | 98 | nM |
| B281 | 10.7 | nM |
| B282 | 15.5 | nM |
| B283 | 84.6 | nM |
| B284 | 11.6 | nM |
| B285 | 25 | nM |
| B286 | 20.5 | nM |
| B287 | 21.9 | nM |
| B288 | 13.4 | nM |
| B289 | 16.5 | nM |
| B290 | 175 | nM |
| B291 | 8.84 | nM |
| B292 | 30.1 | nM |
| B293 | 16.2 | nM |
| B294 | 56.2 | nM |
| B295 | 7.22 | nM |
| B296 | 17.5 | nM |
| B297 | 15.9 | nM |
| B298 | 26 | nM |
| B299 | 11.8 | nM |
| B300 | 10.9 | nM |
| B301 | 31.6 | nM |
| B302 | 458 | nM |
| B303 | 32.6 | nM |
| B304 | 43 | nM |
| B305 | 63.5 | nM |
| B306 | 65.4 | nM |
| B307 | 3.37 | μM |
| B308 | 1.82 | μM |
| B309 | 3 | μM |
| B310 | 877 | nM |
| B311 | 2 | μM |
| B312 | 3.17 | μM |
| B313 | 4.01 | μM |
| B314 | 716 | nM |
| B315 | 1.07 | μM |
| B316 | 1.94 | μM |
| B317 | 1.01 | μM |
| B318 | 2.18 | μM |
| B319 | 34.3 | nM |
| B320 | 26.3 | nM |
| B321 | 70.5 | nM |
| B322 | 73.5 | nM |
| B323 | 24.8 | nM |
| B324 | 78 | nM |
| B325 | 42.7 | nM |
| B326 | 11.7 | nM |
| B327 | 92.1 | nM |
| B328 | 37 | nM |
| B329 | 156 | nM |
| B330 | 36.7 | nM |
| B331 | 19.6 | nM |
| B332 | 17.9 | nM |
| B333 | 9.02 | nM |
| B334 | 27.3 | nM |
| B335 | 12.1 | nM |
| B336 | 15.3 | nM |
| B337 | 18.6 | nM |
| B338 | 47.1 | nM |
| B339 | 42.4 | nM |
| B340 | 30.9 | nM |
| B341 | 28.5 | nM |
| B342 | 42.6 | nM |
| B343 | 27.7 | nM |
| B344 | 42.7 | nM |
| B345 | 107 | nM |
| B346 | 126 | nM |
| B347 | 33.5 | nM |
| B348 | 36.7 | nM |
| B349 | 67.9 | nM |
| B350 | 21.8 | nM |
| B351 | 38.6 | nM |
| B352 | 167 | nM |
| B353 | 58 | nM |
| B354 | 35.2 | nM |
| B355 | 17.2 | nM |
| B356 | 16.9 | nM |
| B357 | 15.7 | nM |
| B358 | 16.1 | nM |
| B359 | 17.9 | nM |
| B360 | 20.5 | nM |
| B361 | 22.1 | nM |
| B362 | 27.5 | nM |
| B363 | 123 | nM |
| B364 | 144 | nM |
| B365 | 20.7 | nM |
| B366 | 27.5 | nM |
| B367 | 31.2 | nM |
| B368 | 35.2 | nM |
| B369 | 14.6 | nM |
| B370 | 47.8 | nM |
| B371 | 42 | nM |
| B372 | 41.8 | nM |
| B373 | 24.4 | nM |
| B374 | 15.7 | nM |
| B375 | 25.4 | nM |
| B376 | 499 | nM |
| B377 | 18.9 | nM |
| B378 | 131 | nM |
| B379 | 11.1 | nM |
| B380 | 102 | nM |
| B381 | 357 | nM |
| B382 | 21 | nM |
| B383 | 21.8 | nM |
| B384 | 11 | nM |
| B385 | 11 | nM |
| B386 | 57.9 | nM |
| B387 | 30.8 | nM |
| B388 | 79.4 | nM |
| B389 | 475 | nM |
| B390 | 153 | nM |
| B391 | 446 | nM |
| B392 | 168 | nM |
| B393 | 141 | nM |
| B394 | 46.9 | nM |
| B395 | 206 | nM |
| B396 | 30.3 | nM |
| B397 | 115 | nM |
| B398 | 51.3 | nM |
| B399 | 20.2 | nM |
| B400 | 33.1 | nM |
| B401 | 25.3 | nM |
| B402 | 20.4 | nM |
| B403 | 80.6 | nM |
| B404 | 13 | nM |
| B405 | 51.8 | nM |
| B406 | 31.1 | nM |
| B407 | 23 | nM |
| B408 | 39.1 | nM |
| B409 | 180 | nM |
| B410 | 389 | nM |
| B411 | 9.84 | nM |
| B412 | 49.6 | nM |
| B413 | 14 | nM |
| B414 | 16.8 | nM |
| B415 | 19.5 | nM |
| B416 | 262 | nM |
| B417 | 112 | nM |
| B418 | 172 | nM |
| B419 | 9.69 | nM |
| B420 | 10.2 | nM |
| B421 | 16.9 | nM |
| B422 | 16.7 | nM |
| B423 | 12.6 | nM |
| B424 | 51.3 | nM |
| B425 | 216 | nM |
| B426 | 98.6 | nM |
| B427 | 184 | nM |
| B428 | 61.5 | nM |
| B429 | 149 | nM |
| B430 | 93.4 | nM |
| B431 | 1.16 | μM |
| B432 | 196 | nM |
| B433 | 352 | nM |
| B434 | 578 | nM |
| B435 | 191 | nM |
| B436 | 367 | nM |
| B437 | 472 | nM |
| B438 | 20.5 | nM |
| B439 | 27.2 | nM |
| B440 | 112 | nM |
| B441 | 81.1 | nM |
| B442 | 367 | nM |
| B443 | 36.9 | nM |
| B444 | 31.8 | nM |
| B445 | 30.4 | nM |
| B446 | 11.9 | nM |
| B447 | 19.9 | nM |
| B448 | 32 | nM |
| B449 | 12.2 | nM |
| B450 | 28.2 | nM |
| B451 | 2.13 | μM |
| B452 | 25.7 | nM |
| B453 | 115 | nM |
| B454 | 453 | nM |
| B455 | 25.1 | nM |
| B456 | 16.4 | nM |
| B457 | 29.6 | nM |
| B458 | 45.7 | nM |
| B459 | 160 | nM |
| B460 | 3.02 | μM |
| B461 | 31.9 | nM |
| B462 | 107 | nM |
| B463 | 24.3 | nM |
| B464 | 13.9 | nM |
| B465 | 26.3 | nM |
| B466 | 101 | nM |
| B467 | 33.4 | nM |
| B468 | 31.1 | nM |
| B469 | 31.3 | nM |
| B470 | 35.8 | nM |
| B471 | 69 | nM |
| B472 | 35.4 | nM |
| B473 | 59.2 | nM |
| B474 | 44.2 | nM |
| B475 | 33.2 | nM |
| B476 | 38.9 | nM |
| B477 | 52.3 | nM |
| B478 | 204 | nM |
| B479 | 32.1 | nM |
| B480 | 75.3 | nM |
| B481 | 31.1 | nM |
| B482 | 1.51 | μM |
| B483 | 41.8 | nM |
| B484 | 278 | nM |
| B485 | 35.8 | nM |
| B486 | 41.5 | nM |
| B487 | 25.2 | nM |
| B488 | 38.1 | nM |
| B489 | 33.9 | nM |
| B490 | 37.3 | nM |
| B491 | 24.4 | nM |
| B492 | 33 | nM |
| B493 | 10.6 | nM |
| B494 | 62.4 | nM |
| B495 | 50.6 | nM |
| B496 | 541 | nM |
| B497 | 16.4 | nM |
| B498 | 117 | nM |
| B499 | 73 | nM |
| B500 | 28.8 | nM |
| B501 | 22.9 | nM |
| B502 | 18.9 | nM |
| B503 | 28.9 | nM |
| B504 | 217 | nM |
| B505 | 8.46 | nM |
| B506 | 11.9 | nM |
| B507 | 58.8 | nM |
| B508 | 211 | nM |
| B509 | 6.95 | nM |
| B510 | 4.89 | nM |
| B511 | 20.4 | nM |
| B512 | 6.62 | nM |
| B513 | 6.36 | nM |
| B514 | 176 | nM |
| B515 | 74.9 | nM |
| B516 | 166 | nM |
| B517 | 7.74 | nM |
| B518 | 133 | nM |
| B519 | 68.4 | nM |
| B520 | 14.6 | nM |
| B521 | 98.3 | nM |
| B522 | 22.2 | nM |
| B523 | 273 | nM |
| B524 | 87.2 | nM |
| B525 | 21.6 | nM |
| B526 | 6.97 | nM |
| B527 | 9.03 | nM |
| B528 | 10.2 | nM |
| B529 | 124 | nM |
| B530 | 214 | nM |
| B531 | 50.5 | nM |
| B532 | 12.7 | nM |
| B533 | 24.4 | nM |
| B534 | 20.8 | nM |
| B535 | 43.9 | nM |
| B536 | 166 | nM |
| B537 | 101 | nM |
| B538 | 54.2 | nM |
| B539 | 26 | nM |
| B540 | 32.7 | nM |
| B541 | 13.4 | nM |
| B542 | 15.6 | nM |
| B543 | 66.7 | nM |
| B544 | 32.7 | nM |
| B545 | 121 | nM |
| B546 | 53 | nM |
| B547 | 179 | nM |
| B548 | 14.3 | nM |
| B549 | 140 | nM |
| B550 | 40.3 | nM |
| B551 | 74.7 | nM |
| B552 | 17.8 | nM |
| B553 | 23.6 | nM |
| B554 | 34.6 | nM |
| B555 | 21.3 | nM |
| B556 | 48.5 | nM |
| B557 | 83.9 | nM |
| B558 | 13.6 | nM |
| B559 | 34.7 | nM |
| B560 | 19.2 | nM |
| B561 | 429 | nM |
| B562 | 16.3 | nM |
| B563 | 26.4 | nM |
| B564 | 27 | nM |
| B565 | 51.2 | nM |
| B566 | 78.6 | nM |
| B567 | 17.9 | nM |
| B568 | 411 | nM |
| B569 | 427 | nM |
| B570 | 143 | nM |
| B571 | 237 | nM |
| B572 | 65.6 | nM |
| B573 | 115 | nM |
| B574 | 609 | nM |
| B575 | 216 | nM |
| B576 | 528 | nM |
| B577 | 102 | nM |
| B578 | 424 | nM |
| B579 | 340 | nM |
| B580 | 262 | nM |
| B581 | 788 | nM |
| B582 | 100 | μM |
| B583 | 1.84 | μM |
| B584 | 266 | nM |
| B585 | 202 | nM |
| B586 | 762 | nM |
| B587 | 2.39 | μM |
| B588 | 1.96 | μM |
| B589 | 1.45 | μM |
| B590 | 17.4 | nM |
| B591 | 149 | nM |
| B592 | 21 | nM |

### Example 2: Ki Determination by Radioligand Binding

Radioligand binding assays are performed using the commercially available adrenergic receptor agonist [¹²⁵I]Cyanopindolol as the radioligand and non-specific binding is determined in the presence of unlabeled L-748,337 at a saturating concentration of 10 μM. For the beta-3 adrenergic receptor, the radioligand is used in the assay at a final concentration of 0.4 nM. Membrane pellets prepared from CHO-K1 cells stably expressing recombinant beta-3 adrenergic receptors are prepared using standard methods and stored at −80° C. Membranes are thawed on ice and resuspended in Assay Buffer (20 mM HEPES, pH 7.4, 10 mM MgCl₂) by dounce homogenization. Competition experiments consist of addition of 145 μL of membranes, 50 μL of radioligand stock, and 5 μL of test compound diluted in DMSO to 96-well microtiter plates. Plates are incubated for one hour at room temperature and the assay terminated by rapid filtration through Perkin Elmer GF/C filtration, plates pretreated with 0.5% PEI, under vacuum pressure using a 96-well Packard filtration apparatus. Plates are rapidly washed several times with ice-cold Assay Buffer and then dried overnight at 45° C. Finally, 25 μL of BetaScint scintillation cocktail is added to each well and plates counted in a Packard TopCount scintillation counter. In each competition study, test compounds are dosed at eight to ten concentrations with triplicate determinations at each test concentration. A reference compound, typically isoproterenol, is included in every experiment for quality control purposes.

Raw counts from scintillation counters are fit to a nonlinear least squares curve fitting program to obtain IC₅₀ values. Ki values are determined from IC₅₀ values using the Cheng-Prusoff equation and the radioligand Kd. Mean Ki values and 95% confidence intervals are calculated from the mean log(Ki) value.

### Example 3: Mouse Model of Polycystic Kidney Disease

The compounds, pharmaceutically acceptable salts, solvates, and hydrates described herein can be tested in an animal model of polycystic kidney disease. See, e.g., Tao Y, et al. Rapamycin markedly slows disease progression in a rat model of polycystic kidney disease. J Am Soc Nephrol. 2005; 16(1):46-51, which is incorporated herein by reference in its entirety. See, also, Zafar et al. Long-term rapamycin therapy in the Han:SPRD rat model of polycystic kidney disease (PKD). Nephrol Dial Transplant. 2009; 24(8):2349-2353, and Wahl et al. Inhibition of mTOR with sirolimus slows disease progression in Han:SPRD rats with autosomal dominant polycystic kidney disease (ADPKD), Nephrol Dial Transplant. 2006; 21:598-604, each which is incorporated herein by reference in its entirety.

A study will be conducted in heterozygous (Cy/+) and normal littermate control (+/+) Han:SPRD rats. All of the normal rats and Cy/+ rats studied will be male since the disease progresses faster in male compared with female rat. The Cy/+ Han:SPRD rat develops clinically detectable PKD by 8 wk of age as evidenced by a doubling of kidney size and kidney failure compared with +/+ control rats. The Han:SPRD rat is a well-known model of ADPKD.

Male Cy/+ and +/+ rats will be weaned at 3 wk of age and then treated with test compound at a dose of 0.2 mg/kg per d intraperitoneally or vehicle (ethanol) for 5 wk. See, e.g., FDA Guidance for Industry: Estimating the Maximum Safe Starting Dose in Initial Clinical Trials for Therapeutics in Adult Healthy Volunteers (2005). A 1 mg/ml stock solution of test compound in 100% ethanol will be kept at 4° C. At the end of the eighth week of age, rats will be anesthetized by intraperitoneal injection of pentobarbital sodium (50 mg/kg body wt), and kidneys will be removed and weighed. The left kidney will be fixed in 4% paraformaldehyde in PBS for 120 min and then put into 70% ethanol and embedded in paraffin for histologic examinations.

Hematoxylin-eosin-stained sections will be used to determine the cyst volume density. This will be performed by a reviewer who will be blinded to the identity of the treatment modality, using point counting stereology. Areas of the cortex at 90, 180, and 270 degrees from the hilum of each section will be selected to guard against field selection variation.

Immunohistochemical detection of proliferating cell nuclear antigen (PCNA) staining will be performed using an anti-PCNA antibody (Santa Cruz Biotechnology, Santa Cruz, CA; 1:50). The sections will be incubated with alkaline-phosphatase-labeled polymer (DAKO EnVision System, Cat #K4016; DAKO, Carpinteria, CA) and visualized with the substrate chromogen fast red.

The number of PCNA-positive cells per tubule will be counted using a Nikon Eclipse E400 microscope equipped with a digital camera connected to Spot Advanced imaging software (Version 3.5) by an observer who will be blinded to the treatment modality. Noncystic tubules will be defined as tubules that were <50 μm in diameter. At least 10 noncystic tubules or cysts in the cortex and medulla per sample will be randomly selected and counted.

To avoid confusion between noncystic tubules and small cysts as well as potential changes in tubular cells lining massive cysts, PCNA-positive tubular cells will be counted in “medium-sized cysts” of approximately 250 μm in diameter. At least 10 cysts per sample in the cortex will be randomly selected and counted.

Serum urea nitrogen levels will be measured using a Beckman autoanalyzer (Beckman Instruments, Fullerton, CA).

Nonnormally distributed data w will be ere analyzed by the nonparametric unpaired Mann-Whitney test. Multiple group comparisons were performed using a one-way ANOVA with posttest according to Newman-Keuls. P<0.05 was considered statistically significant. Values will be expressed as means±SEM.

### Example 3: Clinical Trial

A pilot open-label randomized clinical trial in ADPKD patients with estimated GFR>45 min (Stage 1-3a CKD) comparing: control versus test compound over one year. During the study period, through study visits along with serial blood draws and urinary measurements, the investigators will evaluate safety and tolerability of these treatment regimens, follow renal function and investigate the role of these treatments on acidosis, inflammatory and metabolic biomarkers in patients enrolled at an outpatient facility.

Inclusion criteria may include:
Patients has ADPKD diagnosed by unified criteria using a combination of ultrasound results, genotyping and MRI as needed. Kidney ultrasound is usually used for screening because it is safe, effective, and inexpensive. Diagnostic criteria are based upon whether the genotype is known. Disease severity varies between the different genotypes. The great majority of patients at risk for ADPKD are from families with an unknown genotype. This diagnosis will take place prior to recruitment/inclusion into the study. The following ultrasonographic criteria for the diagnosis of ADPKD are for at-risk patients from families of where the genotype is not known:
   a. If the patient is between 18 and 39 years of age, at least three unilateral or bilateral kidney cysts. The specificity and positive predictive value at this age-range is 100 percent. (sensitivity of 82 and 96 percent for individuals between 15 and 29 years and between 30 to 39 years of age, respectively).
   b. If the patient is 40 to 59 years of age, at least two cysts in each kidney (sensitivity, specificity, and positive predictive value of 90, 100, and 100 percent, respectively).
   c. Among individuals 60 years or older, at least four cysts in each kidney. (100 percent sensitivity and specificity).

Exclusion Criteria may include.
Acute coronary disease, liver disease, muscle disease, or a history of pulmonary edema
Patients with systemic disease that impacting kidney per Investigator's decision
Patients with known unstable cerebral aneurysm per Investigator's decision

Primary endpoints may include:
Changes in kidney function in patients enrolled in different arms of the study [Time Frame: 12 months]
   The effect of test compound on kidney function in patients with ADPKD compared to Standard therapy will be assessed: serum creatinine and serum blood urea nitrogen, serum electrolytes contribute to evaluate kidney function.
Changes in liver function in patients enrolled in different arms of the study [Time Frame: 12 months]
   The liver function test panels in patients with ADPKD compared to test compound alone or Standard therapy will be tested. The liver function panel should be within normal limits for enrollment and continuation in the study. Liver function tests include AST>3ULN, ALT>3ULN, Total Bilirubin>2 ULN, or increase in prothrombin time to abnormal level INR>1.5 repeated two weeks apart.
Changes in blood pressure in patients enrolled in different arms of the study [Time Frame: 12 months]
   The effect of test compound on blood pressure (systolic and diastolic) in patients with ADPKD compared to Standard therapy will be estimated.
Changes in muscle injury marker function in patients enrolled in different arms of the study [Time Frame: 12 months]
   The effect of test compound on creatine phospho kinase (CPK) in patients with ADPKD compared to Standard therapy will be estimated.
Changes in muscle tenderness in patients enrolled in the study [Time Frame: 12 months]
   The effect of test compound on muscle tenderness in patients with ADPKD compared to Standard therapy will be estimated. The physical exam will evaluate tenderness to palpation in major muscle groups such as leg, arm and back muscles. It will be graded as presence or absence. The patients will only be enrolled if there is absence of tenderness in muscles upon palpation on physical exam. If there is tenderness on exam or the patient reports tenderness that is then confirmed by exam the patient will be removed from the study.

Secondary Outcome Measure may include:
Urinary alkalinization changes [Time Frame: 12 months]
   These parameters will be ascertained by measurements of urinary pH and serum electrolytes.
Inflammatory markers in blood and urine [Time Frame: 12 months]
   Inflammatory markers (interleukins, prostaglandins and other cytokines) in leukocytes, plasma, and urine in the different study groups will be assessed. The biomarkers that will be measured are HETE/HODE species phospho-AMPK Inflammatory and metabolic biomarkers: NGAL, KIM1 in blood and urine.
AMPK pathway activation [Time Frame: 12 months]
   The investigators will determine whether and to what extent the AMPK pathway is activated in leukocytes and urine derived from patients in the study groups at the different study visits, and correlate AMPK activation with biological effects.

Those skilled in the art will recognize that various modifications, additions, substitutions, and variations to the illustrative examples set forth herein can be made without departing from the spirit of the invention and are, therefore, considered within the scope of the invention.

1. A method of treating renal cystic disease and/or cardiorenal syndrome in an individual, comprising administering to the individual in need thereof, a therapeutically effective amount of a compound selected from compounds of Formula (Ia) and pharmaceutically acceptable salts, solvates, and hydrates thereof: 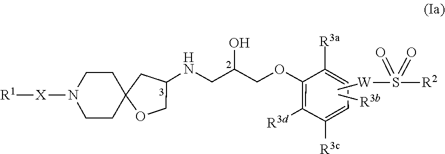
wherein:
X is —SO₂—, —C(═O)—, or —CH₂C(═O)—;
W is absent or C₁-C₃ alkylene;
R¹ is aryl or heteroaryl, wherein each is optionally substituted with one or more substituents selected from: C₁-C₆ alkoxy, C₁-C₆ alkyl, amino, cyano, C₃-C₇ cycloalkyl, C₁-C₆ haloalkyl, halogen, hydroxyl, oxo, and sulfamoyl; and wherein said C₁-C₆ alkyl and C₃-C₇ cycloalkyl are each optionally substituted with one or more substituents selected from: amino, C₁-C₆ alkoxy, C₁-C₆ alkylcarboxamide, —Y—C₃-C₇-cycloalkyl, —Y—C₁-C₆-alkylene-Z,
C₁-C₆ alkylamino, C₁-C₆ haloalkylamino, and heterocyclyl;
Y is independently selected from: —O—, —NH—, and —N—(C₁-C₄ alkyl)-;
Z is independently selected from: hydroxyl, C₁-C₆ alkoxy, amino, C₁-C₆ alkylamino, and C₂-C₆ dialkylamino;
R² is selected from: C₂-C₆ alkenyl, C₁-C₆ alkyl, C₃-C₇ cycloalkyl, heterocyclyl, and C₁-C₆ haloalkyl; each optionally substituted with one or more substituents selected from: C₁-C₆ alkoxy, C₁-C₆ alkylenehydroxyl, amino, aryl, C₃-C₇ cycloalkyl, cyano, C₃-C₇ halocycloalkyl, hydroxyl, and oxo; and
R^{3a}, R^{3b}, R^{3c}, and R^{3d} are each independently H or halogen.

2. The method according to claim 1, wherein X is —SO₂—.

3. The method according to claim 1, wherein W is absent.

4. The method according to claim 1, wherein R¹ is selected from: (R)-1,3-dimethyl-2,3-dihydro-1H-pyrido[2,3-b][1,4]oxazin-7-yl, (S)-1,3-dimethyl-2,3-dihydro-1H-pyrido[2,3-b][1,4]oxazin-7-yl, 1-(2-methoxyethyl)-2,3-dihydro-1H-pyrido[2,3-b][1,4]oxazin-7-yl, 1,3,3-trimethyl-2,3-dihydro-1H-pyrido[2,3-b][1,4]oxazin-6-yl, 1,4-dimethyl-1,2,3,4-tetrahydropyrido[3,2-b]pyrazin-7-yl, 1,6-dimethyl-2,3-dihydro-1H-pyrido[2,3-b][1,4]oxazin-7-yl, 1,8-dimethyl-2,3-dihydro-1H-pyrido[2,3-b][1,4]oxazin-7-yl, 1-ethyl-2,3-dihydro-1H-pyrido[2,3-b][1,4]oxazin-7-yl, 1-ethyl-4-oxo-1,4-dihydroquinolin-3-yl, 1-ethyl-5-methyl-1H-pyrazol-4-yl, 1-ethyl-6-fluoro-4-oxo-1,4-dihydroquinolin-3-yl, 1-ethyl-6-methyl-4-oxo-1,4-dihydroquinolin-3-yl, 1-ethyl-7-fluoro-4-oxo-1,4-dihydroquinolin-3-yl, 1-ethyl-7-methyl-4-oxo-1,4-dihydroquinolin-3-yl, 1-ethyl-8-fluoro-4-oxo-1,4-dihydroquinolin-3-yl, 1-ethyl-8-methyl-4-oxo-1,4-dihydroquinolin-3-yl, 1H-benzo[d]imidazol-5-yl, 1H-indazol-5-yl, 1H-indol-2-yl, 1H-indol-3-yl, 1H-indol-5-yl, 1H-indol-6-yl, 1H-pyrazolo[4,3-b]pyridin-6-yl, 1H-pyrrolo[2,3-b]pyridin-3-yl, 1H-pyrrolo[3,2-b]pyridin-6-yl, 1-methyl-2,3-dihydro-1H-pyrido[2,3-b][1,4]oxazin-7-yl, 1-methyl-4-oxo-1,4-dihydroquinolin-3-yl, 2,3-dihydro-[1,4]dioxino[2,3-b]pyridin-7-yl, 2,3-dihydro-1H-pyrido[2,3-b][1,4]oxazin-7-yl, 2,3-dihydrobenzofuran-5-yl, 2-aminothiazol-4-yl, 2-oxo-2,3-dihydro-1H-imidazo[4,5-b]pyridin-6-yl, 2-oxo-2,3-dihydro-1H-pyrrolo[2,3-b]pyridin-5-yl, 3-(1-cyclopropyl-1H-pyrazol-4-yl)phenyl, 3-(1-ethyl-1H-pyrazol-4-yl)phenyl, 3-(1H-pyrazol-4-yl)phenyl, 3-(1-methyl-1H-pyrazol-4-yl)phenyl, 3-(1-propyl-1H-pyrazol-4-yl)phenyl, 3-(2-methylpyridin-4-yl)phenyl, 3-(3-fluoropyridin-2-yl)phenyl, 3-(4-methylpyridin-2-yl)phenyl, 3-(5-methylpyridin-2-yl)phenyl, 3-(6-(trifluoromethyl)pyridin-2-yl)phenyl, 3-(6-aminopyridin-3-yl)phenyl, 3-(6-fluoropyridin-2-yl)phenyl, 3-(6-methylpyridin-2-yl)phenyl, 3-(pyridin-2-yl)phenyl, 3-(pyridin-3-yl)phenyl, 3-(pyridin-4-yl)phenyl, 3-(pyrimidin-5-yl)phenyl, 3,4-dihydro-2H-pyrano[2,3-b]pyridin-6-yl, 3,5-dimethylisoxazol-4-yl, 3-methyl-3H-imidazo[4,5-b]pyridin-5-yl, 3-methyl-3H-imidazo[4,5-b]pyridin-6-yl, 3-oxo-3,4-dihydro-2H-benzo[b][1,4]oxazin-6-yl, 4-(pyridin-2-yl)phenyl, 4-(pyridin-3-yl)phenyl, 4-(pyridin-4-yl)phenyl, 4-hydroxy-6-methylquinolin-3-yl, 4-hydroxy-7-methylquinolin-3-yl, 4-hydroxy-8-methylquinolin-3-yl, 4-hydroxyquinolin-3-yl, 4-methoxyquinolin-3-yl, 4-methyl-2-oxo-1,2-dihydroquinolin-6-yl, 4-methyl-3,4-dihydro-2H-benzo[b][1,4]oxazin-6-yl, 4-methyl-3,4-dihydro-2H-benzo[b][1,4]oxazin-7-yl, 4-methyl-3,4-dihydro-2H-pyrido[3,2-b][1,4]oxazin-7-yl, 4-oxo-1,4-dihydroquinolin-3-yl, 5-(1-methyl-1H-pyrazol-4-yl)pyridin-3-yl, 5-(4-(aminomethyl)phenyl)pyridin-3-yl, 5,6,7,8-tetrahydroquinolin-3-yl, 5-bromo-6-chloropyridin-3-yl, 5-bromopyridin-3-yl, 5-oxo-6,7-dihydro-5H-pyrrolo[3,4-b]pyridin-3-yl, 5-phenylthiophen-2-yl, 6-fluoro-4-hydroxyquinolin-3-yl, 7-chlorobenzo[c][1,2,5]oxadiazol-4-yl, 7-fluoro-4-hydroxyquinolin-3-yl, 8-fluoro-4-hydroxyquinolin-3-yl, benzofuran-2-yl, benzofuran-5-yl, chroman-6-yl, chroman-7-yl, isoquinolin-5-yl, pyridin-2-yl, pyridin-3-yl, pyrrolo[1,2-a]pyrimidin-3-yl, quinolin-3-yl, quinolin-6-yl, and quinolin-7-yl.

5. The method according to claim 1, R² is selected from: 1,1-difluoroethyl, 1-fluoroethyl, 2-methylpropan-2-yl, 3,3,3-trifluoropropyl, 4,4,4-trifluorobutyl, azetidin-3-yl, cyclobutyl, cyclopentyl, cyclopropyl, ethyl, fluoromethyl, isobutyl, isopentyl, isopropyl, methyl, oxetan-3-yl, propan-1-yl, sec-butyl, and vinyl; each optionally substituted with one or more substituents selected from: 2,2-difluorocyclopropyl, amino, cyano, cyclobutyl, cyclohexyl, cyclopropyl, ethoxy, hydroxy, hydroxymethyl, methoxy, oxo, and phenyl.

6. The method according to claim 1, wherein R^{3a}, R^{3b}, R^{3c}, and R^{3d} are each H.

7. The method according to claim 1, selected from compounds of Formula (Ig) and pharmaceutically acceptable salts, solvates, and hydrates thereof: 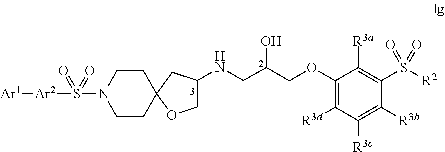
wherein:
Ar¹ and Ar² are independently 1H-pyrazolyl, phenyl, pyridinyl, pyrimidinyl, and thiophenyl, wherein each is optionally substituted with one or more substituents selected from: C₁-C₆ alkoxy, C₁-C₆ alkyl, amino, C₃-C₇ cycloalkyl, C₁-C₆ haloalkyl, halogen, and sulfamoyl; and wherein said C₁-C₆ alkyl and C₃-C₇ cycloalkyl are each optionally substituted with one or more substituents selected from: amino, C₁-C₆ alkylcarboxamide, —NH—C₃-C₇-cycloalkyl, —NH—C₁-C₆-alkylene-NH₂, —NH—C₁-C₆-alkylene-O—C₁-C₆-alkyl, C₁-C₆ alkylamino, C₁-C₆ haloalkylamino, and heterocyclyl;
R² is selected from: C₁-C₆ alkyl, C₃-C₇ cycloalkyl, and C₁-C₆ haloalkyl; each optionally substituted with one or more substituents selected from: C₁-C₆ alkoxy, C₁-C₆ alkylenehydroxyl, amino, hydroxyl, and oxo; and
R^{3a}, R^{3b}, R^{3c}, and R^{3d} are each independently H or halogen.

8. The method according to claim 1, selected from compounds of Formula (Ii) and pharmaceutically acceptable salts, solvates, and hydrates thereof: 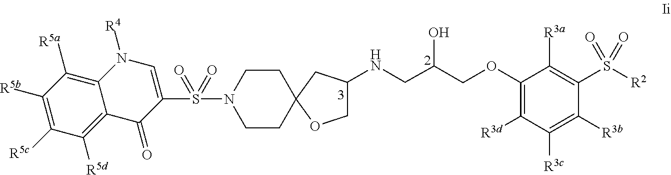
wherein:
R² is selected from: C₁-C₆ alkyl, C₃-C₇ cycloalkyl, and C₁-C₆ haloalkyl; each optionally substituted with one or more substituents selected from: C₁-C₆ alkoxy, C₁-C₆ alkylenehydroxyl, and hydroxyl;
R^{3a}, R^{3b}, R^{3c}, and R^{3d} are each independently H or halogen;
R⁴ is H or C₁-C₆ alkyl; and
R^{5a}, R^{5b}, R^{5c}, and R^{5d} are independently H, C₁-C₆ alkyl, and halogen.

9. The method according to claim 1, selected from compounds of Formula (Ii) and pharmaceutically acceptable salts, solvates, and hydrates thereof: 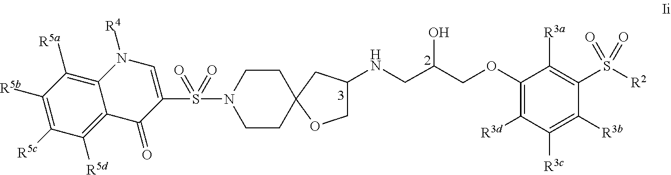
wherein:
R² is selected from: 1,1-difluoroethyl, 2-methylpropan-2-yl, cyclopropyl, ethyl, 1-fluoroethyl, isopropyl, and methyl; each optionally substituted with one or more substituents selected from: hydroxy, hydroxymethyl, and methoxy;
R^{3a}, R^{3b}, R^{3c}, and R^{3d} are each H;
R⁴ is selected from: H, methyl, and ethyl; and
R^{5a}, R^{5b}, R^{5c}, and R^{5d} are independently H, methyl, and fluoro.

10. The method according to claim 1, selected from compounds of Formula (Ii) and pharmaceutically acceptable salts, solvates, and hydrates thereof: 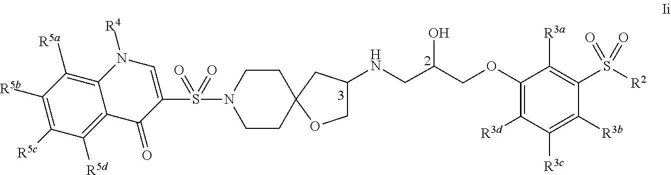
wherein:
R² is selected from: 1-(hydroxymethyl)cyclopropyl, 1,1-difluoro-2-hydroxyethyl, 1-fluoroethyl, 1-hydroxy-2-methylpropan-2-yl, cyclopropyl, isopropyl, methoxymethyl, and methyl;
R^{3a}, R^{3b}, R^{3c}, and R^{3d} are each H;
R⁴ is selected from: H, methyl, and ethyl;
R^{5a}, R^{5b}, and R^{5c} are independently H, methyl, and fluoro; and
R^{5d} is H.

11. The method according to claim 1, wherein the stereochemistry for the C(3) carbon of the oxa-azaspiro[4.5]decanyl group bonded to the nitrogen is (R) and the stereochemistry for the C(2) carbon of the propyl group bonded to the hydroxyl group is (S).

12. The method according to claim 1, selected from the following compounds and pharmaceutically acceptable salts, solvates, and hydrates thereof:
(2S)-1-(3-(2-hydroxyethylsulfonyl)phenoxy)-3-(8-(naphthalen-2-ylsulfonyl)-1-oxa-8-azaspiro[4.5]decan-3-ylamino)propan-2-ol (Compound A5);
(S)-1-(3-(2-hydroxyethylsulfonyl)phenoxy)-3-((R)-8-(quinolin-3-ylsulfonyl)-1-oxa-8-azaspiro[4.5]decan-3-ylamino)propan-2-ol (Compound A88);
2-(3-((S)-2-hydroxy-3-((R)-8-(quinolin-3-ylsulfonyl)-1-oxa-8-azaspiro[4.5]decan-3-ylamino)propoxy)phenylsulfonyl)acetamide (Compound A123);
(S)-1-(3-(1-(hydroxymethyl)cyclopropylsulfonyl)phenoxy)-3-((R)-8-(quinolin-3-ylsulfonyl)-1-oxa-8-azaspiro[4.5]decan-3-ylamino)propan-2-ol (Compound A136);
(S)-1-(3-(cyclopropylsulfonyl)phenoxy)-3-((R)-8-(1-methyl-2,3-dihydro-1H-pyrido[2,3-b][1,4]oxazin-7-ylsulfonyl)-1-oxa-8-azaspiro[4.5]decan-3-ylamino)propan-2-ol (Compound A154);
(S)-1-((R)-8-(4′-(aminomethyl)-4-ethoxybiphenyl-3-ylsulfonyl)-1-oxa-8-azaspiro[4.5]decan-3-ylamino)-3-(3-(1,1-difluoro-2-hydroxyethylsulfonyl)phenoxy)propan-2-ol (Compound A161);
(S)-1-((S)-8-(4′-(aminomethyl)-4-ethoxybiphenyl-3-ylsulfonyl)-1-oxa-8-azaspiro[4.5]decan-3-ylamino)-3-(3-(1-(hydroxymethyl)cyclopropylsulfonyl)phenoxy)propan-2-ol (Compound A163);
(S)-1-((R)-8-(4′-(aminomethyl)-4-fluorobiphenyl-3-ylsulfonyl)-1-oxa-8-azaspiro[4.5]decan-3-ylamino)-3-(3-(1-(hydroxymethyl)cyclopropylsulfonyl)phenoxy)propan-2-ol (Compound A169);
(S)-1-((R)-8-(4′-(1-aminocyclopropyl)-6-methoxybiphenyl-3-ylsulfonyl)-1-oxa-8-azaspiro[4.5]decan-3-ylamino)-3-(3-(1-(hydroxymethyl)cyclopropylsulfonyl)phenoxy)propan-2-ol (Compound A199);
(S)-1-((S)-8-(4′-(2-aminoethyl)biphenyl-3-ylsulfonyl)-1-oxa-8-azaspiro[4.5]decan-3-ylamino)-3-(3-(1,1-difluoro-2-hydroxyethylsulfonyl)phenoxy)propan-2-ol (Compound A210);
(S)-1-((S)-8-(4′-(2-aminoethyl)biphenyl-3-ylsulfonyl)-1-oxa-8-azaspiro[4.5]decan-3-ylamino)-3-(3-(1-(hydroxymethyl)cyclopropylsulfonyl)phenoxy)propan-2-ol (Compound A211);
(S)-1-((R)-8-(4′-(1-aminocyclopropyl)-6-fluorobiphenyl-3-ylsulfonyl)-1-oxa-8-azaspiro[4.5]decan-3-ylamino)-3-(3-(1-(hydroxymethyl)cyclopropylsulfonyl)phenoxy)propan-2-ol (Compound A217);
(S)-1-((S)-8-(4′-(aminomethyl)-4-ethoxy-3′-fluorobiphenyl-3-ylsulfonyl)-1-oxa-8-azaspiro[4.5]decan-3-ylamino)-3-(3-(1-(hydroxymethyl)cyclopropylsulfonyl)phenoxy)propan-2-ol (Compound A220);
(S)-1-((S)-8-(4′-(1-aminocyclopropyl)-6-methoxybiphenyl-3-ylsulfonyl)-1-oxa-8-azaspiro[4.5]decan-3-ylamino)-3-(3-(1-(hydroxymethyl)cyclopropylsulfonyl)phenoxy)propan-2-ol (Compound A225);
(S)-1-((S)-8-(4′-(aminomethyl)-6-methoxybiphenyl-3-ylsulfonyl)-1-oxa-8-azaspiro[4.5]decan-3-ylamino)-3-(3-(cyclopropylsulfonyl)phenoxy)propan-2-ol (Compound A227);
(S)-1-((S)-8-(4′-(aminomethyl)-5-methoxybiphenyl-3-ylsulfonyl)-1-oxa-8-azaspiro[4.5]decan-3-ylamino)-3-(3-(cyclopropylsulfonyl)phenoxy)propan-2-ol (Compound A229);
(S)-1-((S)-8-(4′-(aminomethyl)-4-ethoxybiphenyl-3-ylsulfonyl)-1-oxa-8-azaspiro[4.5]decan-3-ylamino)-3-(3-(methoxymethylsulfonyl)phenoxy)propan-2-ol (Compound A230);
(S)-1-((S)-8-(4′-(aminomethyl)-4-ethoxybiphenyl-3-ylsulfonyl)-1-oxa-8-azaspiro[4.5]decan-3-ylamino)-3-(3-(isopropylsulfonyl)phenoxy)propan-2-ol (Compound A232);
(2S)-1-(3-(1-fluoroethylsulfonyl)phenoxy)-3-((R)-8-(quinolin-6-ylsulfonyl)-1-oxa-8-azaspiro[4.5]decan-3-ylamino)propan-2-ol (Compound A234);
(S)-1-((S)-8-(4′-((tert-butylamino)methyl)biphenyl-3-ylsulfonyl)-1-oxa-8-azaspiro[4.5]decan-3-ylamino)-3-(3-(1-(hydroxymethyl)cyclopropylsulfonyl)phenoxy)propan-2-ol (Compound A240);
(S)-1-(3-(1-(hydroxymethyl)cyclopropylsulfonyl)phenoxy)-3-((S)-8-(4′-((tert-pentylamino)methyl)biphenyl-3-ylsulfonyl)-1-oxa-8-azaspiro[4.5]decan-3-ylamino)propan-2-ol (Compound A241);
(S)-1-((S)-8-(4′-(azetidin-1-ylmethyl)biphenyl-3-ylsulfonyl)-1-oxa-8-azaspiro[4.5]decan-3-ylamino)-3-(3-(1-(hydroxymethyl)cyclopropylsulfonyl)phenoxy)propan-2-ol (Compound A243);
(S)-1-(3-(1-(hydroxymethyl)cyclopropylsulfonyl)phenoxy)-3-((S)-8-(4′-((propylamino)methyl)biphenyl-3-ylsulfonyl)-1-oxa-8-azaspiro[4.5]decan-3-ylamino)propan-2-ol (Compound A244);
(S)-1-((S)-8-(4′-((butylamino)methyl)biphenyl-3-ylsulfonyl)-1-oxa-8-azaspiro[4.5]decan-3-ylamino)-3-(3-(1-(hydroxymethyl)cyclopropylsulfonyl)phenoxy)propan-2-ol (Compound A245);
(S)-1-(3-(1-(hydroxymethyl)cyclopropylsulfonyl)phenoxy)-3-((S)-8-(4′-((2-methoxyethylamino)methyl)biphenyl-3-ylsulfonyl)-1-oxa-8-azaspiro[4.5]decan-3-ylamino)propan-2-ol (Compound A247);
3-((R)-3-((S)-3-(3-(cyclopropylsulfonyl)phenoxy)-2-hydroxypropylamino)-1-oxa-8-azaspiro[4.5]decan-8-ylsulfonyl)-1-ethylquinolin-4(1H)-one (Compound A297);
(S)-1-(3-(1-(hydroxymethyl)cyclopropylsulfonyl)phenoxy)-3-((R)-8-(naphthalen-2-ylsulfonyl)-1-oxa-8-azaspiro[4.5]decan-3-ylamino)propan-2-ol (Compound A300);
(S)-1-((R)-8-(1H-pyrrolo[3,2-b]pyridin-6-ylsulfonyl)-1-oxa-8-azaspiro[4.5]decan-3-ylamino)-3-(3-(cyclopropylsulfonyl)phenoxy)propan-2-ol (Compound A309);
1-ethyl-3-((R)-3-((S)-2-hydroxy-3-(3-(methylsulfonyl)phenoxy)propylamino)-1-oxa-8-azaspiro[4.5]decan-8-ylsulfonyl)quinolin-4(1H)-one (Compound A310);
(S)-1-((R)-8-(1H-pyrrolo[3,2-b]pyridin-6-ylsulfonyl)-1-oxa-8-azaspiro[4.5]decan-3-ylamino)-3-(3-(methylsulfonyl)phenoxy)propan-2-ol (Compound A320);
(S)-1-((R)-8-(1H-pyrrolo[3,2-b]pyridin-6-ylsulfonyl)-1-oxa-8-azaspiro[4.5]decan-3-ylamino)-3-(3-(isopropylsulfonyl)phenoxy)propan-2-ol (Compound A321);
1-ethyl-8-fluoro-3-((R)-3-((S)-2-hydroxy-3-(3-(methylsulfonyl)phenoxy)propylamino)-1-oxa-8-azaspiro[4.5]decan-8-ylsulfonyl)quinolin-4(1H)-one (Compound A322);
3-((R)-3-((S)-3-(3-(cyclopropylsulfonyl)phenoxy)-2-hydroxypropylamino)-1-oxa-8-azaspiro[4.5]decan-8-ylsulfonyl)quinolin-4(1H)-one (Compound A326);
3-((R)-3-((S)-3-(3-(cyclopropylsulfonyl)phenoxy)-2-hydroxypropylamino)-1-oxa-8-azaspiro[4.5]decan-8-ylsulfonyl)-8-methylquinolin-4-ol (Compound A327);
3-((R)-3-((S)-3-(3-(cyclopropylsulfonyl)phenoxy)-2-hydroxypropylamino)-1-oxa-8-azaspiro[4.5]decan-8-ylsulfonyl)-7-fluoroquinolin-4-ol (Compound A329); and
1-ethyl-8-fluoro-3-((R)-3-((S)-2-hydroxy-3-(3-(isopropylsulfonyl)phenoxy)propylamino)-1-oxa-8-azaspiro[4.5]decan-8-ylsulfonyl)quinolin-4(1H)-one (Compound A331).

13. The method according to claim 1, wherein the compound is selected from 1-ethyl-3-((R)-3-((S)-2-hydroxy-3-(3-(methylsulfonyl)phenoxy)propylamino)-1-oxa-8-azaspiro[4.5]decan-8-ylsulfonyl)quinolin-4(1H)-one (Compound A310) and pharmaceutically acceptable salts, solvates, and hydrates thereof.

14. A method of treating renal cystic disease and/or cardiorenal syndrome in an individual, comprising administering to the individual in need thereof, a therapeutically effective amount of a compound selected from compounds of Formula (IIa) and pharmaceutically acceptable salts, solvates, hydrates, and N-oxides thereof: 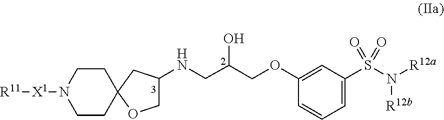
wherein:
X¹ is —SO₂— or absent;
R¹¹ is selected from: aryl, C₁-C₆-alkylene-aryl, C₁-C₆-alkylene-heteroaryl, C₃-C₇ cycloalkyl, heteroaryl, and heterocyclyl; each optionally substituted with one or more substituents selected from: C₁-C₆ alkoxy, C₁-C₆ alkoxycarbonyl, C₁-C₇ alkyl, C₁-C₆ alkylamino, C₁-C₆ alkylcarboxamide, C₁-C₆ alkylsulfonamido, C₁-C₆ alkylsulfonyl, amino, aryloxy, arylsulfonyl, carboxamide, carbamimidoyl, carboxy, cyano, C₃-C₇ cycloalkyl, C₂—C dialkylamino, C₂—C dialkylsulfamoyl, C₁-C₆ haloalkoxy, C₁-C₆ haloalkyl, halogen, heterocyclyl, hydroxycarbamimidoyl, hydroxyl, oxo, and sulfamoyl; and wherein said C₁-C₆ alkoxy, C₁-C₇ alkyl, C₁-C₆ alkylamino, aryloxy, C₃-C₇ cycloalkyl, and C₂—C dialkylamino are each optionally substituted with one or more substituents selected from: amino, C₁-C₆ alkoxy, C₁-C₆ alkylamino, C₁-C₆ alkylcarboxamide, carboxy, —Y¹—C₁-C₆-alkylene-Z¹ optionally substituted with oxo, C₃-C₇ cycloalkyl, cyano, C₂-C₆ dialkylamino, C₁-C₆ haloalkyl, C₁-C₆ haloalkylamino, heterocyclyl, hydroxyl, oxo, and phenyl;
Y¹ is selected from: —O— and —NH—;
Z¹ is selected from: C₁-C₆ alkoxy, amino, C₁-C₆ alkylamino, cyano, C₂-C₆ dialkylamino, hydroxyl, and phenyl;
R^{12a} is H or selected from: C₁-C₆ alkoxy, C₁-C₆ alkyl, C₃-C₇ cycloalkyl, and heterocyclyl; each optionally substituted with one or more substituents selected from: C₁-C₆ alkoxy, C₁-C₆ alkylamino, C₁-C₆ alkyl, C₁-C₆ alkylenehydroxyl, amino, C₃-C₇ cycloalkyl, cyano, C₂-C₈ dialkylamino, heterocyclyl optionally substituted with one oxo group, halogen, hydroxyl, and oxo; and
R^{12b} is H or C₁-C₆ alkyl.

15. The method according to claim 14 selected from compounds of Formula (IIc) and pharmaceutically acceptable salts, solvates, and hydrates thereof: 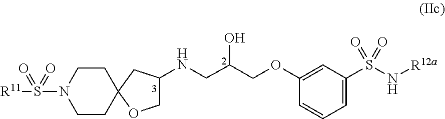
wherein:
R¹¹ is selected from: aryl, heteroaryl, and heterocyclyl; each optionally substituted with one or more substituents selected from: (2-ethyl)(methyl)amino, acetamido, amino, bromo, carbamimidoyl, carboxamide, carboxy, chloro, cyano, cyclopropyl, dimethylamino, dimethylcarbamoyl, ethoxy, ethyl, ethylamino, fluoro, hydroxycarbamimidoyl, hydroxyl, isobutyl, isopropoxy, isopropyl, isopropyl(methyl)amino, methoxy, methyl, methyl(propyl)amino, methylamino, methylsulfonamido, methylsulfonyl, morpholino, N,N-dimethylsulfamoyl, oxo, phenoxy, phenylsulfonyl, piperazin-1-yl, piperidin-1-yl, propoxy, propyl, sec-butyl, sulfamoyl, tert-butyl, tert-pentyl, trifluoromethoxy, and trifluoromethyl; and wherein (2-ethyl)(methyl)amino, cyclopropyl, ethoxy, ethyl, ethylamino, isopropyl(methyl)amino, methoxy, methyl, methyl(propyl)amino, phenoxy, and propoxy are each optionally substituted with one or more substituents selected from: 2-(dimethylamino)ethylamino, 2,2,2-trifluoroethylamino, 2,2-difluoroethylamino, 2-amino-2-oxoacetamido, 2-aminoacetamido, 2-fluoroethylamino, 2-hydroxyethylamino, 2-methoxyethylamino, acetamido, amino, benzyloxy, carboxy, cyano, cyanomethylamino, cyclopropyl, dimethylamino, ethylamino, hydroxyl, isobutylamino, isopentylamino, isopropylamino, methoxy, methylamino, morpholino, oxo, phenyl, pyrrolidin-1-yl, thiazolidin-3-yl, and trifluoromethyl; and
R^{12a} is H, ethyl, or methyl.

16. The method according to claim 15, wherein the compound is selected from the following compounds and pharmaceutically acceptable salts, solvates, and hydrates thereof:
3-((2S)-2-hydroxy-3-(8-(3-(1-methyl-1H-pyrazol-4-yl)phenylsulfonyl)-1-oxa-8-azaspiro[4.5]decan-3-ylamino)propoxy)-N-methylbenzenesulfonamide, (Compound B61);
3-((2S)-3-(8-(benzylsulfonyl)-1-oxa-8-azaspiro[4.5]decan-3-ylamino)-2-hydroxypropoxy)-N-methylbenzenesulfonamide, (Compound B172);
3-((S)-2-hydroxy-3-((R)-8-(4-methyl-3,4-dihydro-2H-benzo[b][1,4]oxazin-6-ylsulfonyl)-1-oxa-8-azaspiro[4.5]decan-3-ylamino)propoxy)-N-methylbenzenesulfonamide, (Compound B300);
3-((2S)-2-hydroxy-3-(8-(4′-((isopropylamino)methyl)biphenyl-3-ylsulfonyl)-1-oxa-8-azaspiro[4.5]decan-3-ylamino)propoxy)-N-methylbenzenesulfonamide, (Compound B303);
3-((2S)-2-hydroxy-3-(8-(4′-((isopentylamino)methyl)biphenyl-3-ylsulfonyl)-1-oxa-8-azaspiro[4.5]decan-3-ylamino)propoxy)-N-methylbenzenesulfonamide, (Compound B320);
3-((S)-2-hydroxy-3-((R)-8-(quinolin-3-ylsulfonyl)-1-oxa-8-azaspiro[4.5]decan-3-ylamino)propoxy)-N-methylbenzenesulfonamide, (Compound B336);
3-((2S)-3-(8-(4′-((2,2-difluoroethylamino)methyl)biphenyl-3-ylsulfonyl)-1-oxa-8-azaspiro[4.5]decan-3-ylamino)-2-hydroxypropoxy)-N-methylbenzenesulfonamide, (Compound B339);
3-((S)-2-hydroxy-3-((R)-8-(quinolin-6-ylsulfonyl)-1-oxa-8-azaspiro[4.5]decan-3-ylamino)propoxy)-N-methylbenzenesulfonamide, (Compound B344);
N¹-((3′-(3-((S)-2-hydroxy-3-(3-(N-methylsulfamoyl)phenoxy)propylamino)-1-oxa-8-azaspiro[4.5]decan-8-ylsulfonyl)biphenyl-4-yl)methyl)oxalamide, (Compound B354);
3-((S)-3-((S)-8-(4′-(aminomethyl)-4-ethoxybiphenyl-3-ylsulfonyl)-1-oxa-8-azaspiro[4.5]decan-3-ylamino)-2-hydroxypropoxy)-N-methylbenzenesulfonamide, (Compound B419);
3-((S)-2-hydroxy-3-((R)-8-(1-methyl-2,3-dihydro-1H-pyrido[2,3-b][1,4]oxazin-7-ylsulfonyl)-1-oxa-8-azaspiro[4.5]decan-3-ylamino)propoxy)-N-((1R,2S)-2-hydroxycyclopentyl)benzenesulfonamide, (Compound B437);
3-((S)-3-((R)-8-((R)-1,3-dimethyl-2,3-dihydro-1H-pyrido[2,3-b][1,4]oxazin-7-ylsulfonyl)-1-oxa-8-azaspiro[4.5]decan-3-ylamino)-2-hydroxypropoxy)-N-methylbenzenesulfonamide, (Compound B468);
3-((S)-3-((R)-8-(3-cyano-1H-pyrrolo[2,3-b]pyridin-5-ylsulfonyl)-1-oxa-8-azaspiro[4.5]decan-3-ylamino)-2-hydroxypropoxy)-N-methylbenzenesulfonamide, (Compound B493);
3-((R)-3-((S)-2-hydroxy-3-(3-(N-methylsulfamoyl)phenoxy)propylamino)-1-oxa-8-azaspiro[4.5]decan-8-ylsulfonyl)pyridine 1-oxide, (Compound B496);
3-((S)-3-((R)-8-(1H-pyrrolo[3,2-b]pyridin-6-ylsulfonyl)-1-oxa-8-azaspiro[4.5]decan-3-ylamino)-2-hydroxypropoxy)-N-methylbenzenesulfonamide, (Compound B505);
3-((S)-2-hydroxy-3-((R)-8-(3-(trifluoromethyl)-1H-pyrrolo[2,3-b]pyridin-5-ylsulfonyl)-1-oxa-8-azaspiro[4.5]decan-3-ylamino)propoxy)-N-methylbenzenesulfonamide, (Compound B509);
3-((S)-3-((R)-8-(1H-pyrrolo[3,2-b]pyridin-6-ylsulfonyl)-1-oxa-8-azaspiro[4.5]decan-3-ylamino)-2-hydroxypropoxy)benzenesulfonamide, (Compound B532);
3-((S)-2-hydroxy-3-((R)-8-(4-hydroxyquinolin-3-ylsulfonyl)-1-oxa-8-azaspiro[4.5]decan-3-ylamino)propoxy)-N-methylbenzenesulfonamide, (Compound B541);
3-((S)-2-hydroxy-3-((R)-8-(4-hydroxyquinolin-3-ylsulfonyl)-1-oxa-8-azaspiro[4.5]decan-3-ylamino)propoxy)benzenesulfonamide, (Compound B548);
3-((S)-3-((R)-8-(1-ethyl-4-oxo-1,4-dihydroquinolin-3-ylsulfonyl)-1-oxa-8-azaspiro[4.5]decan-3-ylamino)-2-hydroxypropoxy)benzenesulfonamide, (Compound B550);
3-((S)-2-hydroxy-3-((R)-8-(4-methoxypyrimidin-2-yl)-1-oxa-8-azaspiro[4.5]decan-3-ylamino)propoxy)-N-methylbenzenesulfonamide, (Compound B568); and
3-((S)-3-((R)-8-(4-benzylpyrimidin-2-yl)-1-oxa-8-azaspiro[4.5]decan-3-ylamino)-2-hydroxypropoxy)-N-methylbenzenesulfonamide, (Compound B573).

17. The method of claim 1, wherein the renal cystic disease is chosen from polycystic kidney disease, unilateral renal cystic disease (localized cystic disease), renal simple cysts, multicystic dysplastic kidney, pluricystic kidney of the multiple malformation syndromes, juvenile nephronophthisis and medullary cystic disease, medullary sponge kidney, primary glomerulocystic kidney disease, glomerulocystic kidney associated with several systemic disorders mainly of genetic or chromosomal etiology, cystic kidney in tuberous sclerosis, and in von Hippel-Lindau syndrome, cystic nephroma, cystic variant of congenital mesoblastic nephroma, mixed epithelial stromal tumor of the kidney, renal lymphangioma, pyelocalyceal cyst, peripylic cyst and perinephric pseudocyst, acquired renal cystic disease of long-term dialysis, and cystic renal cell carcinoma and sarcoma.

18. The method of claim 17, wherein the renal cystic disease is polycystic kidney disease.

19. The method of claim 18, wherein the polycystic kidney disease is autosomal-dominant polycystic kidney disease.

20. The method of claim 1, wherein the cardiorenal syndrome is cardiorenal syndrome type 1, cardiorenal syndrome type 2, cardiorenal syndrome type 3, or cardiorenal syndrome type 4.

21-23. (canceled)
